SEQUENCE LISTING (2 sequences)

SEQ ID NO: 1 - protein, 182 aa, synthetic construct
  REGION 1..182: note = Synthetic polypeptide
MATGSRTSLLLAFGLLCLPWLQEGSAGSDYKDDDDKGSGSGSLEVLFQGPGITIPRNPGCPNSEDKNFPRTVMVNLNIHNRNTNTNPKRSSDYYNRSTSPWNLHRNEDPERYPSVIWEAQCRHLGCINADGNVDYHMNSVPIQQEILVLRREPPHCPNSFRLEKILVSVGCTCVTPIVHHVQ

SEQ ID NO: 2 - protein, 323 aa, synthetic construct
  REGION 1..323: note = Synthetic polypeptide
MKFLVNVALVFMVVYISYIYALRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGSGGHHHHHHHHHH

(19) United States
(12) Patent Application Publication
(10) Pub. No.: US 2025/0122181 A1
(43) Pub. Date: Apr. 17, 2025

(54) BENZIMIDAZOLES AS MODULATORS OF IL-17

(71) Applicant: Janssen Pharmaceutica NV, Beerse (BE)

(72) Inventors: Steven D. Goldberg, San Diego, CA (US); Douglas C. Behenna, SanJuan Capistrano, CA (US); Steven A. Loskot, San Diego, CA (US); Stefan J. McCarver, San Diego, CA (US); Timothy B. Rhorer, San Diego, CA (US); Kristin G. Song, San Diego, CA (US); Alexander E. Valdes, San Diego, CA (US); Craig R. Woods, San Diego, CA (US); Xiahua Xue, San Diego, CA (US); Brock T. Shireman, Poway, ID (US); Virginia M. Tanis, Vista, CA (US); Deane Gordan, San Diego, CA (US)

(73) Assignee: Janssen Pharmaceutica NV, Beerse (BE)

(21) Appl. No.: 18/693,202

(22) PCT Filed: Sep. 26, 2022
(86) PCT No.: PCT/US2022/076999

§ 371 Date: Mar. 19, 2024

Related U.S. Application Data
(60) Provisional application No. 63/248,561, filed on Sep. 27, 2021.
(60) Provisional application No. 63/273,395, filed on Oct. 29, 2021.

(51) Int. Cl. C07D 413/14; A61K 31/4184; A61K 31/4196; A61K 31/4245; A61K 31/437; A61K 31/513; C07D 403/14; C07D 471/04

(52) U.S. Cl. CPC C07D 413/14; A61K 31/4184; A61K 31/4196; A61K 31/4245; A61K 31/437; A61K 31/513; C07D 403/14; C07D 471/04

(57) ABSTRACT

The present application discloses compounds having the following formula (I): or pharmaceutically acceptable salts thereof, wherein R¹, R², R³, R⁴, and X are defined in the specification, as well as methods of making and using the compounds disclosed herein for treating or ameliorating an IL-17 mediated syndrome, disorder and/or disease.

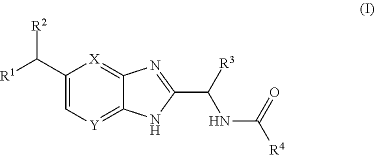

### REFERENCE TO SEQUENCE LISTING SUBMITTED ELECTRONICALLY

This application contains a sequence listing, which is submitted electronically as an ST.26 XML formatted sequence listing with a file name “PRD4154WOPCT1_SL.xml, creation date of Sep. 7, 2022 and having a size of 4.00 KB. The sequence listing is part of the specification and is herein incorporated by reference in its entirety.

### FIELD

Disclosed herein are benzimidazole and azabenzimidazole compounds, and pharmaceutical compositions thereof, which modulate Interleukin-17A. Also disclosed herein is the therapeutic use of such compounds, for example, in treating and/or ameliorating an IL-17A mediated inflammatory syndrome, disorder, or disease.

### BACKGROUND

Interleukin-17 (“IL-17”), also known as IL-17A and CTLA-8, is produced mainly by CD4+Th17 cells, and also by other immune cells such as CD8+ T cells, 76 T cells, NK cells, NKT cells, and innate lymphoid cells (ILCs). IL-17A exists as a homodimer (A/A) or as a heterodimer (A/F) with IL-17F and signals through binding to dimeric receptor complex IL-17RA and IL-17RC. IL-17RA is ubiquitously expressed at particularly high levels by haematopoietic cell types, whereas IL-17RC is preferentially expressed by non-haematopoietic cells (Gaffen, S. Structure and signaling in the IL-17 receptor family. Nat. Rev. Immunol. 2009, 9, 556-567). IL-17A/IL-17R signaling induces de novo gene transcription by triggering NF-kB, C/EBP and MAPK pathways through ACT1-TRAF6-TRAF4. It can also stabilize target mRNA transcripts through the ACT1-TRAF2-TRAF5 complex (Amatya N. et al., Trends in Immunology, 2017, 38, 310-322). IL-17A stimulates the release of inflammatory mediators including IL-6, IL-8, G-CSF, TNF-α, and IL-1β that recruit and activate lymphocytes to the site of injury or inflammation and maintain a proinflammatory state.

As discussed below, preclinical and clinical data have demonstrated the significant pathological role of IL-17A in multiple autoimmune and inflammatory diseases.

For psoriasis: IL-17A mRNA and/or protein levels are elevated in the lesional skin and blood of patients with psoriasis and correlate with disease severity. IL-17A acts directly in synergy with other cytokines (such as TNFα, IFNγ or IL-22) on keratinocytes triggering a self-amplifying inflammatory response in the skin and leading to the formation of psoriatic plaques. The blockade of IL-17A by means of antibodies to IL-17A or IL-23 results in complete reversal of the molecular and clinical disease features in majority of psoriasis patients, manifesting the significant role of IL-17A and IL-17-producing T-cells in the immunopathogenesis of psoriasis. (Hawkes et al., Psoriasis Pathogenesis and the Development of Novel, Targeted Immune Therapies. J Allergy Clin Immunol. 2017, 140(3): 645-653). The development and approval of IL-17 monoclonal antibodies such as secukinumab, ixekizumab, and brodalumab and their transformational efficacy for psoriasis have demonstrated IL-17A as a valid target for psoriasis treatments. (Blauvelt A. and Chiricozzi A. The Immunologic Role of IL-17 in Psoriasis and Psoriatic Arthritis Pathogenesis. Clin Rev Allergy Immunol. 2018, 55(3):379-390).

For psoriatic arthritis (PsA): IL-17A is mechanistically relevant to PsA through NFκB activation that triggers transcription of several PsA related genes including the receptor activator of nuclear factor KB ligand (RANKL). RANKL triggers the differentiation of osteoclast precursor cells into activated osteoclasts, resulting in bone resorption and subsequently joint deformity in PsA (Adamopoulos I. and Mellins E. Nature reviews Rheumatology 2015; 11:189-94). PsA joint is enriched for IL-17+CD8+ T cells, and the levels of this T cell subset are correlated with disease activity (Menon B. et al., Arthritis & Rheumatology 2014; 66: 1272-81). Synovial fibroblasts isolated from PsA patients also contain elevated IL-17R expression and secrete increased IL-6, CXCL8 and MMP3 ex vivo compared to osteoarthritis patients. Both secukinumab and ixekizumab are FDA approval drugs for PsA. In matching-adjusted indirect comparison analysis, secukinumab was associated with higher ACR 20/50/70 response rates in patients with active PsA than anti-TNFα antibodies (Mease P. et al., Eur. J. Rheumatol. 2019 Jul. 1; 6(3):113-121; Strand V. et al., J. Comp. Eff. Res. 2019, 8(7):497-510; Nash P. et al., Rheumatol. Ther. 2018, 5(1):99-122). In a recent head-to-head study, ixekizumab was superior to adalimumab in achieving simultaneous improvement of joint and skin disease (ACR50 and PASI100) in patients with PsA and inadequate response to conventional synthetic disease-modifying antirheumatic drug (Mease, P. et al. Ann Rheum Diss 2020; 79:123-131). By hitting the same target, IL-17A small molecule inhibitor compounds may exert similar or better efficacy than biologics considering that small molecules generally have better tissue penetration.

For rheumatoid arthritis (RA): IL-17A has been recognized as critical to the progression of rheumatoid arthritis. “The recognition of IL-17 as a pro-inflammatory T cell derived cytokine, and its abundance within rheumatoid joints, provides the strongest candidate mechanism to date through which T cells can capture and localize macrophage effector functions in rheumatoid arthritis” Stamp, L. et al., Immunol. Cell Biol. 2004, 82(1): 1-9. Moreover, in rheumatoid arthritis IL-17A acts locally on synoviocytes and osteoblasts contributing to synovitis and joint destruction. Robert and Miossec have proposed the use of synovial biopsies and/or biomarkers to precisely identify patients that would respond to IL-17A inhibition. Their work concludes that IL-17 inhibitors should now be considered in the development of precision medicine in RA. (Robert M. and Miossec P., Front. Med., 2019, 5:364).

For Ankylosing Spondylitis (AS): Various studies have reported elevated IL-17A and Th17 and other cells producing IL-17 in AS blood samples (Wendling D. et al., Joint Bone Spine. 2007; 74:304-305; Shen H. et al., Arthritis Rheum. 2009; 60(6):1647-56; Zhang L. et al., PLoS One. 2012; 7(4):e31000; Jansen D. et al., Rheumatology (Oxford). 2015 April; 54(4): 728-735). In situ analysis of AS spine has revealed increased IL-17A-producing cells in bone of facet (zygapophyseal) joints (Appel H. et al., Arthritis Res. Ther. 2011; 13(3):R95). Two advanced IL-17A neutralizing antibodies, secukinumab, approved by FDA for AS, and ixekizumab, have demonstrated efficacy over placebo even in anti-TNF inadequate responders. In contrast, anti-IL-23 p40 and p19 biologics failed to demonstrate beneficial effect (Deodhar A. et al., Arthritis Rheumatol. 2019, 71(2):258-270; Baeten D. et al., Ann. Rheum. Dis. 2018, 77(9):1295-1302), indicating the differential underling mechanism along IL-23/IL-17 pathway in AS and providing a strong evidence to support continuing developing IL-17A inhibitors.

For hidradenitis suppurativa (HS): Increased IL-17 and IL-17-producing T helper cells in the skin lesions of HS patients were reported and molecular proteomics and gene expression data indicate that the IL-23/Th17 pathway is upregulated in HS lesions (Schlapbach C. et al., J. Am. Acad. Dermatol. 2011; 65(4):790; Kelly G. et al., British J. Dermatol. 2015 December; 173(6):1431-9; Moran B. et al., J. Invest. Dermatol. 2017; 137(11):2389; Thomi R. et al., JAMA Dermatol. 2018; 154(5):592). Seven of nine (78%) patients with moderate-to-severe HS achieved HiSCR in an open-label pilot-trial with Secukinumab (Prussick L. et al., British J. Dermatol. 2019 September; 181(3):609-611), and more clinical trials with anti-IL-17 mAbs in HS are on-going.

For bullous pemphigoid (BP): IL-17 is elevated in the blister fluid and perilesional skin of BP patients. (Le Jan S. et al., J. Invest. Dermatol. 2014; 134 (12):2908-2917.; Chakievska L. J Autoimmun. 2019, 96:104-112). Exome sequencing of BP patients revealed mutations in twelve IL-17-related genes in one third of patients, providing the genetic link between IL-17 pathway and BP (Chakievska L. J Autoimmun. 2019, 96:104-112). In experimental murine BP, IL-17A−/− mice are protected, and anti-IL-17A treatment significantly reduced skin lesions in wild type (Chakievska L. J Autoimmun. 2019, 96:104-112). Ixekizumab Phase 2 of treatment naive and refractory BP patients is on-going (NCT03099538).

For atopic dermatitis (AD): IL-17 was found to be elevated in peripheral blood and lesions in AD patients and Th17 cells infiltrated more markedly in acute than chronic lesions, suggesting its role in acute phase of AD (Koga C. et al., J. Invest. Dermatol. 2008, 128, 2625-2630). Molecular profile analysis from ustekinumab Phase II suggest likely contribution of IL-23/Th17/IL-17 pathway in AD (Khattri S. et al., Exp. Dermatol. 2017 January; 26(1):28-35).

For vitiligo: Many studies in vitiligo patients have demonstrated an increased frequency of Th17 cells and higher levels of IL-17 in both circulation and lesions that positively correlates with disease duration, extent, and activity (Singh R. et al., Autoimmun. Rev 2016, April; 15(4):397-404). Mouse studies demonstrated that depigmentation correlates with greater IL-17 expression/secretion, which modulates vitiligo development (Eby J. et al., Pigment Cell & Melanoma Res. 2014, November; 27(6):1075-85).

For multiple sclerosis (MS): IL-17 expression is increased in PBMCs, cerebrospinal fluid (CSF) as well as in brain lesions and cells from MS patients (Lock, C. et al., Nat. Med. 2002, 8: 500-508; Matusevicius, D. et al., Mult. Scler. 1999, 5: 101-104; Tzartos, J. et al., Am. J. Pathol. 2008, 172: 146-155). IL-17-producing T cells are enriched in active MS lesions (Tzartos, J. et al., Am. J. Pathol. 2008, 172: 146-155; Willing A. et al., J. Immunol. 2018, 200(3):974-982). IL-17A levels were elevated in the CSF of relapsing-remitting MS (RRMS) patients and correlated with the CSF/serum albumin quotient, a measure of blood-brain barrier (BBB) dysfunction, together with in vitro data that IL-17A in combination with IL-6 reduced the expression of tight junction-associated genes and disrupted monolayer integrity in a BBB cell line, highlighting the potential importance of targeting IL-17A in preserving BBB integrity in RRMS (Setiadi A F et al., J Neuroimmunol. 2019, 332:147-154). Secukinumab yielded promising first results in a proof-of-concept study in MS patients (Havrdovi, E. et al., J. Neurol. 2016, 263: 1287-1295).

For Asthma: IL-17 expression is increased in the lung, sputum, bronchoalveolar lavage fluid, and sera in patients with asthma, and the severity of airway hyperresponsiveness is positively correlated with IL-17 expression levels. (Chakir J. et al., J. Allergy Clin. Immunol. 2003,111(6):1293-8). IL-17 was reported to be increased in asthmatic airways and induce human bronchial fibroblasts to produce cytokines (Molet S. et al., J. Allergy Clin. Immunol. 2001, 108(3):430-8). Anti-IL-17 antibody modulates airway responsiveness, inflammation, tissue remodeling, and oxidative stress in chronic mouse asthma models (Camargo LdN. et al., Front Immunol. 2018; 8:1835; dos Santos T. et al., Front. Physiol. 2018, 9:1183).

For Chronic Obstructive Pulmonary Disease (COPD): An increase in Th17 cells was observed in patients with COPD compared with current smokers without COPD and healthy subjects, and inverse correlations were found between Th17 cells with lung function (Vargas-Rojas M. et al., Respir. Med. 2011 November; 105(11):1648-54). In three recent human COPD studies, gene expression profile in bronchial epithelia showed that higher IL-17 signature expression is associated with a lack of response to inhaled corticosteroid, suggesting that there is a COPD subgroup that may benefit from IL-17 inhibitor therapy (Christenson S. et al., J. Clin. Invest. 2019; 129(1):169-181).

For Uveitis: IL-17 promotes the release of inflammatory mediators from retinal pigment epithelium cell line, disrupting the retinal pigment epithelium barrier function (Chen Y. et al., PLoS One. 2011; 6:e18139). IL-17 levels were elevated in the serum or aqueous humor of uveitis patients (El-Asrar A. et al., Clin. Immunol. 2011; 139(2):177-84; Jawad S. et al., Ocul. Immunol. Inflamm. 2013; 21(6):434-9; Kuiper J. et al., Am. J. Ophthalmol. 2011; 152(2):177-182.). Anti-IL-17 antibody delayed the onset of ocular inflammation and markedly inhibited the development of experimental autoimmune uveitis in rats (Zhang R. et al., Curr. Eye Res. 2009 April; 34(4):297-303). The analysis of secondary efficacy data from subcutaneous (sc) secukinumab phase 3 trials in uveitis suggested a beneficial effect of secukinumab in reducing the use of concomitant immunosuppressive medication (Dick A. et al., Ophthalmology 2013; 120(4):777-87). Later study of intravenous secukinumab in uveitis demonstrated greater efficacy than sc dosing, suggesting requiring optimal exposure for efficacy and confirming the therapeutic potential of IL-17A inhibition (Letko E. et al., Ophthalmology 2015, 122(5), 939-948). Ustekinumab that blocks IL-23/IL-17 pathway was also reported to successfully treat a noninfectious uveitis patient who had severe concomitant psoriasis and PsA and failed to respond to conventional immune suppressants (Mugheddu C. et al., Dermatol. Ther. 2017 September; 30(5); e12527.).

For multiple myeloma (MM): IL-17A serum levels were significantly higher in MM patients and also in patients with advanced stage compared with healthy subjects (Lemancewicz D. et al., Med. Sci. Monit. 2012; 18(1): BR54-BR59). Administration of secukinumab in the SCIDhu model of human myeloma weekly for 4 weeks after the first detection of tumor in mice led to a significant inhibition of tumor growth and reduced bone damage compared to isotype control mice (Prabhala R. et al., Leukemia. 2016 February; 30(2): 379-389).

For systemic lupus erythematosus (SLE): Increased serum or plasma levels of IL-17, expansion of IL-17-producing T cells in the peripheral blood, and infiltration of Th17 cells in target organs like the kidneys was observed in SLE patients (Wong C. et al., Lupus. 2000; 9(8):589-593; Wong C. et al., Clinical Immunology. 2008; 127(3):385-393; Zhao X-F. et al., Mol. Biol. Rep. 2010 January; 37(1):81-5; Chen X. et al., J. Clin. Immunol. 2010 March; 30(2):221-5; Xing Q. et al., Rheumatol. Int. 2012 April; 32(4):949-58). Imbalance between Th17 cells and regulatory T (Treg) cells has been observed in SLE patients including quiescent stage (Ma J. et al., Clin. Rheumatol. 2010; 29(11):1251-1258; Dolff S. et al., Clin. Immunol. 2011, 141(2):197-204). Overexpression of IL-17A using adenovirus enhanced the severity of lupus nephritis, while blockade of IL-17A using neutralizing antibody resulted in decreased severity of lupus nephritis (Wen, Z. et al., PLoS One. 2013, 8: e58161). In a phase 2 study, ustekinumab, an anti-IL-12/23 p40 monoclonal antibody blocking IL-23/IL-17 pathway, has demonstrated efficacy in SLE patients (van Vollenhoven R. et al., Lancet 2018; 392: 1330-39). Human expression studies, animal models, and clinical trials indicate that IL-17 blockade may become a promising therapeutic strategy for SLE (Koga T. et al., Expert Rev. Clin. Immunol. 2019, 15 (6) 629-637).

In summary, animal and human studies have shown that IL-17A plays crucial role in pathogenesis of the multiple diseases and/or conditions discussed above. The significance of targeting IL-17A has been demonstrated by the transformational efficacy of injectable IL-17A neutralizing antibodies in patients.

Despite the advances achieved with injectable IL-17A antagonist antibodies, there is a long-felt need for the development of an oral small molecule IL-17A inhibitor as it may broaden treatment options for many patients without access to biologics. In addition, a safe and efficacious small molecule IL-17A inhibitor may offer significant benefits to patients over the injectable IL-17A neutralizing antibodies such as convenient dosing regimens and cost savings, which in turn may provide effective long-term disease management.

However, the development of an oral small molecule treatment has remained challenging. For example, no oral small molecule IL-17A inhibitor has progressed into late-stage clinical trials yet, and only two oral small molecule IL-17A inhibitors have progressed into phase I clinical trials (NCT04586920 and NCT04883333) as of Sep. 28, 2021. Additionally, as of December 2021, one of these clinical trials (NCT04586920) was suspended due to safety review. Accordingly, there is a need for new small molecule IL-17A modulators (e.g., inhibitors).

### SUMMARY

The present application discloses a compound of Formula I:

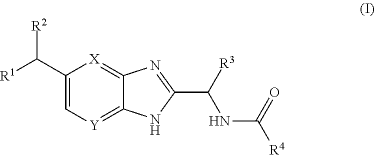

or a pharmaceutically acceptable salt thereof, wherein:
R¹ is:

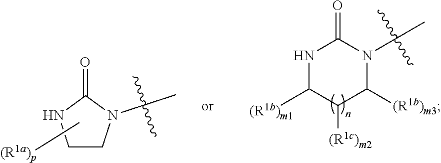

R^{1a} independently for each occurrence is —C₍₁₋₃₎alkyl or —C₍₃₋₅₎cycloalkyl, wherein the —C₍₁₋₃₎alkyl is unsubstituted or substituted with one to five fluorine atoms;
R^{1b} independently for each occurrence is —C₍₁₋₃₎alkyl or C₍₃₋₅₎cycloalkyl, wherein the —C₍₁₋₃₎alkyl is unsubstituted or substituted with one to five fluorine atoms;
R^{1c} independently for each occurrence is halo, —C₍₁₋₃₎alkyl, or C₍₃₋₅₎cycloalkyl, wherein the —C₍₁₋₃₎alkyl is unsubstituted or substituted with one to five fluorine atoms;
p is 0, 1, 2, 3, or 4;
n is 1 or 2;
m1, m2, and m3, are each independently 0, 1, or 2;
R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to six R^{2a} groups;
R^{2a} independently for each occurrence is fluorine, —CN, or —O—C₍₁₋₃₎alkyl;
R³ is C₍₁₋₈₎alkyl, —C₍₁₋₆₎alkyl-O—C₍₁₋₆₎alkyl, or —C₍₁₋₆₎alkyl-O—C₍₃₋₅₎cycloalkyl, wherein the C₍₁₋₈₎alkyl and —C₍₁₋₆₎alkyl-O—C₍₁₋₆₎alkyl are unsubstituted or substituted with one to six fluorine atoms, and wherein the —C₍₁₋₆₎alkyl-O—C₍₃₋₅₎cycloalkyl is unsubstituted or substituted with one to four fluorine atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, and —C₍₀₋₂₎alkyl-C₍₃₋₆₎cycloalkyl are unsubstituted or substituted with one to six substituents independently selected from fluorine, CH₃, CH₂F, CHF₂, CF₃, and —CN;
X is CH, CF or N; and
Y is CH or CF.

The present application also discloses a pharmaceutical composition comprising a compound of Formula I, or a pharmaceutically acceptable salt thereof, and a pharmaceutically acceptable carrier.

The present application also discloses a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof. In some embodiments, the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

### DETAILED DESCRIPTION

### Definitions

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

The term “administering” with respect to the methods of the invention, means a method for therapeutically or prophylactically preventing, treating or ameliorating a syndrome, disorder or disease as described herein by using a compound of the disclosure, or pharmaceutically acceptable salt thereof, composition thereof, or medicament thereof. Such methods include administering a therapeutically effective amount of a compound of the disclosure, or pharmaceutically acceptable salt thereof, composition thereof, or medicament thereof, at different times during the course of a therapy or concurrently or sequentially as a combination therapy.

The term “subject” refers to a patient, which may be an animal, preferably a mammal, most preferably a human, whom will be or has been treated by a method according to an embodiment of the application. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

The term “therapeutically effective amount” or “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

As used herein, “IL-17” or “IL-17A” refers to interleukin 17A. It is also named IL17, CTLA8, CTLA-8. Interleukin 17A is a pro-inflammatory cytokine. This cytokine is produced by a group of immune cells in response to their stimulation. An exemplary amino acid sequence of human IL-17 is represented in GenBank Accession No. NP_002181.1, which can be encoded by a nucleic acid sequence such as that of GenBank Accession No. NM_002190.3.

The term “modulator” as used herein refers to any agents or molecules that can bind to IL-17, including small molecule compounds.

“Active moiety” refers to a molecule or ion responsible for a physiological or pharmacological action. A compound of formula (I), as exemplified in the Examples and also described herein, is an active moiety.

As used herein, the term “composition” is intended to encompass a product comprising the specified ingredients in the specified amounts, as well as any product which results, directly or indirectly, from combinations of the specified ingredients in the specified amounts.

As used herein, the term “treat,” “treating,” or “treatment” of any disease, condition, syndrome or disorder refers, in one embodiment, to ameliorating the disease, condition, syndrome or disorder (i.e., slowing or arresting or reducing the development of the disease or at least one of the clinical symptoms thereof). In another embodiment, “treat,” “treating,” or “treatment” refers to alleviating or ameliorating at least one physiological or biochemical parameter associated with or causative of the disease, condition, syndrome or disorder, including those which may not be discernible by the patient. In a further embodiment, “treat”, “treating”, or “treatment” refers to modulating the disease, condition, syndrome or disorder either physically (e.g. stabilization of a discernible symptom), physiologically, (e.g. stabilization of a physical parameter), or both. In yet another embodiment, “treat,” “treating,” or “treatment” refers to preventing or delaying the onset or development or progression of the disease, condition, syndrome or disorder.

As used herein, the term “QD” means once daily.

As used herein, the term “BID” means twice daily.

The term “alkyl” is a straight or branched saturated hydrocarbon. For example, an alkyl group can have 1 to 12 carbon atoms (i.e., (C₁-C₁₂)alkyl) or 1 to 6 carbon atoms (i.e., (C₁-C₆)alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₃)₂), 1-butyl (n-bu, n-butyl, —CH₂CH₂CH₂CH₃), 2-butyl (s-bu, s-butyl, —CH(CH₃)CH₂CH₃), tert-butyl (t-bu, t-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). Any alkyl group may be unsubstituted or substituted.

The term “C_{(a-b)}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “cycloalkyl” refers to a saturated or partially unsaturated all carbon ring system having 3 to 8 carbon atoms (i.e., C₍₃₋₈₎cycloalkyl), and preferably 3 to 6 carbon atoms (i.e., C₍₃₋₆₎cycloalkyl), wherein the cycloalkyl ring system has a single ring or multiple rings in a spirocyclic or bicyclic form. Exemplary cycloalkyls include, but are not limited to, cyclopropyl, cyclobutyl, cyclopentyl, cyclohexyl, cycloheptyl, and cyclooctyl. Unless otherwise stated specifically in the specification, a cycloalkyl group may be unsubstituted or substituted. Some cycloalkyl groups may exist as spirocycloalkyls, wherein two cycloalkyl rings are fused through a single carbon atom; for example and without limitation, an example of a spiropentyl group is

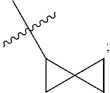 [SMILES: *C1CC12CC2]

for example and without limitation, examples of spirohexyl groups include

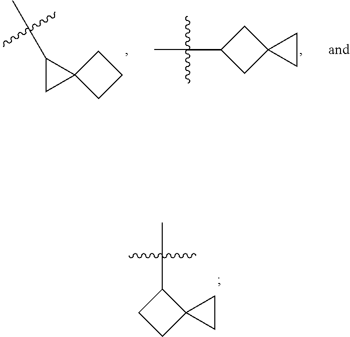 [SMILES: *C1CC12CCC2 ; *C1CC2(CC2)C1 ; *C1CCC12CC2]

for example and without limitation examples of cycloheptyl groups include

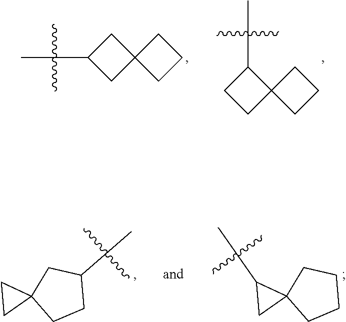 [SMILES: *C1CC2(CCC2)C1 ; *C1CCC12CCC2 ; *C1CCC2(CC2)C1 ; *C1CC12CCCC2]

for example and without limitation examples of cyclooctyl groups include

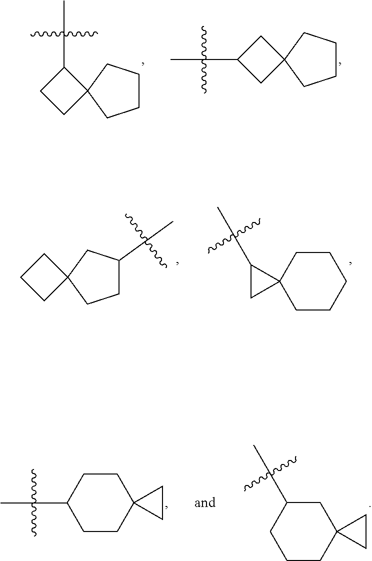 [SMILES: *C1CCC12CCCC2 ; *C1CC2(CCCC2)C1 ; *C1CCC2(CCC2)C1 ; *C1CC12CCCCC2 ; *C1CCC2(CC1)CC2 ; *C1CCCC2(CC2)C1]

Unless otherwise stated specifically in the specification, a siprocycloalkyl group may be unsubstituted or substituted. Bicyclic cycloalkyl ring systems also include or

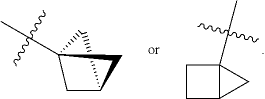

The term “heteroaryl” refers to a single aromatic ring that has at least one atom other than carbon in the ring, wherein the atom is selected from the group consisting of oxygen, nitrogen and sulfur. The term “heteroaryl” includes single aromatic rings of from 1 to 6 carbon atoms and 1 to 4 heteroatoms selected from the group consisting of oxygen, nitrogen and sulfur. Exemplary heteroaryl ring systems include but are not limited to pyridinyl, pyrimidinyl, pyrazinyl, pyridazinyl, pyrimidinyl, pyrazolyl, oxazolyl, oxadiazolyl, isoxazolyl, triazolyl, imidazolyl, tetrazolyl, thienyl, thiazolyl, isothiazolyl, thiadiazolyl, or furyl.

The term “halogen” refers to bromo (—Br), chloro (—Cl), fluoro (—F) or iodo (—I).

Where the compounds disclosed herein have at least one stereocenter, they may accordingly exist as enantiomers or diastereomers. It is to be understood that all such isomers and mixtures thereof are encompassed within the scope of the present invention.

“Diastereoisomers” are stereoisomers that have at least two asymmetric atoms, but which are not mirror images of each other.

“Enantiomers” are a pair of stereoisomers that are non-superimposable mirror images of each other. A “racemic” mixture is a 1:1 mixture of a pair of enantiomers. A “scalemic” mixture of enantiomers is mixture of enantiomers at a ratio other than 1:1.

Where the processes for the preparation of the compounds according to the invention give rise to mixture of stereoisomers, these isomers may be separated by conventional techniques such as preparative chromatography. The compounds may be prepared in racemic form, a scalemic mixture, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral column vial HPLC or SFC. In some instances rotamers of compounds may exist which are observable by ¹H NMR leading to complex multiplets and peak integration in the ¹H NMR spectrum.

The absolute stereochemistry is specified according to the Cahn-Ingold-Prelog R—S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30). Certain examples contain chemical structures that are depicted or labelled as an (R*) or (S*). When (R*) or (S*) is used in the name of a compound or in the chemical representation of the compound, it is intended to convey that the compound is a pure single isomer at that stereocenter; however, absolute configuration of that stereocenter has not been established. Thus, a compound designated as (R*) refers to a compound that is a pure single isomer at that stereocenter with an absolute configuration of either (R) or (S), and a compound designated as (S*) refers to a compound that is a pure single isomer at that stereocenter with an absolute configuration of either (R) or (S). For example, 4-Methyl-N—((R*)-1-(5-((R)-1-((S)-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-1,2,5-oxadiazole-3-carboxamide:

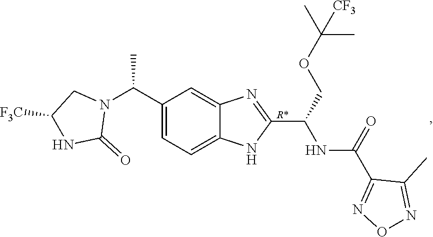 [SMILES: Cc1nonc1C(=O)N[C@@H](COC(C)(C)C(F)(F)F)c1nc2cc([C@@H](C)N3C[C@@H](C(F)(F)F)NC3=O)ccc2[nH]1]

refers to a compound that is either:

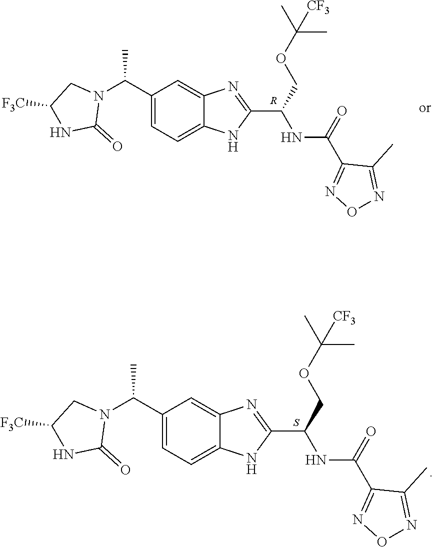

Pseudoasymmetric stereogenic centers are treated in the same way as chiral centers, but are given lower-case symbols, r or s (Angew. Chem. Int. Ed. Engl. 1982, 21, 567-583).

During any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Furthermore, it is intended that within the scope of the present invention, any element, in particular when mentioned in relation to a compound of the disclosure, or pharmaceutically acceptable salt thereof, shall comprise all isotopes and isotopic mixtures of said element, either naturally occurring or synthetically produced, either with natural abundance or in an isotopically enriched form. For example, a reference to hydrogen includes within its scope ¹H, ²H (i.e., deuterium or D), and ³H (i.e., tritium or T). In some embodiments, the compounds described herein include a ²H (i.e., deuterium) isotope. By way of example, the group denoted —C₍₁₋₆₎alkyl includes not only —CH₃, but also CD₃; not only CH₂CH₃, but also CD₂CD₃, etc. Similarly, references to carbon and oxygen include within their scope respectively ¹²C, ¹³C and ¹⁴C and ¹⁵O and ¹⁶O and ¹⁷O and ¹⁸O. The isotopes may be radioactive or non-radioactive. Radiolabelled compounds of the disclosure may include a radioactive isotope selected from the group comprising ³H, ¹¹C, ¹⁸F, ³⁵S, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

### Compounds of the Disclosure

The present application discloses a compound of Formula I:

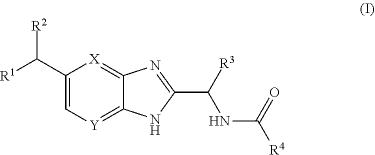

or a pharmaceutically acceptable salt thereof, wherein:
R¹ is:

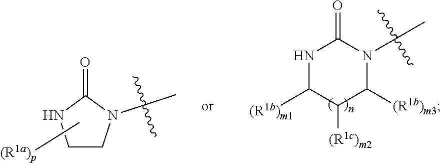

R^{1a} independently for each occurrence is —C₍₁₋₃₎alkyl or —C₍₃₋₅₎cycloalkyl, wherein the —C₍₁₋₃₎alkyl is unsubstituted or substituted with one to five fluorine atoms;
R^{1b} independently for each occurrence is —C₍₁₋₃₎alkyl or C₍₃₋₅₎cycloalkyl, wherein the —C₍₁₋₃₎alkyl is unsubstituted or substituted with one to five fluorine atoms;
R^{1c} independently for each occurrence is halo, —C₍₁₋₃₎alkyl, or C₍₃₋₅₎cycloalkyl, wherein the —C₍₁₋₃₎alkyl is unsubstituted or substituted with one to five fluorine atoms;
p is 0, 1, 2, 3, or 4;
n is 1 or 2;
m1, m2, and m3, are each independently 0, 1, or 2;
R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to six R^{2a} groups;
R^{2a} independently for each occurrence is fluorine, —CN, or —O—C₍₁₋₃₎alkyl;
R³ is C₍₁₋₆₎alkyl, —C₍₁₋₆₎alkyl-O—C₍₁₋₆₎alkyl, or —C₍₁₋₆₎alkyl-O—C₍₃₋₅₎cycloalkyl, wherein the C₍₁₋₈₎alkyl and —C₍₁₋₆₎alkyl-O—C₍₁₋₆₎alkyl are unsubstituted or substituted with one to six fluorine atoms, and wherein the —C₍₁₋₆₎alkyl-O—C₍₃₋₅₎cycloalkyl is unsubstituted or substituted with one to four fluorine atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, and —C₍₀₋₂₎alkyl-C₍₃₋₆₎cycloalkyl are unsubstituted or substituted with one to six substituents independently selected from fluorine, CH₃, CH₂F, CHF₂, CF₃, and —CN;
X is CH, CF or N; and
Y is CH or CF.

The present application also discloses a compound of Formula I-1:

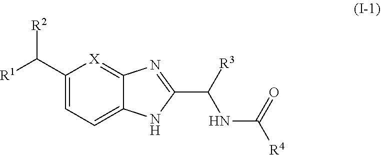

or a pharmaceutically acceptable salt thereof, wherein:
R¹ is:

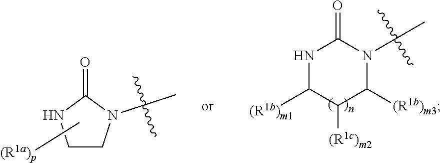

R^{1a} independently for each occurrence is —C₍₁₋₃₎alkyl or —C₍₃₋₅₎cycloalkyl, wherein the —C₍₁₋₃₎alkyl is unsubstituted or substituted with one to five fluorine atoms;
R^{1b} independently for each occurrence is —C₍₁₋₃₎alkyl or C₍₃₋₅₎cycloalkyl, wherein the —C₍₁₋₃₎alkyl is unsubstituted or substituted with one to five fluorine atoms;
R^{1c} independently for each occurrence is halo, —C₍₁₋₃₎alkyl, or C₍₃₋₅₎cycloalkyl, wherein the —C₍₁₋₃₎alkyl is unsubstituted or substituted with one to five fluorine atoms;
p is 0, 1, 2, 3, or 4;
n is 1 or 2;
m1, m2, and m3, are each independently 0, 1, or 2;
R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to six R^{2a} groups;
R^{2a} independently for each occurrence is fluorine, —CN, or —O—C₍₁₋₃₎alkyl;
R³ is C₍₁₋₈₎alkyl, —C₍₁₋₆₎alkyl-O—C₍₁₋₆₎alkyl, or —C₍₁₋₆₎alkyl-O—C₍₃₋₅₎cycloalkyl, wherein the C₍₁₋₈₎alkyl and —C₍₁₋₆₎alkyl-O—C₍₁₋₆₎alkyl are unsubstituted or substituted with one to six fluorine atoms, and wherein the —C₍₁₋₆₎alkyl-O—C₍₃₋₅₎cycloalkyl is unsubstituted or substituted with one to four fluorine atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, and —C₍₀₋₂₎alkyl-C₍₃₋₆₎cycloalkyl are unsubstituted or substituted with one to six substituents independently selected from fluorine, CH₃, CH₂F, CHF₂, CF₃, and —CN; and
X is CH or N.

In some embodiments, disclosed herein is a compound of Formula I, or a pharmaceutically acceptable salt thereof, wherein:
R¹ is:

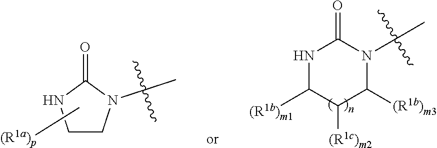

R^{1a} independently for each occurrence is —C₍₁₋₃₎alkyl or —C₍₃₋₅₎cycloalkyl, wherein the —C₍₁₋₃₎alkyl is unsubstituted or substituted with one to five fluorine atoms;
R^{1b} independently for each occurrence is —C₍₁₋₃₎alkyl or C₍₃₋₅₎cycloalkyl, wherein the —C₍₁₋₃₎alkyl is unsubstituted or substituted with one to five fluorine atoms;
R^{1c} independently for each occurrence is halo, —C₍₁₋₃₎alkyl, or C₍₃₋₅₎cycloalkyl, wherein the —C₍₁₋₃₎alkyl is unsubstituted or substituted with one to five fluorine atoms;
p is 0, 1, 2, 3, or 4;
n is 1 or 2;
m1, m2, and m3, are each independently 0, 1, or 2;
R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one —CN group;
R³ is C₍₁₋₈₎alkyl, —C₍₁₋₆₎alkyl-O—C₍₁₋₆₎alkyl, or —C₍₁₋₆₎alkyl-O—C₍₃₋₅₎cycloalkyl, wherein the C₍₁₋₈₎alkyl and —C₍₁₋₆₎alkyl-O—C₍₁₋₆₎alkyl are unsubstituted or substituted with one to six fluorine atoms, and wherein the —C₍₁₋₆₎alkyl-O—C₍₃₋₅₎cycloalkyl is unsubstituted or substituted with one to four fluorine atoms;
R⁴ is a 5-membered heteroaryl that is unsubstituted or substituted with one to two R^{4a} groups;
R^{4a} is halo, —C₍₁₋₆₎alkyl, or —C₍₀₋₂₎alkyl-C₍₃₋₆₎cycloalkyl, wherein the —C₍₁₋₆₎alkyl and —C₍₀₋₂₎alkyl-C₍₃₋₆₎cycloalkyl are unsubstituted or substituted with one to six substituents independently selected from fluorine and —CN; and
X is CH, CF or N; and
Y is CH or CF.

In some embodiments, disclosed herein is a compound of Formula I, or a pharmaceutically acceptable salt thereof, wherein:
R¹ is:

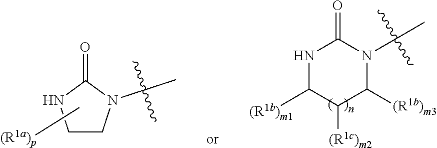

R^{1a} independently for each occurrence is —C₍₁₋₃₎alkyl, CH₂F, CHF₂, CF₃, or —C₍₃₋₄₎cycloalkyl;
R^{1b} independently for each occurrence is —C₍₁₋₃₎alkyl, CH₂F, CHF₂, CF₃, or —C₍₃₋₄₎cycloalkyl;
R^{1c} independently for each occurrence is halo, —C₍₁₋₃₎alkyl, CH₂F, CHF₂, CF₃, or —C₍₃₋₄₎cycloalkyl;
p is 0, 1, 2, 3, or 4;
n is 1 or 2;
m1, m2, and m3, are each independently 0, 1, or 2;
R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to four R^{2a} groups; and
R^{2a} independently for each occurrence is fluorine or —CN;
R³ is C₍₁₋₈₎alkyl, —C₍₁₋₆₎alkyl-O—C₍₁₋₆₎alkyl, or —C₍₁₋₆₎alkyl-O—C₍₃₋₅₎cycloalkyl, wherein the C₍₁₋₈₎alkyl and —C₍₁₋₆₎alkyl-O—C₍₁₋₆₎alkyl are unsubstituted or substituted with one to six fluorine atoms, and wherein the —C₍₁₋₆₎alkyl-O—C₍₃₋₅₎cycloalkyl is unsubstituted or substituted with one to four fluorine atoms;
R⁴ is a 5-membered heteroaryl that is unsubstituted or substituted with one to two R^{4a} groups;
R^{4a} is halo, —C₍₁₋₆₎alkyl, or —C₍₀₋₂₎alkyl-C₍₃₋₆₎cycloalkyl, wherein the —C₍₁₋₆₎alkyl and —C₍₀₋₂₎alkyl-C₍₃₋₆₎cycloalkyl are unsubstituted or substituted with one to six substituents independently selected from fluorine and —CN; and
X is CH, CF or N; and
Y is CH or CF.

In some embodiments, disclosed herein is a compound of Formula I, or a pharmaceutically acceptable salt thereof, wherein:
R¹ is:

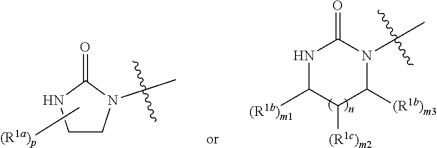

R^{1a} independently for each occurrence is CH₃, CH₂F, CHF₂, CF₃, or cyclopropyl;
R^{1b} independently for each occurrence is CH₃, CH₂F, CHF₂, CF₃, or cyclopropyl;
R^{1c} independently for each occurrence is fluorine, CH₃, CH₂F, CHF₂, CF₃, or cyclopropyl;
p is 0, 1, 2, 3, or 4;
n is 1 or 2;
m1, m2, and m3, are each independently 0, 1, or 2;
R² is H, —C₍₁₋₃₎alkyl, —C₍₃₋₄₎cycloalkyl, —C₍₁₋₃₎alkyl-O—C₍₁₋₃₎alkyl, or —C₍₁₋₃₎alkyl-O—C₍₃₋₄₎cycloalkyl, wherein the —C₍₃₋₄₎cycloalkyl is unsubstituted or substituted with one —CN group;
R³ is C₍₁₋₆₎alkyl, —C₍₁₋₄₎alkyl-O—C₍₁₋₄₎alkyl, or —C₍₁₋₄₎alkyl-O—C₍₃₋₄₎cycloalkyl, wherein the C₍₁₋₆₎alkyl and —C₍₁₋₄₎alkyl-O—C₍₁₋₄₎alkyl are unsubstituted or substituted with one to six fluorine atoms, and wherein the —C₍₁₋₄₎alkyl-O—C₍₃₋₄₎cycloalkyl is unsubstituted or substituted with one to four fluorine atoms;
R⁴ is a 5-membered heteroaryl that is unsubstituted or substituted with one to two R^{4a} groups;
R^{4a} is fluorine, —C₍₁₋₆₎alkyl, or —C₍₀₋₁₎alkyl-C₍₃₋₄₎cycloalkyl, wherein the —C₍₁₋₆₎alkyl and —C₍₀₋₁₎alkyl-C₍₃₋₄₎cycloalkyl are unsubstituted or substituted with one to six substituents independently selected from fluorine and —CN; and
X is CH, CF or N; and
Y is CH or CF.

In some embodiments, disclosed herein is a compound of Formula I, or a pharmaceutically acceptable salt thereof, wherein:
R¹ is

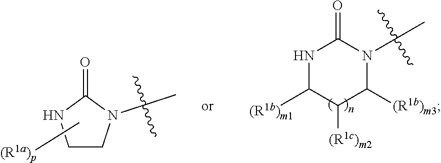

R^{1a} independently for each occurrence is CH₃, CH₂F, CHF₂, CF₃, or cyclopropyl;
R^{1b} independently for each occurrence is CH₃, CH₂F, CHF₂, CF₃, or cyclopropyl;
R^{1c} independently for each occurrence is fluorine, CH₃, CH₂F, CHF₂, CF₃, or cyclopropyl;
p is 0, 1, or 2;
n is 1 or 2;
m1, m2, and m3, are each independently 0, 1, or 2;
R² is H, —C₍₁₋₃₎alkyl, —C₍₃₋₄₎cycloalkyl, —C₍₁₋₃₎alkyl-O—C₍₁₋₃₎alkyl, or —C₍₁₋₃₎alkyl-O—C₍₃₋₄₎cycloalkyl, wherein the —C₍₃₋₄₎cycloalkyl is unsubstituted or substituted with one —CN group;
R³ is:

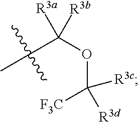

R^{3a}, R^{3b}, R^{3c}, and R^{3d} are each independently H or CH₃;
R⁴ is a 5-membered heteroaryl that is unsubstituted or substituted with one to two R^{4a} groups;
R^{4a} is fluorine, —C₍₁₋₃₎alkyl, or —C₍₃₋₄₎cycloalkyl; and
X is CH, CF or N; and
Y is CH or CF.

In some embodiments, disclosed herein is a compound of Formula I, or a pharmaceutically acceptable salt thereof, wherein:
R¹ is

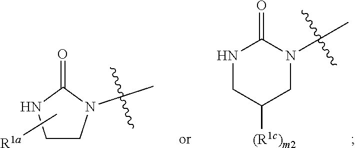

R^{1a} is —C₍₁₋₃₎alkyl, CH₂F, CHF₂, CF₃, or cyclopropyl;
R^{1c} independently for each occurrence is fluorine, —C₍₁₋₃₎alkyl, CH₂F, CHF₂, CF₃, or cyclopropyl;
m2 is 1, or 2;
R² is H, —C₍₁₋₃₎alkyl, —C₍₃₋₄₎cycloalkyl, —C₍₁₋₃₎alkyl-O—C₍₁₋₃₎alkyl, or —C₍₁₋₃₎alkyl-O—C₍₃₋₄₎cycloalkyl, wherein the —C₍₃₋₄₎cycloalkyl is unsubstituted or substituted with one —CN group;
R³ is:

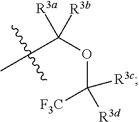

R^{3a}, R^{3b}, R^{3c}, and R^{3d} are each independently H or CH₃;
R⁴ is a 5-membered heteroaryl that is unsubstituted or substituted with one to two R^{4a} groups;
R^{4a} is fluorine, —C₍₁₋₆₎alkyl, or —C₍₀₋₁₎alkyl-C₍₃₋₄₎cycloalkyl, wherein the —C₍₁₋₆₎alkyl and —C₍₀₋₁₎alkyl-C₍₃₋₄₎cycloalkyl are unsubstituted or substituted with one to six substituents independently selected from fluorine and —CN; and
X is CH, CF or N; and Y is CH or CF.

In some embodiments, disclosed herein is a compound of Formula I, or a pharmaceutically acceptable salt thereof, wherein:
R¹ is

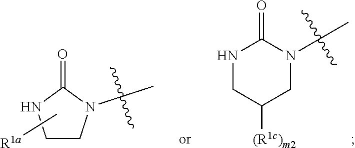

R^{1a} is —C₍₁₋₃₎alkyl, CH₂F, CHF₂, CF₃, or cyclopropyl;
R^{1c} independently for each occurrence is fluorine, —C₍₁₋₃₎alkyl, CH₂F, CHF₂, CF₃, or cyclopropyl;
m2 is 1, or 2;
R² is H, —C₍₁₋₃₎alkyl, —C₍₃₋₄₎cycloalkyl, —C₍₁₋₃₎alkyl-O—C₍₁₋₃₎alkyl, or —C₍₁₋₃₎alkyl-O—C₍₃₋₄₎cycloalkyl, wherein the —C₍₃₋₄₎cycloalkyl is unsubstituted or substituted with one —CN group;
R³ is:

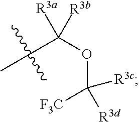

R^{3a}, R^{3b}, R^{3c}, and R^{3d} are each independently H or CH₃;
R⁴ is:

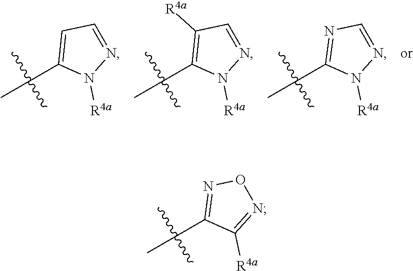

R^{4a} is fluorine, —C₍₁₋₆₎alkyl, or —C₍₀₋₁₎alkyl-C₍₃₋₄₎cycloalkyl; and
X is CH, CF or N; and
Y is CH or CF.

In some embodiments, disclosed herein is a compound of Formula I, or a pharmaceutically acceptable salt thereof, wherein R¹ is:

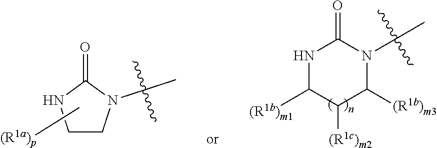

R^{1a} independently for each occurrence is —C₍₁₋₃₎alkyl, CH₂F, CHF₂, CF₃, or —C₍₃₋₄₎cycloalkyl;
R^{1b} independently for each occurrence is —C₍₁₋₃₎alkyl, CH₂F, CHF₂, CF₃, or —C₍₃₋₄₎cycloalkyl;
R^{1c} independently for each occurrence is halo, —C₍₁₋₃₎alkyl, CH₂F, CHF₂, CF₃, or —C₍₃₋₄₎cycloalkyl;
p is 0, 1, 2, 3, or 4;
n is 1 or 2; and
m1, m2, and m3, are each independently 0, 1, or 2.

In some embodiments, disclosed herein is a compound of Formula I, or a pharmaceutically acceptable salt thereof, wherein R¹ is:

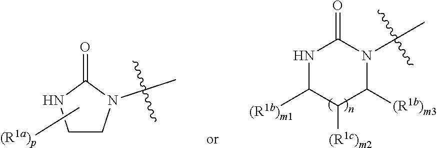

R^{1a} independently for each occurrence is CH₃, CH₂F, CHF₂, CF₃, or cyclopropyl;
R^{1b} independently for each occurrence is CH₃, CH₂F, CHF₂, CF₃, or cyclopropyl;
R^{1c} independently for each occurrence is fluorine, CH₃, CH₂F, CHF₂, CF₃, or cyclopropyl;
p is 0, 1, or 2;
n is 1 or 2; and
m1, m2, and m3, are each independently 0, 1, or 2.

In some embodiments, disclosed herein is a compound of Formula I, or a pharmaceutically acceptable salt thereof, wherein R¹ is:

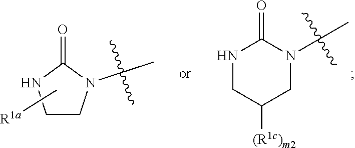

R^{1a} is —C₍₁₋₃₎alkyl, CH₂F, CHF₂, CF₃, or —C₍₃₋₄₎cycloalkyl;
R^{1c} independently for each occurrence is halo, —C₍₁₋₃₎alkyl, CH₂F, CHF₂, CF₃, or —C₍₃₋₄₎cycloalkyl;
m2 is 1 or 2.

In some embodiments, disclosed herein is a compound of Formula I, or a pharmaceutically acceptable salt thereof, wherein R¹ is:

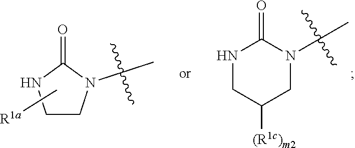

R^{1a} is CH₃, CH₂F, CHF₂, CF₃, or cyclopropyl;
R^{1c} independently for each occurrence is fluorine, CH₃, CH₂F, CHF₂, CF₃, or cyclopropyl;
m2 is 1 or 2.

In some embodiments, disclosed herein is a compound of Formula I, or a pharmaceutically acceptable salt thereof, wherein R¹ is:

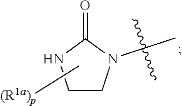

R^{1a} independently for each occurrence is —C₍₁₋₃₎alkyl or —C₍₃₋₅₎cycloalkyl, wherein the —C₍₁₋₃₎alkyl is unsubstituted or substituted with one to five fluorine atoms; and
p is 0, 1, 2, 3, or 4.

In some embodiments, disclosed herein is a compound of Formula I, or a pharmaceutically acceptable salt thereof, wherein R^{1a} independently for each occurrence is —C₍₁₋₃₎alkyl or —C₍₃₋₅₎cycloalkyl, wherein the —C₍₁₋₃₎alkyl is unsubstituted or substituted with one to five fluorine atoms; and p is 0, 1, or 2.

In some embodiments, disclosed herein is a compound of Formula I, or a pharmaceutically acceptable salt thereof, wherein R^{1a} independently for each occurrence is —C₍₁₋₃₎alkyl, CH₂F, CHF₂, CF₃, or —C₍₃₋₄₎cycloalkyl; and p is 0, 1, 2, 3, or 4. In some embodiments, disclosed herein is a compound of Formula I, or a pharmaceutically acceptable salt thereof, wherein R^{1a} independently for each occurrence is —C₍₁₋₃₎alkyl, CH₂F, CHF₂, CF₃, or —C₍₃₋₄₎cycloalkyl; and p is 0, 1, or 2.

In some embodiments, disclosed herein is a compound of Formula I, or a pharmaceutically acceptable salt thereof, wherein R^{1a} independently for each occurrence is CH₃, CH₂F, CHF₂, CF₃, or cyclopropyl; and p is 0, 1, 2, 3, or 4. In some embodiments, disclosed herein is a compound of Formula I, or a pharmaceutically acceptable salt thereof, wherein R^{1a} independently for each occurrence is CH₃, CH₂F, CHF₂, CF₃, or cyclopropyl; and p is 0, 1, or 2.

In some embodiments, disclosed herein is a compound of Formula I, or a pharmaceutically acceptable salt thereof, wherein R¹ is:

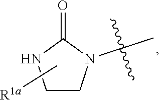

and
R^{1a} is —C₍₁₋₃₎alkyl or —C₍₃₋₅₎cycloalkyl, wherein the —C₍₁₋₃₎alkyl is unsubstituted or substituted with one to five fluorine atoms. In some embodiments, disclosed herein is a compound of Formula I, or a pharmaceutically acceptable salt thereof, wherein R^{1a} is —C₍₁₋₃₎alkyl, CH₂F, CHF₂, CF₃, or —C₍₃₋₄₎cycloalkyl. In some embodiments, disclosed herein is a compound of Formula I, or a pharmaceutically acceptable salt thereof, wherein R^{1a} is CH₃, CH₂F, CHF₂, CF₃, or cyclopropyl.

In some embodiments, disclosed herein is a compound of Formula I, or a pharmaceutically acceptable salt thereof, wherein R¹ is:

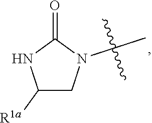

and
R^{1a} is —C₍₁₋₃₎alkyl or —C₍₃₋₅₎cycloalkyl, wherein the —C₍₁₋₃₎alkyl is unsubstituted or substituted with one to five fluorine atoms. In some embodiments, disclosed herein is a compound of Formula I, or a pharmaceutically acceptable salt thereof, wherein R^{1a} is —C₍₁₋₃₎alkyl, CH₂F, CHF₂, CF₃, or —C₍₃₋₄₎cycloalkyl. In some embodiments, disclosed herein is a compound of Formula I, or a pharmaceutically acceptable salt thereof, wherein R^{1a} is CH₃, CH₂F, CHF₂, CF₃, or cyclopropyl.

In some embodiments, disclosed herein is a compound of Formula I, or a pharmaceutically acceptable salt thereof, wherein R¹ is:

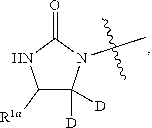

and
R^{1a} is —C₍₁₋₃₎alkyl or —C₍₃₋₅₎cycloalkyl, wherein the —C₍₁₋₃₎alkyl is unsubstituted or substituted with one to five fluorine atoms. In some embodiments, disclosed herein is a compound of Formula I, or a pharmaceutically acceptable salt thereof, wherein R^{1a} is —C₍₁₋₃₎alkyl, CH₂F, CHF₂, CF₃, or —C₍₃₋₄₎cycloalkyl. In some embodiments, disclosed herein is a compound of Formula I, or a pharmaceutically acceptable salt thereof, wherein R^{1a} is CH₃, CH₂F, CHF₂, CF₃, or cyclopropyl.

In some embodiments, disclosed herein is a compound of Formula I, or a pharmaceutically acceptable salt thereof, wherein R¹ is:

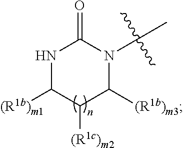

R^{1b} independently for each occurrence is —C₍₁₋₃₎alkyl or C₍₃₋₅₎cycloalkyl, wherein the —C₍₁₋₃₎alkyl is unsubstituted or substituted with one to five fluorine atoms;
R^{1c} independently for each occurrence is halo, —C₍₁₋₃₎alkyl, or C₍₃₋₅₎cycloalkyl, wherein the —C₍₁₋₃₎alkyl is unsubstituted or substituted with one to five fluorine atoms;
n is 1 or 2; and
m1, m2, and m3, are each independently 0, 1, or 2.

It is to be understood that when any one of m1, m2, or m3 is 2, the corresponding ring-bound carbon atom is geminally disubstituted. For example, when m1 is 2, R¹ has the following structure:

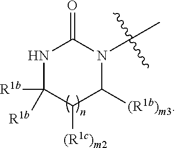

In some embodiments, disclosed herein is a compound of Formula I, or a pharmaceutically acceptable salt thereof, wherein R^{1b} independently for each occurrence is —C₍₁₋₃₎alkyl, CH₂F, CHF₂, CF₃, or —C₍₃₋₄₎cycloalkyl; and R^{1c} independently for each occurrence is halo, —C₍₁₋₃₎alkyl, CH₂F, CHF₂, CF₃, or —C₍₃₋₄₎cycloalkyl. In some embodiments, disclosed herein is a compound of Formula I, or a pharmaceutically acceptable salt thereof, wherein Rib independently for each occurrence is CH₃, CH₂F, CHF₂, CF₃, or cyclopropyl; and R^{1c} independently for each occurrence is fluorine, CH₃, CH₂F, CHF₂, CF₃, or cyclopropyl. In some embodiments, n is 1. In some embodiments, m1 is 0 or 1. In some embodiments, m2 is 1 or 2. In some embodiments, m3 is 0 or 1.

In some embodiments, disclosed herein is a compound of Formula I, or a pharmaceutically acceptable salt thereof, wherein R¹ is:

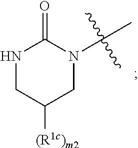

R^{1c} independently for each occurrence is halo, —C₍₁₋₃₎alkyl, or C₍₃₋₅₎cycloalkyl, wherein the —C₍₁₋₃₎alkyl is unsubstituted or substituted with one to five fluorine atoms; and m2 is 1 or 2. In some embodiments, disclosed herein is a compound of Formula I, or a pharmaceutically acceptable salt thereof, wherein R^{1c} independently for each occurrence is halo, —C₍₁₋₃₎alkyl, CH₂F, CHF₂, CF₃, or —C₍₃₋₄₎cycloalkyl; and m2 is 1 or 2. In some embodiments, disclosed herein is a compound of Formula I, or a pharmaceutically acceptable salt thereof, wherein R^{1c} independently for each occurrence is fluorine, CH₃, CH₂F, CHF₂, CF₃, or cyclopropyl; and m2 is 1 or 2. In some embodiments, m2 is 2.

In some embodiments, disclosed herein is a compound of Formula I, or a pharmaceutically acceptable salt thereof, wherein R¹ is:

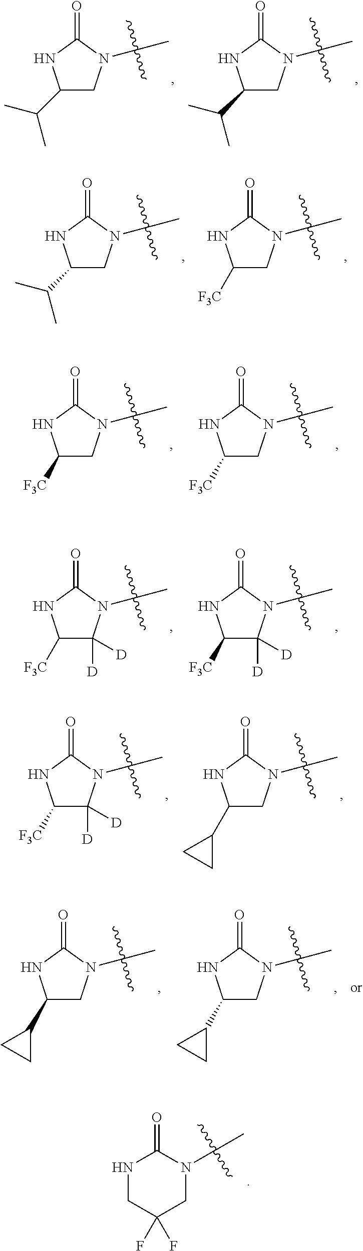

In some embodiments, disclosed herein is a compound of Formula I, or a pharmaceutically acceptable salt thereof, wherein R¹ is:

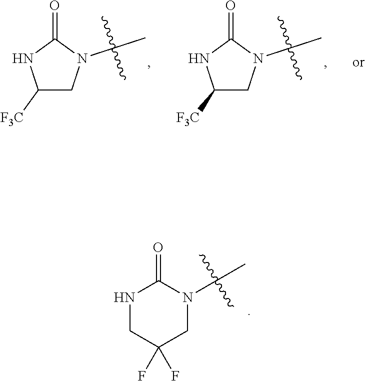 [SMILES: *N1CC(C(F)(F)F)NC1=O ; *N1C[C@@H](C(F)(F)F)NC1=O ; *N1CC(F)(F)CNC1=O]

In some embodiments, disclosed herein is a compound of Formula I, or a pharmaceutically acceptable salt thereof, wherein R¹ is

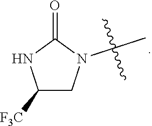 [SMILES: *N1C[C@@H](C(F)(F)F)NC1=O]

In some embodiments, disclosed herein is a compound of Formula I, or a pharmaceutically acceptable salt thereof, wherein R¹ is

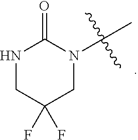 [SMILES: *N1CC(F)(F)CNC1=O]

In some embodiments, disclosed herein is a compound of Formula I, or a pharmaceutically acceptable salt thereof, wherein R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to four R^{2a} groups; and R^{2a} independently for each occurrence is fluorine or —CN.

In some embodiments, disclosed herein is a compound of Formula I, or a pharmaceutically acceptable salt thereof, wherein R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one —CN group.

In some embodiments, disclosed herein is a compound of Formula I, or a pharmaceutically acceptable salt thereof, wherein R² is H, —C₍₁₋₃₎alkyl, —C₍₃₋₄₎cycloalkyl, —C₍₁₋₃₎alkyl-O—C₍₁₋₃₎alkyl, or —C₍₁₋₃₎alkyl-O—C₍₃₋₄₎cycloalkyl, wherein the —C₍₃₋₄₎cycloalkyl is unsubstituted or substituted with one —CN group.

In some embodiments, disclosed herein is a compound of Formula I, or a pharmaceutically acceptable salt thereof, wherein R² is H, —C₍₁₋₃₎alkyl, —C₍₃₋₄₎cycloalkyl, or —C₍₁₋₃₎alkyl-O—C₍₁₋₃₎alkyl, or —CH₂—O—C₍₃₋₄₎cycloalkyl wherein the —C₍₃₋₄₎cycloalkyl is unsubstituted or substituted with one —CN group.

In some embodiments, disclosed herein is a compound of Formula I, or a pharmaceutically acceptable salt thereof, wherein R² is H, methyl,

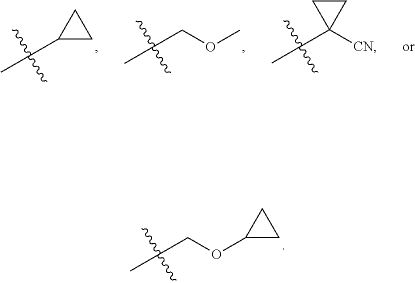

In some embodiments, disclosed herein is a compound of Formula I, or a pharmaceutically acceptable salt thereof, wherein R² is

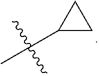 [SMILES: *C1CC1]

In some embodiments, disclosed herein is a compound of Formula I, or a pharmaceutically acceptable salt thereof, wherein R² is

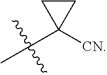 [SMILES: *C1(C#N)CC1]

In some embodiments, disclosed herein is a compound of Formula I, or a pharmaceutically acceptable salt thereof, which is a compound of Formula Ia:

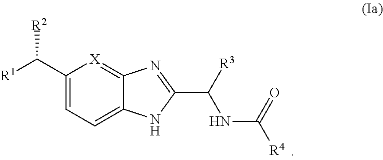

In some embodiments, disclosed herein is a compound of Formula I, or a pharmaceutically acceptable salt thereof, which is a compound of Formula Iaa:

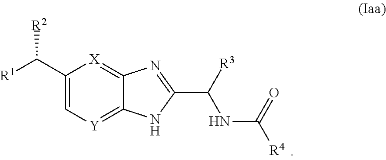

In some embodiments, disclosed herein is a compound of Formula I, or a pharmaceutically acceptable salt thereof, which is a compound of Formula Ib:

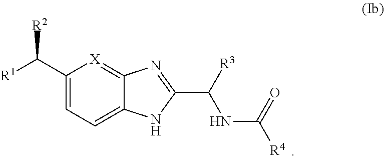

In some embodiments, disclosed herein is a compound of Formula I, or a pharmaceutically acceptable salt thereof, which is a compound of Formula Ibb:

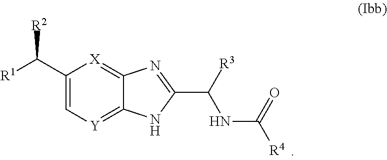

In some embodiments, disclosed herein is a compound of Formula I, or a pharmaceutically acceptable salt thereof, wherein R³ is C₍₁₋₆₎alkyl, —C₍₁₋₄₎alkyl-O—C₍₁₋₄₎alkyl, or —C₍₁₋₄₎alkyl-O—C₍₃₋₄₎cycloalkyl, wherein the C₍₁₋₆₎alkyl and —C₍₁₋₄₎alkyl-O—C₍₁₋₄₎alkyl are unsubstituted or substituted with one to six fluorine atoms, and wherein the —C₍₁₋₄₎alkyl-O—C₍₃₋₄₎cycloalkyl is unsubstituted or substituted with one to four fluorine atoms.

In some embodiments, disclosed herein is a compound of Formula I, or a pharmaceutically acceptable salt thereof, wherein R³ is C₍₁₋₈₎alkyl that is unsubstituted or substituted with one to six fluorine atoms. In some embodiments, disclosed herein is a compound of Formula I, or a pharmaceutically acceptable salt thereof, wherein R³ is C₍₁₋₆₎alkyl that is unsubstituted or substituted with one to six fluorine atoms.

In some embodiments, disclosed herein is a compound of Formula I, or a pharmaceutically acceptable salt thereof, wherein R³ is —C₍₁₋₆₎alkyl-O—C₍₁₋₆₎alkyl or —C₍₁₋₆₎alkyl-O—C₍₃₋₅₎cycloalkyl, wherein the —C₍₁₋₆₎alkyl-O—C₍₁₋₆₎alkyl is unsubstituted or substituted with one to six fluorine atoms, and wherein the —C₍₁₋₆₎alkyl-O—C₍₃₋₅₎cycloalkyl is unsubstituted or substituted with one to four fluorine atoms.

In some embodiments, disclosed herein is a compound of Formula I, or a pharmaceutically acceptable salt thereof, wherein R³ is —C₍₁₋₄₎alkyl-O—C₍₁₋₄₎alkyl or —C₍₁₋₄₎alkyl-O—C₍₃₋₄₎cycloalkyl, wherein the —C₍₁₋₄₎alkyl-O—C₍₁₋₄₎alkyl is unsubstituted or substituted with one to six fluorine atoms, and wherein the —C₍₁₋₄₎alkyl-O—C₍₃₋₄₎cycloalkyl is unsubstituted or substituted with one to four fluorine atoms.

In some embodiments, disclosed herein is a compound of Formula I, or a pharmaceutically acceptable salt thereof, wherein R³ is —C₍₁₋₄₎alkyl-O—C₍₁₋₄₎alkyl or —C₍₁₋₄₎alkyl-O—C₍₃₋₄₎cycloalkyl, wherein the —C₍₁₋₄₎alkyl-O—C₍₁₋₄₎alkyl is unsubstituted or substituted with one to six fluorine atoms.

In some embodiments, disclosed herein is a compound of Formula I, or a pharmaceutically acceptable salt thereof, wherein R³ is —C₍₁₋₄₎alkyl-O—C₍₁₋₆₎alkyl that is unsubstituted or substituted with one to six fluorine atoms.

In some embodiments, disclosed herein is a compound of Formula I, or a pharmaceutically acceptable salt thereof, wherein R³ is —C₍₁₋₆₎alkyl-O—C₍₁₋₅₎alkyl-CF₃ or -C₍₂₋₆₎alkyl-O-cyclopropyl. In some embodiments, disclosed herein is a compound of Formula I, or a pharmaceutically acceptable salt thereof, wherein R³ is —C₍₁₋₄₎alkyl-O—C₍₁₋₃₎alkyl-CF₃ or -C₍₁₋₄₎alkyl-O-cyclopropyl.

In some embodiments, disclosed herein is a compound of Formula I, or a pharmaceutically acceptable salt thereof, wherein R³ is —C₍₁₋₆₎alkyl-O—C₍₁₋₅₎alkyl-CF₃. In some embodiments, disclosed herein is a compound of Formula I, or a pharmaceutically acceptable salt thereof, wherein R³ is —C₍₁₋₄₎alkyl-O—C₍₁₋₃₎alkyl-CF₃.

In some embodiments, disclosed herein is a compound of Formula I, or a pharmaceutically acceptable salt thereof, wherein R³ is —C₍₁₋₆₎alkyl-O-cyclopropyl. In some embodiments, disclosed herein is a compound of Formula I, or a pharmaceutically acceptable salt thereof, wherein R³ is —C₍₁₋₄₎alkyl-O-cyclopropyl.

In some embodiments, disclosed herein is a compound of Formula I, or a pharmaceutically acceptable salt thereof, wherein R³ is

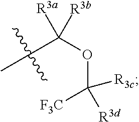

and
R^{3a}, R^{3b}, R^{3c}, and R^{3d} are each independently H or CH₃. In some embodiments, disclosed herein is a compound of Formula I, or a pharmaceutically acceptable salt thereof, wherein at least one of R^{3a}, R^{3b}, R^{3c}, and R^{3d} is CH₃. In some embodiments, disclosed herein is a compound of Formula I, or a pharmaceutically acceptable salt thereof, wherein at least two of R^{3a}, R^{3b}, R^{3c}, and R^{3d} is CH₃.

In some embodiments, disclosed herein is a compound of Formula I, or a pharmaceutically acceptable salt thereof, wherein R³ is

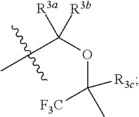

and
R^{3a}, R^{3b}, and R^{3c} are each independently H or CH₃. In some embodiments, disclosed herein is a compound of Formula I, or a pharmaceutically acceptable salt thereof, wherein at least one of R^{3a}, R^{3b}, and R^{3c} is CH₃.

In some embodiments, disclosed herein is a compound of Formula I, or a pharmaceutically acceptable salt thereof, wherein R³ is:

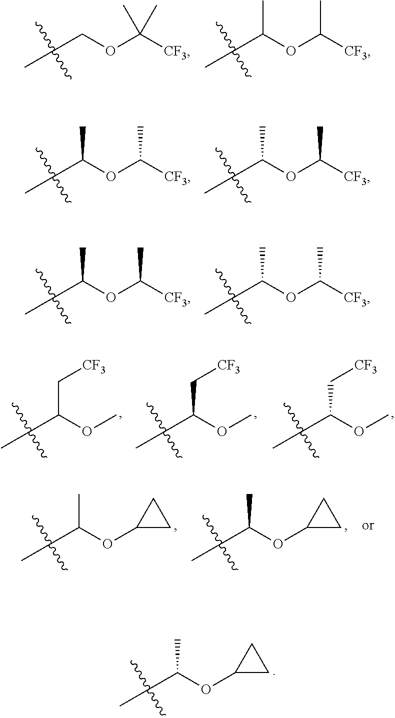

In some embodiments, disclosed herein is a compound of Formula I, or a pharmaceutically acceptable salt thereof, wherein R³ is:

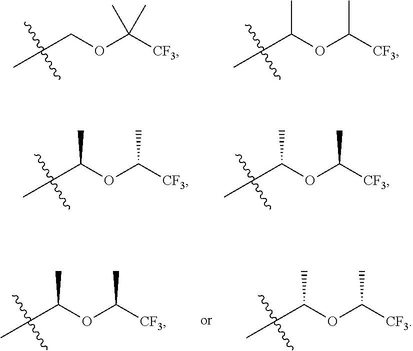

In some embodiments, disclosed herein is a compound of Formula I, or a pharmaceutically acceptable salt thereof, wherein R³ is

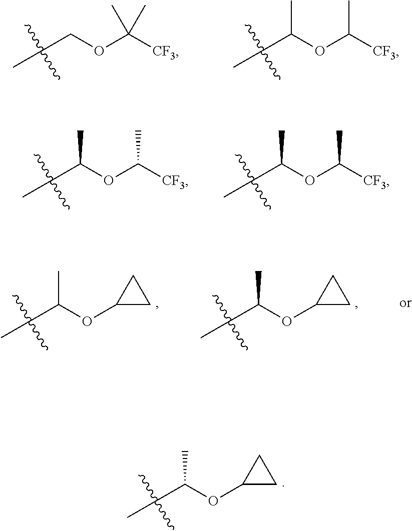

In some embodiments, disclosed herein is a compound of Formula I, or a pharmaceutically acceptable salt thereof, wherein R³ is

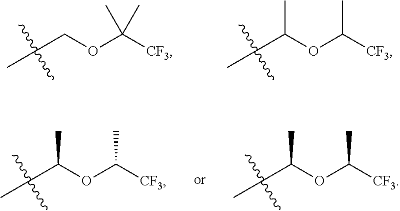

In some embodiments, disclosed herein is a compound of Formula I, or a pharmaceutically acceptable salt thereof, wherein R³ is

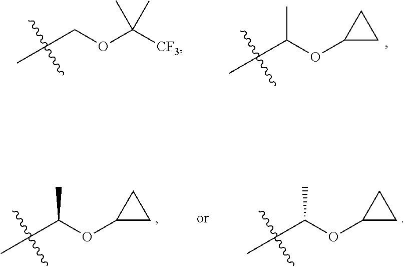

In some embodiments, disclosed herein is a compound of Formula I, or a pharmaceutically acceptable salt thereof, wherein R³ is

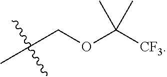 [SMILES: *COC(C)(C)C(F)(F)F]

In some embodiments, disclosed herein is a compound of Formula I, or a pharmaceutically acceptable salt thereof, wherein R³ is

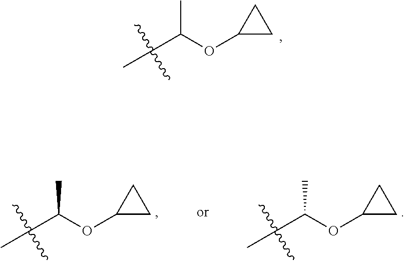

In some embodiments, disclosed herein is a compound of Formula I, or a pharmaceutically acceptable salt thereof, wherein R³ is —C₍₁₋₆₎alkyl-O—C₍₃₋₅₎cycloalkyl that is unsubstituted or substituted with one to four fluorine atoms. In some embodiments, disclosed herein is a compound of Formula I, or a pharmaceutically acceptable salt thereof, wherein R³ is —C₍₁₋₄₎alkyl-O—C₍₃₋₄₎cycloalkyl that is unsubstituted or substituted with one to four fluorine atoms.

In some embodiments, disclosed herein is a compound of Formula I, or a pharmaceutically acceptable salt thereof, which is a compound of Formula Ic:

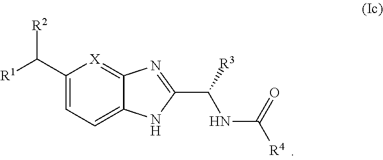

In some embodiments, disclosed herein is a compound of Formula I, or a pharmaceutically acceptable salt thereof, which is a compound of Formula Icc:

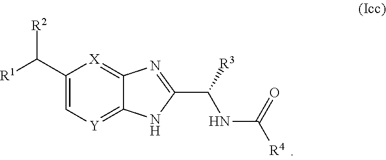

In some embodiments, disclosed herein is a compound of Formula I, or a pharmaceutically acceptable salt thereof, which is a compound of Formula Ic-1:

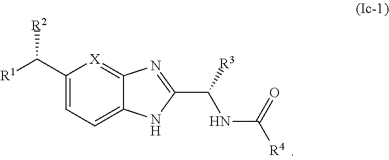

In some embodiments, disclosed herein is a compound of Formula I, or a pharmaceutically acceptable salt thereof, which is a compound of Formula Icc-1:

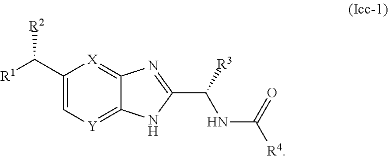

In some embodiments, disclosed herein is a compound of Formula I, or a pharmaceutically acceptable salt thereof, which is a compound of Formula Id:

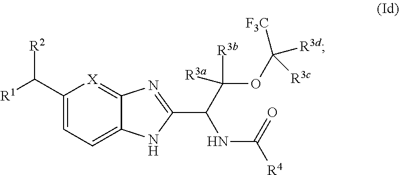

wherein R^{3a}, R^{3b}, R^{3c}, and R^{3d} are each independently H or CH₃.

In some embodiments, disclosed herein is a compound of Formula I, or a pharmaceutically acceptable salt thereof, which is a compound of Formula Idd:

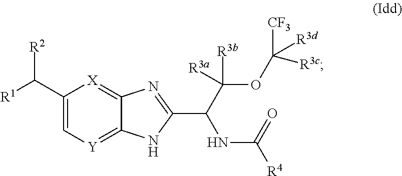

wherein R^{3a}, R^{3b}, R^{3c}, and R^{3d} are each independently H or CH₃.

In some embodiments, disclosed herein is a compound of Formula I, or a pharmaceutically acceptable salt thereof, which is a compound of Formula Id-1:

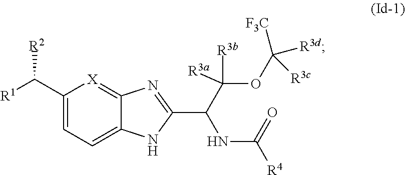

wherein R^{3a}, R^{3b}, R^{3c}, and R^{3d} are each independently H or CH₃.

In some embodiments, disclosed herein is a compound of Formula I, or a pharmaceutically acceptable salt thereof, which is a compound of Formula Idd-1:

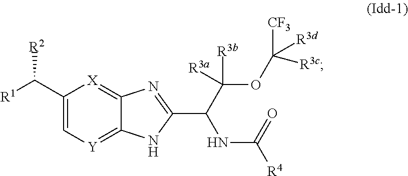

wherein R^{3a}, R^{3b}, R^{3c}, and R^{3d} are each independently H or CH₃.

In some embodiments, disclosed herein is a compound of Formula I, or a pharmaceutically acceptable salt thereof, which is a compound of Formula Id-2:

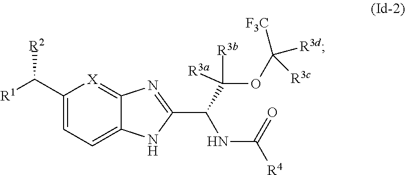

wherein R^{3a}, R^{3b}, R^{3c}, and R^{3d} are each independently H or CH₃.

In some embodiments, disclosed herein is a compound of Formula I, or a pharmaceutically acceptable salt thereof, which is a compound of Formula Idd-2:

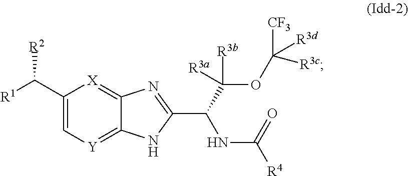

wherein R^{3a}, R^{3b}, R^{3c}, and R^{3d} are each independently H or CH₃.

In some embodiments, disclosed herein is a compound of Formula I, or a pharmaceutically acceptable salt thereof, wherein R⁴ is a 5-membered heteroaryl that is substituted with one to two R^{4a} groups.

In some embodiments, disclosed herein is a compound of Formula I, or a pharmaceutically acceptable salt thereof, wherein R⁴ is a 5-membered heteroaryl comprising one to three heteroatoms selected from O and N, wherein the 5-membered heteroaryl is unsubstituted or substituted with one to two R^{4a} groups.

In some embodiments, disclosed herein is a compound of Formula I, or a pharmaceutically acceptable salt thereof, wherein R⁴ is pyrrolyl, pyrazolyl, imidazolyl, triazolyl, oxazolyl, isoxazolyl, or oxadiazolyl, each of which is unsubstituted or substituted with one to two R^{4a} groups. In some embodiments, disclosed herein is a compound of Formula I, or a pharmaceutically acceptable salt thereof, wherein R⁴ is pyrazolyl, triazolyl, or oxadiazolyl each of which is unsubstituted or substituted with one to two R^{4a} groups.

In some embodiments, disclosed herein is a compound of Formula I, or a pharmaceutically acceptable salt thereof, wherein R⁴ is pyrrolyl, pyrazolyl, imidazolyl, 1,2,3-triazolyl, 1,2,4-triazolyl, oxazolyl, isoxazolyl, 1,2,3-oxadiazolyl, 1,2,4-oxadizaolyl, 1,2,5-oxadiazolyl, or 1,3,4-oxadiazolyl, each of which is unsubstituted or substituted with one to two R^{4a} groups. In some embodiments, disclosed herein is a compound of Formula I, or a pharmaceutically acceptable salt thereof, wherein R⁴ is pyrazolyl, 1,2,4-triazolyl, or 1,2,5-oxadiazolyl, each of which is unsubstituted or substituted with one to two R^{4a} groups.

In some embodiments, disclosed herein is a compound of Formula I, or a pharmaceutically acceptable salt thereof, wherein R⁴ is:

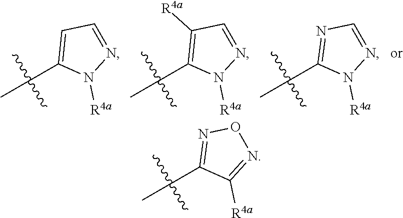

In some embodiments, disclosed herein is a compound of Formula I, or a pharmaceutically acceptable salt thereof, wherein R^{4a} is halo, —C₍₁₋₆₎alkyl, or —C₍₀₋₂₎alkyl-C₍₃₋₆₎cycloalkyl, wherein the —C₍₁₋₆₎alkyl and —C₍₀₋₂₎alkyl-C₍₃₋₆₎cycloalkyl are unsubstituted or substituted with one to six substituents independently selected from fluorine and —CN.

In some embodiments, disclosed herein is a compound of Formula I, or a pharmaceutically acceptable salt thereof, wherein R^{4a} is fluorine, —C₍₁₋₆₎alkyl, or —C₍₀₋₁₎alkyl-C₍₃₋₄₎cycloalkyl, wherein the —C₍₁₋₆₎alkyl and —C₍₀₋₁₎alkyl-C₍₃₋₄₎cycloalkyl are unsubstituted or substituted with one to six substituents independently selected from fluorine and —CN.

In some embodiments, disclosed herein is a compound of Formula I, or a pharmaceutically acceptable salt thereof, wherein R^{4a} is fluorine, —C₍₁₋₆₎alkyl, or —C₍₀₋₁₎alkyl-C₍₃₋₄₎cycloalkyl. In some embodiments, disclosed herein is a compound of Formula I, or a pharmaceutically acceptable salt thereof, wherein R^{4a} is fluorine, —C₍₁₋₃₎alkyl, or —C₍₃₋₄₎cycloalkyl.

In some embodiments, disclosed herein is a compound of Formula I, or a pharmaceutically acceptable salt thereof, wherein R^{4a} is fluorine, methyl, isopropyl, —CD₂CD₃, or cyclopropyl.

In some embodiments, disclosed herein is a compound of Formula I, or a pharmaceutically acceptable salt thereof, wherein R⁴ is:

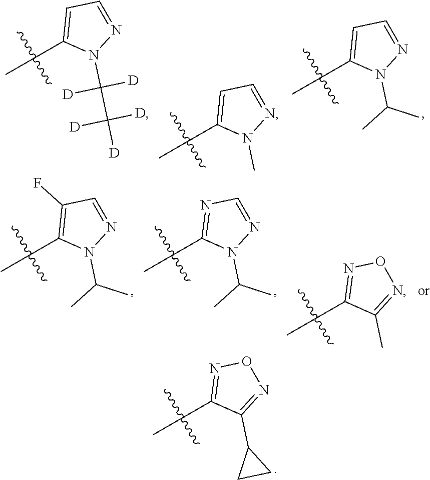

In some embodiments, disclosed herein is a compound of Formula I, or a pharmaceutically acceptable salt thereof, wherein X is C.

In some embodiments, disclosed herein is a compound of Formula I, or a pharmaceutically acceptable salt thereof, wherein X is N.

In some embodiments, disclosed herein is a compound of Formula I, or a pharmaceutically acceptable salt thereof, which is a compound of Formula Ie:

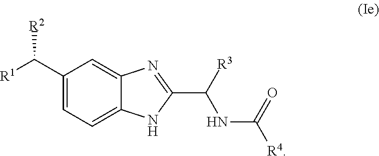

In some embodiments, disclosed herein is a compound of Formula I, or a pharmaceutically acceptable salt thereof, which is a compound of Formula Ie-1:

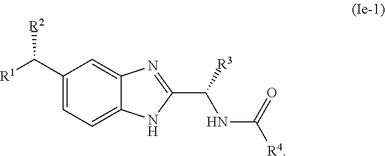

In some embodiments, disclosed herein is a compound of Formula I, or a pharmaceutically acceptable salt thereof, which is a compound of Formula Ie-2:

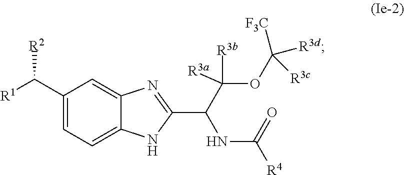

wherein R^{3a}, R^{3b}, R^{3c}, and R^{3d} are each independently H or CH₃.

In some embodiments, disclosed herein is a compound of Formula I, or a pharmaceutically acceptable salt thereof, which is a compound of Formula If:

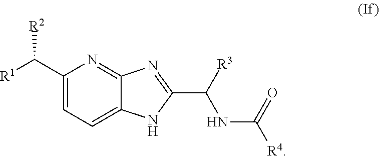

In some embodiments, disclosed herein is a compound of Formula I, or a pharmaceutically acceptable salt thereof, which is a compound of Formula If-1:

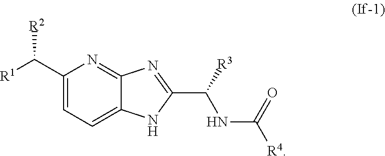

In some embodiments, disclosed herein is a compound of Formula I, or a pharmaceutically acceptable salt thereof, which is a compound of Formula If-2:

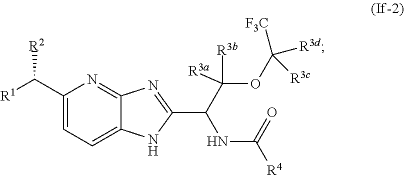

wherein R^{3a}, R^{3b}, R^{3c}, and R^{3d} are each independently H or CH₃.

In some embodiments, disclosed herein is a compound of Formula I, or a pharmaceutically acceptable salt thereof, that is a deuterated isotope comprising one to thirty deuterium atoms. In some embodiments, disclosed herein is a compound of Formula I, or a pharmaceutically acceptable salt thereof, that is a deuterated isotope comprising one to fifteen deuterium atoms. In some embodiments, disclosed herein is a compound of Formula I, or a pharmaceutically acceptable salt thereof, that is a deuterated isotope comprising one to seven deuterium atoms.

In some embodiments, disclosed herein is a compound of Formula (I), or a pharmaceutically acceptable salt thereof, having a structure as shown in Table 1A, Table 1B, Table 1C, Table 1D or Table 1E.

TABLE 1A

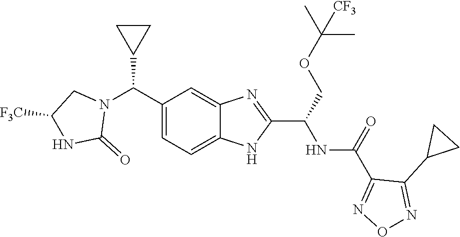 [SMILES: CC(C)(OC[C@H](NC(=O)c1nonc1C1CC1)c1nc2cc([C@@H](C3CC3)N3C[C@@H](C(F)(F)F)NC3=O)ccc2[nH]1)C(F)(F)F]

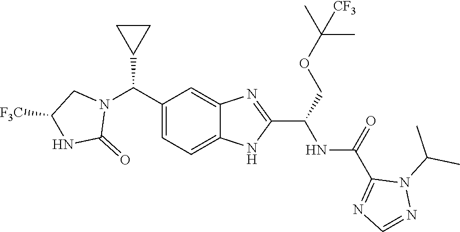 [SMILES: CC(C)n1ncnc1C(=O)N[C@@H](COC(C)(C)C(F)(F)F)c1nc2cc([C@@H](C3CC3)N3C[C@@H](C(F)(F)F)NC3=O)ccc2[nH]1]

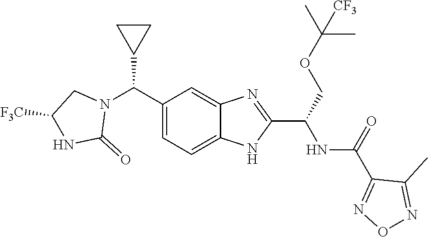 [SMILES: Cc1nonc1C(=O)N[C@@H](COC(C)(C)C(F)(F)F)c1nc2cc([C@@H](C3CC3)N3C[C@@H](C(F)(F)F)NC3=O)ccc2[nH]1]

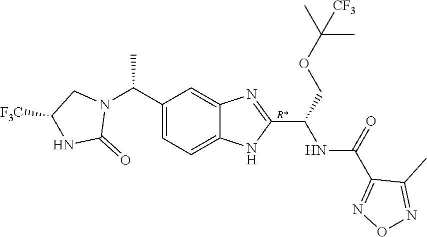 [SMILES: Cc1nonc1C(=O)N[C@@H](COC(C)(C)C(F)(F)F)c1nc2cc([C@@H](C)N3C[C@@H](C(F)(F)F)NC3=O)ccc2[nH]1]

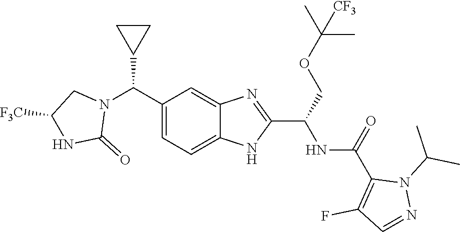 [SMILES: CC(C)n1ncc(F)c1C(=O)N[C@@H](COC(C)(C)C(F)(F)F)c1nc2cc([C@@H](C3CC3)N3C[C@@H](C(F)(F)F)NC3=O)ccc2[nH]1]

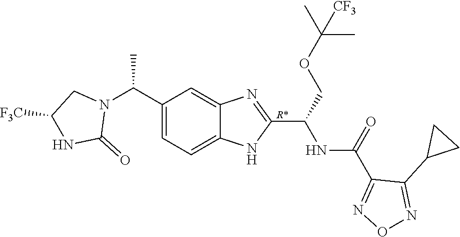 [SMILES: C[C@H](c1ccc2[nH]c([C@H](COC(C)(C)C(F)(F)F)NC(=O)c3nonc3C3CC3)nc2c1)N1C[C@@H](C(F)(F)F)NC1=O]

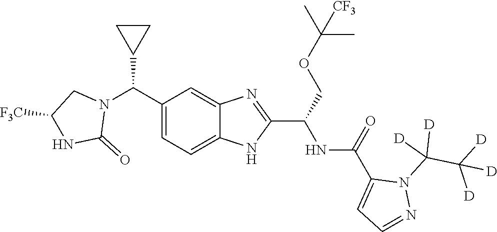 [SMILES: [2H]C([2H])([2H])C([2H])([2H])n1nccc1C(=O)N[C@@H](COC(C)(C)C(F)(F)F)c1nc2cc([C@@H](C3CC3)N3C[C@@H](C(F)(F)F)NC3=O)ccc2[nH]1]

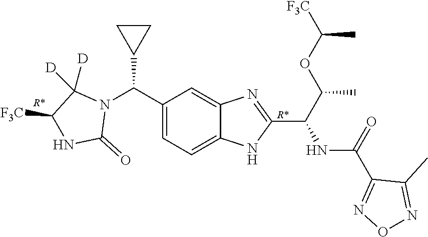 [SMILES: [2H]C1([2H])[C@H](C(F)(F)F)NC(=O)N1[C@@H](c1ccc2[nH]c([C@@H](NC(=O)c3nonc3C)[C@@H](C)O[C@H](C)C(F)(F)F)nc2c1)C1CC1]

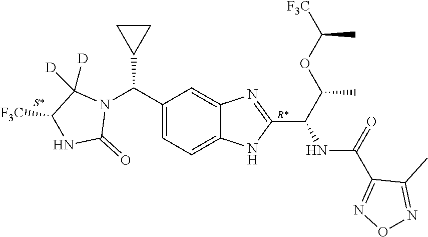 [SMILES: [2H]C1([2H])[C@@H](C(F)(F)F)NC(=O)N1[C@@H](c1ccc2[nH]c([C@@H](NC(=O)c3nonc3C)[C@@H](C)O[C@H](C)C(F)(F)F)nc2c1)C1CC1]

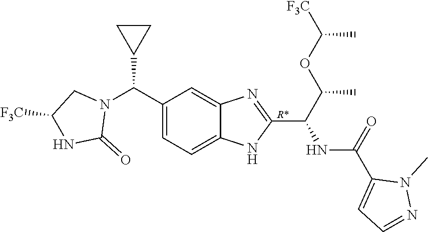 [SMILES: C[C@H](O[C@H](C)[C@H](NC(=O)c1ccnn1C)c1nc2cc([C@@H](C3CC3)N3C[C@@H](C(F)(F)F)NC3=O)ccc2[nH]1)C(F)(F)F]

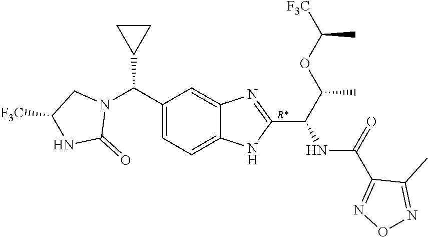 [SMILES: Cc1nonc1C(=O)N[C@H](c1nc2cc([C@@H](C3CC3)N3C[C@@H](C(F)(F)F)NC3=O)ccc2[nH]1)[C@@H](C)O[C@H](C)C(F)(F)F]

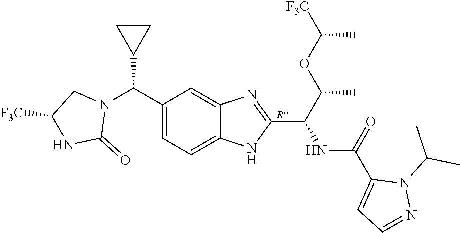 [SMILES: CC(C)n1nccc1C(=O)N[C@H](c1nc2cc([C@@H](C3CC3)N3C[C@@H](C(F)(F)F)NC3=O)ccc2[nH]1)[C@@H](C)O[C@@H](C)C(F)(F)F]

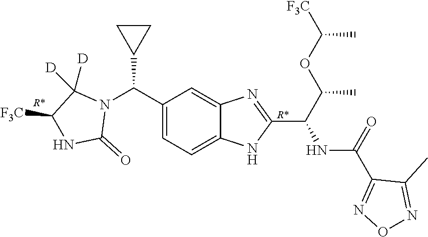 [SMILES: [2H]C1([2H])[C@H](C(F)(F)F)NC(=O)N1[C@@H](c1ccc2[nH]c([C@@H](NC(=O)c3nonc3C)[C@@H](C)O[C@@H](C)C(F)(F)F)nc2c1)C1CC1]

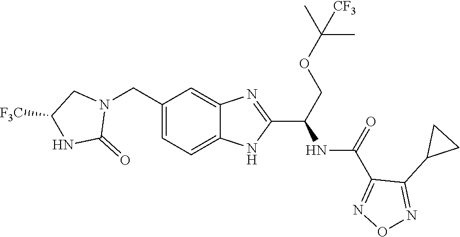 [SMILES: CC(C)(OC[C@@H](NC(=O)c1nonc1C1CC1)c1nc2cc(CN3C[C@@H](C(F)(F)F)NC3=O)ccc2[nH]1)C(F)(F)F]

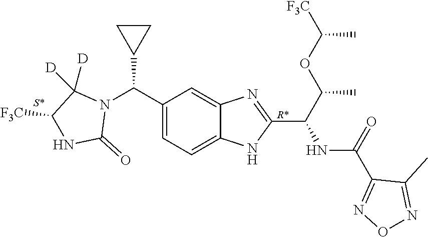 [SMILES: [2H]C1([2H])[C@@H](C(F)(F)F)NC(=O)N1[C@@H](c1ccc2[nH]c([C@@H](NC(=O)c3nonc3C)[C@@H](C)O[C@@H](C)C(F)(F)F)nc2c1)C1CC1]

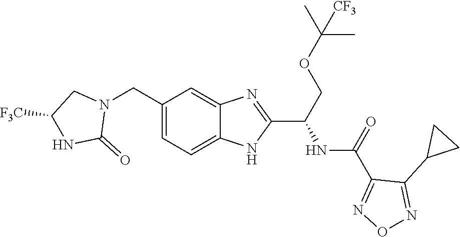 [SMILES: CC(C)(OC[C@H](NC(=O)c1nonc1C1CC1)c1nc2cc(CN3C[C@@H](C(F)(F)F)NC3=O)ccc2[nH]1)C(F)(F)F]

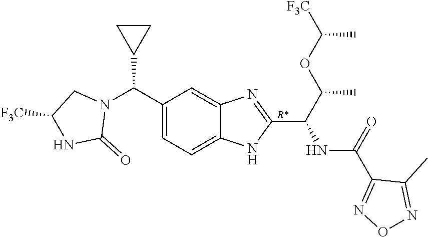 [SMILES: Cc1nonc1C(=O)N[C@H](c1nc2cc([C@@H](C3CC3)N3C[C@@H](C(F)(F)F)NC3=O)ccc2[nH]1)[C@@H](C)O[C@@H](C)C(F)(F)F]

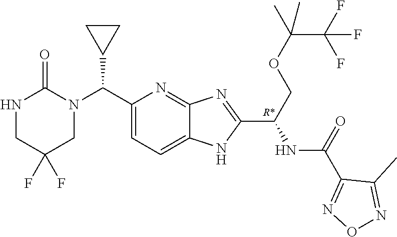 [SMILES: Cc1nonc1C(=O)N[C@@H](COC(C)(C)C(F)(F)F)c1nc2nc([C@@H](C3CC3)N3CC(F)(F)CNC3=O)ccc2[nH]1]

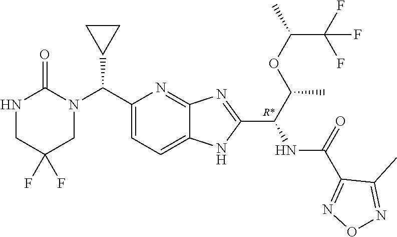 [SMILES: Cc1nonc1C(=O)N[C@H](c1nc2nc([C@@H](C3CC3)N3CC(F)(F)CNC3=O)ccc2[nH]1)[C@@H](C)O[C@H](C)C(F)(F)F]

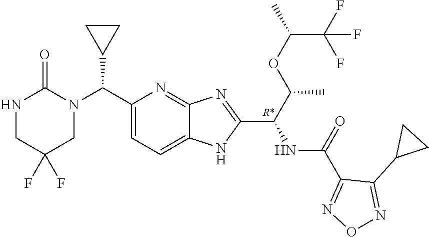 [SMILES: C[C@@H](O[C@H](C)C(F)(F)F)[C@H](NC(=O)c1nonc1C1CC1)c1nc2nc([C@@H](C3CC3)N3CC(F)(F)CNC3=O)ccc2[nH]1]

TABLE 1B

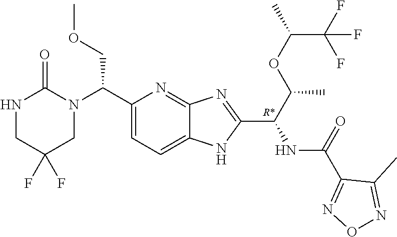 [SMILES: COC[C@H](c1ccc2[nH]c([C@@H](NC(=O)c3nonc3C)[C@@H](C)O[C@H](C)C(F)(F)F)nc2n1)N1CC(F)(F)CNC1=O]

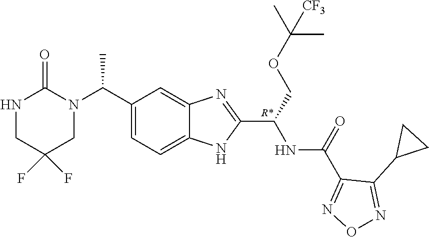 [SMILES: C[C@H](c1ccc2[nH]c([C@H](COC(C)(C)C(F)(F)F)NC(=O)c3nonc3C3CC3)nc2c1)N1CC(F)(F)CNC1=O]

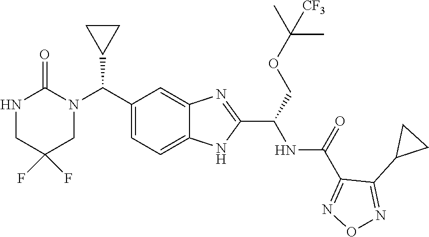 [SMILES: CC(C)(OC[C@H](NC(=O)c1nonc1C1CC1)c1nc2cc([C@@H](C3CC3)N3CC(F)(F)CNC3=O)ccc2[nH]1)C(F)(F)F]

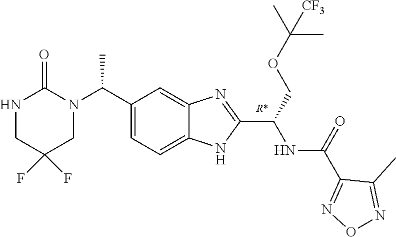 [SMILES: Cc1nonc1C(=O)N[C@@H](COC(C)(C)C(F)(F)F)c1nc2cc([C@@H](C)N3CC(F)(F)CNC3=O)ccc2[nH]1]

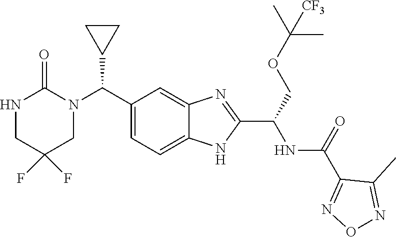 [SMILES: Cc1nonc1C(=O)N[C@@H](COC(C)(C)C(F)(F)F)c1nc2cc([C@@H](C3CC3)N3CC(F)(F)CNC3=O)ccc2[nH]1]

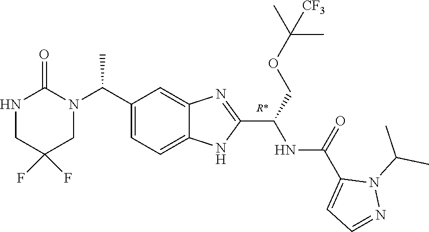 [SMILES: CC(C)n1nccc1C(=O)N[C@@H](COC(C)(C)C(F)(F)F)c1nc2cc([C@@H](C)N3CC(F)(F)CNC3=O)ccc2[nH]1]

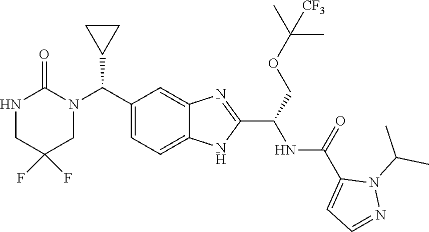 [SMILES: CC(C)n1nccc1C(=O)N[C@@H](COC(C)(C)C(F)(F)F)c1nc2cc([C@@H](C3CC3)N3CC(F)(F)CNC3=O)ccc2[nH]1]

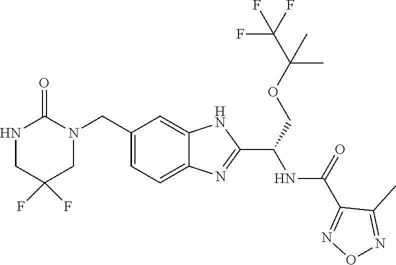 [SMILES: Cc1nonc1C(=O)N[C@@H](COC(C)(C)C(F)(F)F)c1nc2ccc(CN3CC(F)(F)CNC3=O)cc2[nH]1]

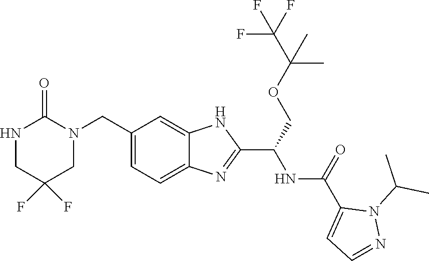 [SMILES: CC(C)n1nccc1C(=O)N[C@@H](COC(C)(C)C(F)(F)F)c1nc2ccc(CN3CC(F)(F)CNC3=O)cc2[nH]1]

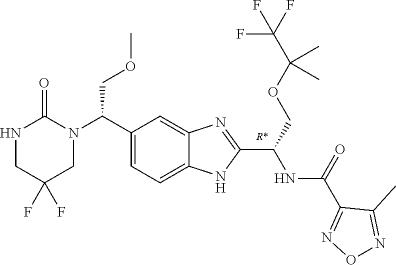 [SMILES: COC[C@H](c1ccc2[nH]c([C@H](COC(C)(C)C(F)(F)F)NC(=O)c3nonc3C)nc2c1)N1CC(F)(F)CNC1=O]

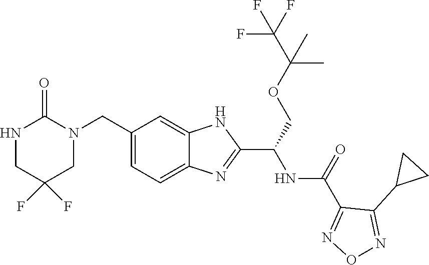 [SMILES: CC(C)(OC[C@H](NC(=O)c1nonc1C1CC1)c1nc2ccc(CN3CC(F)(F)CNC3=O)cc2[nH]1)C(F)(F)F]

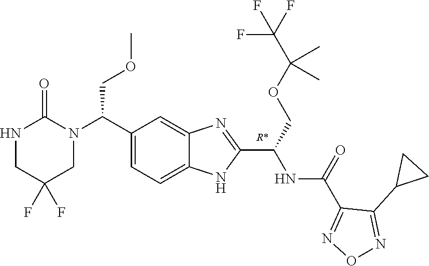 [SMILES: COC[C@H](c1ccc2[nH]c([C@H](COC(C)(C)C(F)(F)F)NC(=O)c3nonc3C3CC3)nc2c1)N1CC(F)(F)CNC1=O]

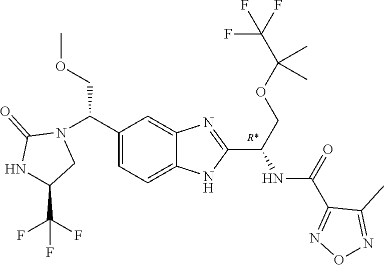 [SMILES: COC[C@H](c1ccc2[nH]c([C@H](COC(C)(C)C(F)(F)F)NC(=O)c3nonc3C)nc2c1)N1C[C@@H](C(F)(F)F)NC1=O]

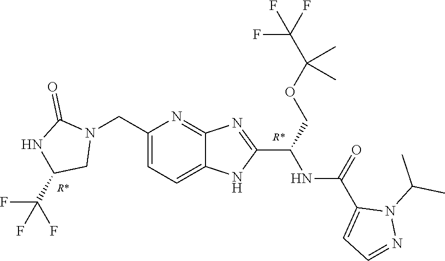 [SMILES: CC(C)n1nccc1C(=O)N[C@@H](COC(C)(C)C(F)(F)F)c1nc2nc(CN3C[C@H](C(F)(F)F)NC3=O)ccc2[nH]1]

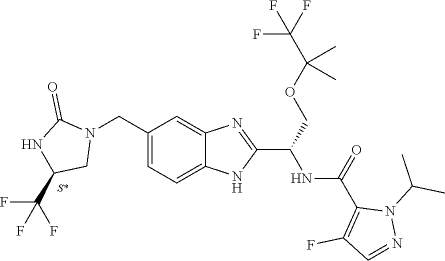 [SMILES: CC(C)n1ncc(F)c1C(=O)N[C@@H](COC(C)(C)C(F)(F)F)c1nc2cc(CN3C[C@@H](C(F)(F)F)NC3=O)ccc2[nH]1]

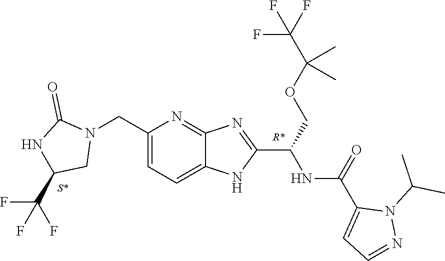 [SMILES: CC(C)n1nccc1C(=O)N[C@@H](COC(C)(C)C(F)(F)F)c1nc2nc(CN3C[C@@H](C(F)(F)F)NC3=O)ccc2[nH]1]

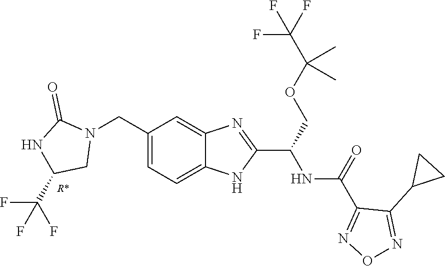 [SMILES: CC(C)(OC[C@H](NC(=O)c1nonc1C1CC1)c1nc2cc(CN3C[C@H](C(F)(F)F)NC3=O)ccc2[nH]1)C(F)(F)F]

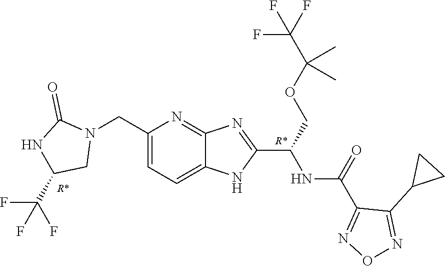 [SMILES: CC(C)(OC[C@H](NC(=O)c1nonc1C1CC1)c1nc2nc(CN3C[C@H](C(F)(F)F)NC3=O)ccc2[nH]1)C(F)(F)F]

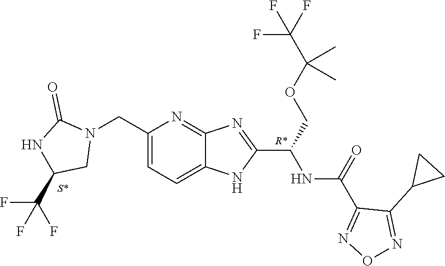 [SMILES: CC(C)(OC[C@H](NC(=O)c1nonc1C1CC1)c1nc2nc(CN3C[C@@H](C(F)(F)F)NC3=O)ccc2[nH]1)C(F)(F)F]

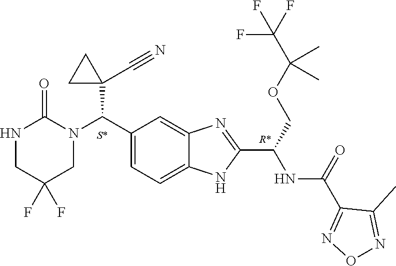 [SMILES: Cc1nonc1C(=O)N[C@@H](COC(C)(C)C(F)(F)F)c1nc2cc([C@H](N3CC(F)(F)CNC3=O)C3(C#N)CC3)ccc2[nH]1]

TABLE 1C

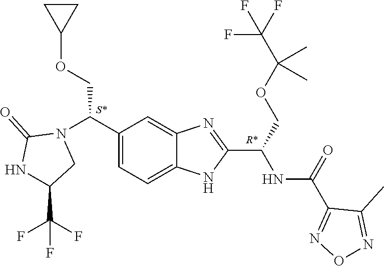 [SMILES: Cc1nonc1C(=O)N[C@@H](COC(C)(C)C(F)(F)F)c1nc2cc([C@@H](COC3CC3)N3C[C@@H](C(F)(F)F)NC3=O)ccc2[nH]1]

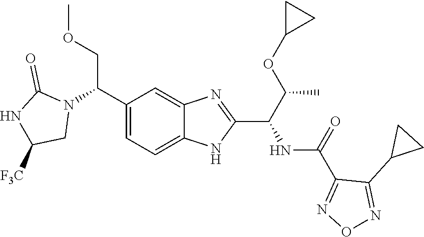 [SMILES: COC[C@H](c1ccc2[nH]c([C@@H](NC(=O)c3nonc3C3CC3)[C@@H](C)OC3CC3)nc2c1)N1C[C@@H](C(F)(F)F)NC1=O]

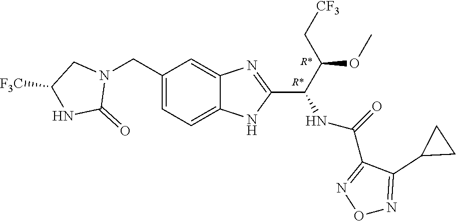 [SMILES: CO[C@H](CC(F)(F)F)[C@H](NC(=O)c1nonc1C1CC1)c1nc2cc(CN3C[C@@H](C(F)(F)F)NC3=O)ccc2[nH]1]

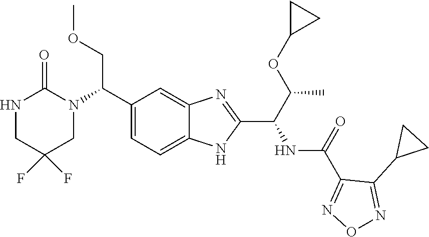 [SMILES: COC[C@H](c1ccc2[nH]c([C@@H](NC(=O)c3nonc3C3CC3)[C@@H](C)OC3CC3)nc2c1)N1CC(F)(F)CNC1=O]

TABLE 1D

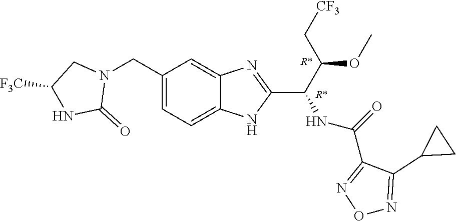 [SMILES: CO[C@H](CC(F)(F)F)[C@H](NC(=O)c1nonc1C1CC1)c1nc2cc(CN3C[C@@H](C(F)(F)F)NC3=O)ccc2[nH]1]

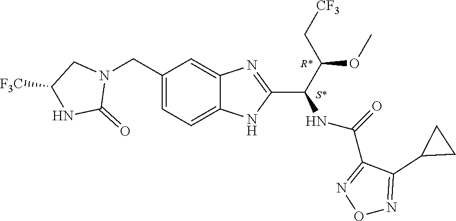 [SMILES: CO[C@H](CC(F)(F)F)[C@@H](NC(=O)c1nonc1C1CC1)c1nc2cc(CN3C[C@@H](C(F)(F)F)NC3=O)ccc2[nH]1]

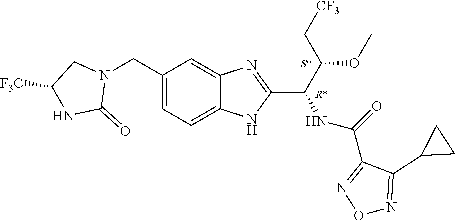 [SMILES: CO[C@@H](CC(F)(F)F)[C@H](NC(=O)c1nonc1C1CC1)c1nc2cc(CN3C[C@@H](C(F)(F)F)NC3=O)ccc2[nH]1]

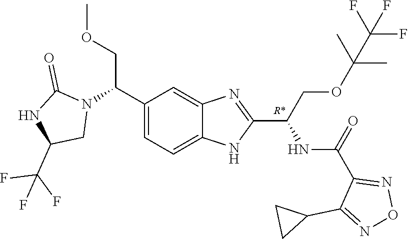 [SMILES: COC[C@H](c1ccc2[nH]c([C@H](COC(C)(C)C(F)(F)F)NC(=O)c3nonc3C3CC3)nc2c1)N1C[C@@H](C(F)(F)F)NC1=O]

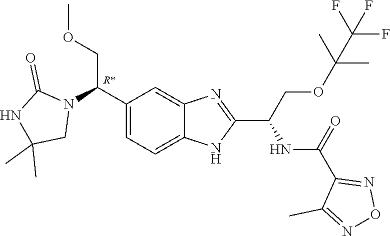 [SMILES: COC[C@@H](c1ccc2[nH]c([C@H](COC(C)(C)C(F)(F)F)NC(=O)c3nonc3C)nc2c1)N1CC(C)(C)NC1=O]

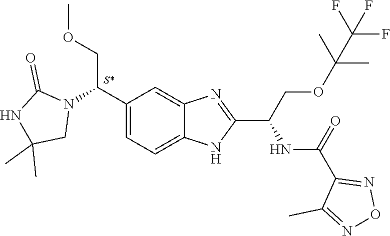 [SMILES: COC[C@H](c1ccc2[nH]c([C@H](COC(C)(C)C(F)(F)F)NC(=O)c3nonc3C)nc2c1)N1CC(C)(C)NC1=O]

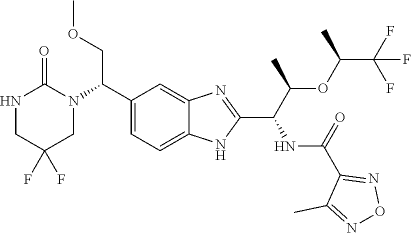 [SMILES: COC[C@H](c1ccc2[nH]c([C@@H](NC(=O)c3nonc3C)[C@@H](C)O[C@@H](C)C(F)(F)F)nc2c1)N1CC(F)(F)CNC1=O]

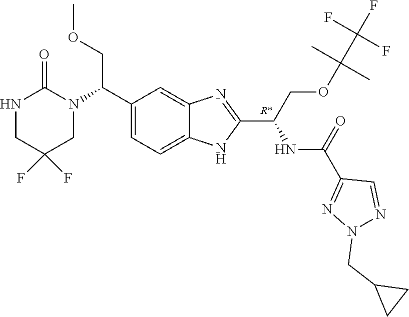 [SMILES: COC[C@H](c1ccc2[nH]c([C@H](COC(C)(C)C(F)(F)F)NC(=O)c3cnn(CC4CC4)n3)nc2c1)N1CC(F)(F)CNC1=O]

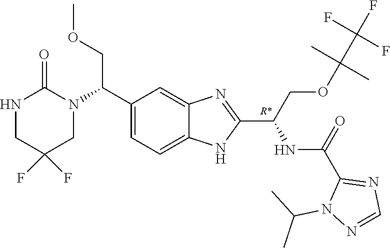 [SMILES: COC[C@H](c1ccc2[nH]c([C@H](COC(C)(C)C(F)(F)F)NC(=O)c3ncnn3C(C)C)nc2c1)N1CC(F)(F)CNC1=O]

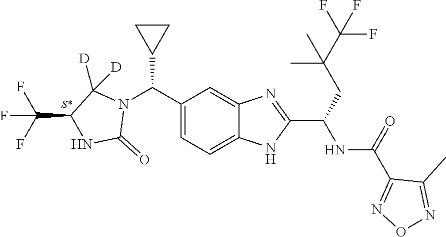 [SMILES: [2H]C1([2H])[C@H](C(F)(F)F)NC(=O)N1[C@@H](c1ccc2[nH]c([C@H](CC(C)(C)C(F)(F)F)NC(=O)c3nonc3C)nc2c1)C1CC1]

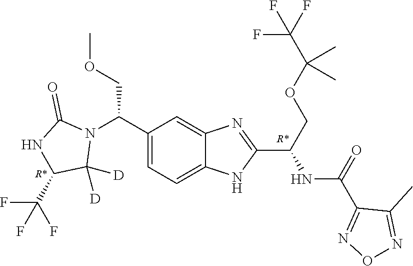 [SMILES: [2H]C1([2H])[C@H](C(F)(F)F)NC(=O)N1[C@H](COC)c1ccc2[nH]c([C@H](COC(C)(C)C(F)(F)F)NC(=O)c3nonc3C)nc2c1]

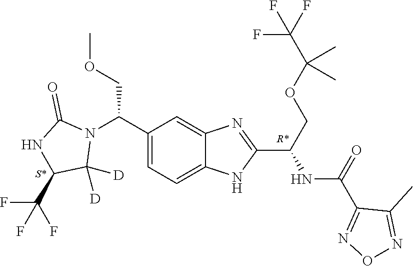 [SMILES: [2H]C1([2H])[C@@H](C(F)(F)F)NC(=O)N1[C@H](COC)c1ccc2[nH]c([C@H](COC(C)(C)C(F)(F)F)NC(=O)c3nonc3C)nc2c1]

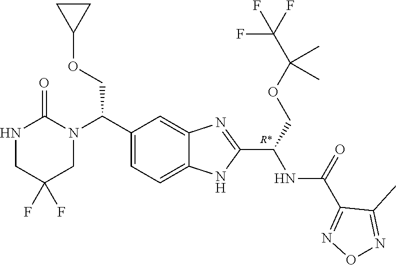 [SMILES: Cc1nonc1C(=O)N[C@@H](COC(C)(C)C(F)(F)F)c1nc2cc([C@@H](COC3CC3)N3CC(F)(F)CNC3=O)ccc2[nH]1]

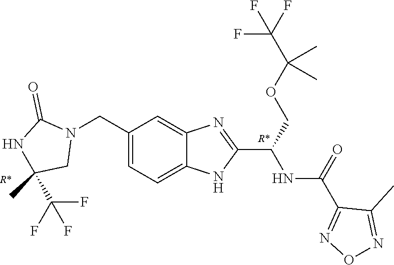

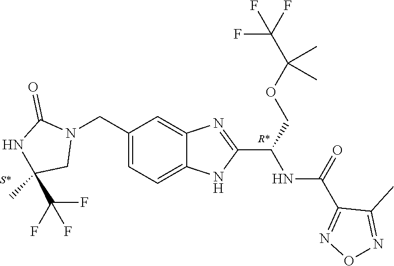

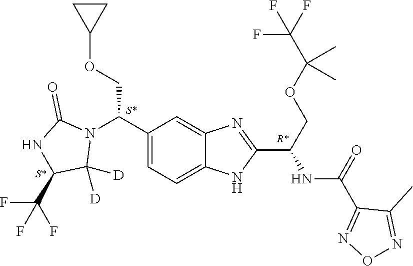 [SMILES: [2H]C1([2H])[C@@H](C(F)(F)F)NC(=O)N1[C@H](COC1CC1)c1ccc2[nH]c([C@H](COC(C)(C)C(F)(F)F)NC(=O)c3nonc3C)nc2c1]

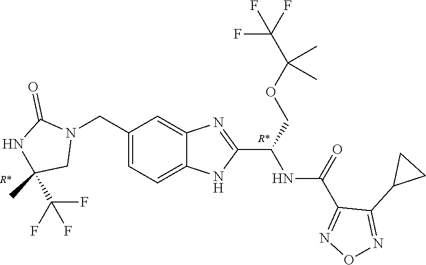 [SMILES: CC(C)(OC[C@H](NC(=O)c1nonc1C1CC1)c1nc2cc(CN3C[C@](C)(C(F)(F)F)NC3=O)ccc2[nH]1)C(F)(F)F]

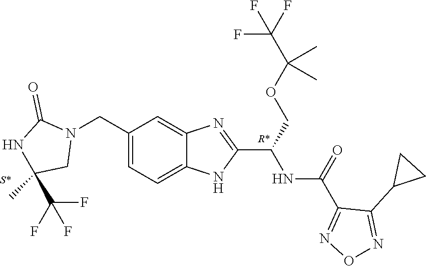 [SMILES: CC(C)(OC[C@H](NC(=O)c1nonc1C1CC1)c1nc2cc(CN3C[C@@](C)(C(F)(F)F)NC3=O)ccc2[nH]1)C(F)(F)F]

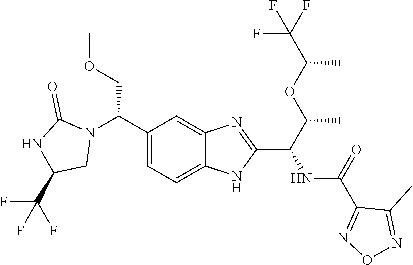 [SMILES: COC[C@H](c1ccc2[nH]c([C@@H](NC(=O)c3nonc3C)[C@@H](C)O[C@@H](C)C(F)(F)F)nc2c1)N1C[C@@H](C(F)(F)F)NC1=O]

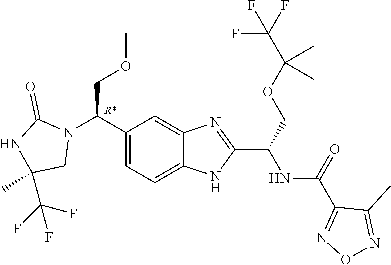

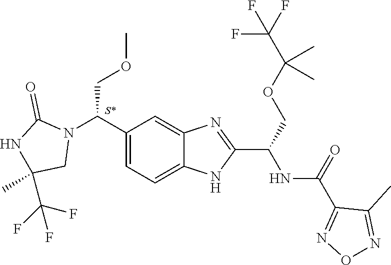

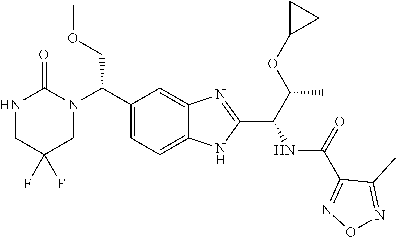 [SMILES: COC[C@H](c1ccc2[nH]c([C@@H](NC(=O)c3nonc3C)[C@@H](C)OC3CC3)nc2c1)N1CC(F)(F)CNC1=O]

TABLE 1E

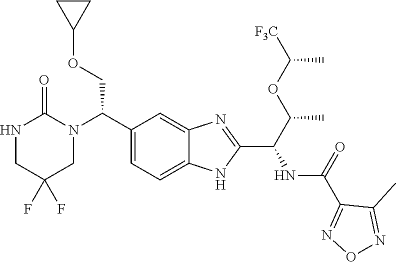 [SMILES: Cc1nonc1C(=O)N[C@H](c1nc2cc([C@@H](COC3CC3)N3CC(F)(F)CNC3=O)ccc2[nH]1)[C@@H](C)O[C@@H](C)C(F)(F)F]

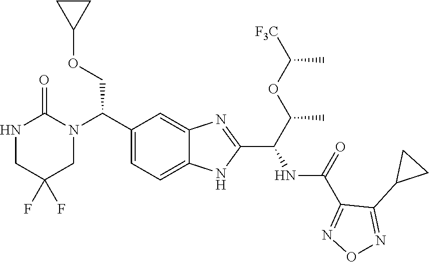 [SMILES: C[C@H](O[C@H](C)[C@H](NC(=O)c1nonc1C1CC1)c1nc2cc([C@@H](COC3CC3)N3CC(F)(F)CNC3=O)ccc2[nH]1)C(F)(F)F]

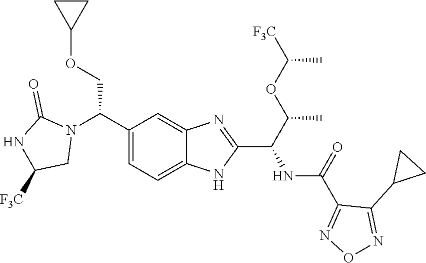 [SMILES: C[C@H](O[C@H](C)[C@H](NC(=O)c1nonc1C1CC1)c1nc2cc([C@@H](COC3CC3)N3C[C@@H](C(F)(F)F)NC3=O)ccc2[nH]1)C(F)(F)F]

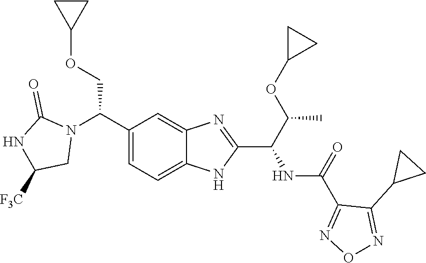 [SMILES: C[C@@H](OC1CC1)[C@H](NC(=O)c1nonc1C1CC1)c1nc2cc([C@@H](COC3CC3)N3C[C@@H](C(F)(F)F)NC3=O)ccc2[nH]1]

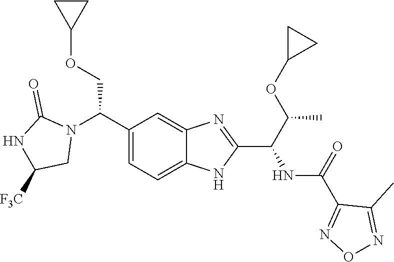 [SMILES: Cc1nonc1C(=O)N[C@H](c1nc2cc([C@@H](COC3CC3)N3C[C@@H](C(F)(F)F)NC3=O)ccc2[nH]1)[C@@H](C)OC1CC1]

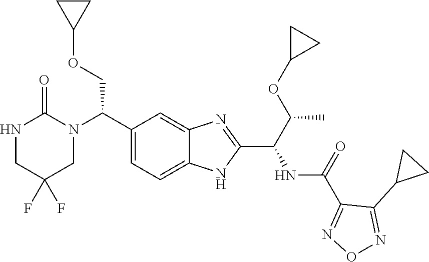 [SMILES: C[C@@H](OC1CC1)[C@H](NC(=O)c1nonc1C1CC1)c1nc2cc([C@@H](COC3CC3)N3CC(F)(F)CNC3=O)ccc2[nH]1]

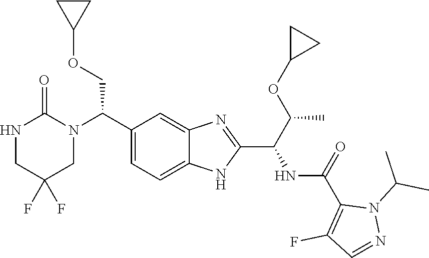 [SMILES: CC(C)n1ncc(F)c1C(=O)N[C@H](c1nc2cc([C@@H](COC3CC3)N3CC(F)(F)CNC3=O)ccc2[nH]1)[C@@H](C)OC1CC1]

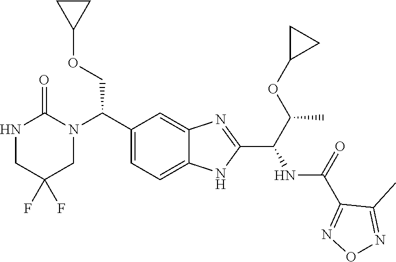 [SMILES: Cc1nonc1C(=O)N[C@H](c1nc2cc([C@@H](COC3CC3)N3CC(F)(F)CNC3=O)ccc2[nH]1)[C@@H](C)OC1CC1]

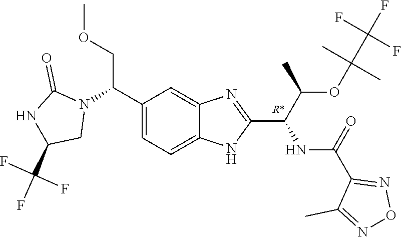 [SMILES: COC[C@H](c1ccc2[nH]c([C@@H](NC(=O)c3nonc3C)[C@@H](C)OC(C)(C)C(F)(F)F)nc2c1)N1C[C@@H](C(F)(F)F)NC1=O]

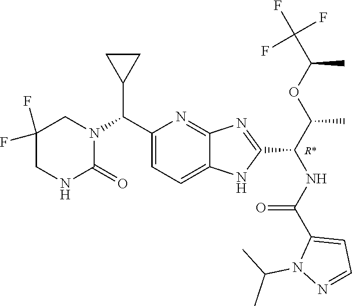 [SMILES: CC(C)n1nccc1C(=O)N[C@H](c1nc2nc([C@@H](C3CC3)N3CC(F)(F)CNC3=O)ccc2[nH]1)[C@@H](C)O[C@H](C)C(F)(F)F]

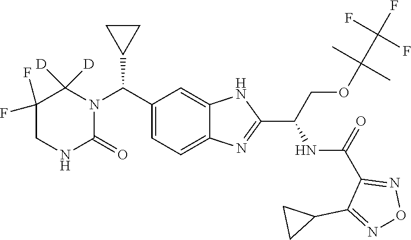 [SMILES: [2H]C1([2H])N([C@@H](c2ccc3nc([C@H](COC(C)(C)C(F)(F)F)NC(=O)c4nonc4C4CC4)[nH]c3c2)C2CC2)C(=O)NCC1(F)F]

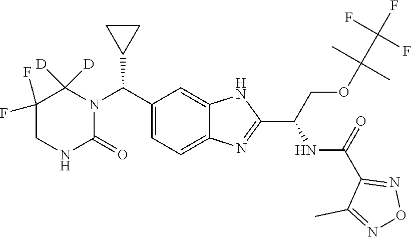 [SMILES: [2H]C1([2H])N([C@@H](c2ccc3nc([C@H](COC(C)(C)C(F)(F)F)NC(=O)c4nonc4C)[nH]c3c2)C2CC2)C(=O)NCC1(F)F]

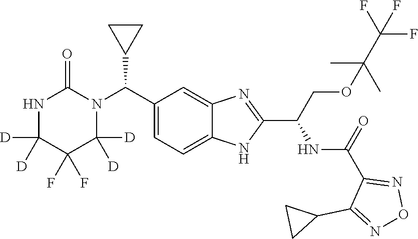 [SMILES: [2H]C1([2H])NC(=O)N([C@@H](c2ccc3[nH]c([C@H](COC(C)(C)C(F)(F)F)NC(=O)c4nonc4C4CC4)nc3c2)C2CC2)C([2H])([2H])C1(F)F]

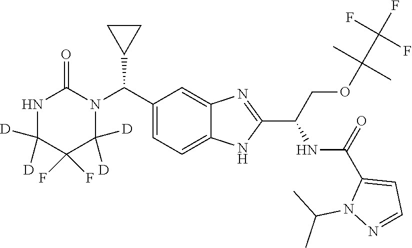 [SMILES: [2H]C1([2H])NC(=O)N([C@@H](c2ccc3[nH]c([C@H](COC(C)(C)C(F)(F)F)NC(=O)c4ccnn4C(C)C)nc3c2)C2CC2)C([2H])([2H])C1(F)F]

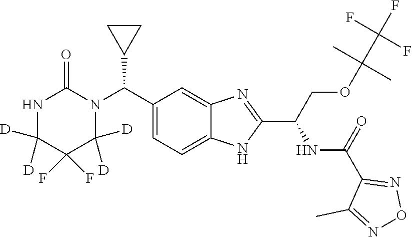 [SMILES: [2H]C1([2H])NC(=O)N([C@@H](c2ccc3[nH]c([C@H](COC(C)(C)C(F)(F)F)NC(=O)c4nonc4C)nc3c2)C2CC2)C([2H])([2H])C1(F)F]

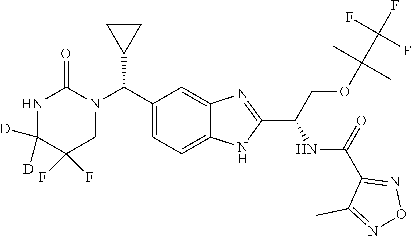 [SMILES: [2H]C1([2H])NC(=O)N([C@@H](c2ccc3[nH]c([C@H](COC(C)(C)C(F)(F)F)NC(=O)c4nonc4C)nc3c2)C2CC2)CC1(F)F]

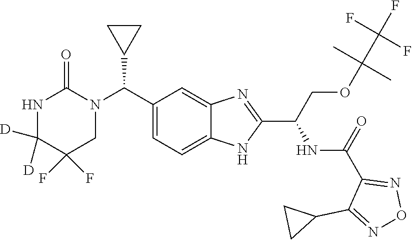 [SMILES: [2H]C1([2H])NC(=O)N([C@@H](c2ccc3[nH]c([C@H](COC(C)(C)C(F)(F)F)NC(=O)c4nonc4C4CC4)nc3c2)C2CC2)CC1(F)F]

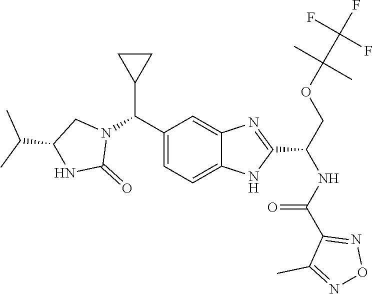 [SMILES: Cc1nonc1C(=O)N[C@@H](COC(C)(C)C(F)(F)F)c1nc2cc([C@@H](C3CC3)N3C[C@@H](C(C)C)NC3=O)ccc2[nH]1]

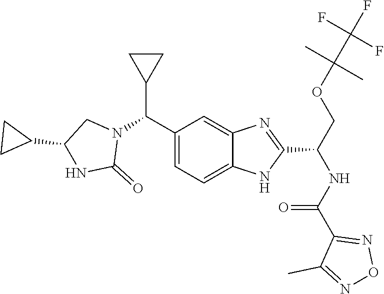 [SMILES: Cc1nonc1C(=O)N[C@@H](COC(C)(C)C(F)(F)F)c1nc2cc([C@@H](C3CC3)N3C[C@@H](C4CC4)NC3=O)ccc2[nH]1]

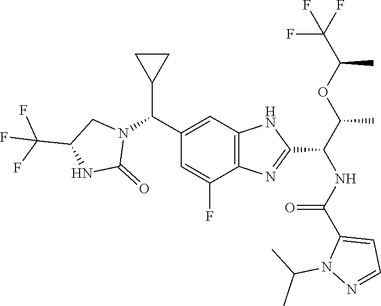 [SMILES: CC(C)n1nccc1C(=O)N[C@H](c1nc2c(F)cc([C@@H](C3CC3)N3C[C@@H](C(F)(F)F)NC3=O)cc2[nH]1)[C@@H](C)O[C@H](C)C(F)(F)F]

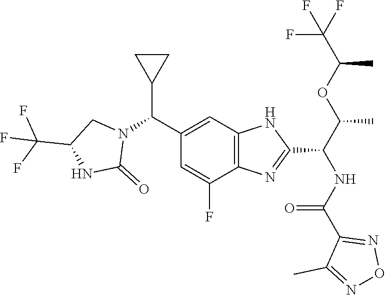 [SMILES: Cc1nonc1C(=O)N[C@H](c1nc2c(F)cc([C@@H](C3CC3)N3C[C@@H](C(F)(F)F)NC3=O)cc2[nH]1)[C@@H](C)O[C@H](C)C(F)(F)F]

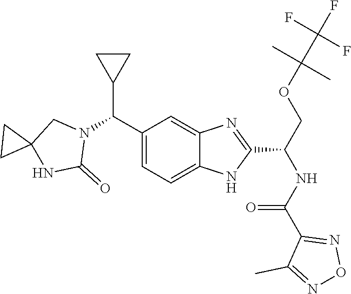 [SMILES: Cc1nonc1C(=O)N[C@@H](COC(C)(C)C(F)(F)F)c1nc2cc([C@@H](C3CC3)N3CC4(CC4)NC3=O)ccc2[nH]1]

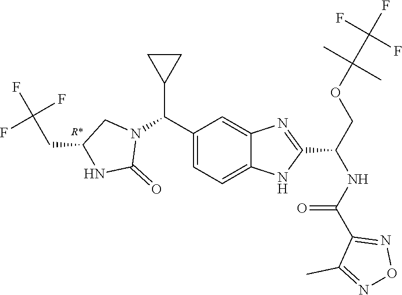 [SMILES: Cc1nonc1C(=O)N[C@@H](COC(C)(C)C(F)(F)F)c1nc2cc([C@@H](C3CC3)N3C[C@@H](CC(F)(F)F)NC3=O)ccc2[nH]1]

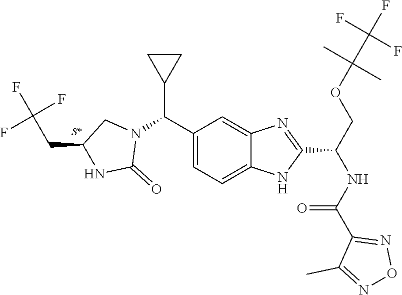 [SMILES: Cc1nonc1C(=O)N[C@@H](COC(C)(C)C(F)(F)F)c1nc2cc([C@@H](C3CC3)N3C[C@H](CC(F)(F)F)NC3=O)ccc2[nH]1]

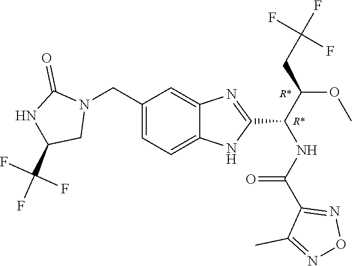 [SMILES: CO[C@H](CC(F)(F)F)[C@H](NC(=O)c1nonc1C)c1nc2cc(CN3C[C@@H](C(F)(F)F)NC3=O)ccc2[nH]1]

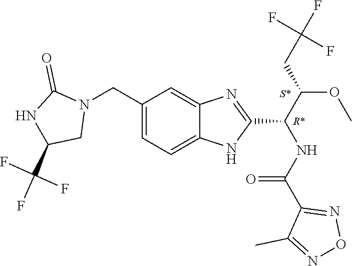 [SMILES: CO[C@@H](CC(F)(F)F)[C@H](NC(=O)c1nonc1C)c1nc2cc(CN3C[C@@H](C(F)(F)F)NC3=O)ccc2[nH]1]

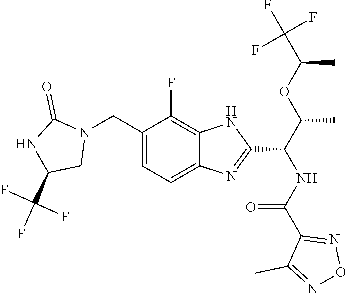 [SMILES: Cc1nonc1C(=O)N[C@H](c1nc2ccc(CN3C[C@@H](C(F)(F)F)NC3=O)c(F)c2[nH]1)[C@@H](C)O[C@H](C)C(F)(F)F]

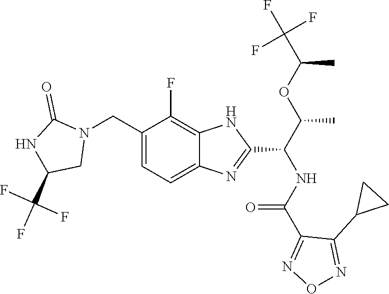 [SMILES: C[C@@H](O[C@H](C)C(F)(F)F)[C@H](NC(=O)c1nonc1C1CC1)c1nc2ccc(CN3C[C@@H](C(F)(F)F)NC3=O)c(F)c2[nH]1]

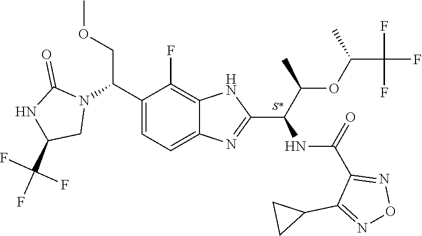 [SMILES: COC[C@H](c1ccc2nc([C@H](NC(=O)c3nonc3C3CC3)[C@@H](C)O[C@H](C)C(F)(F)F)[nH]c2c1F)N1C[C@@H](C(F)(F)F)NC1=O]

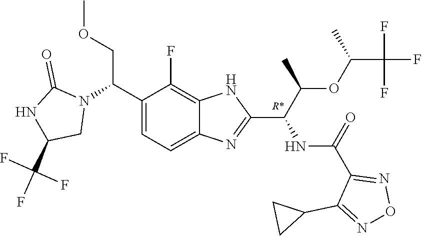 [SMILES: COC[C@H](c1ccc2nc([C@@H](NC(=O)c3nonc3C3CC3)[C@@H](C)O[C@H](C)C(F)(F)F)[nH]c2c1F)N1C[C@@H](C(F)(F)F)NC1=O]

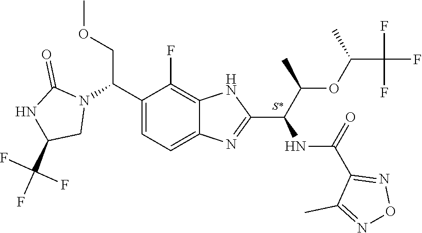 [SMILES: COC[C@H](c1ccc2nc([C@H](NC(=O)c3nonc3C)[C@@H](C)O[C@H](C)C(F)(F)F)[nH]c2c1F)N1C[C@@H](C(F)(F)F)NC1=O]

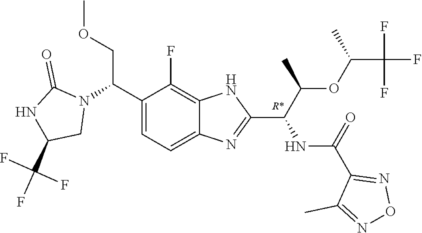 [SMILES: COC[C@H](c1ccc2nc([C@@H](NC(=O)c3nonc3C)[C@@H](C)O[C@H](C)C(F)(F)F)[nH]c2c1F)N1C[C@@H](C(F)(F)F)NC1=O]

In some embodiments, disclosed herein is a compound of Formula I, or a pharmaceutically acceptable salt thereof, selected from the group consisting of:

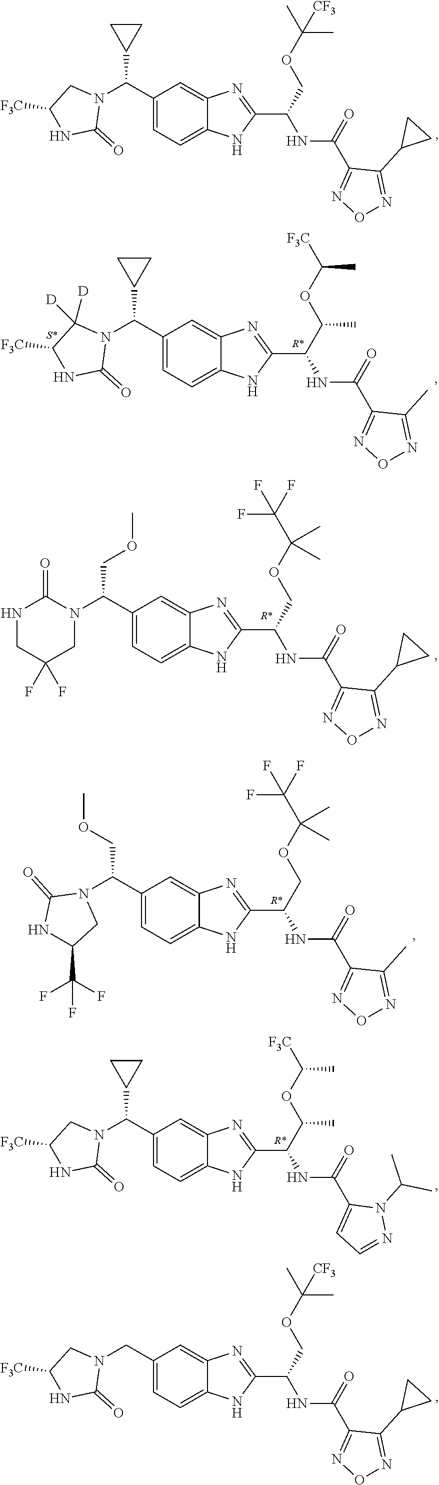

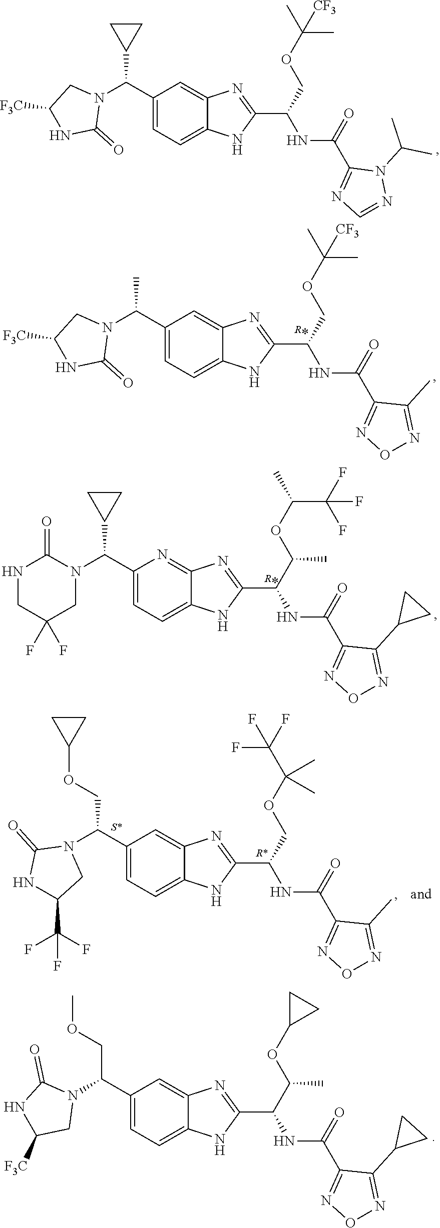

In some embodiments, disclosed herein is a compound of Formula I, or a pharmaceutically acceptable salt thereof, selected from the group consisting of:

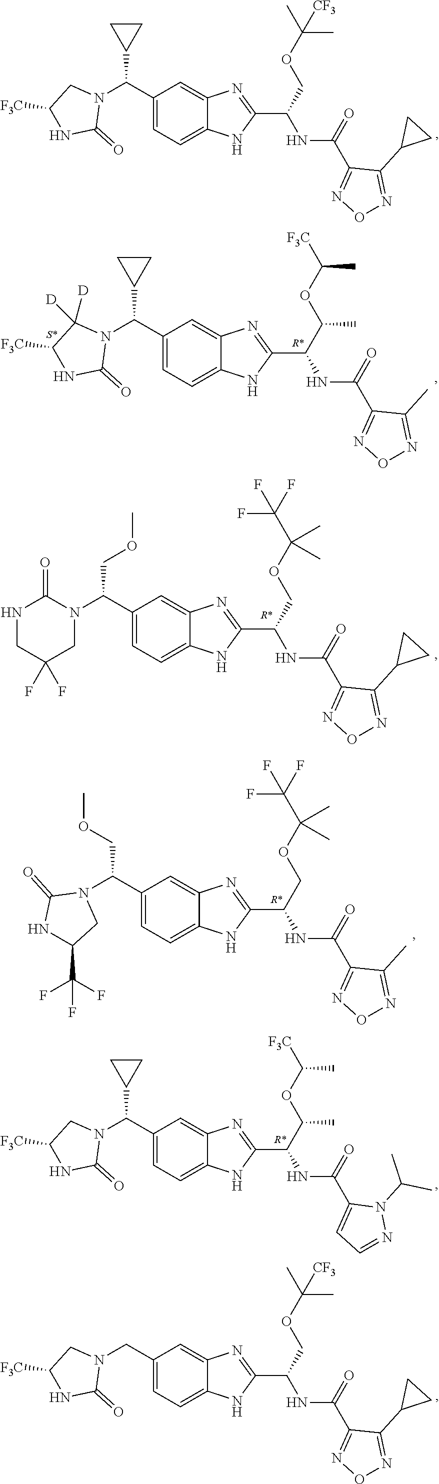

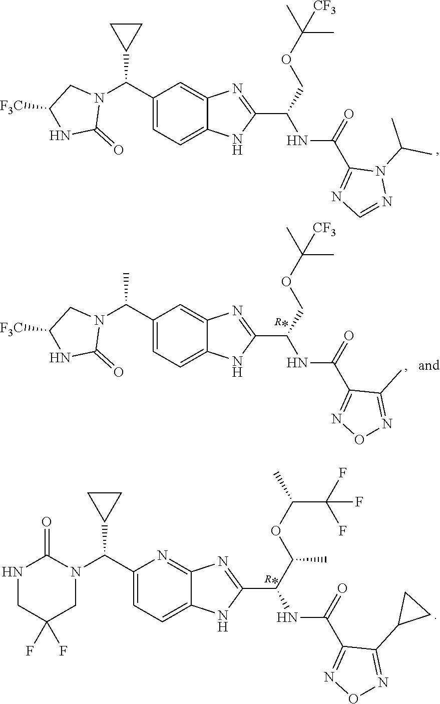

In some embodiments, disclosed herein is a compound of Formula I having the following structure:

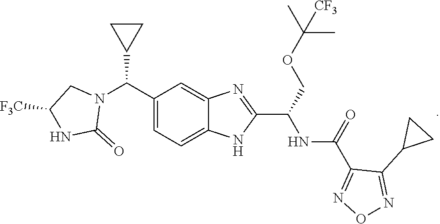 [SMILES: CC(C)(OC[C@H](NC(=O)c1nonc1C1CC1)c1nc2cc([C@@H](C3CC3)N3C[C@@H](C(F)(F)F)NC3=O)ccc2[nH]1)C(F)(F)F]

In some embodiments, disclosed herein is a compound of Formula I having the following structure:

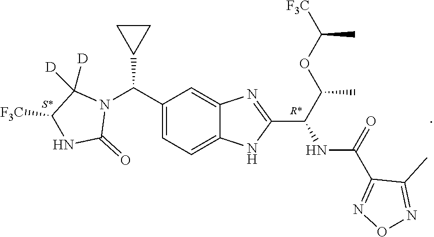 [SMILES: [2H]C1([2H])[C@@H](C(F)(F)F)NC(=O)N1[C@@H](c1ccc2[nH]c([C@@H](NC(=O)c3nonc3C)[C@@H](C)O[C@H](C)C(F)(F)F)nc2c1)C1CC1]

In some embodiments, disclosed herein is a compound of Formula I having the following structure:

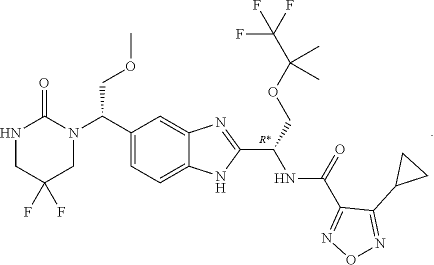 [SMILES: COC[C@H](c1ccc2[nH]c([C@H](COC(C)(C)C(F)(F)F)NC(=O)c3nonc3C3CC3)nc2c1)N1CC(F)(F)CNC1=O]

In some embodiments, disclosed herein is a compound of Formula I having the following structure:

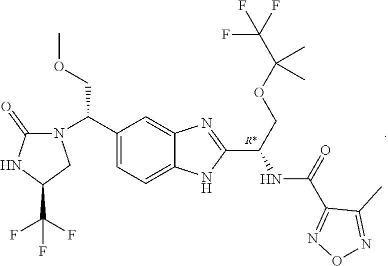 [SMILES: COC[C@H](c1ccc2[nH]c([C@H](COC(C)(C)C(F)(F)F)NC(=O)c3nonc3C)nc2c1)N1C[C@@H](C(F)(F)F)NC1=O]

In some embodiments, disclosed herein is a compound of Formula I having the following structure:

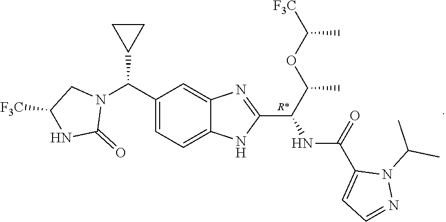 [SMILES: CC(C)n1nccc1C(=O)N[C@H](c1nc2cc([C@@H](C3CC3)N3C[C@@H](C(F)(F)F)NC3=O)ccc2[nH]1)[C@@H](C)O[C@@H](C)C(F)(F)F]

In some embodiments, disclosed herein is a compound of Formula I having the following structure:

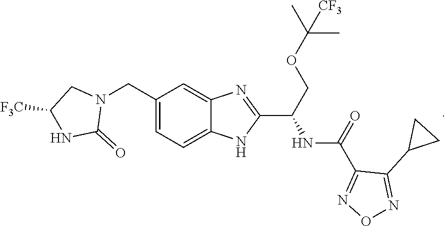 [SMILES: CC(C)(OC[C@H](NC(=O)c1nonc1C1CC1)c1nc2cc(CN3C[C@@H](C(F)(F)F)NC3=O)ccc2[nH]1)C(F)(F)F]

In some embodiments, disclosed herein is a compound of Formula I having the following structure:

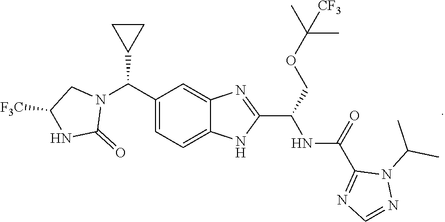 [SMILES: CC(C)n1ncnc1C(=O)N[C@@H](COC(C)(C)C(F)(F)F)c1nc2cc([C@@H](C3CC3)N3C[C@@H](C(F)(F)F)NC3=O)ccc2[nH]1]

In some embodiments, disclosed herein is a compound of Formula I having the following structure:

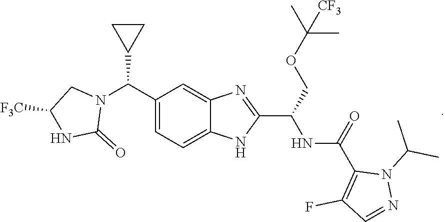 [SMILES: CC(C)n1ncc(F)c1C(=O)N[C@@H](COC(C)(C)C(F)(F)F)c1nc2cc([C@@H](C3CC3)N3C[C@@H](C(F)(F)F)NC3=O)ccc2[nH]1]

In some embodiments, disclosed herein is a compound of Formula I having the following structure:

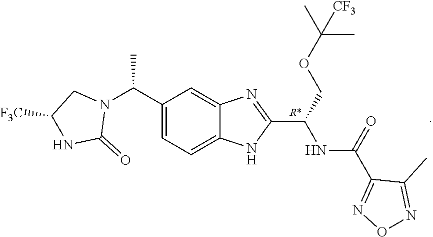 [SMILES: Cc1nonc1C(=O)N[C@@H](COC(C)(C)C(F)(F)F)c1nc2cc([C@@H](C)N3C[C@@H](C(F)(F)F)NC3=O)ccc2[nH]1]

In some embodiments, disclosed herein is a compound of Formula I having the following structure:

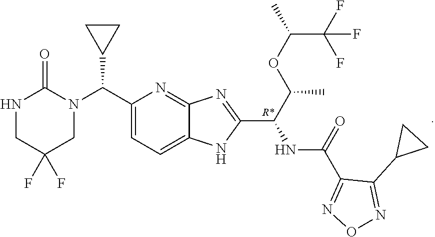 [SMILES: C[C@@H](O[C@H](C)C(F)(F)F)[C@H](NC(=O)c1nonc1C1CC1)c1nc2nc([C@@H](C3CC3)N3CC(F)(F)CNC3=O)ccc2[nH]1]

In some embodiments, disclosed herein is a compound of Formula I having the following structure:

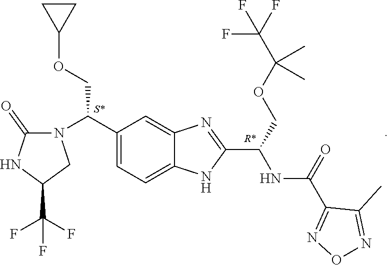 [SMILES: Cc1nonc1C(=O)N[C@@H](COC(C)(C)C(F)(F)F)c1nc2cc([C@@H](COC3CC3)N3C[C@@H](C(F)(F)F)NC3=O)ccc2[nH]1]

In some embodiments, disclosed herein is a compound of Formula I having the following

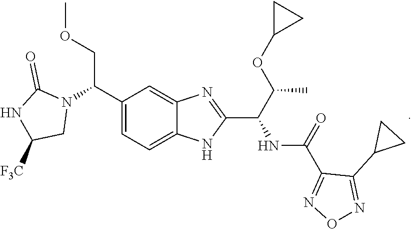 [SMILES: COC[C@H](c1ccc2[nH]c([C@@H](NC(=O)c3nonc3C3CC3)[C@@H](C)OC3CC3)nc2c1)N1C[C@@H](C(F)(F)F)NC1=O]

In some embodiments, disclosed herein is a pharmaceutical composition, comprising a compound of Formula I, or a pharmaceutically acceptable salt thereof, and a pharmaceutically acceptable carrier. In some embodiments, the pharmaceutical composition is formulated for oral administration (e.g., a tablet or capsule).

In some embodiments, disclosed herein is a pharmaceutical composition made by mixing a compound of Formula I, or a pharmaceutically acceptable salt thereof, and a pharmaceutically acceptable carrier.

In some embodiments, disclosed herein is a process for making a pharmaceutical composition comprising mixing a compound of Formula I, or a pharmaceutically acceptable salt thereof, and a pharmaceutically acceptable carrier.

### Therapeutic Use

The present application is also directed to a method for treating and/or ameliorating a IL-17 mediated inflammatory syndrome, disorder or disease comprising administering to a subject in need thereof an effective amount of a compound of Formula I, or pharmaceutically acceptable salt thereof, composition thereof, or medicament thereof.

In some embodiments, disclosed herein is a method for treating or ameliorating an IL-17A mediated inflammatory syndrome, disorder, or disease comprising administering to a subject in need thereof a therapeutically effective amount of a compound of Formula I, or a pharmaceutically acceptable salt thereof.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is psoriasis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is psoriatic arthritis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is rheumatoid arthritis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is ankylosing spondylitis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is hidradenitis suppurativa.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is bullous pemphigoid.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is atopic dermatitis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is vitiligo.

In some embodiments, disclosed herein is a method for treating or ameliorating and/an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is multiple sclerosis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is systemic lupus erythematosus.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is asthma.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is uveitis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is chronic obstructive pulmonary disorder.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is multiple myeloma.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus, wherein the compound of Formula I or the pharmaceutically acceptable salt thereof is administered orally (e.g., as a tablet or capsule).

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus, wherein the therapeutically effective amount is a dose of about 10 mg to 300 mg QD. In some embodiments, the therapeutically effective amount is a dose of about 20 mg to 200 mg QD. In some embodiments, the therapeutically effective amount is a dose of about 50 mg to 100 mg QD.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus, wherein the therapeutically effective amount is a dose of about 10 mg to 300 mg BID. In some embodiments, the therapeutically effective amount is a dose of about 20 mg to 200 mg BID. In some embodiments, the therapeutically effective amount is a dose of about 50 mg to 100 mg BID.

In some embodiments, disclosed herein is the use of a therapeutically effective amount of compound of Formula I, or a pharmaceutically acceptable salt thereof, for treating and/or ameliorating an IL-17A mediated inflammatory syndrome, disorder, or disease selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

In some embodiments, disclosed herein is the use of a compound of Formula I, or pharmaceutically acceptable salt thereof, in the manufacture of a medicament for treating and/or ameliorating an IL-17A mediated inflammatory syndrome, disorder, or disease selected from the group consisting of psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

In some embodiments, disclosed herein is a method for treating and/or ameliorating an IL-17 mediated inflammatory syndrome, disorder or disease comprising administering to a subject in need thereof a therapeutically effective amount of a compound of Formula I, or pharmaceutically acceptable salt thereof, a composition thereof, or a medicament thereof.

In some embodiments, disclosed herein is a method of treating and/or ameliorating an IL-17 mediated inflammatory syndrome, disorder or disease, wherein the syndrome, disorder or disease is selected from the group consisting of: psoriasis, psoriatic arthritis, rheumatoid arthritis, ankylosing spondylitis, hidradenitis suppurativa, atopic dermatitis, vitiligo, multiple sclerosis, asthma, allergic asthma, steroid resistant asthma, neutrophilic asthma, chronic obstructive pulmonary disease, uveitis, multiple myeloma, and systemic lupus erythematosus, comprising administering to a subject in need thereof a therapeutically effective amount of a compound of Formula I, or pharmaceutically acceptable salt thereof, a composition thereof, or a medicament thereof.

In some embodiments, disclosed herein is a method of treating or ameliorating an IL-17 mediated inflammatory syndrome, disorder or disease, wherein the syndrome, disorder or disease is selected from the group consisting of: psoriasis, psoriatic arthritis, and ankylosing spondylitis, comprising administering to a subject in need thereof a therapeutically effective amount of a compound of Formula I, or pharmaceutically acceptable salt thereof, a composition thereof, or a medicament thereof.

In some embodiments, disclosed herein are methods of modulating IL-17 activity in a mammal by administration of a therapeutically effective amount of at least one compound of Formula I, or pharmaceutically acceptable salt thereof.

Also disclosed herein is a method of inhibiting production of interleukin-17, comprising administering to a subject in need thereof a therapeutically effective amount of a compound of Formula I, or pharmaceutically acceptable salt thereof.

### Combination Therapy

A compound of Formula I, or pharmaceutically acceptable salt thereof, a composition thereof, or a medicament thereof may also be used in combination with one or more additional therapeutic agents.

In some embodiments, the one or more additional therapeutic agents is selected from the group consisting of anti-inflammatory agents, immunomodulatory agents, and immunosuppressive agents.

In some embodiments, the one or more additional therapeutic agents is selected from the group consisting of: anti-TNFalpha agents such as infliximab (Remicade®), adalimumab (Humira®), certolizumab pegol (Cimzia®), golimumab (Simponi®), etanercept (Enbrel®), thalidomide (Immunoprin®), lenalidomide (Revlimid®), and pomalidomide (Pomalyst®/Imnovid®); anti-p40 antibody agents such as ustekinumab (Stelara®); and anti-p19 antibody agents such as guselkumab (Tremfya®), tildrakizumab (Ilumya™/Ilumetri), risankizumab (Skyrizi™), and mirikizumab.

In some embodiments, disclosed herein is a method of treating and/or ameliorating an IL-17 mediated inflammatory syndrome, disorder or disease, in a subject in need thereof comprising administering to the subject a therapeutically effective amount of the compound of Formula I, or pharmaceutically acceptable salt thereof, composition thereof, or medicament thereof in a combination therapy with one or more additional therapeutic agents, such as anti-inflammatory agents, immunomodulatory agents, or immunosuppressive agents, wherein said syndrome, disorder or disease is selected from the group consisting of psoriasis, psoriatic arthritis, rheumatoid arthritis, ankylosing spondylitis, hidradenitis suppurativa, atopic dermatitis, vitiligo, multiple sclerosis, asthma, allergic asthma, steroid resistant asthma, neutrophilic asthma, chronic obstructive pulmonary disease, uveitis, multiple myeloma, and systemic lupus erythematosus.

In some embodiments, disclosed herein is a method of treating and/or ameliorating an IL-17 mediated inflammatory syndrome, disorder or disease, in a subject in need thereof comprising administering to the subject a therapeutically effective amount of the compound of Formula I, or pharmaceutically acceptable salt thereof, composition thereof, or medicament thereof in a combination therapy with one or more additional therapeutic agents, such as anti-inflammatory agents, or immunosuppressive agents, wherein said syndrome, disorder or disease is psoriasis, psoriatic arthritis, ankylosing spondylitis. In some embodiments, the IL-17 mediated inflammatory syndrome, disorder or disease is psoriasis. In some embodiments, the IL-17 mediated inflammatory syndrome, disorder or disease is psoriatic arthritis. In some embodiments, the IL-17 mediated inflammatory syndrome, disorder or disease is ankylosing spondylitis.

### Dosage Regimen

When employed as IL-17A modulators, the compounds disclosed herein may be administered in an effective amount within the dosage range of about 0.5 mg to about 1 g, preferably between about 0.5 mg to about 500 mg, in single or divided daily doses. In some embodiments, the dosage amount is about 5 mg to 400 mg. In some embodiments, the dosage amount is about 10 mg to 300 mg. In some embodiments, the dosage amount is about 0.5, 1, 5, 10, 15, 20, 25, 30, 35, 40, 45, 50, 55, 60, 65, 70, 75, 80, 85, 90, 95, or 100 mg of a compound of Formula I, or pharmaceutically acceptable salt thereof. In some embodiments, the dosage amount is about 100, 105, 110, 115, 120, 125, 130, 135, 140, 145, 150, 155, 160, 165, 170, 175, 180, 185, 190, 195, or 200 mg of a compound of Formula I, or pharmaceutically acceptable salt thereof. In some embodiments, the dosage amount is about 200, 205, 210, 215, 220, 225, 230, 235, 240, 245, 250, 255, 260, 265, 270, 275, 280, 285, 290, 295, or 300 mg of a compound of Formula I, or pharmaceutically acceptable salt thereof. In some embodiments, the dosage amount is about 300, 315, 320, 325, 330, 335, 340, 345, 350, 355, 360, 365, 370, 375, 380, 385, 390, 395, or 400 mg of a compound of Formula I, or pharmaceutically acceptable salt thereof. In some embodiments, the dosage amount is about 400, 405, 410, 415, 420, 425, 430, 435, 440, 445, 450, 455, 460, 465, 470, 475, 480, 485, 490, 495, or 500 mg of a compound of Formula I, or pharmaceutically acceptable salt thereof.

In some embodiments, a compound of Formula I, or pharmaceutically acceptable salt thereof, may be administered in an effective amount within the dosage range of about 10 mg to 300 mg QD. In some embodiments, a compound of Formula I, or pharmaceutically acceptable salt thereof, may be administered in an effective amount within the dosage range of about 20 mg to 200 mg QD. In some embodiments, a compound of Formula I, or pharmaceutically acceptable salt thereof, may be administered in an effective amount within the dosage range of about 50 mg to 100 mg QD.

In some embodiments, a compound of Formula I, or pharmaceutically acceptable salt thereof, may be administered in an effective amount within the dosage range of about 10 mg to 300 mg BID. In some embodiments, a compound of Formula I, or pharmaceutically acceptable salt thereof, may be administered in an effective amount within the dosage range of about 20 mg to 200 mg BID. In some embodiments, a compound of Formula I, or pharmaceutically acceptable salt thereof, may be administered in an effective amount within the dosage range of about 50 mg to 100 mg BID.

The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present invention or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

### Pharmaceutically Acceptable Salts

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate, sodium hydroxide, triethanolamine, or zinc.

### Pharmaceutical Compositions

The compounds of Formula I, or pharmaceutically acceptable salt thereof, may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds of Formula I include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamino salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, topical, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

Also disclosed herein is a method of making a pharmaceutical composition comprising mixing a pharmaceutically acceptable carrier with any of the compounds of Formula I, or pharmaceutically acceptable salt thereof. Additionally, the present application includes pharmaceutical compositions made by mixing a pharmaceutically acceptable carrier with any of the compounds of the present invention.

### EXAMPLES

### Abbreviations

Herein and throughout the application, the following abbreviations may be used.
Å Angstrom
Ac acetyl
atm atmosphere
Boc tert-butyloxycarbonyl
br broad
BTEAC benzyltriethylammonium chloride
Bu butyl
Cbz benzyloxycarbonyl
CDI 1,1′-carbonyldiimidazole
δ NMR chemical shift in parts per million downfield from a standard
d doublet
d day(s)
DCM dichloromethane
DEA diethylamine
DIPEA N,N-diisopropylethylamine (Hünig's base)
DMF N,N-dimethylformamide
DMSO dimethyl sulfoxide
DMAP 4-dimethylaminopyridine
EDCI 1-ethyl-3-(3-dimethylaminopropyl) carbodiimide hydrochloride
ESI electrospray ionization
Et ethyl
EtOAc ethyl acetate
FA formic acid
g gram(s)
h hour(s)
Hantzsch ester diethyl 1,4-dihydro-2,6-dimethyl-3,5-pyridinedicarboxylate
HATU O-(7-azabenzotriazol-1-yl)-N,N,N′,N′-tetramethyluronium hexafluorophosphate
HOBt 1-hydroxybenzotriazole
HPLC high pressure liquid chromatography
Hz Hertz
i iso
IPA isopropanol
J coupling constant (NMR spectroscopy)
L liter(s)
LAH Lithium aluminum hydride
LC liquid chromatography
LDA lithium diisopropylamide
LED light-emitting diode
m milli or multiplet
m/z mass-to-charge ratio
M⁺ parent molecular ion
M molar (moles/liter) or mega
Me methyl
MeCN acetonitrile
min minute(s)
μ micro
MS mass spectrometry
MTBE tert-butyl methyl ether
n normal
n nano
N normal (equivalent concentration)
NMR nuclear magnetic resonance
NMP N-methyl-2-pyrrolidone
Pd/C palladium on carbon
p para
Ph phenyl
PPTS pyridinium p-toluenesulfonate
Pr propyl
Psi pounds per square inch
rt room temperature
RuPhos Pd G3 (2-Dicyclohexylphosphino-2′,6′-diisopropoxy-1,1′-biphenyl)[2-(2′-amino-1,1′-biphenyl)]palladium(II) methanesulfonate
s singlet
SEM 2-(Trimethylsilyl)ethoxymethyl
SFC supercritical fluid chromatography
t tertiary
t triplet
T₃P 1-propanephosphonic anhydride
TEA triethylamine
tert tertiary
TFA trifluoroacetic acid
THF tetrahydrofuran
TMEDA N,N,N′,N′-Tetramethylethylenediamine
Ts para-toluenesulfonyl (tosyl)

In some embodiments, provided herein are processes and intermediates disclosed herein that are useful for preparing a compound of the disclosure or pharmaceutically acceptable salts thereof.

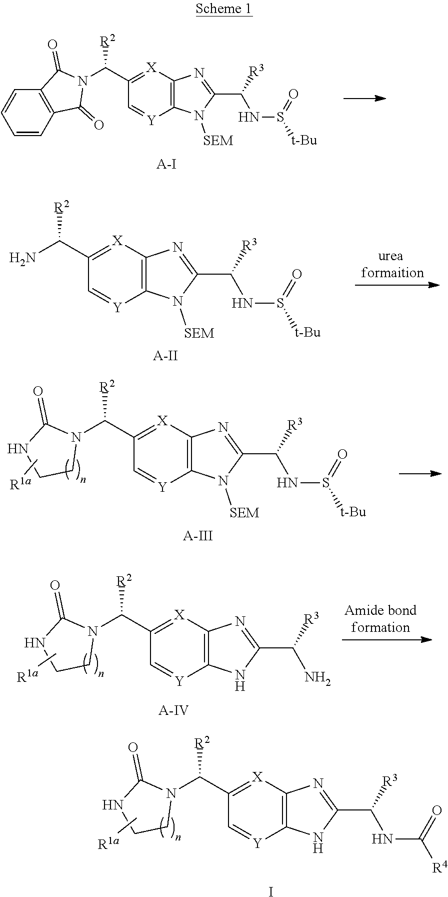

The compounds of Formula I in the present invention can be prepared as shown in Scheme 1. Deprotection of the phthalimide group within compounds A-I using a reagent such as hydrazine in a solvent such as ethanol affords amines A-II. These conditions will be known going forward as “Phthalimide Deprotection Conditions.” Compounds A-II can be converted to compounds A-III through a sequence of reactions which will be known as “Urea Formation Conditions.” This can be achieved by preparing a diamine by, for example, (1) reacting an amine as present in compounds A-II with an activated amino alcohol, such as (S)-benzyl 4-(trifluoromethyl)-1,2,3-oxathiazolidine-3-carboxylate 2,2-dioxide. Then (2), the adduct formed from this reaction is subjected to reduction conditions such as hydrogenation conditions to afford the diamine. The resulting diamine can then be treated with CDI or triphosgene in a solvent such as THF or DCM to afford compounds A-III. Deprotection of the sulfinamide and the SEM protecting groups using a reagent such as hydrochloric acid in solvents such as methanol, 1,4-dioxane or EtOAc, or mixtures thereof, yields amines A-IV. These conditions will be known as “SEM Deprotection Conditions.” Compounds of Formula I can be prepared from amines A-IV through a variety of methods, several of which are described below. Amide bond formation between amines A-IV and carboxylic acids (R⁴CO₂H) can be achieved through the use of a coupling agent, such as HATU, T₃P or EDCI, in the presence of a base, such as DIPEA, in a solvent, such as DMF, MeCN, or DCM, with or without an additive, such as HOBt to yield compounds of Formula I. Alternatively, amide bond formation can be achieved by treatment of amines A-IV with a reagent such as a carboxylic acid chloride (R⁴CO₂Cl) in the presence of additives such as DIPEA or DMAP in solvents such as DCM or THF to yield compounds of Formula I. In addition, amines A-IV can be treated with N-hydroxysuccinate esters in the presence of reagents such as DIPEA in a solvent such as acetonitrile to provide compounds of Formula I.

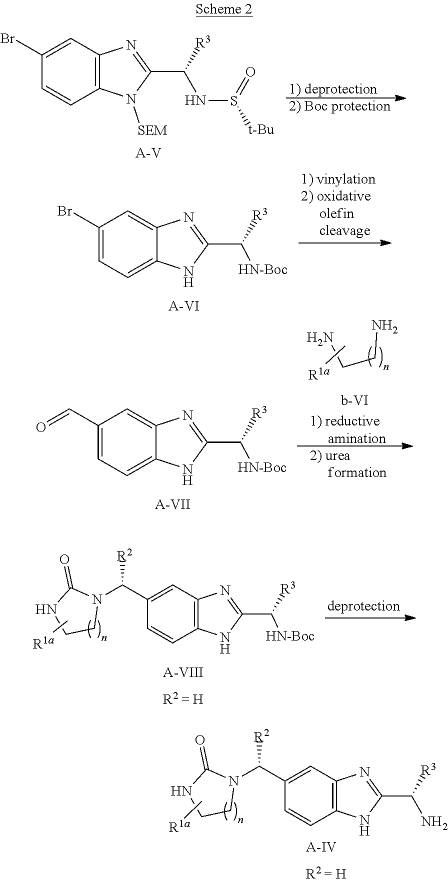

Amines A-IV can also be prepared as shown in Scheme 2. Deprotection of the SEM and sulfinimide protecting groups within A-V using SEM Deprotection Conditions followed by protection of the resulting amine with a Boc group using reagents such as di-tert-butyl dicarbonate in the presence of a base such as sodium carbonate then yields compounds A-VI. Intermediates A-VI can be converted to aldehydes A-VII via a two step sequence of 1) vinylation with a reagent such as potassium trifluoro(vinyl)boranide, an additive such as K₃PO₄, and a catalyst such as [1,1′-bis(diphenylphosphino)ferrocene]dichloropalladium(II) complex in a solvent such as dichloromethane, and 2) oxidative cleavage of the olefin prepared in the previous step with reagents such as K₂OsO₄·2H₂O and NaIO₄. Treatment of aldehydes A-VII with diamines (b-VI) under reductive amination conditions such as NaCNBH₃ in a solvent such as methanol in the presence of additives such as acetic acid, followed by treatment with a reagent such as triphosgene or CDI in a solvent such as DCM affords the corresponding cyclic ureas A-VIII. Boc deprotection of A-VIII using reagents such as TFA in solvents such as DCM then yields amines A-IV.

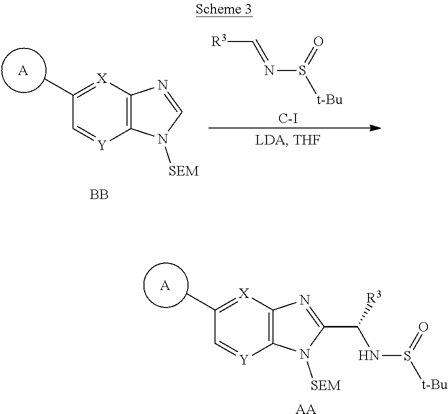

Sulfinamides AA may be prepared as shown in Scheme 3. Deprotonation of compounds BB using a base such as LDA in a solvent such as THF, followed treatment with sulfinimines C-I affords sulfinamides AA. Wherein

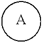

is H, halo,

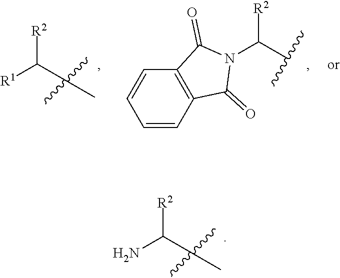

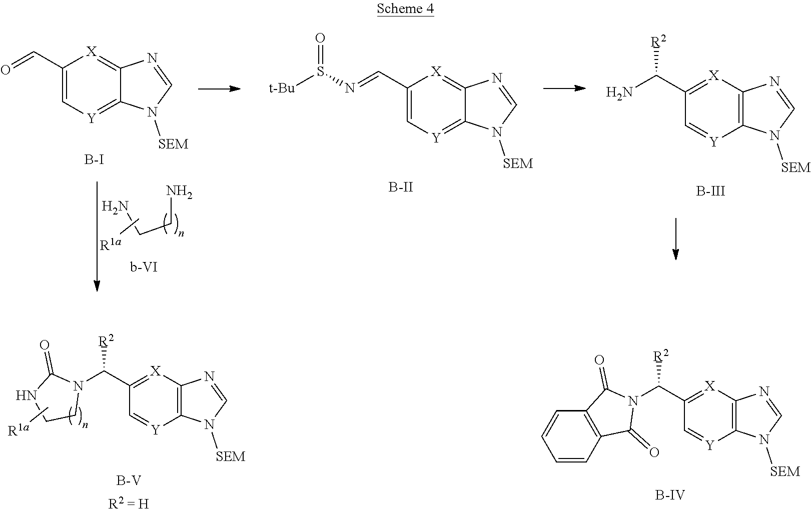

Compounds B-V and B-IV can be prepared as shown in Scheme 4. Aldehydes B-I can be treated with 2-methylpropane-2-sulfinamide in the presence of additives such as PPTS and copper sulfate in solvents such as DCM to provide sulfinimides B-II. Addition of suitable nucleophiles, such as R²M where M is Li, MgCl, or MgBr, to compounds B-II provides the corresponding adduct (structure not shown), followed by removal of the sulfinamide using reagents such as HCl in a solvent, such as a solvent mixture of 1,4-dioxane:MeOH then affords the corresponding amines B-III. Protection of the amine within B-III as a phthalimide using a reagent such as N-carbethoxyphthalimide or ethyl 1,3-dioxoisoindoline-2-carboxylate in a solvent such as THF in the presence of a base such as triethylamine then generates imides B-IV. Alternatively, aldehydes B-I can be initially treated with a diamine (e.g, diamines b-VI) under reductive amination conditions such as NaCNBH₃ in a solvent such as methanol in the presence of additives such as acetic acid, followed by treatment with a reagent such as triphosgene or CDI in a solvent such as DCM to afford the corresponding cyclic ureas B-V.

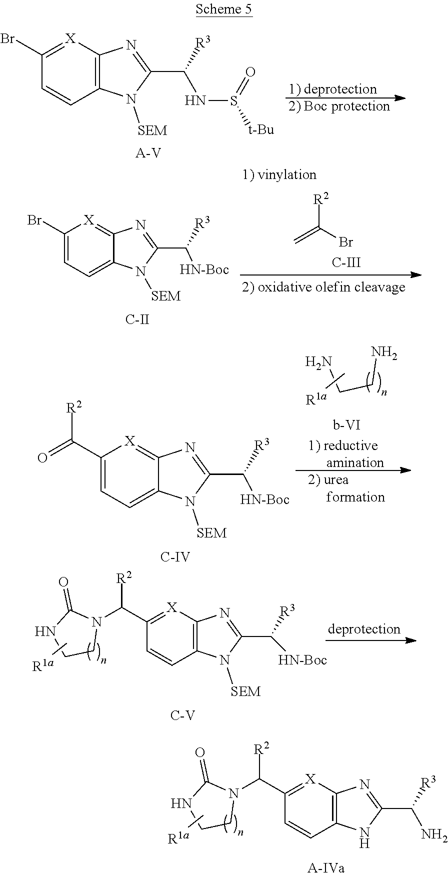

Amines A-IVa can be prepared as shown in Scheme 5. Deprotection of the sulfinimide protecting group within A-V using reagents such as iodine in solvents such as THF and water followed by protection of the resulting amine (structure not shown) with a Boc group using reagents such as di-tert-butyl dicarbonate in the presence of a base such as sodium carbonate or TEA then yields compounds C-II. Halogenated intermediates C-II can be converted to ketones C-IV via a two-step sequence of 1) vinylation with a reagent such as C-III with additives such as bis(pinacolato)diboron, potassium acetate and K₃PO₄, and a catalyst such as mesylate[(di(1-adamantyl)-n-butylphosphine)-2-(2′-amino-1,1′-biphenyl)]palladium(II) in a solvent such as dichloromethane, and 2) oxidative cleavage of the olefin prepared in the previous step with reagents such as K₂OsO₄·2H₂O and NaIO₄. Ketones C-IV can be converted to compounds C-V via a two-step process: (1) treatment of compounds C-IV with diamines (b-VI) under reductive amination conditions such as NaCNBH₃, with or without an additive such as tetraisopropoxytitanium, in a solvent such as methanol in the presence of additives such as acetic acid and then (2) treatment of the adduct formed in step (1) with a reagent such as triphosgene or carbonyldiimidazole (CDI) in a solvent such as DCM affords the corresponding cyclic ureas C-V. Boc and SEM deprotection of C-V using reagents such as HCl in solvents such as 1,4-dioxane yields amines A-IVa.

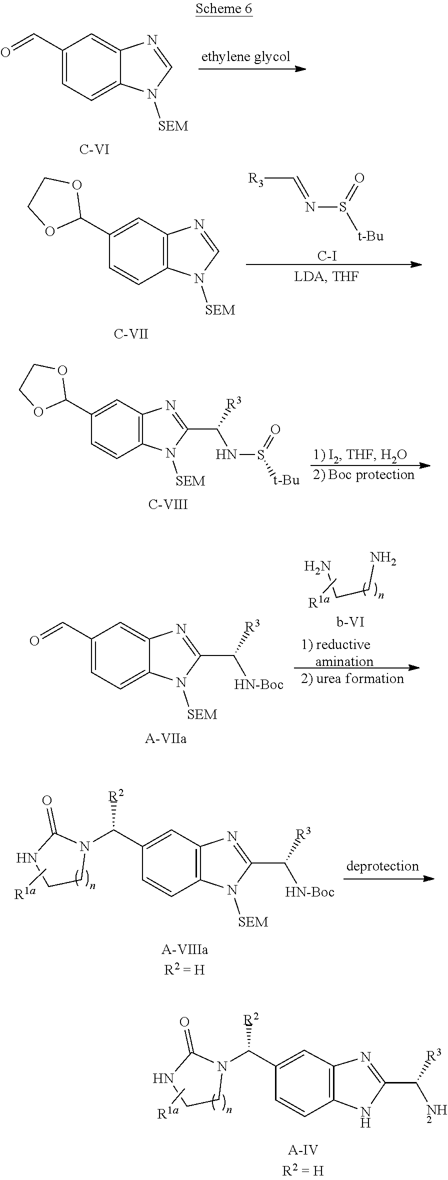

Amines A-IV can also be prepared as shown in Scheme 6. Protection of the aldehyde in compounds C-VI with a reagent, such as ethylene glycol, in the presence of a catalyst such asp-toluenesulfonic acid monohydrate yields compounds C-VII. Deprotonation of compounds C-VII using a base such as LDA in a solvent such as THF, followed treatment with sulfinimines C-I affords sulfinamides C-VIII. Deprotection of the sulfinamide and acetal protecting groups by treatment with a reagent such as iodine in solvents such as THF and water followed by protection of the resulting amine with a Boc group using reagents such as di-tert-butyl dicarbonate in the presence of a base such as sodium carbonate or triethylamine then yields compounds A-VIIa. Treatment of aldehydes A-VIIa with diamines (b-VI) under reductive amination conditions such as NaCNBH₃ in a solvent such as methanol in the presence of additives such as acetic acid, followed by treatment with a reagent such as triphosgene or CDI in a solvent such as DCM affords the corresponding cyclic ureas A-VIIIa. Boc deprotection of compounds A-VIIIa using reagents such as TFA in solvents such as DCM then yields amines A-IV.

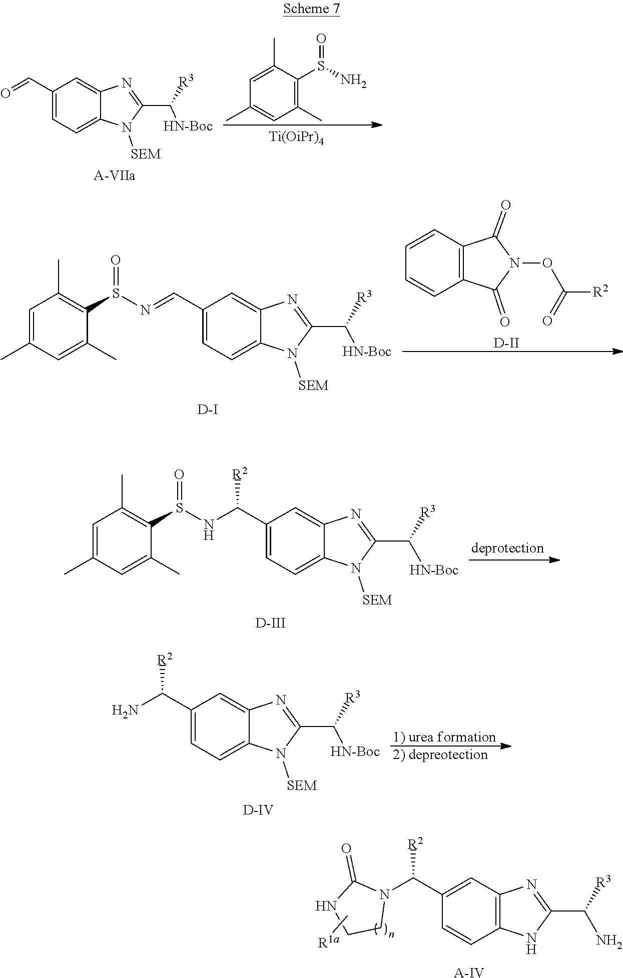

Compounds A-IV can also be prepared as shown in Scheme 7. Treatment of aldehydes A-VIIa with (R)-2,4,6-trimethylbenzenesulfinamide in the presence of additives such as titanium isopropoxide in a solvent such as THF provides sulfinimides D-I. Treatment of sulfinimides D-I with dioxoisoindolines D-II in the presence of a reagent, such as Hantzsch ester, and a base such as DIPEA in a solvent, such as DMSO affords compounds D-III. Treatment od compounds D-III with an acid, such as HCl in a solvent, such as EtOAc provides amines D-IV. Compounds D-IV can be converted to amines A-IV as shown in Scheme 1, employing methods analogous to those describing the conversion of compounds A-II to compounds A-IV.

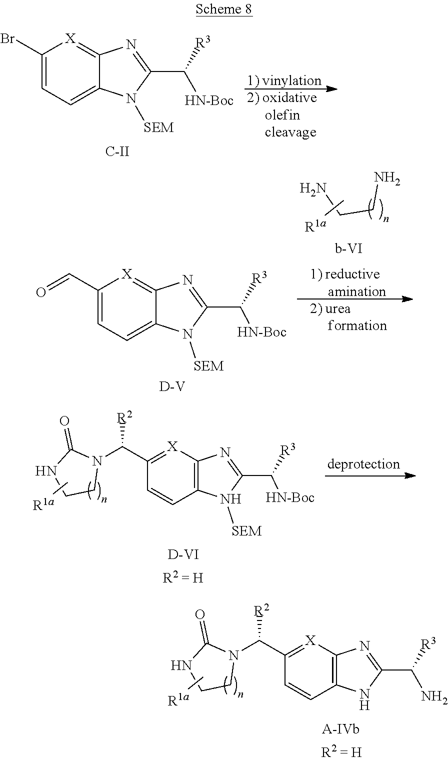

Amines A-IVb can be prepared as shown in Scheme 8. Halogenated intermediates C-II can be converted to aldehydes D-V via a two-step sequence of 1) vinylation with a reagent such as potassium trifluoro(vinyl)boranide, an additive such as K₃PO₄, and a catalyst such as [1,1′-bis(diphenylphosphino)ferrocene]dichloropalladium(II) complex in a solvent such as dichloromethane or 1,4-dioxane, and 2) oxidative cleavage of the olefin prepared in the previous step with reagents such as K₂OsO₄·2H₂O and NaIO₄. Treatment of aldehydes D-V with diamines (b-VI) under reductive amination conditions such as NaCNBH₃ in a solvent such as methanol in the presence of additives such as acetic acid, followed by treatment with a reagent such as triphosgene or CDI in a solvent such as DCM or THF affords the corresponding cyclic ureas D-VI. Boc and SEM deprotection of compounds D-VI using reagents such as HCl in solvents such as 1,4-dioxane then yields amines A-IVb.

### Intermediate 1: (R,E)-2-Methyl-N-(2-((1,1,1-trifluoro-2-methylpropan-2-yl)oxy)ethylidene)propane-2-sulfinamide

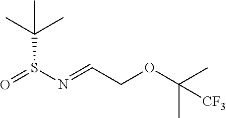 [SMILES: CC(C)(C)[S@@](=O)/N=C/COC(C)(C)C(F)(F)F]

Step A: 3-((1,1,1-Trifluoro-2-methylpropan-2-yl)oxy)prop-1-ene. To a suspension of NaH (6.9 g, 172 mmol, 60% dispersion in mineral oil) in NMP (40 mL) at 0° C. was slowly added 2-trifluoromethyl-2-propanol (20 g, 156 mmol) and the mixture stirred at 0° C. until gas evolution ceased. Allyl bromide (13.3 mL, 156 mmol) was added, and the reaction mixture was stirred for 16 h while gradually warming to rt. This material was purified by distillation (atmospheric pressure, 190° C.) to provide the title compound as a clear oil in 57% yield.

Step B: (R,E)-2-Methyl-N-(2-((1,1,1-trifluoro-2-methylpropan-2-yl)oxy)ethylidene)propane-2-sulfinamide. A solution of 3-((1,1,1-trifluoro-2-methylpropan-2-yl)oxy)prop-1-ene (15.0 g, 89.2 mmol, Step A) in CH₂Cl₂ (300 mL) was cooled to −78° C. then treated with ozone for 20 min. The reaction headspace was purged with N₂ then dimethyl sulfide (7.3 mL, 98 mmol) was added, and the mixture allowed to warm to rt. After stirring for 4 h, (R)-2-methylpropane-2-sulfinamide (10.8 g, 89.2 mmol) and copper sulfate (43.6 g, 268 mmol) were added and then stirring was continued for 16 h. The mixture was filtered through diatomaceous earth (e.g., Celite©), concentrated under reduced pressure and purified by silica gel chromatography (0-60% EtOAc/hexanes) to provide the title compound in 34% yield.

### Intermediate 2: (R)-2-(((R)-1,1,1-Trifluoropropan-2-yl)oxy)propanoic acid

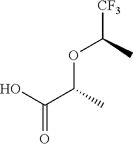

Sodium hydride (3.5 g, 88 mmol, 60% dispersion in mineral oil) was added in portions to a solution of (R)-1,1,1-trifluoropropan-2-ol (5.0 g, 44 mmol) in DMF (70 mL) at 0° C. The resultant mixture was stirred for 30 min at 25° C. before cooling to 0° C. again. A separate solution of (S)-2-bromopropanoic acid (6.0 g, 40 mmol) in DMF (5 mL) was added at 0° C. and the resulting mixture was allowed to warm to rt and stir for 12 h. After this time, the reaction mixture was poured into ice chilled water (100 mL) and extracted with MTBE (25 mL). The pH of the aqueous layer was adjusted by the addition of 2 N aqueous HCl (15 mL) until the pH of the mixture was pH=5-6. This aqueous layer was extracted with MTBE (80 mL×3). The combined organic layers were washed with brine, dried over anhydrous Na₂SO₄, filtered, and concentrated under reduced pressure to afford the title compound in 93% yield as a yellow oil that was used without further purification.

### Intermediate 3: (R)—N-Methoxy-N-methyl-2-(((R)-1,1,1-trifluoropropan-2-yl)oxy)propanamide

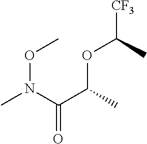

A round-bottom flask was charged with (R)-2-(((R)-1,1,1-trifluoropropan-2-yl)oxy)propanoic acid (7.6 g, 41 mmol, Intermediate 2), DMF (70 mL), HATU (20 g, 53 mmol), and DIPEA (18 mL, 102 mmol). The mixture was stirred for 5 min before the addition of N,O-dimethylhydroxylamine hydrochloride (6.0 g, 61 mmol). The resulting solution stirred for 12 h, after which time, the reaction was quenched with water (30 mL) and diluted with MTBE (50 mL). The organic layer was separated, and the aqueous layer was extracted with MTBE (80 mL×2). The combined organic layers were washed with water and brine, dried over anhydrous Na₂SO₄, filtered, and concentrated under reduced pressure. The product was purified by silica gel chromatography (9-17% EtOAc/petroleum ether) to afford the title compound in 58% yield as a yellow oil.

### Intermediate 4: (R)-2-(((R)-1,1,1-Trifluoropropan-2-yl)oxy)propanal

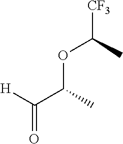

A round-bottom flask was charged with (R)—N-methoxy-N-methyl-2-(((R)-1,1,1-trifluoropropan-2-yl)oxy)propanamide (5.4 g, 24 mmol, Intermediate 3) and THF (300 mL), cooled to −78° C. and LAH (4.5 g, 120 mmol) was added portion wise. The resulting mixture was stirred for about 1 h at −78° C. and was subsequently quenched with water (20 mL) dropwise at −78° C. A saturated solution of sodium potassium tartrate (120 mL) was added, and the solution was stirred for 30 min and then extracted with MTBE (120 mL×3). The combined organic layers were washed with brine, dried over anhydrous Na₂SO₄, filtered, and concentrated under reduced pressure to afford the title compound in 100% yield that was used without further purification.

### Intermediate 5: (R,E)-2-Methyl-N—((R)-2-(((R)-1,1,1-trifluoropropan-2-yl)oxy)propylidene)propane-2-sulfinamide

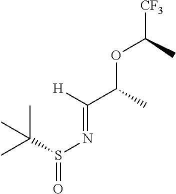 [SMILES: C[C@H](/C=N/[S@](=O)C(C)(C)C)O[C@H](C)C(F)(F)F]

A round-bottom flask was charged with (R)-2-(((R)-1,1,1-trifluoropropan-2-yl)oxy)propanal (4.0 g, 24 mmol, Intermediate 4), DCM (200 mL), CuSO₄ (15 g, 94 mmol), 4 Å molecular sieves (4.0 g), (R)-2-methylpropane-2-sulfinamide (5.7 g, 47 mmol), and PPTS (0.59 g, 2.4 mmol), and the resulting mixture was stirred at rt for 12 h. The suspension was filtered through diatomaceous earth (e.g., Celite©), the filter cake was rinsed with EtOAc (100 mL) and the filtrate was concentrated under reduced pressure. This product was purified by silica gel chromatography (17-25% EtOAc/petroleum ether) to afford the title compound in 40% yield as a yellow oil.

### Intermediate 6: (R)-2-(((S)-1,1,1-Trifluoropropan-2-yl)oxy)propanoic acid

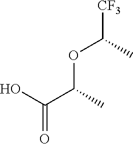

The title compound was prepared as described for the synthesis of Intermediate 2, using (S)-1,1,1-trifluoropropan-2-ol in place of (R)-1,1,1-trifluoropropan-2-ol in 80% yield.

### Intermediate 7: (R)—N-Methoxy-N-methyl-2-(((S)-1,1,1-trifluoropropan-2-yl)oxy)propanamide

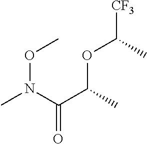

The title compound was prepared as described for the synthesis of Intermediate 3, using (R)-2-(((S)-1,1,1-trifluoropropan-2-yl)oxy)propanoic acid (Intermediate 6) in place of (R)-2-(((R)-1,1,1-trifluoropropan-2-yl)oxy)propanoic acid in 46% yield.

### Intermediate 8: (R)-2-(((S-1,1,1-Trifluoropropan-2-yl)oxy)propanal

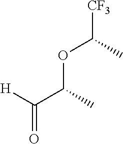

The title compound was prepared as described for the synthesis of Intermediate 4, using (R)—N-methoxy-N-methyl-2-(((S)-1,1,1-trifluoropropan-2-yl)oxy)propanamide (Intermediate 7) in place of (R)—N-methoxy-N-methyl-2-(((R)-1,1,1-trifluoropropan-2-yl)oxy)propanamide in 80% yield.

### Intermediate 9: (R)-2-Methyl-N—((R,E)-2-(((S)-1,1,1-trifluoropropan-2-yl)oxy)propylidene)propane-2-sulfinamide

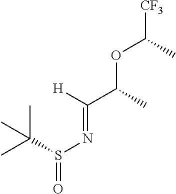 [SMILES: C[C@H](/C=N/[S@](=O)C(C)(C)C)O[C@@H](C)C(F)(F)F]

The title compound was prepared as described for the synthesis of Intermediate 5, using (R)-2-(((S-1,1,1-trifluoropropan-2-yl)oxy)propanal (Intermediate 8) in place of (R)-2-(((R)-1,1,1-trifluoropropan-2-yl)oxy)propanal in 51% yield.

### Intermediate 10: 1-((2-(Trimethylsilyl)ethoxy)methyl)-1H-benzo[d]imidazole-5-carbaldehyde

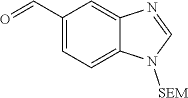 [SMILES: C[Si](C)(C)CCOCn1cnc2cc(C=O)ccc21]

Step A: 2-(4-Fluoro-3-nitrophenyl)-1,3-dioxolane. 4-Fluoro-3-nitrobenzaldehyde (50.0 g, 296 mmol) and toluene (1.0 L) were added to a 2 L one-necked round-bottom flask equipped with a Dean-Stark trap under N₂. Ethane-1,2-diol (55.0 g, 886 mmol) and TsOH (1.0 g, 5.8 mmol) were sequentially added with stirring at 25° C., and the resulting mixture was stirred for 5 h at 110° C. under N₂. The mixture was allowed to cool to 25° C. and concentrated under reduced pressure. The resulting mixture was dissolved with EtOAc (1 L), washed with water (3×1 L) and brine (1 L), dried over anhydrous Na₂SO₄, filtered, and concentrated under reduced pressure to afford the title compound in 93% yield as an off-white solid.

Step B: N-(4-(1,3-Dioxolan-2-yl)-2-nitrophenyl)formamide. A 3-necked 2-L round-bottom flask was charged with t-BuOK (78.9 g, 703 mmol) and NMP (720 mL) under N₂. A solution of 2-(4-fluoro-3-nitrophenyl)-1,3-dioxolane (60.0 g, 281 mmol, Step A) and formamide (57.0 g, 1.27 mol) in NMP (480 mL) was added dropwise over 10 min with stirring at 5° C. The reaction was stirred at 5° C. for 30 min then cooled to 0° C., quenched with 7 L of saturated aqueous NH₄Cl solution and the resulting mixture was extracted with EtOAc (2×2 L). The combined organic layers were washed with brine (5×4 L), dried over anhydrous MgSO₄, filtered, and concentrated under reduced pressure to afford a yellow solid. A mixture of MTBE/EtOAc (10:1, 45 mL) was added to the solid and the resulting slurry stirred for 16 h. After filtration, the filter cake was collected to afford the title compound in 62% yield as a yellow solid

Step C: N-(4-(1,3-Dioxolan-2-yl)-2-nitrophenyl)-N-((2-(trimethylsilyl)ethoxy)methyl)formamide. A 2 L, 3-necked round-bottom flask was charged with N-(4-(1,3-dioxolan-2-yl)-2-nitrophenyl)formamide (40.8 g, 171 mmol, Step B), SEM-Cl (32.9 g, 197 mmol), BTEAC (11.2 g, 49.1 mmol), and DCM (400 mL). The mixture was cooled to 5° C. and an aqueous solution of NaOH (10% w/v, 400 mL) was added dropwise over 10 min. The reaction was allowed to warm to 25° C. and stirred for 3 h. The layers were separated, and the organic layer was washed with water (3×600 mL) and brine (600 mL), dried over anhydrous Na₂SO₄, filtered, and concentrated under reduced pressure to afford the title compound in 83% yield as a brown oil that was used without further purification.

Step D: 1-((2-(Trimethylsilyl)ethoxy)methyl)-1H-benzo[d]imidazole-5-carbaldehyde. A 2 L 3-necked round-bottom flask was charged with N-(4-(1,3-dioxolan-2-yl)-2-nitrophenyl)-N-((2-(trimethylsilyl)ethoxy)methyl)formamide (73.0 g, 198 mmol, Step C), Fe^{o} (14.4 g, 259 mmol), AcOH (300 mL), and EtOH (730 mL). The resulting mixture was stirred for 16 h at 80° C., after which time, the reaction was cooled to rt and concentrated under reduced pressure. The residue was dissolved in EtOAc (1 L) and filtered. The filtrate was washed with water (5×1 L), saturated aqueous Na₂CO₃ solution (1 L), and brine (1 L), sequentially, dried over anhydrous Na₂SO₄, filtered, and concentrated under reduced pressure to afford a brown oil, which was used directly in the next step.

### Intermediate 11: (S,E)-2-Methyl-N-((1-((2-(trimethylsilyl)ethoxy)methyl)-1H-benzo[d]imidazol-5-yl)methylene)propane-2-sulfinamide

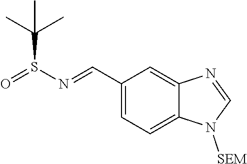 [SMILES: CC(C)(C)[S@](=O)/N=C/c1ccc2c(c1)ncn2COCC[Si](C)(C)C]

A 2 L 3-necked round-bottom flask was charged with 1-((2-(trimethylsilyl)ethoxy)methyl)-1H-benzo[d]imidazole-5-carbaldehyde (44.0 g, 95.8 mmol, Intermediate 10), (S)-2-methylpropane-2-sulfinamide (13.9 g, 115 mmol), KHSO₄ (43.4 g, 319 mmol), and toluene (800 mL). The resulting mixture was stirred for 4 h at 40° C., after which time, the reaction was cooled to 25° C. The reaction mixture was washed with water (3×1 L) and brine (1 L), dried over anhydrous Na₂SO₄, filtered, and concentrated under reduced pressure. The residue was purified by silica gel chromatography (1:1 petroleum ether/EtOAc), concentrated, slurried in n-heptane (10V) for 4 h, and filtered. The resulting filter cake was collected to afford the title compound in 70% yield as a yellow solid.

### Intermediate 12: (R,E)-2-Methyl-N-((1-((2-(trimethylsilyl)ethoxy)methyl)-1H-benzo[d]imidazol-5-yl)methylene)propane-2-sulfinamide

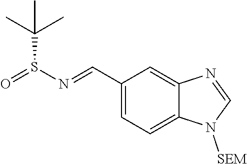 [SMILES: CC(C)(C)[S@@](=O)/N=C/c1ccc2c(c1)ncn2COCC[Si](C)(C)C]

To a solution of 1-((2-(trimethylsilyl)ethoxy)methyl)-1H-benzo[d]imidazole-5-carbaldehyde (16.4 g, 59.3 mmol, Intermediate 10) and (R)-2-methylpropane-2-sulfinamide (14.4 g, 119 mmol) dissolved in THF (500 mL) was added pyridin-1-ium 4-methylbenzenesulfonate (1.81 g, 6.12 mmol) and copper(II) sulfate (29.4 g, 184 mmol). The reaction mixture was heated at 80° C. for 16 h. After that time, the reaction was cooled to rt, filtered through a pad of diatomaceous earth (e.g, Celite©) and the filter cake washed with EtOAc (2×200 mL). The filtrate was concentrated under reduced pressure and the isolated material was purified by silica gel chromatography (0-100% EtOAc/petroleum ether) to afford the title compound as a colorless oil (59% yield).

### Intermediate 13: 5-Bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole

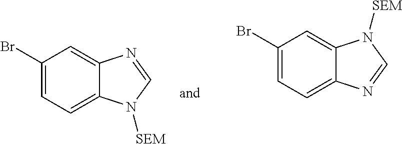 [SMILES: C[Si](C)(C)CCOCn1cnc2cc(Br)ccc21 ; C[Si](C)(C)CCOCn1cnc2ccc(Br)cc21]

To a solution of 5-bromo-1H-benzo[d]imidazole (20.0 g, 102 mmol) in THF (135 mL) was added NaH (6.09 g, 152 mmol, 60% suspension in mineral oil) and the resulting mixture was stirred at 0° C. for 30 min. Then the purple mixture was cooled to 0° C. and SEM-Cl (46.5 g, 279 mmol, 49.4 mL) was added in portions over 1 h. The yellow mixture was then allowed to slowly warm to 20° C. then stirred at that temperature for 4 h. The reaction mixture was cooled down to 0° C., quenched with water slowly, and extracted with EtOAc (3×75 mL). The combined organic layers were washed with brine, dried over anhydrous MgSO₄, filtered, and concentrated under reduced pressure. The residue was purified by silica gel chromatography (0-100% acetone/hexanes) to provide a mixture of title compounds in 89% yield as a yellow oil.

### Intermediate 14: 1-((2-(Trimethylsilyl)ethoxy)methyl)-5-vinyl-1H-benzo[d]imidazole and 1-((2-(trimethylsilyl)ethoxy)methyl)-6-vinyl-1H-benzo[d]imidazole

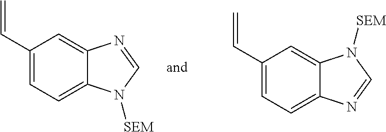 [SMILES: C=Cc1ccc2c(c1)ncn2COCC[Si](C)(C)C ; C=Cc1ccc2ncn(COCC[Si](C)(C)C)c2c1]

A solution of 1,4-dioxane (100 mL) and water (20 mL) was sparged with nitrogen for 10 min, followed by the addition of 5-bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole (7.00 g, 21.4 mmol, Intermediate 13), potassium trifluoro(vinyl)boranide (5.40 g, 40.3 mmol), K₃PO₄ (13.6 g, 64.1 mmol) and [1,1′-bis(diphenylphosphino)ferrocene]dichloropalladium(II) complex with dichloromethane (875 mg, 1.07 mmol). The brown mixture was stirred at 85° C. for 3 h. Then the reaction mixture was cooled to rt, diluted with H₂O (100 mL), and extracted with EtOAc (2×300 mL). The combined organic layers were washed with brine, dried over anhydrous sodium sulfate, filtered, and concentrated under reduced pressure to provide the mixture of title compounds in 91% yield as a black oil that was used directly without purification.

### Intermediate 15: 1-((2-(Trimethylsilyl)ethoxy)methyl)-1H-benzo[d]imidazole-5-carbaldehyde and 1-((2-(trimethylsilyl)ethoxy)methyl)-1H-benzo[d]imidazole-6-carbaldehyde

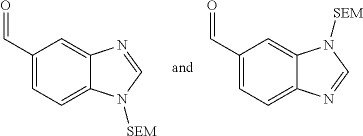 [SMILES: C[Si](C)(C)CCOCn1cnc2cc(C=O)ccc21 ; C[Si](C)(C)CCOCn1cnc2ccc(C=O)cc21]

To a solution of 1-((2-(trimethylsilyl)ethoxy)methyl)-5-vinyl-1H-benzo[d]imidazole and 1-((2-(trimethylsilyl)ethoxy)methyl)-6-vinyl-1H-benzo[d]imidazole (560. g, 2.04 mol, Intermediate 14) and NaIO₄ (1.75 kg, 8.16 mol) in 1,4-dioxane (5.60 L) and H₂O (2.24 L) was added K₂OsO₄·2H₂O (2.42 g, 6.56 mmol) in H₂O (100 mL) dropwise at 5-10° C. over 30 min. The yellow suspension was stirred at 25° C. for 5 h. Then the reaction was diluted with H₂O (5 L) and was extracted with EtOAc (3×5 L). The combined organic layers were washed with brine (3×5 L), dried over anhydrous sodium sulfate, filtered, and concentrated under reduced pressure onto silica gel. The residue was purified by silica gel chromatography (33% EtOAc/petroleum ether) to provide the mixture of title compounds in 78% yield as a yellow solid.

### Intermediate 16: (S,E)-2-Methyl-N-((1-((2-(trimethylsilyl)ethoxy)methyl)-1H-benzo[d]imidazol-5-yl)methylene)propane-2-sulfinamide and (S,E)-2-methyl-N-((1-((2-(trimethylsilyl)ethoxy)methyl)-1H-benzo[d]imidazol-6-yl)methylene)propane-2-sulfinamide

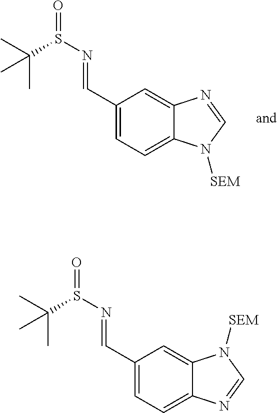 [SMILES: CC(C)(C)[S@](=O)/N=C/c1ccc2c(c1)ncn2COCC[Si](C)(C)C ; CC(C)(C)[S@](=O)/N=C/c1ccc2ncn(COCC[Si](C)(C)C)c2c1]

1-((2-(Trimethylsilyl)ethoxy)methyl)-1H-benzo[d]imidazole-5-carbaldehyde and 1-((2-(trimethylsilyl)ethoxy)methyl)-1H-benzo[d]imidazole-6-carbaldehyde (3.0 g, 11 mmol, Intermediate 15), (S)-2-methylpropane-2-sulfinamide (2.0 g, 17 mmol), CuSO₄ (5.0 g, 31 mmol) and DCM (20 mL) were combined followed by the addition of PPTS (300 g, 1.2 mmol). The blue mixture was stirred at 35° C. for 12 h. After that time, the reaction mixture was filtered through diatomaceous earth (e.g, Celite©), and the filter cake was washed with DCM (20 mL). The filtrate was concentrated to dryness and the residue was purified by silica gel chromatography (0-100% EtOAc/petroleum ether) to provide a mixture of title compounds in 47% yield as a yellow gum.

### Intermediate 17: (R,E)-2-Methyl-N-((1-((2-(trimethylsilyl)ethoxy)methyl)-1H-benzo[d]imidazol-5-yl)methylene)propane-2-sulfinamide and (R,E)-2-methyl-N-((1-((2-(trimethylsilyl)ethoxy)methyl)-1H-benzo[d]imidazol-6-yl)methylene)propane-2-sulfinamide

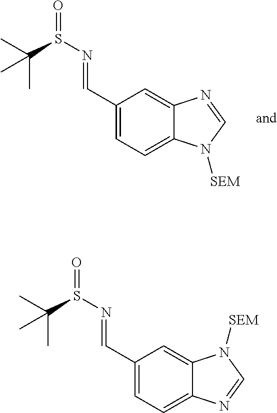 [SMILES: CC(C)(C)[S@@](=O)/N=C/c1ccc2c(c1)ncn2COCC[Si](C)(C)C ; CC(C)(C)[S@@](=O)/N=C/c1ccc2ncn(COCC[Si](C)(C)C)c2c1]

The title compounds were prepared as described for Intermediate 16, using (R)-2-methylpropane-2-sulfinamide in place of (S)-2-methylpropane-2-sulfinamide in a 60% yield.

### Intermediate 18: 4-Methyl-1,2,5-oxadiazole-3-carbonyl chloride

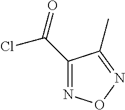

A flame-dried, round bottom flask was charged with 4-methyl-1,2,5-oxadiazole-3-carboxylic acid (5.0 g, 39 mmol), DCM (78 mL), and oxalyl chloride (6.7 mL, 78 mmol). The solution was cooled to 0° C. and to the solution was added DMF (0.30 mL, 4.0 mmol). The mixture was stirred for 4 h as it warmed to rt. Then, the mixture was concentrated into a yellow oil and dissolved in DCM to result in a 2 M solution of the title compound that was used in subsequent reactions without further purification assuming 100% yield.

### Intermediate 19: 2,5-Dioxopyrrolidin-1-yl 4-methyl-1,2,5-oxadiazole-3-carboxylate

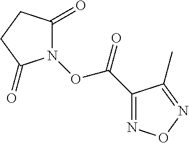 [SMILES: Cc1nonc1C(=O)ON1C(=O)CCC1=O]

A flame-dried round bottom flask was charged with N-hydroxysuccinimide (6.95 g, 58.6 mmol), DCM (98 mL), and DIPEA (10.1 mL, 58.6 mmol). The reaction mixture was cooled to 0° C. and 4-methyl-1,2,5-oxadiazole-3-carbonyl chloride (19.5 mL, 39.0 mmol, Intermediate 18) was added dropwise. The reaction was stirred at rt overnight. Without adding additional solvent, the reaction mixture was washed with water and brine, dried over anhydrous MgSO₄, filtered, and concentrated to dryness. The residue was purified by silica gel chromatography (0-100% EtOAc (with 10% MeOH)/hexanes) to afford the title compound in 50% yield as a colorless solid.

### Intermediate 20: Ethyl 1-(ethyl-d₅)-1H-pyrazole-5-carboxylate

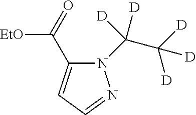 [SMILES: [2H]C([2H])([2H])C([2H])([2H])n1nccc1C(=O)OCC]

To a mixture of ethyl 1H-pyrazole-3-carboxylate (6.2 g, 44 mmol), K₂CO₃ (9.1 g, 66 mmol) and DMF (55 mL) was added bromoethane-d₅ (5.0 g, 44 mmol) and the resulting mixture was stirred at rt for 15 h. The reaction mixture was partitioned between EtOAc and water. The layers were separated, and the aqueous layer was further extracted with EtOAc. The organic layers were combined, washed with brine, dried over anhydrous Na₂SO₄, filtered, and concentrated to dryness to afford the title compound and its regioisomer. This residue was purified by silica gel chromatography (10-60% EtOAc/hexanes) to afford the title compound as the first eluting fraction in 47% yield, as a colorless oil.

### Intermediate 21: 1-(Ethyl-d₅)-1H-pyrazole-5-carboxylic acid

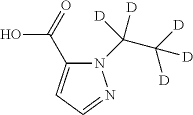 [SMILES: [2H]C([2H])([2H])C([2H])([2H])n1nccc1C(=O)O]

To a mixture of ethyl 1-(ethyl-d₅)-1H-pyrazole-5-carboxylate (3.51 g, 20.2 mmol, Intermediate 20) in THF (61 mL) was added 2 M aqueous NaOH (61 mL, 122 mmol) and the mixture was stirred at rt for 3 h. After that time, the mixture was concentrated to remove the THF and then washed with DCM (2×50 mL). The aqueous layer was then acidified to pH 1 by the addition of 1 N aqueous HCl and extracted with DCM (3×50 mL). The organic layers were combined, washed with brine (50 mL), dried over anhydrous Na₂SO₄, filtered, and concentrated to dryness to provide the title compound in 85% yield as a white solid.

### Intermediate 22: 4-Cyclopropyl-1,2,5-oxadiazole-3-carbonyl chloride

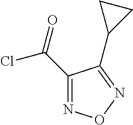 [SMILES: O=C(Cl)c1nonc1C1CC1]

The title compound was prepared as described for the synthesis of Intermediate 18, using 4-cyclopropyl-1,2,5-oxadiazole-3-carboxylic acid in place of 4-methyl-1,2,5-oxadiazole-3-carboxylic acid, assuming 100% yield.

### Intermediate 23: 2,5-Dioxopyrrolidin-1-yl 4-cyclopropyl-1,2,5-oxadiazole-3-carboxylate

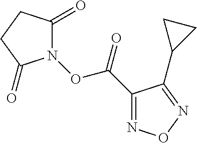 [SMILES: O=C(ON1C(=O)CCC1=O)c1nonc1C1CC1]

The title compound was prepared as described for the synthesis of Intermediate 19 using 4-cyclopropyl-1,2,5-oxadiazole-3-carbonyl chloride (Intermediate 22) in place of 4-methyl-1,2,5-oxadiazole-3-carbonyl chloride in 76% yield.

### Intermediate 24: Benzyl (S)-4-(trifluoromethyl)-1,2,3-oxathiazolidine-3-carboxylate 2,2-dioxide

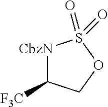 [SMILES: O=C(OCc1ccccc1)N1[C@H](C(F)(F)F)COS1(=O)=O]

Step A: (S)-Benzyl (1,1,1-trifluoro-3-hydroxypropan-2-yl)carbamate. (S)-2-Amino-3,3,3-trifluoropropan-1-ol hydrochloride (5.0 g, 30 mmol), NaHCO₃ (7.6 g, 91 mmol), a stir bar, and H₂O (50 mL) were added to a round-bottomed flask, and the resulting solution treated with CbzCl (6.2 g, 36 mmol). The mixture was stirred at 30° C. for 16 h under N₂. After that time, the mixture was diluted with water (100 mL) and extracted with ethyl acetate (50 mL×3). The combined organic extracts were washed with brine (100 mL), dried over anhydrous Na₂SO₄, filtered, and concentrated to dryness under reduced pressure. The resulting material was purified by silica gel chromatography (0-20% EtOAc/DCM) to provide the title compound in 80% yield as a white solid.

Step B: Benzyl (4S)-4-(trifluoromethyl)-1,2,3-oxathiazolidine-3-carboxylate 2-oxide. A stir bar, CH₃CN (70 mL), and SOCl₂ (4.5 mL, 62 mmol) were added to a dry round-bottomed flask under nitrogen, and the resulting solution cooled to −30° C. A solution consisting of (S)-benzyl (1,1,1-trifluoro-3-hydroxypropan-2-yl)carbamate (6.3 g, 24 mmol, Step A) and CH₃CN (30 mL) was then added dropwise to the reaction vessel over 10 min, followed by dropwise addition of pyridine (10.3 mL, 127 mmol), also over the course of 10 min. The mixture was stirred for 2 h at −30° C., and then poured over crushed ice (100 g) and treated with aqueous 10% KHSO₄ until the pH of the reaction mixture was pH 4. The resulting mixture was extracted with DCM (3×50 mL) and the combined organic extracts were washed with saturated aqueous NaHCO₃ (50 mL) and brine (50 mL), dried over anhydrous Na₂SO₄, filtered, and concentrated to dryness under reduced pressure. The residue was purified by silica gel chromatography (0-25% EtOAc/petroleum ether) to provide the title compound in 61% yield.

Step C: Benzyl (S)-4-(trifluoromethyl)-1,2,3-oxathiazolidine-3-carboxylate 2,2-dioxide. Benzyl (4S)-4-(trifluoromethyl)-1,2,3-oxathiazolidine-3-carboxylate 2-oxide (4.5 g, 15 mmol, Step B), a stir bar, and CH₃CN (30 mL) were added to a round-bottom flask, which was then cooled to 0° C., and subsequently charged with RuCl₃ (302 mg, 1.46 mmol), NaIO₄ (3.4 g, 16 mmol), and water (30 mL). The resulting mixture was stirred at 0° C. for 15 min, removed from the ice bath, and then stirred for an additional 4 h. The reaction mixture was then treated with brine (50 mL) and extracted with MTBE (4×50 mL). The combined organic extracts were washed with saturated aqueous NaHCO₃ (50 mL) and brine (100 mL), dried over anhydrous Na₂SO₄, filtered, and concentrated to dryness under reduced pressure. The isolated material was purified by silica gel chromatography (20-30% EtOAc/petroleum ether) to provide the title compound in 46% yield as a white solid.

### Intermediate 25: Benzyl (S*)-(1,1,1-trifluoro-3-hydroxypropan-2-yl-3,3-d₂)carbamate

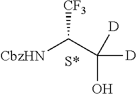 [SMILES: [2H]C([2H])(O)[C@H](NC(=O)OCc1ccccc1)C(F)(F)F]

Step A: Methyl 2-(((benzyloxy)carbonyl)amino)-3,3,3-trifluoropropanoate. A 500 mL round-bottom flask was charged with NaHCO₃ (55 g, 650 mmol) and water (100 mL) and was cooled to 0° C. THF (200 mL) was then added followed by the portion-wise addition of methyl 2-amino-3,3,3-trifluoropropanoate hydrochloride (25 g, 130 mmol). After 5 min, benzyl chloroformate (36.5 mL, 259 mmol) was added dropwise. After 1 h at 0° C., the reaction was diluted with EtOAc and the biphasic solution was separated. The aqueous layer was extracted three times with EtOAc. The combined organic layers were washed with brine, dried over anhydrous MgSO₄, filtered, and concentrated to dryness to afford a colorless solid. The product was purified by silica gel chromatography (0-30% EtOAc/hexanes) to afford the title compound in 40% yield as a white solid.

Step B: Benzyl (S*)-(1,1,1-trifluoro-3-hydroxypropan-2-yl-3,3-d₂)carbamate. A round-bottom flask was charged with methyl 2-(((benzyloxy)carbonyl)amino)-3,3,3-trifluoropropanoate (2.3 g, 7.8 mmol, Step A) and MeOH (30 mL) and cooled to 0° C. NaBD₄ (1.0 g, 24 mmol) was added portion-wise, and the sides of the flask were rinsed with MeOH (5 mL) after complete addition. After 1.5 h at 0° C., the reaction mixture was quenched with a small amount of water and poured into brine. This solution was extracted three times with EtOAc, and the combined organic layers were washed with brine, dried over anhydrous MgSO₄, filtered, and concentrated to produce a colorless oil that solidified over time. The product was purified by silica gel chromatography (0-50%, EtOAc/hexanes) to afford the racemic title compound in 90% yield. The diastereomeric mixture was separated by for SFC using the following conditions: Stationary phase: Lux Cellulose 2, 5 μm 250×21 mm, Mobile phase: 15% methanol:isopropanol (1:1), 85% CO₂), flow rate 96 mL/min. The retention time of the title compound was 1.66 min.

### Intermediate 26: Benzyl (S*)-4-(trifluoromethyl)-1,2,3-oxathiazolidine-3-carboxylate-5,5-d₂ 2,2-dioxide

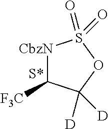 [SMILES: [2H]C1([2H])OS(=O)(=O)N(C(=O)OCc2ccccc2)[C@@H]1C(F)(F)F]

Step A: Benzyl (4S*)-4-(trifluoromethyl)-1,2,3-oxathiazolidine-3-carboxylate-5,5-d₂ 2-oxide. A stir bar, CH₃CN (39 mL), and SOCl₂ (2.0 mL, 27 mmol) were added to a dry round-bottom flask under nitrogen, and the resulting solution cooled to −48° C. A solution consisting of benzyl (S*)-(1,1,1-trifluoro-3-hydroxypropan-2-yl-3,3-d₂)carbamate (2.8 g, 11 mmol, Intermediate 25) and CH₃CN (17 mL) was then added dropwise to the reaction vessel over 10 min, followed by dropwise addition of pyridine (4.5 mL, 56 mmol). The mixture was stirred for 15 min at −45° C. and an additional 2 h at −35° C. The reaction was then poured over crushed ice (100 g) and diluted with CH₂Cl₂ (50 mL). The aqueous layer was saturated with NaCl, the layers were separated, and the organic layer was concentrated to a residue. The residue was redissolved in EtOAc (30 mL), washed with 0.1 N aqueous HCl, dried over anhydrous Na₂SO₄, filtered, and concentrated under reduced pressure to afford the product in 99% yield as a colorless oil which was used without further purification.

Step B: Benzyl (S*)-4-(trifluoromethyl)-1,2,3-oxathiazolidine-3-carboxylate-5,5-d₂ 2,2-dioxide. A round-bottom flask was charged with benzyl (4S*)-4-(trifluoromethyl)-1,2,3-oxathiazolidine-3-carboxylate-5,5-d₂ 2-oxide (1.0 g, 3.3 mmol, Step A) in MeCN (26 mL) and cooled to 0° C. NaIO₄ (0.91 g, 4.3 mmol), RuCl₃·3H₂O (11 mg, 0.043 mmol), and water (20 mL) were sequentially added and the reaction mixture was stirred for 40 min at 0° C. After full consumption of starting material was observed by LCMS analysis, the reaction mixture was diluted with additional water (20 mL), warmed to rt, and stirred for an additional 30 minutes. The biphasic solution was separated, and the aqueous layer was extracted with EtOAc (3×50 mL). The combined organic layers were dried over anhydrous Na₂SO₄, filtered, and concentrated under reduced pressure. The residue was purified by silica gel chromatography (0-50% EtOAc/hexanes) to afford the title compound in 40% yield as a white solid.

### Intermediate 27: Benzyl 4-(trifluoromethyl)-1,2,3-oxathiazolidine-3-carboxylate-5,5-d₂ 2-oxide

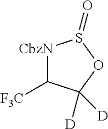 [SMILES: [2H]C1([2H])OS(=O)N(C(=O)OCc2ccccc2)C1C(F)(F)F]

Step A: Benzyl (1,1,1-trifluoro-3-hydroxypropan-2-yl-3,3-d₂)carbamate. The title compound was prepared as described for the synthesis of Intermediate 25. However, in Step B, benzyl (1,1,1-trifluoro-3-hydroxypropan-2-yl-3,3-d₂)carbamate was not subjected to chiral SFC separation.

Step B: Benzyl 4-(trifluoromethyl)-1,2,3-oxathiazolidine-3-carboxylate-5,5-d₂ 2-oxide. A round-bottom flask was charged with imidazole (2.3 g, 33 mmol), Et₃N (2.5 mL, 18 mmol), and DCM (69 mL) and the mixture was cooled to −40° C. Then thionyl chloride (0.70 mL, 9.5 mmol) was added dropwise and the resulting solution was allowed to stir for 5 min. Benzyl (1,1,1-trifluoro-3-hydroxypropan-2-yl-3,3-d₂)carbamate (2.2 g, 8.3 mmol, Step A) in DCM (49 mL) was then added dropwise via addition funnel. Upon complete addition, the reaction was warmed to 0° C. and stirred for 1 h. After this time, water was added, and the biphasic mixture was extracted three times with DCM. The combined organic layers were washed with brine, dried over anhydrous MgSO₄, filtered, and concentrated under reduced pressure. The product was carried on without further purification (assumed 100% yield).

### Intermediate 28: Benzyl (trifluoromethyl)-1,2,3-oxathiazolidine-3-carboxylate-5,5-d₂ 2,2-dioxide

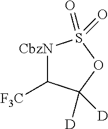 [SMILES: [2H]C1([2H])OS(=O)(=O)N(C(=O)OCc2ccccc2)C1C(F)(F)F]

The title compound was prepared as described for the synthesis of Intermediate 26, using benzyl 4-(trifluoromethyl)-1,2,3-oxathiazolidine-3-carboxylate-5,5-d₂ 2-oxide (Intermediate 27) in place of benzyl (4S)-4-(trifluoromethyl)-1,2,3-oxathiazolidine-3-carboxylate-5,5-d₂ 2-oxide to afford the title compound in 37% yield.

### Intermediate 29: tert-Butyl 4-(trifluoromethyl)-1,2,3-oxathiazolidine-3-carboxylate-5,5-d₂ 2-oxide

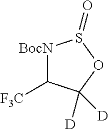 [SMILES: [2H]C1([2H])OS(=O)N(C(=O)OC(C)(C)C)C1C(F)(F)F]

Step A: tert-Butyl (1,1,1-trifluoro-3-hydroxypropan-2-yl-3,3-d₂)carbamate. The title compound was prepared as described for the synthesis of Intermediate 25, using methyl 2-((tert-butoxycarbonyl)amino)-3,3,3-trifluoropropanoate in place of 2-(((benzyloxy)carbonyl)amino)-3,3,3-trifluoropropanoate and was used without further purification.

Step B: tert-Butyl 4-(trifluoromethyl)-1,2,3-oxathiazolidine-3-carboxylate-5,5-d₂ 2-oxide. The title compound was prepared as described for the synthesis of Intermediate 27, using tert-butyl (1,1,1-trifluoro-3-hydroxypropan-2-yl-3,3-d₂)carbamate (Step A) in place of benzyl 4-(trifluoromethyl)-1,2,3-oxathiazolidine-3-carboxylate-5,5-d₂ 2-oxide to afford the title compound in 91% yield.

### Intermediate 30: (R)-2-(Cyclopropyl(1-((2-(trimethylsilyl)ethoxy)methyl)-1H-benzo[d]imidazol-5-yl)methyl)isoindoline-1,3-dione

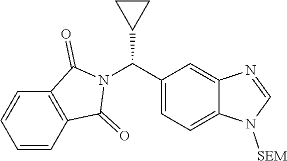 [SMILES: C[Si](C)(C)CCOCn1cnc2cc([C@@H](C3CC3)N3C(=O)c4ccccc4C3=O)ccc21]

Step A: (S)—N—((R)-Cyclopropyl(1-((2-(trimethylsilyl)ethoxy)methyl)-1H-benzo[d]imidazol-5-yl)methyl)-2-methylpropane-2-sulfinamide. A 1 L 3-necked round-bottom flask was charged with cyclopropylmagnesium bromide (118 mL, 1 M in THF). Then a solution of TMEDA (13.4 g, 115 mmol) in THF (64 mL) was added dropwise over 5 min at 25° C., and the resulting mixture was stirred at this temperature for 0.5 h. The reaction mixture was then cooled to −78° C. and a solution of (S,E)-2-methyl-N-((1-((2-(trimethylsilyl)ethoxy)methyl)-1H-benzo[d]imidazol-5-yl)methylene)propane-2-sulfinamide (16.0 g, 42.2 mmol, Intermediate 11) in THF (320 mL) was added dropwise over 30 min and the resulting mixture was allowed to stir for 1 h at −78° C. The reaction mixture was quenched with saturated NH₄Cl (aqueous, 500 mL) at 0° C. and the layers were separated. The organic layer was washed with water (2×500 mL) and brine (500 mL), dried over anhydrous Na₂SO₄, filtered, and concentrated under reduced pressure. The residue was purified by silica gel chromatography (15:1 CH₂Cl₂/MeOH). The product containing fractions were concentrated under reduced pressure and the residue was further purified by crystallization in n-heptane:MTBE (25:1.52V) to afford the title compound in 92% yield as an off-white solid.

Step B: (R)-Cyclopropyl(1-((2-(trimethylsilyl)ethoxy)methyl)-1H-benzo[d]imidazol-5-yl)methanamine. To a solution of (S)—N—((R)-cyclopropyl(1-((2-(trimethylsilyl)ethoxy)methyl)-1H-benzo[d]imidazol-5-yl)methyl)-2-methylpropane-2-sulfinamide (2.0 g, 4.7 mmol, Step A) in EtOAc (20 mL) was added 4 M HCl in 1,4-dioxane (5.0 mL, 20 mmol) at 0° C. under nitrogen. The mixture was stirred at 0° C. for 30 min, then the mixture was warmed to rt and stirred for 16 h. To the reaction was added petroleum ether (200 mL) and the mixture was filtered. To the filtered solids was added saturated aqueous NaHCO₃ solution, then the mixture was extracted with EtOAc (3×100 mL). The combined organic layers were dried over anhydrous Na₂SO₄, filtered, and concentrated under reduced pressure to provide the title compound in 86% yield as a yellow oil.

Step C: (R)-2-(Cyclopropyl(1-((2-(trimethylsilyl)ethoxy)methyl)-1H-benzo[d]imidazol-5-yl)methyl)isoindoline-1,3-dione. (R)-Cyclopropyl(1-((2-(trimethylsilyl)ethoxy)methyl)-1H-benzo[d]imidazol-5-yl)methanamine (13.2 g, 37.29 mmol, Step B) and THF (250 mL, 50 mmol) were combined and stirred at rt under nitrogen followed by the addition of Hunig's base (20 mL, 116 mmol). The reaction mixture was stirred for 5 min at rt then ethyl 1,3-dioxoisoindoline-2-carboxylate (8.57 g, 39.1 mmol) was added, a reflux condenser connected, and the contents heated to reflux for 2 d. The contents were cooled to rt and transferred to a separatory funnel with EtOAc dilution, and then washed twice with deionized water. The organic phase was separated, dried over anhydrous magnesium sulfate, filtered, and concentrated under reduced pressure. Purification by silica gel chromatography (0-100% EtOAc/hexanes) provided the title compound in 79% yield.

### Intermediate 31: (R)—N—((R)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

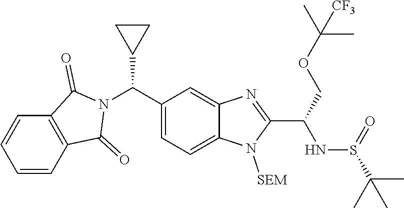 [SMILES: CC(C)(C)[S@@](=O)N[C@@H](COC(C)(C)C(F)(F)F)c1nc2cc([C@@H](C3CC3)N3C(=O)c4ccccc4C3=O)ccc2n1COCC[Si](C)(C)C]

To a −78° C. solution of (R)-2-(cyclopropyl(1-((2-(trimethylsilyl)ethoxy)methyl)-1H-benzo[d]imidazol-5-yl)methyl)isoindoline-1,3-dione (3190 mg, 6.97 mmol, Intermediate 30) and (R,E)-2-methyl-N-(2-((1,1,1-trifluoro-2-methylpropan-2-yl)oxy)ethylidene)propane-2-sulfinamide (2.86 g, 10.5 mmol, Intermediate 1) in THF (70 mL) was added LDA (11 mL, 13 mmol, 1.2 M in THF/hexanes). The reaction mixture was stirred at −78° C. for 30 min then quenched with AcOH (0.8 mL), warmed to rt, and poured into a mixture of saturated aqueous ammonium chloride (20 mL) and brine (50 mL). The mixture was extracted with EtOAc (2×100 mL). Then the combined organic layers were washed with brine, dried over anhydrous Na₂SO₄, filtered, and concentrated under reduced pressure. Purification by silica gel chromatography (0-100% EtOAc/DCM) provided the title compound in 57% yield.

### Intermediate 32: (R)—N—((R)-1-(5-((R)-Amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

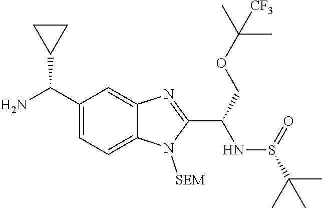 [SMILES: CC(C)(C)[S@@](=O)N[C@@H](COC(C)(C)C(F)(F)F)c1nc2cc([C@H](N)C3CC3)ccc2n1COCC[Si](C)(C)C]

To a solution of (R)—N—((R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (5640 mg, 7.82 mmol, Intermediate 31) in EtOH (78 mL) was added hydrazine monohydrate (3.5 mL, 48 mmol). The reaction mixture was stirred for 4 h at rt then concentrated under reduced pressure. The residue was dissolved in EtOAc, the precipitate removed by filtration, and the filtrate was concentrated under reduced pressure to provide the title compound which was used without further purification (assumed 100% yield).

### Intermediate 33: (R)—N—((R)-1-(5-((R)-Cyclopropyl((S)-2-oxo-4-(trifluoromethyl) imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

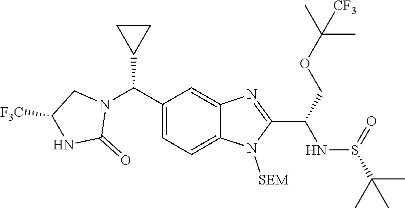 [SMILES: CC(C)(C)[S@@](=O)N[C@@H](COC(C)(C)C(F)(F)F)c1nc2cc([C@@H](C3CC3)N3C[C@@H](C(F)(F)F)NC3=O)ccc2n1COCC[Si](C)(C)C]

Step A: Benzyl ((S)-3-(((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)amino)-1,1,1-trifluoropropan-2-yl)carbamate. A round-bottom flask was charged with (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (1.2 g, 2.0 mmol, Intermediate 32), Cs₂CO₃ (2.0 g, 6.1 mmol), (S)-benzyl 4-(trifluoromethyl)-1,2,3-oxathiazolidine-3-carboxylate 2,2-dioxide (1.8 g, 5.5 mmol, Intermediate 24), and anhydrous DMF (30 mL) and the resulting mixture was heated to 30° C. The reaction mixture was stirred for about 16 h, until the complete consumption of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide was determined by LCMS analysis, at which time, the reaction mixture was diluted with water (100 mL) and extracted with EtOAc (3×30 mL). The combined organic layers were washed with brine, dried over anhydrous Na₂SO₄, filtered, and concentrated under reduced pressure to afford the product, which was purified by preparative HPLC (Boston Uni C18 40×150 mm, 5 μm, 50-80% CH₃CN/H₂O with 0.23% formic acid)) to afford the title compound in 77% yield as a white solid.

Step B: (R)—N—((R)-1-(5-((R)—(((S)-2-Amino-3,3,3-trifluoropropyl)amino) (cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. Benzyl ((S)-3-(((R)-cyclopropyl(2-((R)-1-((R)-1,1-dimethylethylsulfinamido)-2-((1,1,1-trifluoro-2-methylpropan-2-yl)oxy)ethyl)-1-((2-(trimethylsilyl)ethoxy)methyl)-1H-benzo [d]imidazol-5-yl)methyl)amino)-1,1,1-trifluoropropan-2-yl)carbamate (1.3 g, 1.6 mmol, Step A), MeOH (30 mL), and 10% Pd/C (1.3 g, 10% Pd (50% wet with water)) were added to a 75 mL hydrogenation bottle, and the resulting mixture stirred under H₂ (45 Psi) for 1.5 h. The reaction mixture was then filtered through diatomaceous earth (e.g, Celite©), the filter-cake rinsed with MeOH (30 mL), and the filtrate concentrated to dryness under reduced pressure to afford the product. The isolated material was purified by silica gel chromatography (0-8% MeOH/DCM) to afford the title compound in 92% yield as a colorless solid.

Step C: (R)—N—((R)-1-(5-((R)-Cyclopropyl((S)-2-oxo-4-(trifluoromethyl) imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. (R)—N—((R)-1-(5-((R)—(((S)-2-amino-3,3,3-trifluoropropyl)amino) (cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (1.5 g, 2.1 mmol, Step B), DCM (30 mL), and DIPEA (1.1 mL, 6.4 mmol) were added to a nitrogen-purged 100 mL three-necked round-bottom flask, which was subsequently cooled to 0° C. The resulting mixture was then treated with triphosgene (400 mg, 1.35 mmol) and stirred at this temperature for 30 min. The reaction mixture was then treated with water (50 mL), extracted with DCM (2×30 mL), and the combined extracts were concentrated under reduced pressure to afford the product. The isolated material was purified by silica gel chromatography (0-8% MeOH/DCM) to afford the title compound in 58% yield as a yellow solid.

### Intermediate 34: (S)-1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one

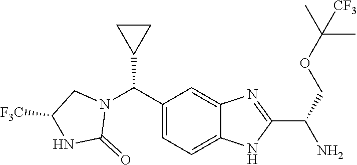 [SMILES: CC(C)(OC[C@H](N)c1nc2cc([C@@H](C3CC3)N3C[C@@H](C(F)(F)F)NC3=O)ccc2[nH]1)C(F)(F)F]

A round-bottom flask was charged with (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide (800 mg, 1.10 mmol, Intermediate 33), anhydrous MeOH (10 mL), and methanolic HCl (4 M, 10 mL, 40 mmol) and the resulting mixture stirred at 55° C. for 3 h. The reaction mixture was then cooled to rt, concentrated to dryness under reduced pressure, treated with saturated aqueous NaHCO₃ solution until the pH of the mixture was pH 8-9 and extracted with EtOAc (30 mL×3). The combined extracts were washed with brine (50 mL), dried over anhydrous Na₂SO₄, filtered, and concentrated to dryness to afford the crude product. The isolated material was purified by silica gel chromatography (0-7% MeOH/DCM) to afford the title compound in 41% yield as a light yellow solid.

### Intermediate 35: tert-Butyl 4-(trifluoromethyl)-1,2,3-oxathiazolidine-3-carboxylate-5,5-d₂ 2,2-dioxide

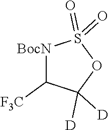 [SMILES: [2H]C1([2H])OS(=O)(=O)N(C(=O)OC(C)(C)C)C1C(F)(F)F]

The title compound was prepared as described for the synthesis of Intermediate 26 using tert-butyl 4-(trifluoromethyl)-1,2,3-oxathiazolidine-3-carboxylate-5,5-d₂ 2-oxide (Intermediate 29) in place of benzyl (4S)-4-(trifluoromethyl)-1,2,3-oxathiazolidine-3-carboxylate-5,5-d₂ 2-oxide to afford the title compound in 43% yield.

### Intermediate 36: (R)—N—((R)-1-(5-((R)-Cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

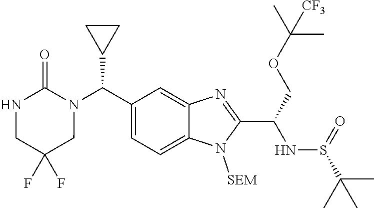 [SMILES: CC(C)(C)[S@@](=O)N[C@@H](COC(C)(C)C(F)(F)F)c1nc2cc([C@@H](C3CC3)N3CC(F)(F)CNC3=O)ccc2n1COCC[Si](C)(C)C]

Step A: (R)—N—((R)-1-(5-((R)-Cyclopropyl((3-(1,3-dioxoisoindolin-2-yl)-2,2-difluoropropyl)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. To a stirred solution of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (300 mg, 0.508 mmol, Intermediate 32), and 3-(1,3-dioxoisoindolin-2-yl)-2,2-difluoropropyl trifluoromethanesulfonate (379 mg, 1.02 mmol, as described in WO2010/011959) in acetonitrile (2.50 mL) was added DIPEA (97.1 μL, 0.559 mmol). The reaction mixture was heated at 50° C. for 36 h. The reaction mixture was cooled to rt, concentrated under reduced pressure, and purified by silica gel chromatography (20-70% acetone/(hexanes with 0.1% TEA)) to give the title compound (306 mg, 74%).

Step B: (R)—N—((R)-1-(5-((R)-((3-Amino-2,2-difluoropropyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. To a solution of (R)—N—((R)-1-(5-((R)-cyclopropyl((3-(1,3-dioxoisoindolin-2-yl)-2,2-difluoropropyl)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (510 mg, 0.627 mmol, Step A) in EtOH (3.13 mL) was added hydrazine monohydrate (93.0 μl, 1.88 mmol). After 1 h at rt, the reaction mixture was warmed to 35° C. and stirred for an additional 14 h, at which time a thick slurry had formed. The reaction mixture was cooled in an ice bath, filtered, the filter cake was washed with 10 mL of ice cold EtOH, and the filtrate concentrated to give the title compound which was used without further purification in the next step.

Step C: (R)—N—((R)-1-(5-((R)-Cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. To a stirred solution of (R)—N—((R)-1-(5-((R)-((3-amino-2,2-difluoropropyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (material from Step B) in THF (12 mL) was added CDI (152 mg, 0.94 mmol) and the reaction mixture was stirred at rt for 18 h. The reaction mixture was then diluted with THF (20 mL) and heated at 60° C. for an additional 3 h. The reaction mixture was cooled to rt, treated with aqueous 3 M NaOH (2 mL) while stirring for 10 min, diluted with brine (10 mL) and the layers separated. The organic layer was washed with brine (10 mL), dried over anhydrous Na₂SO₄, filtered and concentrated to give the title compound that was used without further purification.

### Intermediate 37: 1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one

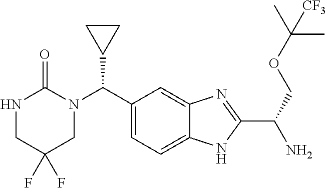 [SMILES: CC(C)(OC[C@H](N)c1nc2cc([C@@H](C3CC3)N3CC(F)(F)CNC3=O)ccc2[nH]1)C(F)(F)F]

To a solution of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (0.625 mmol (assumed), Intermediate 36) in 1,4-dioxane (36.6 mL) and MeOH (3.0 mL) at 65° C. was added HCl (2.35 mL, 4 M in 1,4-dioxane). After 2.5 h at this temperature, the reaction mixture was cooled to rt, concentrated to dryness under reduced pressure, and diluted with water (60 mL). The aqueous layer was washed with hexanes (2×20 mL) and the pH of the aqueous layer was adjusted to pH 11 with saturated aqueous NaHCO₃ solution and aqueous 3 M NaOH. This mixture was then diluted with brine (20 mL) and extracted with EtOAc (4×30 mL). The combined organic extracts were dried over anhydrous Na₂SO₄, filtered and concentrated to give the title compound as a tan solid that was used without further purification.

### Intermediate 38: (S)-2-Methyl-N—((R)-1-(1-((2-(trimethylsilyl)ethoxy)methyl)-1H-benzo[d]imidazol-5-yl)ethyl)propane-2-sulfinamide and (S)-2-methyl-N—((R)-1-(1-((2-(trimethylsilyl)ethoxy)methyl)-1H-benzo[d]imidazol-6-yl)ethyl)propane-2-sulfinamide

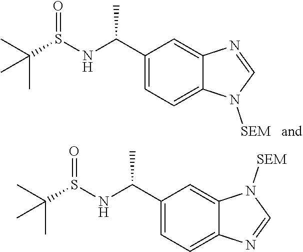

To a mixture of (S,E)-2-methyl-N-((1-((2-(trimethylsilyl)ethoxy)methyl)-1H-benzo[d]imidazol-5-yl)methylene)propane-2-sulfinamide and (S,E)-2-methyl-N-((1-((2-(trimethylsilyl)ethoxy)methyl)-1H-benzo[d]imidazol-6-yl)methylene)propane-2-sulfinamide (120 g, 316 mmol, Intermediate 16) in DCM (1.2 L) at −70° C. was added methyl magnesium bromide (3 M in THF, 948 mL, 2.84 mol). The brown mixture was warmed to 20° C. gradually and stirred for 12 h. The reaction mixture was quenched by the addition of saturated aqueous NH₄Cl solution (1 L). The reaction mixture was extracted with CH₂Cl₂ (2×1000 mL) and the combined organic layers were washed with brine (2×1000 mL), dried over anhydrous Na₂SO₄, filtered, and concentrated under reduced pressure. The residue was triturated from a mixture of MTBE (100 mL) and petroleum ether (400 mL) at 40° C. for 30 min and the precipitate was filtered to afford the title compounds in 30% yield as a white solid.

### Intermediate 39: (R)-1-(1-((2-(Trimethylsilyl)ethoxy)methyl)-1H-benzo[d]imidazol-6-yl)ethan-1-amine and (R)-1-(1-((2-(trimethylsilyl)ethoxy)methyl)-1H-benzo[d]imidazol-5-yl)ethan-1-amine

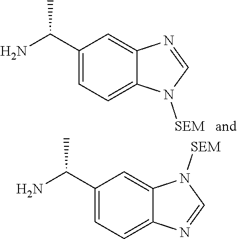

To a suspension of (S)-2-methyl-N—((R)-1-(1-((2-(trimethylsilyl)ethoxy)methyl)-1H-benzo[d]imidazol-6-yl)ethyl)propane-2-sulfinamide and (S)-2-methyl-N—((R)-1-(1-((2-(trimethylsilyl)ethoxy)methyl)-1H-benzo[d]imidazol-5-yl)ethyl)propane-2-sulfinamide (2.00 g, 5.06 mmol, Intermediate 38) in EtOAc (25 mL) was added HCl (5.1 mL, 20 mmol, 4 M in 1,4-dioxane). The reaction was stirred at rt for 1 h then diluted with H₂O (50 mL). The resulting solution was washed twice with hexanes and these hexane extracts were discarded. The pH of the aqueous layer was adjusted to pH 10 with NaOH (0.81 g) in a minimum amount of H₂O and then extracted three times with EtOAc. The combined organic layers were washed with brine, dried over anhydrous Na₂SO₄, filtered, and concentrated under reduced pressure to provide a mixture of the title compounds in 98% yield.

### Intermediate 40: (R)—N—((R*)-1-(6-((R)-1-Aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(5-((R)-1-aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

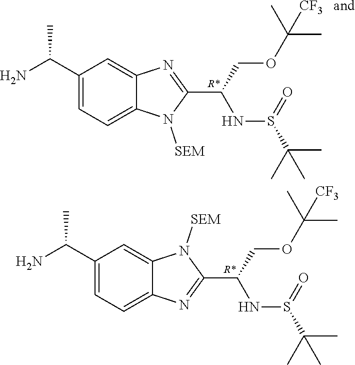

To a −78° C. solution of (R)-1-(1-((2-(trimethylsilyl)ethoxy)methyl)-1H-benzo[d]imidazol-6-yl)ethan-1-amine and (R)-1-(1-((2-(trimethylsilyl)ethoxy)methyl)-1H-benzo[d]imidazol-5-yl)ethan-1-amine (142 mg, 0.488 mmol, Intermediate 39) in THF (5 mL) was added n-BuLi (0.37 mL, 0.59 mmol, 1.6 M in hexanes). After stirring for 30 min at −78° C., (R,E)-2-methyl-N-(2-((1,1,1-trifluoro-2-methylpropan-2-yl)oxy)ethylidene)propane-2-sulfinamide (200 mg, 0.732 mmol, Intermediate 1) was added as a solution in THF (1 mL). The reaction was stirred for 30 min at −78° C. then quenched with EtOH (0.057 mL), diluted with EtOAc, and warmed to rt. The mixture was poured over brine and the aqueous layer extracted twice with EtOAc. Then, the combined organic layers were dried over anhydrous Na₂SO₄, filtered, and concentrated under reduced pressure to provide the mixture of title compounds, which were used without further purification.

### Intermediate 41: (R)-2-Methyl-N—((R*)-1-(5-((R)-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)propane-2-sulfinamide and (R)-2-methyl-N—((R*)-1-(6-((R)-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)propane-2-sulfinamide

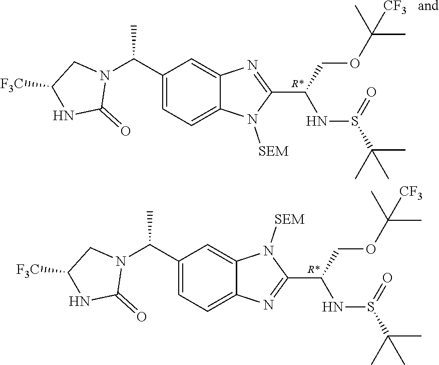

The title compounds were prepared as described for the synthesis of Intermediate 33 using (R)—N—((R*)-1-(6-((R)-1-aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(5-((R)-1-aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 40) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide to afford the title compounds in 23% yield over 3 steps.

### Intermediate 42: (S)-1-((R)-1-(2-((R*)-1-Amino-2-((1,1,1-trifluoro-2-methylpropan-2-yl)oxy)ethyl)-1H-benzo[d]imidazol-6-yl)ethyl)-4-(trifluoromethyl)imidazolidin-2-one

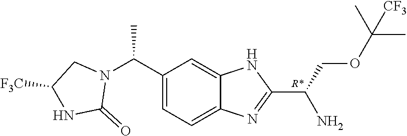

The title compound was prepared as described for the synthesis of Intermediate 34 using (R)-2-methyl-N—((R*)-1-(5-((R)-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)propane-2-sulfinamide and (R)-2-methyl-N—((R*)-1-(6-((R)-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)propane-2-sulfinamide (Intermediate 41) in place of (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide. The product was purified by preparative HPLC (Phenomenex Gemini-NX 3 μm C18 75×30 mm, 28-58% MeCN/water with 0.05% NH₃+10 mM NH₄HCO₃) to afford, after lyophilization, the title compound in 41% yield as a white powder.

### Intermediate 43: (R)—N—((R*)-1-(5-((R)-1-(5,5-Difluoro-2-oxotetrahydropyrimidin-1(2H)-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(6-((R)-1-(5,5-difluoro-2-oxotetrahydropyrimidin-1(2H)-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

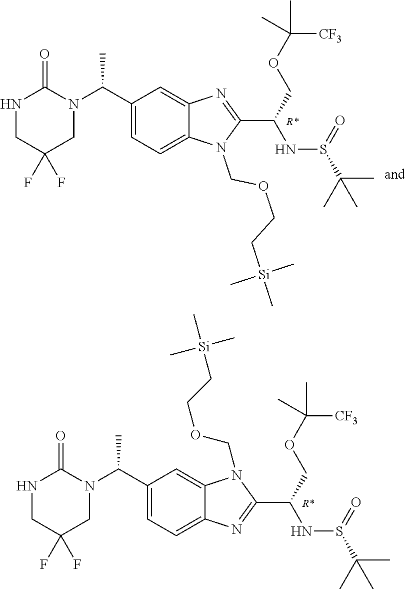

The title compounds (43% yield) were prepared as described for the synthesis of Intermediate 36 using (R)—N—((R*)-1-(6-((R)-1-aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(5-((R)-1-aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 40) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 44: 1-((R)-1-(2-((R*)-1-Amino-2-((1,1,1-trifluoro-2-methylpropan-2-yl)oxy)ethyl)-1H-benzo[d]imidazol-5-yl)ethyl)-5,5-difluorotetrahydropyrimidin-2(1H)-one

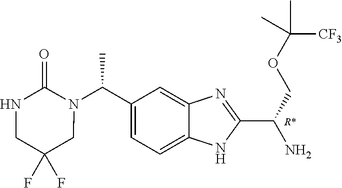 [SMILES: C[C@H](c1ccc2[nH]c([C@@H](N)COC(C)(C)C(F)(F)F)nc2c1)N1CC(F)(F)CNC1=O]

The title compound was prepared as described for Intermediate 37 using (R)—N—((R*)-1-(5-((R)-1-(5,5-difluoro-2-oxotetrahydropyrimidin-1(2H)-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(6-((R)-1-(5,5-difluoro-2-oxotetrahydropyrimidin-1(2H)-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 43) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 74% yield.

### Intermediate 45: (R)—N—((S*)-(1-Cyanocyclopropyl)(1-((2-(trimethylsilyl)ethoxy)methyl)-1H-benzo[d]imidazol-5-yl)methyl)-2-methylpropane-2-sulfinamide and (R)—N—((S*)-(1-cyanocyclopropyl)(1-((2-(trimethylsilyl)ethoxy)methyl)-1H-benzo[d]imidazol-6-yl)methyl)-2-methylpropane-2-sulfinamide

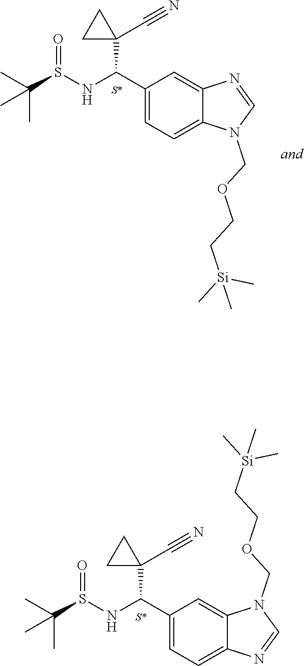 [SMILES: CC(C)(C)[S@@](=O)N[C@@H](c1ccc2c(c1)ncn2COCC[Si](C)(C)C)C1(C#N)CC1 ; CC(C)(C)[S@@](=O)N[C@@H](c1ccc2ncn(COCC[Si](C)(C)C)c2c1)C1(C#N)CC1]

To a 0° C. solution of cyclopropanecarbonitrile (1.1 mL, 15 mmol) in THF (35 mL) was added potassium bis(trimethylsilyl)amide (1 M in THF, 15 mL, 15 mmol). The mixture was stirred for 40 min at this temperature then cooled to −15° C. and a solution of (R,E)-2-methyl-N-((1-((2-(trimethylsilyl)ethoxy)methyl)-1H-benzo[d]imidazol-5-yl)methylene)propane-2-sulfinamide and (R,E)-2-methyl-N-((1-((2-(trimethylsilyl)ethoxy)methyl)-1H-benzo[d]imidazol-6-yl)methylene)propane-2-sulfinamide (2.2 g, 5.8 mmol, Intermediate 17) in THF (15 mL) was added. The reaction was stirred at 0° C. for 30 min then quenched by the addition of saturated aqueous NaHCO₃ solution. The mixture was diluted with EtOAc, then the layers were separated. The organic layer was washed with saturated aqueous NaHCO₃ solution and brine sequentially, dried over anhydrous Na₂SO₄, filtered and concentrated under reduced pressure. Purification by silica gel chromatography (20-100% (10% MeOH in EtOAc)/CH₂Cl₂) provided the title compounds in 47% yield.

### Intermediate 46: (S*)-1-(Amino(1-((2-(trimethylsilyl)ethoxy)methyl)-1H-benzo[d]imidazol-5-yl)methyl)cyclopropane-1-carbonitrile and (S*)-1-(amino(1-((2-(trimethylsilyl)ethoxy)methyl)-1H-benzo[d]imidazol-6-yl)methyl)cyclopropane-1-carbonitrile

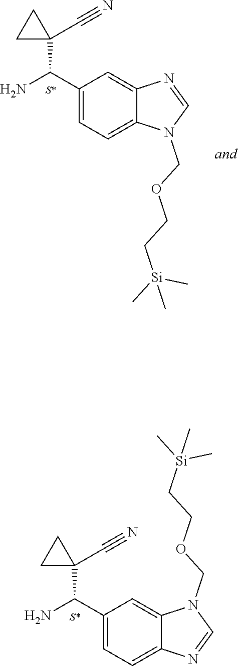 [SMILES: C[Si](C)(C)CCOCn1cnc2cc([C@H](N)C3(C#N)CC3)ccc21 ; C[Si](C)(C)CCOCn1cnc2ccc([C@H](N)C3(C#N)CC3)cc21]

The title compounds were prepared as described for the synthesis of Intermediate 39 using (R)—N—((S*)-(1-cyanocyclopropyl)(1-((2-(trimethylsilyl)ethoxy)methyl)-1H-benzo[d]imidazol-5-yl)methyl)-2-methylpropane-2-sulfinamide and (R)—N—((S*)-(1-cyanocyclopropyl)(1-((2-(trimethylsilyl)ethoxy)methyl)-1H-benzo[d]imidazol-6-yl)methyl)-2-methylpropane-2-sulfinamide (Intermediate 45) in place of (S)-2-methyl-N—((R)-1-(1-((2-(trimethylsilyl)ethoxy)methyl)-1H-benzo[d]imidazol-6-yl)ethyl)propane-2-sulfinamide and (S)-2-methyl-N—((R)-1-(1-((2-(trimethylsilyl)ethoxy)methyl)-1H-benzo[d]imidazol-5-yl)ethyl)propane-2-sulfinamide in 96% yield.

### Intermediate 47: (S*)-1-((1,3-Dioxoisoindolin-2-yl)(1-((2-(trimethylsilyl)ethoxy)methyl)-1H-benzo[d]imidazol-5-yl)methyl)cyclopropane-1-carbonitrile and (S*)-1-((1,3-dioxoisoindolin-2-yl)(1-((2-(trimethylsilyl)ethoxy)methyl)-1H-benzo[d]imidazol-6-yl)methyl)cyclopropane-1-carbonitrile

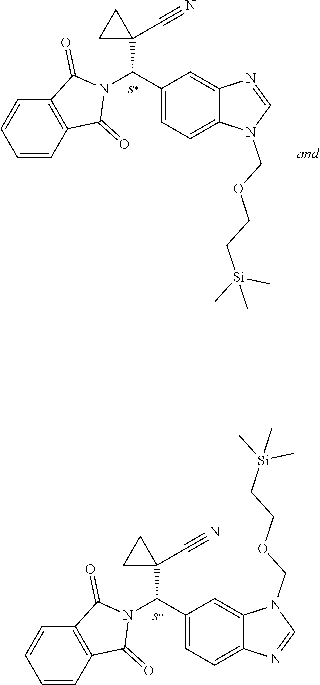 [SMILES: C[Si](C)(C)CCOCn1cnc2cc([C@H](N3C(=O)c4ccccc4C3=O)C3(C#N)CC3)ccc21 ; C[Si](C)(C)CCOCn1cnc2ccc([C@H](N3C(=O)c4ccccc4C3=O)C3(C#N)CC3)cc21]

The title compounds were prepared as described for the synthesis of Intermediate 30 Step C using (S*)-1-(amino(1-((2-(trimethylsilyl)ethoxy)methyl)-1H-benzo[d]imidazol-5-yl)methyl)cyclopropane-1-carbonitrile and (S*)-1-(amino(1-((2-(trimethylsilyl)ethoxy)methyl)-1H-benzo[d]imidazol-6-yl)methyl)cyclopropane-1-carbonitrile (Intermediate 46) in place of (R)-cyclopropyl(1-((2-(trimethylsilyl)ethoxy)methyl)-1H-benzo[d]imidazol-5-yl)methanamine in 75% yield.

### Intermediate 48: (R)—N—((R*)-1-(5-((S*)-(1-Cyanocyclopropyl)(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(6-((S*)-(1-cyanocyclopropyl)(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

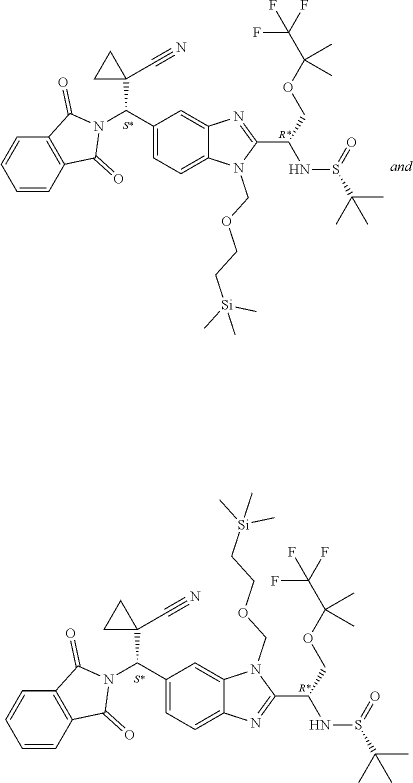

The title compounds were prepared as described for the synthesis of Intermediate 31 using (S*)-1-((1,3-dioxoisoindolin-2-yl)(1-((2-(trimethylsilyl)ethoxy)methyl)-1H-benzo[d]imidazol-5-yl)methyl)cyclopropane-1-carbonitrile and (S*)-1-((1,3-dioxoisoindolin-2-yl)(1-((2-(trimethylsilyl)ethoxy)methyl)-1H-benzo[d]imidazol-6-yl)methyl)cyclopropane-1-carbonitrile (Intermediate 47) in place of (R)-2-(cyclopropyl(1-((2-(trimethylsilyl)ethoxy)methyl)-1H-benzo[d]imidazol-5-yl)methyl)isoindoline-1,3-dione. Purification by silica gel chromatography (10-100% (10% MeOH in EtOAc)/CH₂Cl₂) provided the title compounds in 68% yield.

### Intermediate 49: (R)—N—((R*)-1-(5-((S*)-Amino(1-cyanocyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(6-((S*)-amino(1-cyanocyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

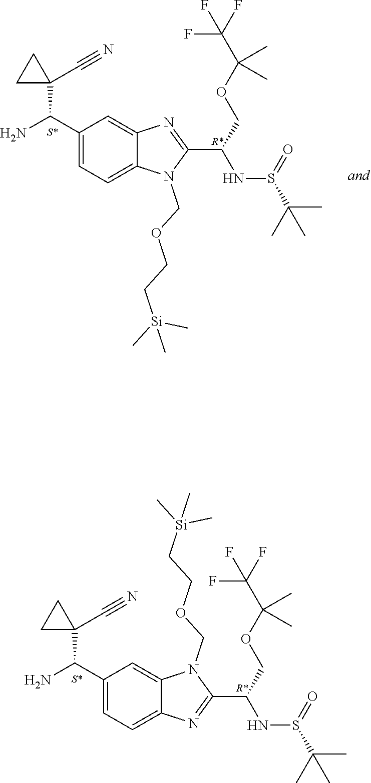

The title compounds were prepared as described for the synthesis of Intermediate 32 using (R)—N—((R*)-1-(5-((S*)-(1-cyanocyclopropyl)(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(6-((S*)-(1-cyanocyclopropyl)(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 48) in place of (R)—N—((R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide, and used without further purification.

### Intermediate 50: (R)—N—((R*)-1-(5-((S*)-(1-Cyanocyclopropyl)(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(6-((S*)-(1-cyanocyclopropyl)(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

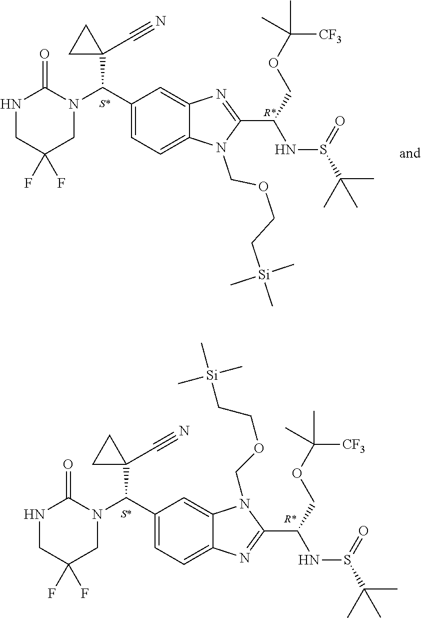

The title compounds (39% yield) were prepared as described for the synthesis of Intermediate 36 using (R)—N—((R*)-1-(5-((S*)-amino(1-cyanocyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(6-((S*)-amino(1-cyanocyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 49) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 51: 1-((S*)-(2-((R*)-1-Amino-2-((1,1,1-trifluoro-2-methylpropan-2-yl)oxy)ethyl)-1H-benzo[d]imidazol-5-yl)(5,5-difluoro-2-oxotetrahydropyrimidin-1(2H)-yl)methyl)cyclopropane-1-carbonitrile

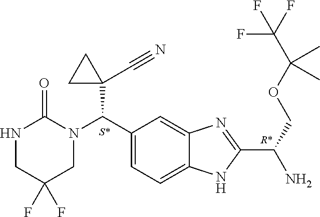 [SMILES: CC(C)(OC[C@H](N)c1nc2cc([C@H](N3CC(F)(F)CNC3=O)C3(C#N)CC3)ccc2[nH]1)C(F)(F)F]

The title compound was prepared as described for Intermediate 37 using (R)—N—((R*)-1-(5-((S*)-(1-cyanocyclopropyl)(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(6-((S*)-(1-cyanocyclopropyl)(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 50) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 31% yield.

### Intermediate 52: (R)—N-((1R*,2R)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

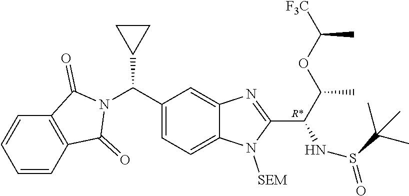 [SMILES: C[C@@H](O[C@H](C)C(F)(F)F)[C@H](N[S@](=O)C(C)(C)C)c1nc2cc([C@@H](C3CC3)N3C(=O)c4ccccc4C3=O)ccc2n1COCC[Si](C)(C)C]

A round-bottom flask was charged with (R)-2-(cyclopropyl(1-((2-(trimethylsilyl)ethoxy)methyl)-1H-benzo[d]imidazol-5-yl)methyl)isoindoline-1,3-dione (2.2 g, 4.9 mmol, Intermediate 30) and THF (40 mL) and cooled to −78° C. Sequentially LDA (11 mL, 11 mmol, 1 M in THF) and a solution of (R,E)-2-methyl-N—((R)-2-(((R)-1,1,1-trifluoropropan-2-yl)oxy)propylidene)propane-2-sulfinamide (2.10 g, 7.72 mmol, Intermediate 5) in THF (10 mL) were added dropwise over 10 min each at −78° C. The resulting mixture was allowed to stir at this temperature for 1 h. The reaction mixture was then quenched with THF (20 mL with 2% AcOH), and the solution was diluted with EtOAc (50 mL) and brine (20 mL). The layers were separated, and the aqueous layer was extracted with EtOAc (30 mL×2). The combined organic layers were washed with brine, dried over anhydrous Na₂SO₄, filtered, and concentrated under reduced pressure. The product was purified by silica gel chromatography (1-5% MeOH/DCM) followed by preparative HPLC (Boston Uni C18 40×150 mm×5 μm, 62-92% MeCN/water (0.23% FA) to afford the title compound in 48% yield, after lyophilization, as an off-white solid.

### Intermediate 53: (R)—N-((1R*,2R)-1-(5-((R)-Amino(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

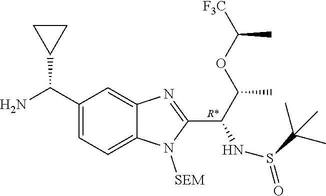 [SMILES: C[C@@H](O[C@H](C)C(F)(F)F)[C@H](N[S@](=O)C(C)(C)C)c1nc2cc([C@H](N)C3CC3)ccc2n1COCC[Si](C)(C)C]

The title compound was prepared as described for the synthesis of Intermediate 32 using (R)—N-((1R*,2R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (Intermediate 52) in place of (R)—N—((R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide to afford the title compound.

### Intermediate 54: (R)—N-((1R*,2R)-1-(5-((R)-Cyclopropyl((S)-2-oxo-4-(trifluoromethyl)imidazolidin-1-yl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

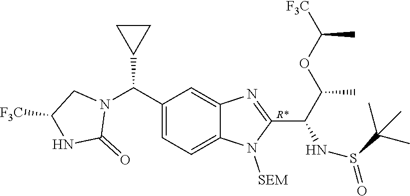 [SMILES: C[C@@H](O[C@H](C)C(F)(F)F)[C@H](N[S@](=O)C(C)(C)C)c1nc2cc([C@@H](C3CC3)N3C[C@@H](C(F)(F)F)NC3=O)ccc2n1COCC[Si](C)(C)C]

Step A: Benzyl ((S)-3-(((R)-(2-((1R*,2R)-1-(((R)-tert-butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)(cyclopropyl)methyl)amino)-1,1,1-trifluoropropan-2-yl)carbamate. A round-bottom flask was charged with (R)—N-((1R*,2R)-1-(5-((R)-amino(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (396 mg, 0.67 mmol, Intermediate 53), Cs₂CO₃ (655 mg, 2.01 mmol), (S)-benzyl 4-(trifluoromethyl)-1,2,3-oxathiazolidine-3-carboxylate 2,2-dioxide (327 mg, 1.0 mmol, Intermediate 24), and anhydrous DMF (6.7 mL) and the resulting mixture was heated to 30° C. The reaction mixture was stirred for about 1 h, until (R)—N-((1R*,2R)-1-(5-((R)-amino(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide had been completely consumed as determined by LCMS analysis. The reaction mixture was then diluted with water (100 mL) and extracted with EtOAc (3×30 mL). The combined organic layers were washed with brine, dried over anhydrous Na₂SO₄, filtered, and concentrated under reduced pressure to afford the product, which was purified by silica gel chromatography (0-100% ethyl acetate (with 10% MeOH) in hexanes) to afford the title compound in 78% yield as an off-white foam.

Step B: (R)—N-((1R*,2R)-1-(5-((R)—(((S)-2-Amino-3,3,3-trifluoropropyl)amino)(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. Benzyl ((S)-3-(((R)-(2-((1R*,2R)-1-(((R)-tert-butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)(cyclopropyl)methyl)amino)-1,1,1-trifluoropropan-2-yl)carbamate (430 mg, 0.46 mmol, Step A), MeOH (18.5 mL), and 10% Pd/C (197 mg, (10% Pd (50% wet with water))) were added to a 75 mL hydrogenation bottle, and the bottle was placed into a Parr-shaker and was shaken under H₂ (45 Psi) for 1.5 h. The reaction mixture was then filtered through diatomaceous earth (e.g., Celite©), the filter-cake was rinsed with MeOH (30 mL), and the filtrate concentrated to dryness under reduced pressure to afford the product that was used in subsequent steps without further purification assuming 100% yield.

Step C: (R)—N-((1R*,2R)-1-(5-((R)-Cyclopropyl((S)-2-oxo-4-(trifluoromethyl)imidazolidin-1-yl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. (R)—N-((1R*,2R)-1-(5-((R)—(((S)-2-Amino-3,3,3-trifluoropropyl)amino)(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (322 mg, 0.459 mmol, Step B) and THF (22.9 mL) were added to a nitrogen-purged round-bottom flask. The resulting mixture was then treated with CDI (223 mg, 1.38 mmol) and stirred for 2 h. After 2 h, additional CDI (446 mg, 2.75 mmol) was added and the reaction temperature was raised to reflux. After 16 h, the reaction mixture was then poured into NaOH (1 M aqueous, 50 mL) and extracted with EtOAc (3×30 mL). The combined organic layers were washed with 0.05 M aqueous HCl and brine, and the combined organic extracts were dried over anhydrous MgSO₄, filtered, and concentrated under reduced pressure. This material was purified by silica gel chromatography (0-75% EtOAc (with 10% MeOH) in hexanes) to afford the title compound in 32% yield.

### Intermediate 55: (S)-1-((R)-(2-((1R*,2R)-1-Amino-2-(((R)-1,1,1-trifluoropropan-2-yl)oxy)propyl)-1H-benzo[d]imidazol-5-yl)(cyclopropyl)methyl)-4-(trifluoromethyl)imidazolidin-2-one

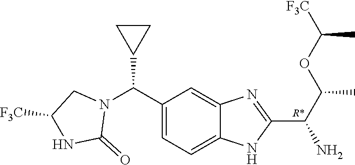 [SMILES: C[C@@H](O[C@H](C)C(F)(F)F)[C@H](N)c1nc2cc([C@@H](C3CC3)N3C[C@@H](C(F)(F)F)NC3=O)ccc2[nH]1]

The title compound was prepared as described for the synthesis of Intermediate 57 using (R)—N-((1R*,2R)-1-(5-((R)-cyclopropyl((S)-2-oxo-4-(trifluoromethyl)imidazolidin-1-yl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (Intermediate 54) in place of (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, to afford the title compound in 84% yield.

### Intermediate 56: (R)—N-((1R*,2R)-1-(5-((1R)-Cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

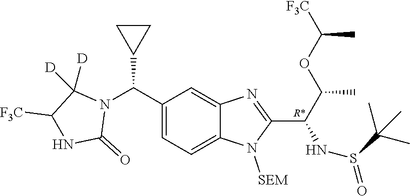 [SMILES: [2H]C1([2H])C(C(F)(F)F)NC(=O)N1[C@@H](c1ccc2c(c1)nc([C@@H](N[S@](=O)C(C)(C)C)[C@@H](C)O[C@H](C)C(F)(F)F)n2COCC[Si](C)(C)C)C1CC1]

Step A: Benzyl (3-(((R)-(2-((1R*,2R)-1-(((R)-tert-butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)(cyclopropyl)methyl)amino)-1,1,1-trifluoropropan-2-yl-3,3-d₂)carbamate. A round-bottom flask was charged with (R)—N-((1R*,2R)-1-(5-((R)-amino(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (791 mg, 1.34 mmol, Intermediate 53), DMF (13.4 mL), Cs₂CO₃ (1.31 g, 4.02 mmol), and benzyl (trifluoromethyl)-1,2,3-oxathiazolidine-3-carboxylate-5,5-d₂ 2,2-dioxide (996 mg, 2.01 mmol, Intermediate 28) and the resulting mixture was stirred for 1 h at 25° C. The reaction was then poured over water and extracted three times with EtOAc. The combined organic layers were washed with aqueous LiCl (10% w/v) and brine, dried over anhydrous MgSO₄, filtered, and concentrated under reduced pressure. The product was purified by silica gel chromatography (0-100% EtOAc (with 10% MeOH)/hexanes) to afford the title compound in 96% yield as a yellow foam.

Step B: (R)—N-((1R*,2R)-1-(5-((1R)-((2-Amino-3,3,3-trifluoropropyl-1,1-d₂)amino)(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. To a 500 mL hydrogenation flask was added benzyl (3-(((R)-(2-((1R*,2R)-1-(((R)-tert-butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)(cyclopropyl)methyl)amino)-1,1,1-trifluoropropan-2-yl-3,3-d₂)carbamate (1.05 g, 1.25 mmol, Step A), MeOH (50 mL), and 10% Pd/C (0.533 g, 0.501 mmol). The bottle was placed into a Parr-shaker and was shaken under H₂ (45 psi) for 1.5 h. After this time, the reaction mixture was filtered through a pad of diatomaceous earth (Celite©) and the filtrate was concentrated to afford the title compound that was used without further purification in 100% yield (assumed).

Step C: (R)—N-((1R*,2R)-1-(5-((1R)-Cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. A round-bottom flask was charged with (R)—N-((1R*,2R)-1-(5-((1R)-((2-amino-3,3,3-trifluoropropyl-1,1-d₂)amino)(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (879 mg, 1.25 mmol Step B), THF (62 mL), and CDI (607 mg, 3.74 mmol) and the solution was heated at 65° C. for 2 h. After this time, additional CDI (1.21 g, 7.48 mmol) was added, and the reaction was heated at 65° C. for 16 h. The solution was cooled to 25° C. and poured into 1 M aqueous NaOH and the biphasic mixture was extracted three times with EtOAc. The combined organic layers were washed with 0.05 M aqueous HCl and brine, dried over anhydrous MgSO₄, filtered, and concentrated under reduced pressure. The product was purified by silica gel chromatography (0-75% EtOAc (10% MeOH)/hexanes) to afford the title compound in 26% yield as an off-white foam.

### Intermediate 57: 1-((R)-(2-((1R*,2R)-1-Amino-2-(((R)-1,1,1-trifluoropropan-2-yl)oxy)propyl)-1H-benzo[d]imidazol-5-yl)(cyclopropyl)methyl)-4-(trifluoromethyl)imidazolidin-2-one-5,5-d₂

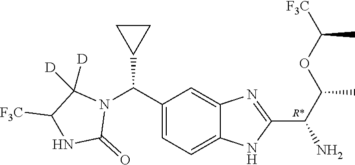 [SMILES: [2H]C1([2H])C(C(F)(F)F)NC(=O)N1[C@@H](c1ccc2[nH]c([C@@H](N)[C@@H](C)O[C@H](C)C(F)(F)F)nc2c1)C1CC1]

A round-bottom flask was charged with (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (239 mg, 0.327 mmol, Intermediate 56), 1,4-dioxane:MeOH (4:1 ratio, 3.3 mL), and 4.0 M HCl in 1,4-dioxane (0.817 mL, 3.27 mmol) and the resulting mixture was heated at 60° C. for 3 h. The reaction was then allowed to cool to rt, diluted with water (10 mL) and washed with 1:1 EtOAc/hexanes (2×15 mL). The organic layers were discarded, and the pH of the aqueous layer was adjusted to pH 10, by the addition of 1 N aqueous NaOH. The aqueous layer was extracted with EtOAc (3×30 mL) and the combined organic layers were dried over anhydrous MgSO₄, filtered, and concentrated under reduced pressure to afford the title compound which was used without further purification (85% yield).

### Intermediate 58: (R)—N-((1R*,2R)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

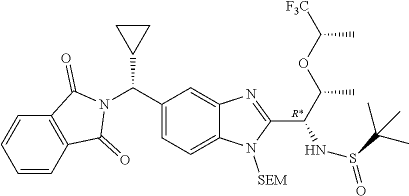 [SMILES: C[C@H](O[C@H](C)[C@H](N[S@](=O)C(C)(C)C)c1nc2cc([C@@H](C3CC3)N3C(=O)c4ccccc4C3=O)ccc2n1COCC[Si](C)(C)C)C(F)(F)F]

The title compound was prepared as described for the synthesis of Intermediate 52 using (R)-2-methyl-N—((R,E)-2-(((S)-1,1,1-trifluoropropan-2-yl)oxy)propylidene)propane-2-sulfinamide (Intermediate 9) in place of (R,E)-2-methyl-N—((R)-2-(((R)-1,1,1-trifluoropropan-2-yl)oxy)propylidene)propane-2-sulfinamide, which provided the title compound in 57% yield.

### Intermediate 59: (R)—N-((1R*,2R)-1-(5-((R)-Amino(cyclopropyl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

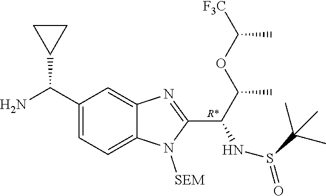 [SMILES: C[C@H](O[C@H](C)[C@H](N[S@](=O)C(C)(C)C)c1nc2cc([C@H](N)C3CC3)ccc2n1COCC[Si](C)(C)C)C(F)(F)F]

The title compound was prepared as described for the synthesis of Intermediate 53 using (R)—N-((1R*,2R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 58) in place of (R)—N-((1R*,2R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, which provided the title compound in 83% yield.

### Intermediate 60: (R)—N-((1R*,2R)-1-(5-((1R)-Cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

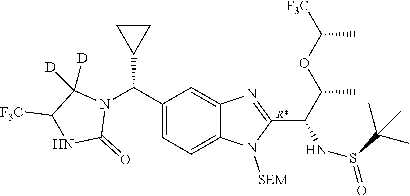 [SMILES: [2H]C1([2H])C(C(F)(F)F)NC(=O)N1[C@@H](c1ccc2c(c1)nc([C@@H](N[S@](=O)C(C)(C)C)[C@@H](C)O[C@@H](C)C(F)(F)F)n2COCC[Si](C)(C)C)C1CC1]

Step A: tert-Butyl (3-(((R)-(2-((1R*,2R)-1-(((R)-tert-butylsulfinyl)amino)-2-(((S)-1,1,1-trifluoropropan-2-yl)oxy)propyl)-1-((2-(trimethylsilyl)ethoxy)methyl)-1H-benzo[d]imidazol-5-yl)(cyclopropyl)methyl)amino)-1,1,1-trifluoropropan-2-yl-3,3-d₂)carbamate. The title compound was prepared as described for the synthesis of Intermediate 56 using (R)—N-((1R*,2R)-1-(5-((R)-amino(cyclopropyl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 59) in place of (R)—N-((1R*,2R)-1-(5-((R)-amino(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide and tert-butyl 4-(trifluoromethyl)-1,2,3-oxathiazolidine-3-carboxylate-5,5-d₂ 2,2-dioxide (Intermediate 35) in place of benzyl (trifluoromethyl)-1,2,3-oxathiazolidine-3-carboxylate-5,5-d₂ 2,2-dioxide to afford the title compound in 78% yield.

Step B: (R)—N-((1R*,2R)-1-(5-((1R)-((2-Amino-3,3,3-trifluoropropyl-1,1-d₂)amino)(cyclopropyl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. A round-bottom flask was charged with tert-butyl (3-(((R)-(2-((1R*,2R)-1-(((R)-tert-butylsulfinyl)amino)-2-(((S)-1,1,1-trifluoropropan-2-yl)oxy)propyl)-1-((2-(trimethylsilyl)ethoxy)methyl)-1H-benzo[d]imidazol-5-yl)(cyclopropyl)methyl)amino)-1,1,1-trifluoropropan-2-yl-3,3-d₂)carbamate (210 mg, 0.261 mmol, Step A), DCM (13 mL), and TFA (1.0 mL) and the resulting solution was allowed to stir for 1.4 h at 25° C. The solution was then diluted with water and extracted with 1:1 hexanes/EtOAc. The pH of the aqueous layer was adjusted to pH 10, by the addition of 1 N aqueous NaOH, and was then extracted with EtOAc (3×20 mL). The combined organic layers were dried over anhydrous MgSO₄, filtered, and concentrated to afford the title compound that was used without further purification, assuming 100% yield.

Step C: (R)—N-((1R*,2R)-1-(5-((1R)-Cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. The title compound was prepared as described for the synthesis of Intermediate 56 Step C using (R)—N-((1R*,2R)-1-(5-((1R)-((2-amino-3,3,3-trifluoropropyl-1,1-d₂)amino)(cyclopropyl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Step B) in place of (R)—N-((1R*,2R)-1-(5-((1R)-((2-amino-3,3,3-trifluoropropyl-1,1-d₂)amino)(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, which provided the title compound, assuming 100% yield, which was used without further purification.

### Intermediate 61: 1-((R)-(2-((1R*,2R)-1-Amino-2-(((S)-1,1,1-trifluoropropan-2-yl)oxy)propyl)-1H-benzo[d]imidazol-5-yl)(cyclopropyl)methyl)-4-(trifluoromethyl)imidazolidin-2-one-5,5-d₂

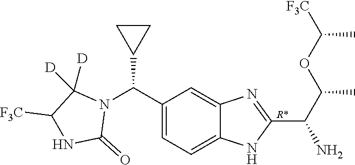 [SMILES: [2H]C1([2H])C(C(F)(F)F)NC(=O)N1[C@@H](c1ccc2[nH]c([C@@H](N)[C@@H](C)O[C@@H](C)C(F)(F)F)nc2c1)C1CC1]

The title compound was prepared as described for the synthesis of Intermediate 57 using (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 60) in place of (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, which provided the title compound in 85% yield.

### Intermediate 62: (R)—N-((1R*,2R)-1-(5-((R)-Cyclopropyl((S)-2-oxo-4-(trifluoromethyl)imidazolidin-1-yl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

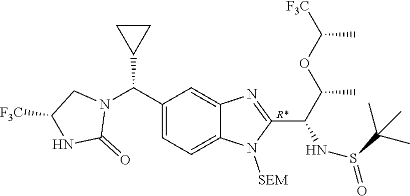 [SMILES: C[C@H](O[C@H](C)[C@H](N[S@](=O)C(C)(C)C)c1nc2cc([C@@H](C3CC3)N3C[C@@H](C(F)(F)F)NC3=O)ccc2n1COCC[Si](C)(C)C)C(F)(F)F]

The title compound was prepared as described for the synthesis of Intermediate 54 using (R)—N-((1R*,2R)-1-(5-((R)-amino(cyclopropyl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 59) in place of (R)—N-((1R*,2R)-1-(5-((R)-amino(cyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, which provided the title compound in 21% yield over 3 steps.

### Intermediate 63: (S)-1-((R)-(2-((1R*,2R)-1-Amino-2-(((S)-1,1,1-trifluoropropan-2-yl)oxy)propyl)-1H-benzo[d]imidazol-5-yl)(cyclopropyl)methyl)-4-(trifluoromethyl)imidazolidin-2-one

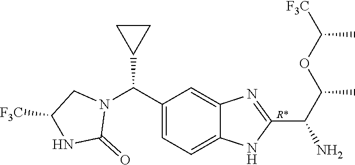 [SMILES: C[C@H](O[C@H](C)[C@H](N)c1nc2cc([C@@H](C3CC3)N3C[C@@H](C(F)(F)F)NC3=O)ccc2[nH]1)C(F)(F)F]

The title compound was prepared as described for the synthesis of Intermediate 57 using (R)—N-((1R*,2R)-1-(5-((R)-cyclopropyl((S)-2-oxo-4-(trifluoromethyl)imidazolidin-1-yl)methyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 62) in place of (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, which provided the title compound in 85% yield.

### Intermediate 64: (S)-3,3,3-Trifluoro-N1-((1-((2-(trimethylsilyl)ethoxy)methyl)-1H-benzo[d]imidazol-5-yl)methyl)propane-1,2-diamine

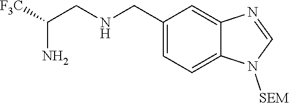 [SMILES: C[Si](C)(C)CCOCn1cnc2cc(CNC[C@H](N)C(F)(F)F)ccc21]

A round-bottom flask was charged with 1-((2-(trimethylsilyl)ethoxy)methyl)-1H-benzo[d]imidazole-5-carbaldehyde (2.5 g, 9.0 mmol, Intermediate 10) and DCM (90 mL). (S)-3,3,3-trifluoropropane-1,2-diamine bis-hydrogen chloride (3.6 g, 18 mmol) and Et₃N (5.5 mL, 40 mmol) were sequentially added, and the reaction mixture stirred at rt for 30 min. The turbid solution was then heated to 40° C. and stirred for 1 h. After this time the reaction was cooled to rt and sodium cyanoborohydride (2.0 g, 32 mmol), MeOH (8.2 mL), and AcOH (2.7 mL) were sequentially added, and the reaction mixture was stirred for 30 min. The reaction mixture was then concentrated under reduced pressure to a residue and the residue was dissolved in DCM and the pH of the solution was adjusted to pH 7 with saturated aqueous NaHCO₃ solution with vigorous stirring. The biphasic mixture was separated, and the aqueous layer was extracted twice with DCM. The combined organic layers were dried over anhydrous MgSO₄ and concentrated under reduced pressure to afford the title compound that was used without further purification.

### Intermediate 65: (S)-4-(Trifluoromethyl)-1-((1-((2-(trimethylsilyl)ethoxy)methyl)-1H-benzo[d]imidazol-5-yl)methyl)imidazolidin-2-one

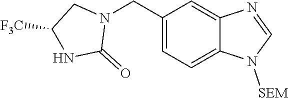 [SMILES: C[Si](C)(C)CCOCn1cnc2cc(CN3C[C@@H](C(F)(F)F)NC3=O)ccc21]

A round-bottom flask was charged with (S)-3,3,3-trifluoro-N1-((1-((2-(trimethylsilyl)ethoxy)methyl)-1H-benzo[d]imidazol-5-yl)methyl)propane-1,2-diamine (3.51 g, 9.05 mmol, Intermediate 64) and THF (100 mL) and the mixture was heated to 65° C. CDI (4.40 g, 27.1 mmol) was then added portion-wise and stirred at 65° C. for 1 h. The reaction was then cooled to rt and quenched with 1 M aqueous NaOH (22.6 mL, 22.6 mmol). The biphasic mixture was concentrated to ⅓ volume and subsequently diluted with water and EtOAc. The biphasic mixture was separated, and the aqueous layer was further extracted three times with EtOAc. The combined organic layers were washed with brine, dried over anhydrous MgSO₄, filtered, and concentrated under reduced pressure. The product was purified by silica gel chromatography (0-10% MeOH/DCM) to afford the title compound in 69% yield.

### Intermediate 66: (R)-2-Methyl-N—((R)-1-(5-(((S)-2-oxo-4-(trifluoromethyl)imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)propane-2-sulfinamide

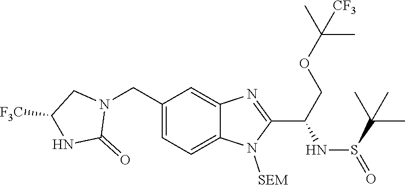 [SMILES: CC(C)(C)[S@@](=O)N[C@@H](COC(C)(C)C(F)(F)F)c1nc2cc(CN3C[C@@H](C(F)(F)F)NC3=O)ccc2n1COCC[Si](C)(C)C]

n-BuLi (3.35 mL, 5.36 mmol, 1.6 M in hexanes) was added to a 0° C. solution of diisopropylamine (0.783 mL, 5.59 mmol) in THF (3 mL) and was stirred at this temperature for 30 min to form a solution of LDA. A separate solution of (S)-4-(trifluoromethyl)-1-((1-((2-(trimethylsilyl)ethoxy)methyl)-1H-benzo[d]imidazol-5-yl)methyl)imidazolidin-2-one (965 mg, 2.33 mmol, Intermediate 65) in THF (23 mL) was cooled to −78° C. and the LDA solution was added dropwise. The resulting solution stirred at −78° C. for 30 min. A solution of (R,E)-2-methyl-N-(2-((1,1,1-trifluoro-2-methylpropan-2-yl)oxy)ethylidene)propane-2-sulfinamide (891 mg, 3.26 mmol, Intermediate 1) in THF (4 mL) was then added dropwise. The reaction mixture was stirred for 1 h at −78° C. and was then quenched with AcOH (0.4 mL) and warmed to rt. The solution was diluted with saturated aqueous NaHCO₃ and extracted three times with EtOAc. The combined organic layers were washed with brine, dried over anhydrous Na₂SO₄, filtered, and concentrated under reduced pressure. The residue was purified by silica gel chromatography (10-80% EtOAc (10% MeOH)/DCM) followed by preparative HPLC ((Xbridge Prep C18, 5 μm, 50×100 mm), 10-100% MeCN/aqueous 20 mM NH₄OH) to afford the title compound in 10% yield.

### Intermediate 67: (S)-1-((2-((R)-1-Amino-2-((1,1,1-trifluoro-2-methylpropan-2-yl)oxy)ethyl)-1H-benzo[d]imidazol-5-yl)methyl)-4-(trifluoromethyl)imidazolidin-2-one

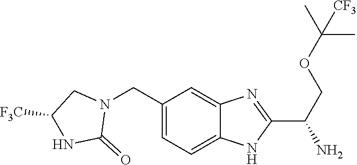 [SMILES: CC(C)(OC[C@H](N)c1nc2cc(CN3C[C@@H](C(F)(F)F)NC3=O)ccc2[nH]1)C(F)(F)F]

The title compound was prepared as described for the synthesis of Intermediate 57 using (R)-2-methyl-N—((R)-1-(5-(((S)-2-oxo-4-(trifluoromethyl)imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)propane-2-sulfinamide (Intermediate 66) in place of (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, which provided the title compound in 66% yield.

### Intermediate 68: (R)—N—((R)-1-(5-Bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R)-1-(6-bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

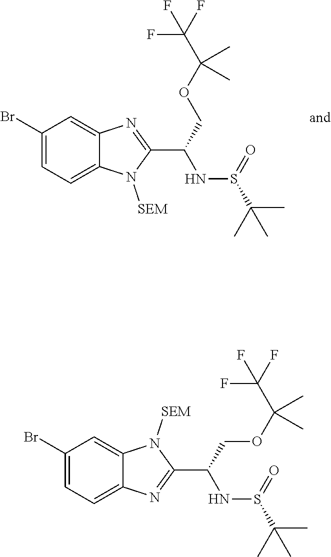 [SMILES: CC(C)(C)[S@@](=O)N[C@@H](COC(C)(C)C(F)(F)F)c1nc2cc(Br)ccc2n1COCC[Si](C)(C)C ; CC(C)(C)[S@@](=O)N[C@@H](COC(C)(C)C(F)(F)F)c1nc2ccc(Br)cc2n1COCC[Si](C)(C)C]

The title compounds (50% yield) were prepared as described for the synthesis of Intermediate 31 using 5-bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole (Intermediate 13) in place of (R)-2-(cyclopropyl(1-((2-(trimethylsilyl)ethoxy)methyl)-1H-benzo[d]imidazol-5-yl)methyl)isoindoline-1,3-dione.

### Intermediate 69: tert-Butyl (R)-(1-(5-bromo-1H-benzo[d]imidazol-2-yl)-2-((1,1,1-trifluoro-2-methylpropan-2-yl)oxy)ethyl)carbamate

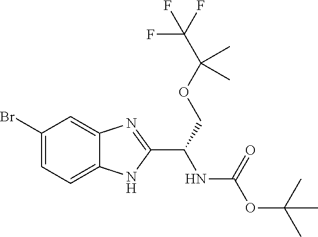 [SMILES: CC(C)(C)OC(=O)N[C@@H](COC(C)(C)C(F)(F)F)c1nc2cc(Br)ccc2[nH]1]

A solution of (R)—N—((R)-1-(5-bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R)-1-(6-bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (1.58 g, 2.63 mmol, Intermediate 68) in 1,4-dioxane:MeOH (ratio 4:1, 26 mL) was heated to 55° C. and HCl (4 M in 1,4-dioxane, 6.6 mL, 26 mmol) was added. The reaction was stirred at this temperature for 16 h. The reaction was then allowed to cool to rt, diluted with water (75 mL) and washed with 1:1 EtOAc/hexanes (2×25 mL). The organic layers were discarded, and the pH of the aqueous layer was adjusted by the addition of Na₂CO₃ (2.8 g). To this mixture was added EtOAc (26 mL) and di-tert-butyl dicarbonate (0.58 g, 2.63 mmol) and the reaction stirred at rt overnight. The layers were separated, and the aqueous layer was further extracted with EtOAc. The combined organic layers were washed with saturated aqueous NH₄Cl and brine, dried over anhydrous sodium sulfate, filtered, and concentrated under reduced pressure to afford the title compound in 98% yield.

### Intermediate 70: tert-Butyl (R)-(2-((1,1,1-trifluoro-2-methylpropan-2-yl)oxy)-1-(5-vinyl-1H-benzo[d]imidazol-2-yl)ethyl)carbamate

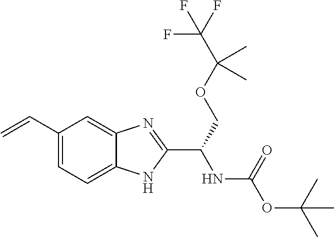 [SMILES: C=Cc1ccc2[nH]c([C@H](COC(C)(C)C(F)(F)F)NC(=O)OC(C)(C)C)nc2c1]

The title compound was prepared as described for the synthesis of Intermediate 14 using tert-butyl (R)-(1-(5-bromo-1H-benzo[d]imidazol-2-yl)-2-((1,1,1-trifluoro-2-methylpropan-2-yl)oxy)ethyl)carbamate (Intermediate 69) in place of 5-bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole and RuPhos Pd G3 in place of [1,1′-bis(diphenylphosphino)ferrocene]dichloropalladium(II) complex with dichloromethane with a reaction temperature of 100° C.

### Intermediate 71: tert-Butyl (R)-(1-(5-formyl-1H-benzo[d]imidazol-2-yl)-2-((1,1,1-trifluoro-2-methylpropan-2-yl)oxy)ethyl)carbamate

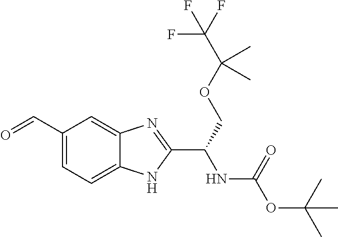 [SMILES: CC(C)(C)OC(=O)N[C@@H](COC(C)(C)C(F)(F)F)c1nc2cc(C=O)ccc2[nH]1]

To a solution of tert-butyl (R)-(2-((1,1,1-trifluoro-2-methylpropan-2-yl)oxy)-1-(5-vinyl-1H-benzo[d]imidazol-2-yl)ethyl)carbamate (1.33 g, 3.22 mmol, Intermediate 70) in 1,4-dioxane (50 mL) and H₂O (50 mL) was added K₂OsO₄·2H₂O (200 mg, 0.54 mmol) and NaIO₄ (13.9 g, 64.8 mmol). The yellow suspension was stirred at 25° C. for 1 h, then the reaction mixture was diluted with H₂O (50 mL) and extracted with EtOAc (3×50 mL). The combined organic layers were washed with saturated aqueous sodium metabisulfite and brine, dried over anhydrous sodium sulfate, filtered, and concentrated under reduced pressure. The residue was purified by silica gel chromatography (10-100% (10% MeOH in EtOAc)/hexanes) to afford the title compound in 97% yield.

### Intermediate 72: tert-Butyl (R)-(1-(5-((5,5-difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)carbamate

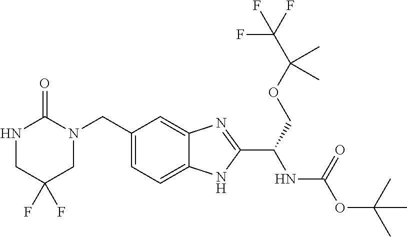 [SMILES: CC(C)(C)OC(=O)N[C@@H](COC(C)(C)C(F)(F)F)c1nc2cc(CN3CC(F)(F)CNC3=O)ccc2[nH]1]

Step A: tert-Butyl (R)-(1-(5-(((3-amino-2,2-difluoropropyl)amino)methyl)-1H-benzo[d]imidazol-2-yl)-2-((1,1,1-trifluoro-2-methylpropan-2-yl)oxy)ethyl)carbamate. To a suspension of tert-butyl (R)-(1-(5-formyl-1H-benzo[d]imidazol-2-yl)-2-((1,1,1-trifluoro-2-methylpropan-2-yl)oxy)ethyl)carbamate (683 mg, 1.64 mmol, Intermediate 71) and 2,2-difluoropropane-1,3-diamine dihydrochloride (602 mg, 3.29 mmol) in CH₂Cl₂ (25 mL) was added triethylamine (1.15 mL, 8.22 mmol). The reaction was heated at 40° C. for 1 h then cooled to rt and MeOH (3.3 mL), AcOH (0.38 mL), and sodium cyanoborohydride (620 mg, 9.87 mmol) were added. The mixture was stirred for 15 min at rt then concentrated under reduced pressure and partitioned between saturated aqueous NaHCO₃ and CH₂Cl₂. The layers were separated then the aqueous further extracted with CH₂Cl₂. The combined organics were dried over anhydrous MgSO₄, filtered, and concentrated under reduced pressure to provide the title compound that was used without further purification.

Step B: tert-Butyl (R)-(1-(5-((5,5-difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)carbamate. tert-Butyl (R)-(1-(5-(((3-amino-2,2-difluoropropyl)amino)methyl)-1H-benzo[d]imidazol-2-yl)-2-((1,1,1-trifluoro-2-methylpropan-2-yl)oxy)ethyl)carbamate (837 mg, 1.64 mmol, Step A) was dissolved in THF (25 mL) then the reaction heated to 65° C. and 1,1′-carbonyldiimidazole (800 mg, 4.93 mmol) was added. After 1 h the reaction was cooled to rt then quenched by slow addition of NaOH (1 M in H₂O, 8.22 mL, 8.22 mmol). The mixture was concentrated under reduced pressure to remove THF then partitioned between saturated aqueous NaHCO₃ and EtOAc. The layers were separated then the aqueous further extracted with EtOAc. The combined organics were washed with brine, dried over anhydrous Na₂SO₄, filtered, and concentrated under reduced pressure. Purification by silica gel chromatography (10-100% (10% MeOH in EtOAc)/hexanes) provided the title compound (74% yield).

### Intermediate 73: (R)-1-((2-(1-Amino-2-((1,1,1-trifluoro-2-methylpropan-2-yl)oxy)ethyl)-1H-benzo[d]imidazol-5-yl)methyl)-5,5-difluorotetrahydropyrimidin-2(1H)-one

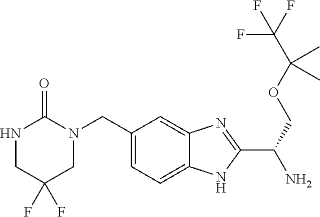 [SMILES: CC(C)(OC[C@H](N)c1nc2cc(CN3CC(F)(F)CNC3=O)ccc2[nH]1)C(F)(F)F]

A round-bottom flask was charged with tert-butyl (R)-(1-(5-((5,5-difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)carbamate (244 mg, 0.455 mmol, Intermediate 72), DCM (2 mL), and 2,2,2-trifluoroacetic acid (0.704 mL. 9.10 mmol). The reaction was stirred for 1 h at rt, then diluted with EtOAc (5 mL) and washed with saturated aqueous NaHCO₃ (2×10 mL). The aqueous layer was washed with 4:1 DCM:iPrOH (3×25 mL). The combined organic layers were dried over anhydrous MgSO₄, filtered, and concentrated under reduced pressure to afford the title compound which was used without further purification.

### Intermediate 74: (R)—N—((S)-2-Methoxy-1-(1-((2-(trimethylsilyl)ethoxy)methyl)-1H-benzo[d]imidazol-5-yl)ethyl)-2-methylpropane-2-sulfinamide

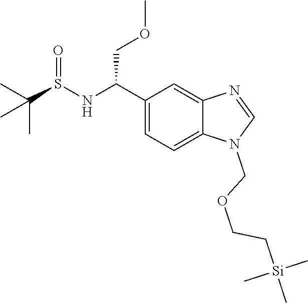 [SMILES: COC[C@@H](N[S@](=O)C(C)(C)C)c1ccc2c(c1)ncn2COCC[Si](C)(C)C]

To tributyl(methoxymethyl)stannane (12.2 g, 36.4 mol) dissolved in THF (200 mL) that had been cooled to −78° C. was added n-BuLi (14.5 mL, 2.5 M in hexanes, 26.2 mmol) slowly over 30 min. This mixture was stirred for 30 min at −78° C. A separate solution of (R,E)-2-methyl-N-((1-((2-(trimethylsilyl)ethoxy)methyl)-1H-benzo[d]imidazol-5-yl)methylene)propane-2-sulfinamide (4.6 g, 12.1 mmol, Intermediate 12) in THF (50 mL) was added to the mixture dropwise over 30 min and stirred at −78° C. until the (R,E)-2-methyl-N-((1-((2-(trimethylsilyl)ethoxy)methyl)-1H-benzo[d]imidazol-5-yl)methylene)propane-2-sulfinamide was completely consumed as judged by LCMS analysis. The reaction was quenched with EtOH (2 mL), warmed to rt, and diluted with a saturated aqueous NaHCO₃ solution (200 mL). The mixture was then extracted with EtOAc (3×100 mL). The combined organic layers were washed with brine (200 mL), dried over anhydrous Na₂SO₄, filtered, and concentrated under reduced pressure. The isolated material was purified by silica gel chromatography (0-3% MeOH/DCM) to afford the title compound (76% yield) as a yellow oil.

### Intermediate 75: (S)-2-Methoxy-1-(1-((2-(trimethylsilyl)ethoxy)methyl)-1H-benzo[d]imidazol-5-yl)ethan-1-amine

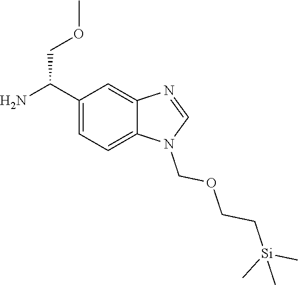 [SMILES: COC[C@@H](N)c1ccc2c(c1)ncn2COCC[Si](C)(C)C]

To a solution of (R)—N—((S)-2-methoxy-1-(1-((2-(trimethylsilyl)ethoxy)methyl)-1H-benzo[d]imidazol-5-yl)ethyl)-2-methylpropane-2-sulfinamide (3.74 g, 8.79 mmol, Intermediate 74) in EtOAc (43 mL) was added HCl (4.39 mL, 17.6 mmol, 4 M in 1,4-dioxane). The reaction mixture was stirred at rt overnight. The reaction mixture was then diluted with water (20 mL) and washed with 1:1 EtOAc/hexanes (2×20 mL). The organic layers were discarded, and the pH of the aqueous layer was adjusted to pH 13 by the addition of 1 M aqueous NaOH. The aqueous layer was extracted with EtOAc (3×50 mL) and the combined organic layers were dried over anhydrous Na₂SO₄, decanted, and concentrated under reduced pressure to afford the title compound that was used without further purification.

### Intermediate 76: (S)-2-(2-Methoxy-1-(1-((2-(trimethylsilyl)ethoxy)methyl)-1H-benzo[d]imidazol-5-yl)ethyl)isoindoline-1,3-dione

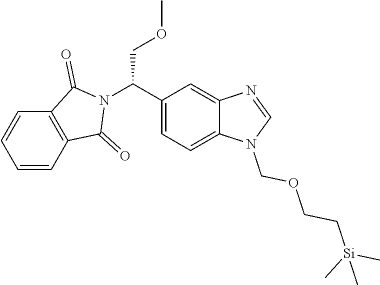 [SMILES: COC[C@H](c1ccc2c(c1)ncn2COCC[Si](C)(C)C)N1C(=O)c2ccccc2C1=O]

The title compound (71% yield) was prepared as described for the synthesis of Intermediate 30 Step C using (S)-2-methoxy-1-(1-((2-(trimethylsilyl)ethoxy)methyl)-1H-benzo[d]imidazol-5-yl)ethan-1-amine (Intermediate 75) in place of (R)-cyclopropyl(1-((2-(trimethylsilyl)ethoxy)methyl)-1H-benzo[d]imidazol-5-yl)methanamine.

### Intermediate 77: (R)—N—((R*)-1-(5-((S)-1-(1,3-Dioxoisoindolin-2-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

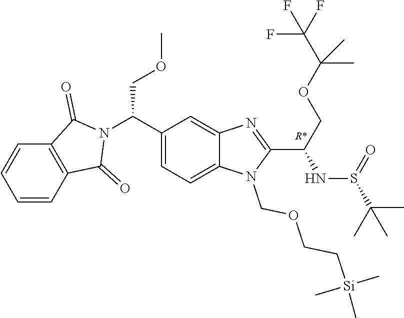 [SMILES: COC[C@H](c1ccc2c(c1)nc([C@H](COC(C)(C)C(F)(F)F)N[S@](=O)C(C)(C)C)n2COCC[Si](C)(C)C)N1C(=O)c2ccccc2C1=O]

The title compound (48% yield) was prepared as described for the synthesis of Intermediate 31 using (S)-2-(2-methoxy-1-(1-((2-(trimethylsilyl)ethoxy)methyl)-1H-benzo[d]imidazol-5-yl)ethyl)isoindoline-1,3-dione (Intermediate 76) in place of (R)-2-(cyclopropyl(1-((2-(trimethylsilyl)ethoxy)methyl)-1H-benzo[d]imidazol-5-yl)methyl)isoindoline-1,3-dione.

### Intermediate 78: (R)—N—((R*)-1-(5-((S)-1-Amino-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

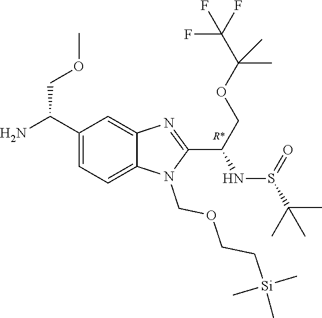 [SMILES: COC[C@@H](N)c1ccc2c(c1)nc([C@H](COC(C)(C)C(F)(F)F)N[S@](=O)C(C)(C)C)n2COCC[Si](C)(C)C]

The title compound (89% yield) was prepared as described for the synthesis of Intermediate 32 using (R)—N—((R*)-1-(5-((S)-1-(1,3-dioxoisoindolin-2-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 77) in place of (R)—N—((R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 79: (R)—N—((R*)-1-(5-((S)-2-Methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

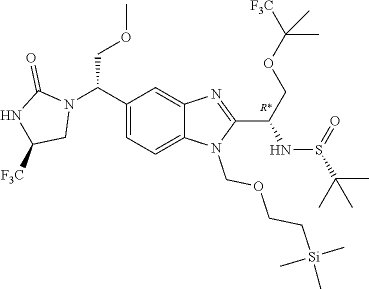 [SMILES: COC[C@H](c1ccc2c(c1)nc([C@H](COC(C)(C)C(F)(F)F)N[S@](=O)C(C)(C)C)n2COCC[Si](C)(C)C)N1C[C@@H](C(F)(F)F)NC1=O]

The title compound (50% yield) was prepared as described for the synthesis of Intermediate 33 using (R)—N—((R*)-1-(5-((S)-1-amino-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 78) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 80: (S)-1-((S)-1-(2-((R*)-1-Amino-2-((1,1,1-trifluoro-2-methylpropan-2-yl)oxy)ethyl)-1H-benzo[d]imidazol-5-yl)-2-methoxyethyl)-4-(trifluoromethyl)imidazolidin-2-one

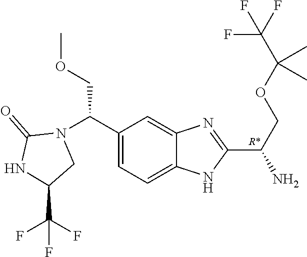 [SMILES: COC[C@H](c1ccc2[nH]c([C@@H](N)COC(C)(C)C(F)(F)F)nc2c1)N1C[C@@H](C(F)(F)F)NC1=O]

The title compound (82% yield) was prepared as described for the synthesis of Intermediate 34 using (R)—N—((R*)-1-(5-((S)-2-methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 79) in place of (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 81: (R)—N—((R*)-1-(5-((S)-1-(5,5-Difluoro-2-oxotetrahydropyrimidin-1(2H)-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

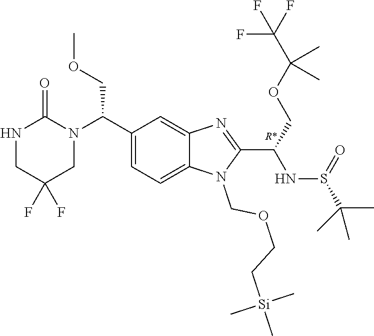 [SMILES: COC[C@H](c1ccc2c(c1)nc([C@H](COC(C)(C)C(F)(F)F)N[S@](=O)C(C)(C)C)n2COCC[Si](C)(C)C)N1CC(F)(F)CNC1=O]

The title compound was prepared as described for the synthesis of Intermediate 36 using (R)—N—((R*)-1-(5-((S)-1-amino-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 78) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and used without further purification.

### Intermediate 82: 1-((S)-1-(2-((R*)-1-Amino-2-((1,1,1-trifluoro-2-methylpropan-2-yl)oxy)ethyl)-1H-benzo[d]imidazol-5-yl)-2-methoxyethyl)-5,5-difluorotetrahydropyrimidin-2(1H)-one

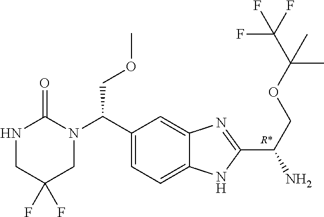 [SMILES: COC[C@H](c1ccc2[nH]c([C@@H](N)COC(C)(C)C(F)(F)F)nc2c1)N1CC(F)(F)CNC1=O]

The title compound was prepared as described for the synthesis of Intermediate 57 using (R)—N—((R*)-1-(5-((S)-1-(5,5-difluoro-2-oxotetrahydropyrimidin-1(2H)-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 81) in place of (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide and used without further purification.

### Intermediate 83: tert-Butyl ((R)-1-(5-(((R*)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)carbamate

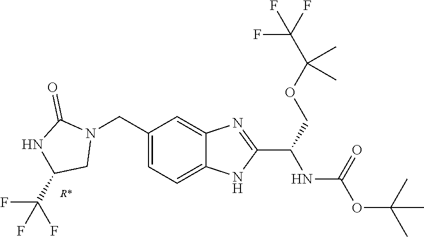 [SMILES: CC(C)(C)OC(=O)N[C@@H](COC(C)(C)C(F)(F)F)c1nc2cc(CN3C[C@H](C(F)(F)F)NC3=O)ccc2[nH]1]

### Intermediate 84: tert-Butyl ((R)-1-(5-(((S*)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)carbamate

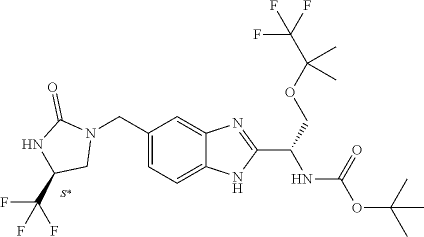 [SMILES: CC(C)(C)OC(=O)N[C@@H](COC(C)(C)C(F)(F)F)c1nc2cc(CN3C[C@@H](C(F)(F)F)NC3=O)ccc2[nH]1]

The title compounds were prepared as described for the synthesis of Intermediate 72 using 3,3,3-trifluoropropane-1,2-diamine dihydrochloride in place of 2,2-difluoropropane-1,3-diamine dihydrochloride. The material was purified by SFC using a chiral stationary phase (Stationary phase: Chiralpak IBN-5, 5 μm 250×21 mm, Mobile phase: 10% methanol, 90% CO₂). Intermediate 83 was the first eluting peak and Intermediate 84 was the second eluting peak (7% and 7% yield, respectively, over 2 steps).

### Intermediate 85: (S*)-1-((2-((R)-1-Amino-2-((1,1,1-trifluoro-2-methylpropan-2-yl)oxy)ethyl)-1H-benzo[d]imidazol-5-yl)methyl)-4-(trifluoromethyl)imidazolidin-2-one

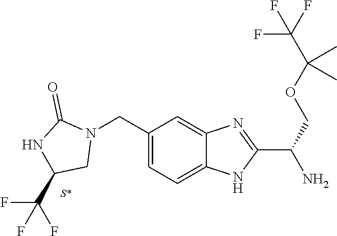 [SMILES: CC(C)(OC[C@H](N)c1nc2cc(CN3C[C@@H](C(F)(F)F)NC3=O)ccc2[nH]1)C(F)(F)F]

tert-Butyl ((R)-1-(5-(((S*)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)carbamate (50 mg, 0.09 mmol, Intermediate 84) was treated with TFA (0.28 mL, 3.6 mmol). The reaction mixture was stirred at rt for 30 min then quenched with saturated aqueous NaHCO₃. The aqueous layer was extracted with EtOAc (2×2 mL) then the combined organic layers were dried over anhydrous Na₂SO₄, filtered, and concentrated under reduced pressure. The material was used without further purification.

### Intermediate 86: (R*)-1-((2-((R)-1-Amino-2-((1,1,1-trifluoro-2-methylpropan-2-yl)oxy)ethyl)-1H-benzo[d]imidazol-5-yl)methyl)-4-(trifluoromethyl)imidazolidin-2-one

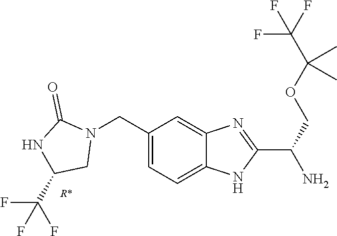 [SMILES: CC(C)(OC[C@H](N)c1nc2cc(CN3C[C@H](C(F)(F)F)NC3=O)ccc2[nH]1)C(F)(F)F]

The title compound was prepared as described for the synthesis of Intermediate 85 using tert-butyl ((R)-1-(5-(((R*)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)carbamate (Intermediate 83) in place of tert-butyl ((R)-1-(5-(((S*)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)carbamate and used without further purification.

### Intermediate 87: 5-Chloro-1-((2-(trimethylsilyl)ethoxy)methyl)-1H-imidazo[4,5-b]pyridine and 5-chloro-3-((2-(trimethylsilyl)ethoxy)methyl)-3H-imidazo[4,5-b]pyridine

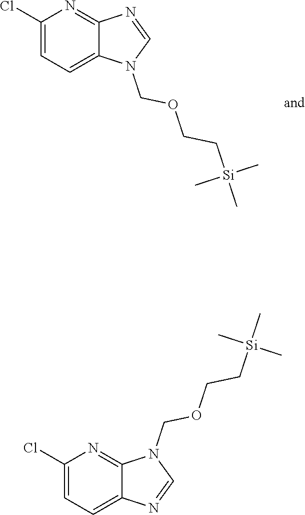 [SMILES: C[Si](C)(C)CCOCn1cnc2nc(Cl)ccc21 ; C[Si](C)(C)CCOCn1cnc2ccc(Cl)nc21]

The title compounds were prepared as described for the synthesis of Intermediate 13 using 5-chloro-3H-imidazo[4,5-b]pyridine in place of 5-bromo-1H-benzo[d]imidazole and using DMF as a solvent instead of THF. Purification by silica gel chromatography (0-100% (10% MeOH in EtOAc)/hexanes) provided the title compounds in 73% yield.

### Intermediate 88: 1-((2-(Trimethylsilyl)ethoxy)methyl)-5-vinyl-1H-imidazo[4,5-b]pyridine and 3-((2-(trimethylsilyl)ethoxy)methyl)-5-vinyl-3H-imidazo[4,5-b]pyridine

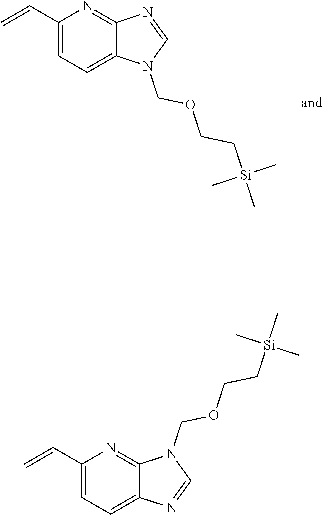

The title compounds were prepared as described for the synthesis of Intermediate 14 using 5-chloro-1-((2-(trimethylsilyl)ethoxy)methyl)-1H-imidazo[4,5-b]pyridine and 5-chloro-3-((2-(trimethylsilyl)ethoxy)methyl)-3H-imidazo[4,5-b]pyridine (Intermediate 87) in place of 5-bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole. The material was purified by silica gel chromatography (0-80% EtOAc/hexanes) to provide the title compounds in 78% yield.

### Intermediate 89: 1-((2-(Trimethylsilyl)ethoxy)methyl)-1H-imidazo[4,5-b]pyridine-5-carbaldehyde and 3-((2-(trimethylsilyl)ethoxy)methyl)-3H-imidazo[4,5-b]pyridine-5-carbaldehyde

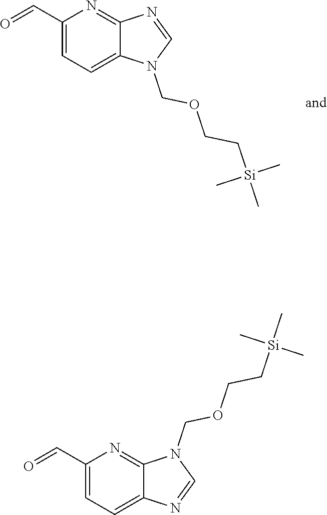 [SMILES: C[Si](C)(C)CCOCn1cnc2nc(C=O)ccc21 ; C[Si](C)(C)CCOCn1cnc2ccc(C=O)nc21]

The title compounds were prepared as described for the synthesis of Intermediate 15 using 1-((2-(trimethylsilyl)ethoxy)methyl)-5-vinyl-1H-imidazo[4,5-b]pyridine and 3-((2-(trimethylsilyl)ethoxy)methyl)-5-vinyl-3H-imidazo[4,5-b]pyridine (Intermediate 88) in place of 1-((2-(trimethylsilyl)ethoxy)methyl)-5-vinyl-1H-benzo[d]imidazole and 1-((2-(trimethylsilyl)ethoxy)methyl)-6-vinyl-1H-benzo[d]imidazole in 67% yield.

### Intermediate 90: 3,3,3-Trifluoro-N1-((1-((2-(trimethylsilyl)ethoxy)methyl)-1H-imidazo[4,5-b]pyridin-5-yl)methyl)propane-1,2-diamine and 3,3,3-trifluoro-N1-((3-((2-(trimethylsilyl)ethoxy)methyl)-3H-imidazo[4,5-b]pyridin-5-yl)methyl)propane-1,2-diamine

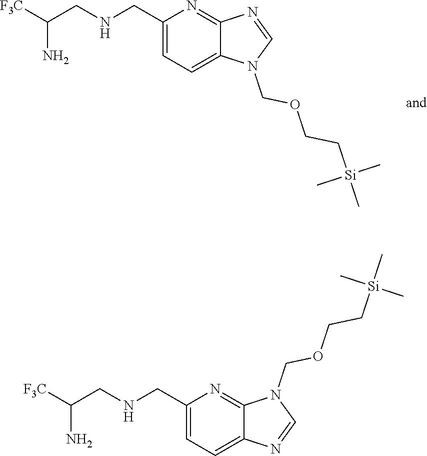 [SMILES: C[Si](C)(C)CCOCn1cnc2nc(CNCC(N)C(F)(F)F)ccc21 ; C[Si](C)(C)CCOCn1cnc2ccc(CNCC(N)C(F)(F)F)nc21]

The title compounds were prepared as described for the synthesis of Intermediate 64 using 1-((2-(trimethylsilyl)ethoxy)methyl)-1H-imidazo[4,5-b]pyridine-5-carbaldehyde and 3-((2-(trimethylsilyl)ethoxy)methyl)-3H-imidazo[4,5-b]pyridine-5-carbaldehyde (Intermediate 89) in place of 1-((2-(trimethylsilyl)ethoxy)methyl)-1H-benzo[d]imidazole-5-carbaldehyde and 3,3,3-trifluoropropane-1,2-diamine bis-hydrogen chloride in place of (S)-3,3,3-trifluoropropane-1,2-diamine bis-hydrogen chloride, and used without further purification.

### Intermediate 91: 4-(Trifluoromethyl)-1-((1-((2-(trimethylsilyl)ethoxy)methyl)-1H-imidazo[4,5-b]pyridin-5-yl)methyl)imidazolidin-2-one and 4-(trifluoromethyl)-1-((3-((2-(trimethylsilyl)ethoxy)methyl)-3H-imidazo[4,5-b]pyridin-5-yl)methyl)imidazolidin-2-one

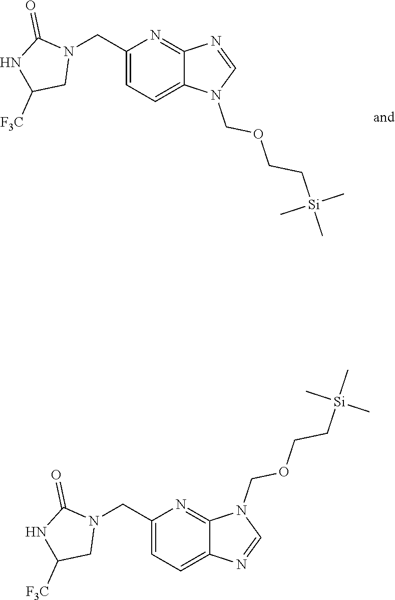 [SMILES: C[Si](C)(C)CCOCn1cnc2nc(CN3CC(C(F)(F)F)NC3=O)ccc21 ; C[Si](C)(C)CCOCn1cnc2ccc(CN3CC(C(F)(F)F)NC3=O)nc21]

The title compounds were prepared as described for the synthesis of Intermediate 65 using 3,3,3-trifluoro-N1-((1-((2-(trimethylsilyl)ethoxy)methyl)-1H-imidazo[4,5-b]pyridin-5-yl)methyl)propane-1,2-diamine and 3,3,3-trifluoro-N1-((3-((2-(trimethylsilyl)ethoxy)methyl)-3H-imidazo[4,5-b]pyridin-5-yl)methyl)propane-1,2-diamine (Intermediate 90) in place of (S)-3,3,3-trifluoro-N1-((1-((2-(trimethylsilyl)ethoxy)methyl)-1H-benzo[d]imidazol-5-yl)methyl)propane-1,2-diamine in 78% yield.

### Intermediate 92: (R)-2-Methyl-N-((1R*)-1-(5-((2-oxo-4-(trifluoromethyl)imidazolidin-1-yl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)propane-2-sulfinamide and (R)-2-methyl-N-((1R*)-1-(5-((2-oxo-4-(trifluoromethyl)imidazolidin-1-yl)methyl)-3-((2-(trimethylsilyl)ethoxy)methyl)-3H-imidazo[4,5-b]pyridin-2-yl)-2-((1,1,1-trifluoro-2-methylpropan-2-yl)oxy)ethyl)propane-2-sulfinamide

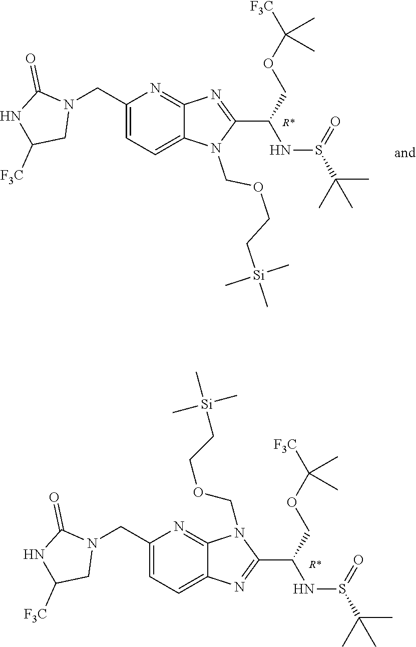

The title compounds were prepared as described for the synthesis of Intermediate 66 using 4-(trifluoromethyl)-1-((1-((2-(trimethylsilyl)ethoxy)methyl)-1H-imidazo[4,5-b]pyridin-5-yl)methyl)imidazolidin-2-one and 4-(trifluoromethyl)-1-((3-((2-(trimethylsilyl)ethoxy)methyl)-3H-imidazo[4,5-b]pyridin-5-yl)methyl)imidazolidin-2-one (Intermediate 91) in place of (S)-4-(trifluoromethyl)-1-((1-((2-(trimethylsilyl)ethoxy)methyl)-1H-benzo[d]imidazol-5-yl)methyl)imidazolidin-2-one in 31% yield.

### Intermediate 93: 1-((2-((R*)-1-Amino-2-((1,1,1-trifluoro-2-methylpropan-2-yl)oxy)ethyl)-1H-imidazo[4,5-b]pyridin-5-yl)methyl)-4-(trifluoromethyl)imidazolidin-2-one

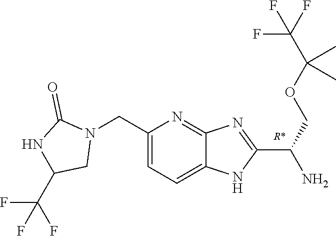 [SMILES: CC(C)(OC[C@H](N)c1nc2nc(CN3CC(C(F)(F)F)NC3=O)ccc2[nH]1)C(F)(F)F]

The title compound was prepared as described for Intermediate 57 using (R)-2-methyl-N-((1R*)-1-(5-((2-oxo-4-(trifluoromethyl)imidazolidin-1-yl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)propane-2-sulfinamide and (R)-2-methyl-N-((1R*)-1-(5-((2-oxo-4-(trifluoromethyl)imidazolidin-1-yl)methyl)-3-((2-(trimethylsilyl)ethoxy)methyl)-3H-imidazo[4,5-b]pyridin-2-yl)-2-((1,1,1-trifluoro-2-methylpropan-2-yl)oxy)ethyl)propane-2-sulfinamide (Intermediate 92) in place of (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide in 99% yield.

### Intermediate 94: (R)—N—((R*)-1-(5-((R)-Cyclopropyl(1,3-dioxoisoindolin-2-yl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide

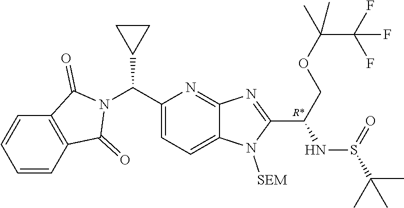 [SMILES: CC(C)(C)[S@@](=O)N[C@@H](COC(C)(C)C(F)(F)F)c1nc2nc([C@@H](C3CC3)N3C(=O)c4ccccc4C3=O)ccc2n1COCC[Si](C)(C)C]

Step A: (R)-Cyclopropyl(1-((2-(trimethylsilyl)ethoxy)methyl)-1H-imidazo[4,5-b]pyridin-5-yl)methanamine. The title compound was prepared as described for the synthesis of Intermediate 39 using (R)—N—((R)-cyclopropyl(1-((2-(trimethylsilyl)ethoxy)methyl)-1H-imidazo[4,5-b]pyridin-5-yl)methyl)-2-methylpropane-2-sulfinamide (Intermediate 115) in place of (S)-2-methyl-N—((R)-1-(1-((2-(trimethylsilyl)ethoxy)methyl)-1H-benzo[d]imidazol-6-yl)ethyl)propane-2-sulfinamide and (S)-2-methyl-N—((R)-1-(1-((2-(trimethylsilyl)ethoxy)methyl)-1H-benzo[d]imidazol-5-yl)ethyl)propane-2-sulfinamide, to afford the title compound as an off white foam. The material was used directly in the next step.

Step B: (R)-2-(Cyclopropyl(1-((2-(trimethylsilyl)ethoxy)methyl)-1H-imidazo[4,5-b]pyridin-5-yl)methyl)isoindoline-1,3-dione. The title compound was prepared as described for Intermediate 30 Step C using (R)-cyclopropyl(1-((2-(trimethylsilyl)ethoxy)methyl)-1H-imidazo[4,5-b]pyridin-5-yl)methanamine in place of (R)-cyclopropyl(1-((2-(trimethylsilyl)ethoxy)methyl)-1H-benzo[d]imidazol-5-yl)methanamine. The resulting material was recrystallized from hot ethyl acetate to afford the title compound as a white solid (88% two-step yield).

Step C: (R)—N—((R*)-1-(5-((R)-Cyclopropyl(1,3-dioxoisoindolin-2-yl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide. A round bottom flask was charged with (R)-2-(cyclopropyl(1-((2-(trimethylsilyl)ethoxy)methyl)-1H-imidazo[4,5-b]pyridin-5-yl)methyl)isoindoline-1,3-dione (2.2 g, 4.9 mmol, Step B), (R,E)-2-methyl-N-(2-((1,1,1-trifluoro-2-methylpropan-2-yl)oxy)ethylidene)propane-2-sulfinamide (2.0 g, 7.3 mmol, Intermediate 1), and THF (97 mL). The suspension was stirred until homogenous, about 5 min, then cooled to −78° C. A cold solution freshly prepared LDA (5.4 mL, 5.4 mmol, 1 M in THF) was added dropwise while maintaining an internal temperature of not more than −70° C. The reaction mixture was stirred for 30 min. To this solution was added acetic acid (2.4 mL, 24.4 mmol, 10 M in THF) dropwise and the solution was allowed to warm to rt. The reaction mixture was diluted with water and ethyl acetate, the layers were separated, and the aqueous phase was further extracted with ethyl acetate twice. The combined organics were washed with brine, dried over anhydrous MgSO₄, filtered and concentrated. This material was suspended in diethyl ether and left to sit for 1 h. The remaining (R)-2-(cyclopropyl(1-((2-(trimethylsilyl)ethoxy)methyl)-1H-imidazo[4,5-b]pyridin-5-yl)methyl)isoindoline-1,3-dione formed a white precipitate and was removed by filtration. The filtrate was concentrated, and the resulting material was purified by silica gel chromatography (0-100% ethyl acetate/DCM). The product containing fractions were concentrated to dryness to afford the title compound as a white solid (43% yield).

### Intermediate 95: (R)—N—((R*)-1-(5-((R)-Cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide

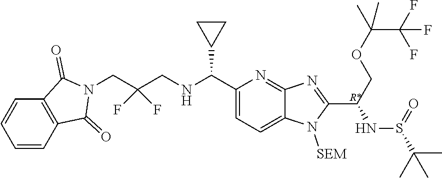 [SMILES: CC(C)(C)[S@@](=O)N[C@@H](COC(C)(C)C(F)(F)F)c1nc2nc([C@H](NCC(F)(F)CN3C(=O)c4ccccc4C3=O)C3CC3)ccc2n1COCC[Si](C)(C)C]

Step A: (R)—N—((R*)-1-(5-((R)-Amino(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide. The title compound was prepared as described for Intermediate 32 using (R)—N—((R*)-1-(5-((R)-cyclopropyl(1,3-dioxoisoindolin-2-yl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide Intermediate 94) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide, to afford the title compound as an off white foam (96% yield).

Step B: (R)—N—((R*)-1-(5-((R)-Cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide. A flask was charged with (R)—N—((R*)-1-(5-((R)-amino(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide (500 mg, 0.85 mmol, Step A), 3-(1,3-dioxoisoindolin-2-yl)-2,2-difluoropropyl trifluoromethanesulfonate (394 mg, 1.1 mmol, WO2010/011959), MeCN (3 mL), and DIPEA (291 μL, 1.7 mmol). The reaction was stirred for 1 h. The reaction was diluted with water and ethyl acetate. The layers were separated, and the aqueous phase was extracted with ethyl acetate twice. The combined organics were washed with brine, dried over anhydrous MgSO₄, filtered and concentrated. The crude material was purified by silica gel chromatography (1-100% ethyl acetate (with 10% MeOH)/hexanes) to afford the title compound as an off-white foam (58% yield).

### Intermediate 96: (R)—N—((R*)-1-(5-((R)-((3-Amino-2,2-difluoropropyl)amino)(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide

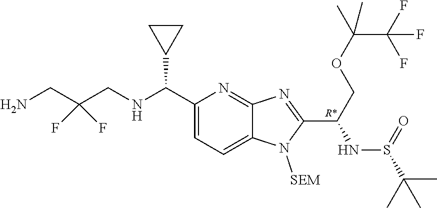 [SMILES: CC(C)(C)[S@@](=O)N[C@@H](COC(C)(C)C(F)(F)F)c1nc2nc([C@H](NCC(F)(F)CN)C3CC3)ccc2n1COCC[Si](C)(C)C]

A flask was charged with (R)—N—((R*)-1-(5-((R)-cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide (385 mg, 0.47 mmol, Intermediate 95), EtOH (2.5 mL), and hydrazine monohydrate (350 μL, 4.7 mmol). The reaction was stirred at rt for 2 h. After that time, the reaction was diluted with water and ethyl acetate, the layers were separated, and the aqueous phase was further extracted with ethyl acetate twice. The combined organic extracts were washed with brine, dried over anhydrous MgSO₄, filtered and concentrated to afford the title compound as a glassy solid (98% yield).

### Intermediate 97: (R)—N—((R*)-1-(5-((R)-Cyclopropyl(5,5-difluoro-2-oxotetrahydropyrimidin-1(2H)-yl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide

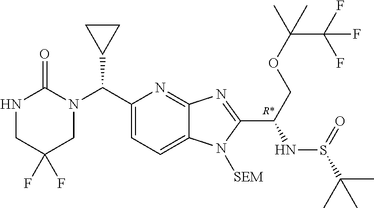

A flask was charged with (R)—N—((R*)-1-(5-((R)-((3-amino-2,2-difluoropropyl)amino)(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide (320 mg, 0.47 mmol, Intermediate 96), THF (9 mL), DIPEA (242 μL, 1.4 mmol) and CDI (227 mg, 1.4 mmol). The reaction mixture was warmed to 60° C. and stirred for 1 h. The reaction was allowed to cool to rt and quenched by the addition of water. The resulting solution was extracted three times with ethyl acetate. The combined organic extracts were washed with brine, dried over anhydrous MgSO₄, filtered and concentrated. This material was purified by silica gel chromatography (0-100% ethyl acetate (with 10% MeOH)/hexanes) to afford the title compound as a glassy solid (99% yield).

### Intermediate 98: 1-((R)-(2-((R*)-1-Amino-2-((1,1,1-trifluoro-2-methylpropan-2-yl)oxy)ethyl)-1H-imidazo[4,5-b]pyridin-5-yl)(cyclopropyl)methyl)-5,5-difluorotetrahydropyrimidin-2(1H)-one

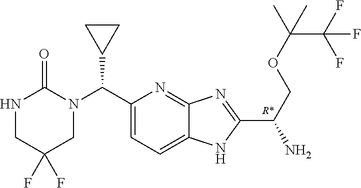 [SMILES: CC(C)(OC[C@H](N)c1nc2nc([C@@H](C3CC3)N3CC(F)(F)CNC3=O)ccc2[nH]1)C(F)(F)F]

A flask was charged with a stir bar, (R)—N—((R*)-1-(5-((R)-cyclopropyl(5,5-difluoro-2-oxotetrahydropyrimidin-1(2H)-yl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide (330 mg, 0.47 mmol, Intermediate 97), ethyl acetate (11 mL), resorcinol (294 mg, 2.7 mmol), and HCl (736 μL, 2.9 mmol, 4 M in 1,4-dioxane). The reaction was warmed to 60° C. and stirred for 1 h. The reaction was allowed to cool to rt. The mixture was concentrated, and the residue was dissolved in water. The aqueous phase was washed with 30% ethyl acetate in hexanes twice and the wash was discarded). The pH of the remaining aqueous layer was adjusted to pH 6 by the addition of 1 N aqueous NaOH and extracted with ethyl acetate three times. The combined extracts were washed with brine, dried over anhydrous MgSO₄, filtered and concentrated. This material was purified by preparative HPLC ((Xbridge Prep C18, 5 μm, 50×100 mm), 10-100% MeCN/aqueous 20 mM NH₄OH). The product containing fractions were lyophilized to afford the title compound as a white solid (25% yield).

### Intermediate 99: (R)—N-((1R*,2R)-1-(5-((R)-Cyclopropyl(1,3-dioxoisoindolin-2-yl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

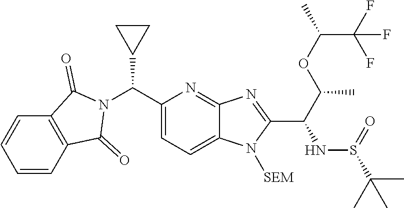 [SMILES: C[C@@H](O[C@H](C)C(F)(F)F)[C@H](N[S@](=O)C(C)(C)C)c1nc2nc([C@@H](C3CC3)N3C(=O)c4ccccc4C3=O)ccc2n1COCC[Si](C)(C)C]

The title compound was prepared as described for Intermediate 94 Step C using (R,E)-2-methyl-N—((R)-2-(((R)-1,1,1-trifluoropropan-2-yl)oxy)propylidene)propane-2-sulfinamide (Intermediate 5) in place of (R,E)-2-methyl-N-(2-((1,1,1-trifluoro-2-methylpropan-2-yl)oxy)ethylidene)propane-2-sulfinamide to afford the title compound as an off white solid.

### Intermediate 100: (R)—N-((1R*,2R)-1-(5-((R)-Amino(cyclopropyl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

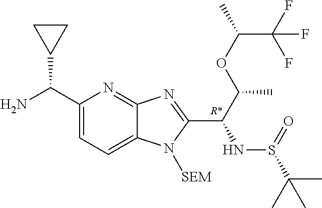

The title compound was prepared as described for Intermediate 32 using (R)—N-((1R*,2R)-1-(5-((R)-cyclopropyl(1,3-dioxoisoindolin-2-yl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 99) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide, to afford the title compound as an off white foam (98% yield).

### Intermediate 101: (R)—N-((1R*,2R)-1-(5-((R)-Cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

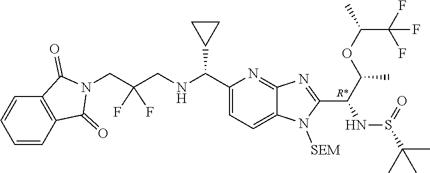 [SMILES: C[C@@H](O[C@H](C)C(F)(F)F)[C@H](N[S@](=O)C(C)(C)C)c1nc2nc([C@H](NCC(F)(F)CN3C(=O)c4ccccc4C3=O)C3CC3)ccc2n1COCC[Si](C)(C)C]

The title compound was prepared as described for Intermediate 95 Step B using (R)—N-((1R*,2R)-1-(5-((R)-amino(cyclopropyl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 100) in place of (R)—N—((R*)-1-(5-((R)-amino(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide to afford the title compound as a white foam (100% yield, assumed).

### Intermediate 102: (R)—N-((1R*,2R)-1-(5-((R)-((3-Amino-2,2-difluoropropyl)amino)(cyclopropyl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

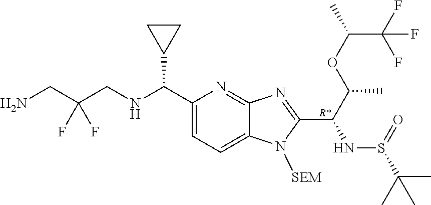 [SMILES: C[C@@H](O[C@H](C)C(F)(F)F)[C@H](N[S@](=O)C(C)(C)C)c1nc2nc([C@H](NCC(F)(F)CN)C3CC3)ccc2n1COCC[Si](C)(C)C]

The title compound was prepared as described for Intermediate 96 using (R)—N-((1R*,2R)-1-(5-((R)-cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 101) in place of (R)—N—((R*)-1-(5-((R)-cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide to afford the title compound as an off white foam (48% yield).

### Intermediate 103: (R)—N-((1R*,2R)-1-(5-((R)-Cyclopropyl(5,5-difluoro-2-oxotetrahydropyrimidin-1(2H)-yl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

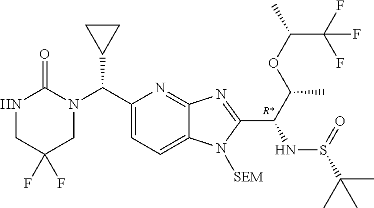 [SMILES: C[C@@H](O[C@H](C)C(F)(F)F)[C@H](N[S@](=O)C(C)(C)C)c1nc2nc([C@@H](C3CC3)N3CC(F)(F)CNC3=O)ccc2n1COCC[Si](C)(C)C]

The title compound was prepared as described for Intermediate 97 using (R)—N-((1R*,2R)-1-(5-((R)-((3-amino-2,2-difluoropropyl)amino)(cyclopropyl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 102) in place of (R)—N—((R*)-1-(5-((R)-((3-amino-2,2-difluoropropyl)amino)(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide to afford the title compound as a glassy solid (99% yield).

### Intermediate 104: 1-((R)-(2-((1R*,2R)-1-Amino-2-(((R)-1,1,1-trifluoropropan-2-yl)oxy)propyl)-1H-imidazo[4,5-b]pyridin-5-yl)(cyclopropyl)methyl)-5,5-difluorotetrahydropyrimidin-2(1H)-one

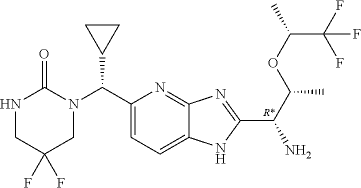 [SMILES: C[C@@H](O[C@H](C)C(F)(F)F)[C@H](N)c1nc2nc([C@@H](C3CC3)N3CC(F)(F)CNC3=O)ccc2[nH]1]

A flask was charged with (R)—N-((1R*,2R)-1-(5-((R)-cyclopropyl(5,5-difluoro-2-oxotetrahydropyrimidin-1(2H)-yl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (490 mg, 0.69 mmol, Intermediate 103), 1,4-dioxane (4 mL), MeOH (1 mL), and HCl (0.69 mL, 2.8 mmol, 4 M in 1,4-dioxane). The reaction was warmed to 60° C. and stirred for 1 h. The reaction was cooled to rt and concentrated. The residue was taken up in water and washed twice with hexanes (wash discarded). The pH of the resulting aqueous solution was adjusted to pH>7 by the careful addition of saturated aqueous sodium bicarbonate. This solution was extracted three times with ethyl acetate. The combined organic extracts were washed with brine, dried over anhydrous MgSO₄, filtered and concentrated into a glassy solid (91% yield).

### Intermediate 105: (R,E)-2-Methyl-N-((1-((2-(trimethylsilyl)ethoxy)methyl)-1H-imidazo[4,5-b]pyridin-5-yl)methylene)propane-2-sulfinamide

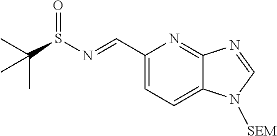 [SMILES: CC(C)(C)[S@@](=O)/N=C/c1ccc2c(ncn2COCC[Si](C)(C)C)n1]

A mixture of 1-((2-(trimethylsilyl)ethoxy)methyl)-1H-imidazo[4,5-b]pyridine-5-carbaldehyde and 3-((2-(trimethylsilyl)ethoxy)methyl)-3H-imidazo[4,5-b]pyridine-5-carbaldehyde (50.0 g, 180 mmol, Intermediate 89), (R)-2-methylpropane-2-sulfinamide (26.2 g, 216 mmol), Cs₂CO₃ (88.1 g, 270 mmol) and anhydrous CH₂Cl₂ (400 mL) were added to a 1 L three-necked round flask. The resultant mixture was stirred at room-temperature for 16 h. The reaction mixture was filtered through a pad of diatomaceous earth (e.g., Celite©) and the filtrate was concentrated under reduced pressure to afford the product (70 g as dark oil). This material was combined with another two of batches for purification by sillica gel chromatography (35-70% ethyl acetate/petroleum ether) to give two products. The first eluting product, (R,E)-2-methyl-N-((3-((2-(trimethylsilyl)ethoxy)methyl)-3H-imidazo[4,5-b]pyridin-5-yl)methylene)propane-2-sulfinamide, was obtained (47 g, 26%) as dark-orange oil. The second eluting product (the title compound, Intermediate 105), (R,E)-2-methyl-N-((1-((2-(trimethylsilyl)ethoxy)methyl)-1H-imidazo[4,5-b]pyridin-5-yl)methylene)propane-2-sulfinamide, was obtained (13%) as a yellow solid.

### Intermediate 106: (R)—N—((S)-2-Methoxy-1-(1-((2-(trimethylsilyl)ethoxy)methyl)-1H-imidazo[4,5-b]pyridin-5-yl)ethyl)-2-methylpropane-2-sulfinamide

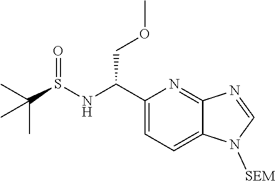 [SMILES: COC[C@@H](N[S@](=O)C(C)(C)C)c1ccc2c(ncn2COCC[Si](C)(C)C)n1]

A three neck round bottom flask was charged with anhydrous THF (150 mL) and tributyl(methoxymethyl)stannane (21.1 g, 63 mmol). The flask was cooled to −78° C. and a solution of n-BuLi (2.5 M in hexanes, 25.2 mL, 63.1 mmol) was added dropwise. The mixture was stirred for an hour before being treated with a solution of (R,E)-2-methyl-N-((1-((2-(trimethylsilyl)ethoxy)methyl)-1H-imidazo[4,5-b]pyridin-5-yl)methylene)propane-2-sulfinamide (8.00 g, 21.0 mmol, Intermediate 105) in 50 mL of anhydrous THF. The reaction mixture was stirred for 2 h before it was quenched with EtOH (10 mL), warmed to room-temperature, then quenched with aq. saturated NaHCO₃ solution (10 mL) and H₂O (10 mL). The combined aqueous phases were extracted with EtOAc (500 mL×3). The combined organic extracts were washed with brine (50 mL) and saturated aqueous KF solution (50 mL), sequentially, dried over anhydrous Na₂SO₄, filtered and concentrated under reduced pressure to afford a yellow oil. This oil was purified by sillica-gel chromatography (0-2% methanol/dichloromethane) to afford product as dark-orange oil which was further purified by HPLC (YMC-Triart Prep C18 column, m, 250×50 mm; 35-70% (v/v) H₂O (0.225% HCOOH)/CH₃CN). The diastereomers were separated by SFC using a chiral stationary phase (DAICEL CHIRALPAK AS column, 10 μm, 250 mm×50 mm; 30% (v/v) EtOH (containing 0.1% of 25% aq. NH₃)/CO₂) to give two products. Intermediate 106, the first eluting compound, was, obtained as a light-yellow solid (61% yield). The stereochemistry of the title compound was determined by comparison to material whose stereochemistry had been unambiguously assigned.

### Intermediate 107: (S)-2-Methoxy-1-(1-((2-(trimethylsilyl)ethoxy)methyl)-1H-imidazo[4,5-b]pyridin-5-yl)ethan-1-amine

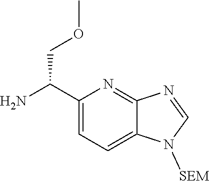 [SMILES: COC[C@@H](N)c1ccc2c(ncn2COCC[Si](C)(C)C)n1]

The title compound was prepared as described for Intermediate 75 using (R)—N—((S)-2-methoxy-1-(1-((2-(trimethylsilyl)ethoxy)methyl)-1H-imidazo[4,5-b]pyridin-5-yl)ethyl)-2-methylpropane-2-sulfinamide (Intermediate 106) in place of (R)—N—((S*)-2-methoxy-1-(1-((2-(trimethylsilyl)ethoxy)methyl)-1H-benzo[d]imidazol-5-yl)ethyl)-2-methylpropane-2-sulfinamide.

### Intermediate 108: (S)-2-(2-Methoxy-1-(1-((2-(trimethylsilyl)ethoxy)methyl)-1H-imidazo[4,5-b]pyridin-5-yl)ethyl)isoindoline-1,3-dione

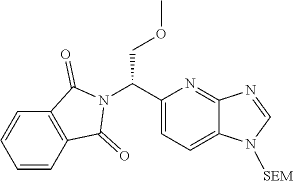 [SMILES: COC[C@H](c1ccc2c(ncn2COCC[Si](C)(C)C)n1)N1C(=O)c2ccccc2C1=O]

The title compound was prepared as described for the synthesis of Intermediate 30 Step C using (S)-2-methoxy-1-(1-((2-(trimethylsilyl)ethoxy)methyl)-1H-imidazo[4,5-b]pyridin-5-yl)ethan-1-amine (Intermediate 107) in place of (R)-cyclopropyl(1-((2-(trimethylsilyl)ethoxy)methyl)-1H-benzo[d]imidazol-5-yl)methanamine.

### Intermediate 109: (R)—N-((1R*,2R)-1-(5-((S)-1-(1,3-Dioxoisoindolin-2-yl)-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

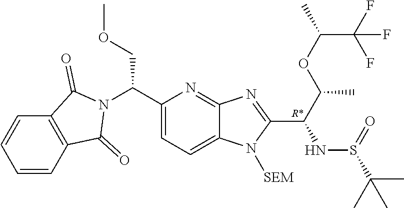 [SMILES: COC[C@H](c1ccc2c(n1)nc([C@@H](N[S@](=O)C(C)(C)C)[C@@H](C)O[C@H](C)C(F)(F)F)n2COCC[Si](C)(C)C)N1C(=O)c2ccccc2C1=O]

The title compound was prepared as described for the synthesis of Intermediate 31 using (S)-2-(2-methoxy-1-(1-((2-(trimethylsilyl)ethoxy)methyl)-1H-imidazo[4,5-b]pyridin-5-yl)ethyl)isoindoline-1,3-dione (Intermediate 108) in place of (R)-2-(cyclopropyl(1-((2-(trimethylsilyl)ethoxy)methyl)-1H-benzo[d]imidazol-5-yl)methyl)isoindoline-1,3-dione, and (R)-2-methyl-N—((R,E)-2-(((R)-1,1,1-trifluoropropan-2-yl)oxy)propylidene)propane-2-sulfinamide (Intermediate 5) in place of (R,E)-2-methyl-N-(2-((1,1,1-trifluoro-2-methylpropan-2-yl)oxy)ethylidene)propane-2-sulfinamide (75% yield).

### Intermediate 110: (R)—N-((1R*,2R)-1-(5-((S)-1-Amino-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

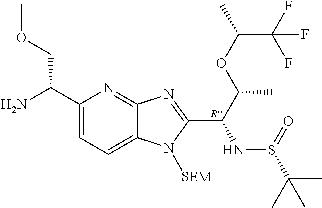 [SMILES: COC[C@@H](N)c1ccc2c(n1)nc([C@@H](N[S@](=O)C(C)(C)C)[C@@H](C)O[C@H](C)C(F)(F)F)n2COCC[Si](C)(C)C]

The title compound was prepared as described for Intermediate 32 using (R)—N-((1R*,2R)-1-(5-((S)-1-(1,3-dioxoisoindolin-2-yl)-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 109) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide, to afford the title compound as an off white foam (99% yield).

### Intermediate 111: (R)—N-((1R*,2R)-1-(5-((S)-1-((3-(1,3-Dioxoisoindolin-2-yl)-2,2-difluoropropyl)amino)-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

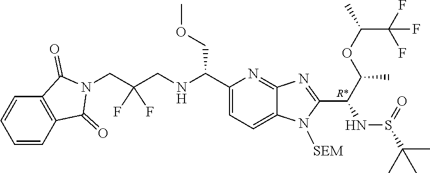 [SMILES: COC[C@@H](NCC(F)(F)CN1C(=O)c2ccccc2C1=O)c1ccc2c(n1)nc([C@@H](N[S@](=O)C(C)(C)C)[C@@H](C)O[C@H](C)C(F)(F)F)n2COCC[Si](C)(C)C]

The title compound was prepared as described for Intermediate 95 Step B using (R)—N-((1R*,2R)-1-(5-((S)-1-amino-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 110) in place of (R)—N—((R*)-1-(5-((R)-amino(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide to afford the title compound as a white foam (99% yield).

### Intermediate 112: (R)—N-((1R*,2R)-1-(5-((S)-1-((3-Amino-2,2-difluoropropyl)amino)-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

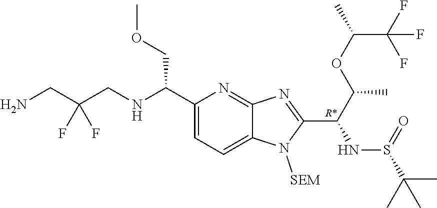 [SMILES: COC[C@@H](NCC(F)(F)CN)c1ccc2c(n1)nc([C@@H](N[S@](=O)C(C)(C)C)[C@@H](C)O[C@H](C)C(F)(F)F)n2COCC[Si](C)(C)C]

The title compound was prepared as described for Intermediate 96 using (R)—N-((1R*,2R)-1-(5-((S)-1-((3-(1,3-dioxoisoindolin-2-yl)-2,2-difluoropropyl)amino)-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 111) in place of (R)—N—((R*)-1-(5-((R)-cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide to afford the title compound as an off white foam (76% yield).

### Intermediate 113: (R)—N-((1R*,2R)-1-(5-((S)-1-(5,5-Difluoro-2-oxotetrahydropyrimidin-1(2H)-yl)-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide

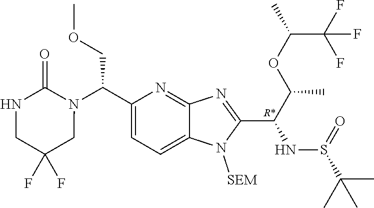 [SMILES: COC[C@H](c1ccc2c(n1)nc([C@@H](N[S@](=O)C(C)(C)C)[C@@H](C)O[C@H](C)C(F)(F)F)n2COCC[Si](C)(C)C)N1CC(F)(F)CNC1=O]

The title compound was prepared as described for Intermediate 97 using (R)—N-((1R*,2R)-1-(5-((S)-1-((3-amino-2,2-difluoropropyl)amino)-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 112) in place of (R)—N—((R*)-1-(5-((R)-((3-amino-2,2-difluoropropyl)amino)(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide to afford the title compound as a glassy solid (60% yield).

### Intermediate 114: 1-((S)-1-(2-((1R*,2R)-1-Amino-2-(((R)-1,1,1-trifluoropropan-2-yl)oxy)propyl)-1H-imidazo[4,5-b]pyridin-5-yl)-2-methoxyethyl)-5,5-difluorotetrahydropyrimidin-2(1H)-one

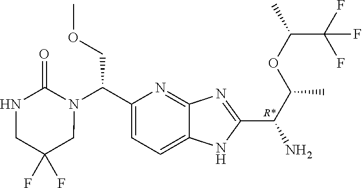 [SMILES: COC[C@H](c1ccc2[nH]c([C@@H](N)[C@@H](C)O[C@H](C)C(F)(F)F)nc2n1)N1CC(F)(F)CNC1=O]

The title compound was prepared as described for Intermediate 104 using (R)—N-((1R*,2R)-1-(5-((S)-1-(5,5-difluoro-2-oxotetrahydropyrimidin-1(2H)-yl)-2-methoxyethyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide (Intermediate 113) in place of (R)—N-((1R,2R)-1-(5-((R)-cyclopropyl(5,5-difluoro-2-oxotetrahydropyrimidin-1(2H)-yl)methyl)-1-((2-(trimethylsilyl)ethoxy)methyl)-1H-imidazo[4,5-b]pyridin-2-yl)-2-(((R)-1,1,1-trifluoropropan-2-yl)oxy)propyl)-2-methylpropane-2-sulfinamide to afford the title compound as a glassy solid (74% yield).

### Intermediate 115: (R)—N—((R)-Cyclopropyl(1-((2-(trimethylsilyl)ethoxy)methyl)-1H-imidazo[4,5-b]pyridin-5-yl)methyl)-2-methylpropane-2-sulfinamide

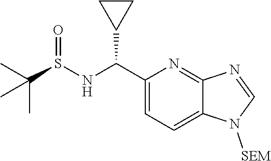 [SMILES: CC(C)(C)[S@@](=O)N[C@@H](c1ccc2c(ncn2COCC[Si](C)(C)C)n1)C1CC1]

A 3-necked round-bottom flask was charged with (R,E)-2-methyl-N-((1-((2-(trimethylsilyl)ethoxy)methyl)-1H-imidazo[4,5-b]pyridin-5-yl)methylene)propane-2-sulfinamide (600 g, 1.57 mol, Intermediate 105) and DCM (6 L). The reaction was cooled to −78° C. and a solution of cyclopropyl magnesium bromide (7.88 L, 7.88 mol, 1 M in THF) was added dropwise into the stirred solution. The reaction was allowed to stir at −78° C. for an additional 2 h. The reaction was allowed to warm to 0° C. and quenched by the addition of water (5 L), the resulting mixture was extracted with DCM (2×3 L). The combined organics were washed with water (2×10 L), and brine (2×10 L). The mixture was dried over anhydrous sodium sulfate and concentrated. The residue was purified by silica gel column chromatography, eluted with petroleum ether/ethyl acetate (1:3). The diastereomers were separated by SFC using a chiral stationary phase. Stationary phase: CHIRALPAK IG, 5×30 cm, 10 μm; Mobile Phase A: CO₂, Mobile Phase B: MeOH Preparative; Flow rate: 160 mL/min; Gradient: isocratic 50% B; Column Temperature (° C.): 35; Retention time (min): 4.13 to afford the title compound as a white solid (30.6% yield).

### Intermediate 116: 2-Cyclopropoxyacetic acid

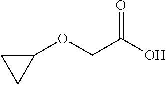

To a 0° C. suspension of NaH (3.4 g, 86 mmol) in THF (50 mL) was added cyclopropanol (2.5 g, 43 mmol) dropwise. The reaction was stirred at 0° C. for 1 h then bromoacetic acid (5.4 g, 39 mmol) was added as a solution in THF (10 mL). The reaction was allowed to warm to rt then stirred at this temperature for 16 h. The mixture was diluted with H₂O (100 mL) then washed with CH₂Cl₂ (2×50 mL) and the organic washes were discarded. The aqueous layer was acidified with HCl (43 mL, 86 mmol, 2 M in H₂O) then extracted with EtOAc (3×75 mL). The organic extracts were combined, dried over anhydrous Na₂SO₄, filtered, and concentrated to dryness to afford the title compound (94%).

### Intermediate 117: 1,3-Dioxoisoindolin-2-yl 2-cyclopropoxyacetate

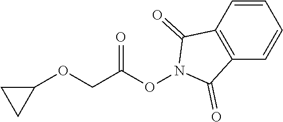 [SMILES: O=C(COC1CC1)ON1C(=O)c2ccccc2C1=O]

To a solution of 2-cyclopropylacetic acid (5.0 g, 43 mmol, Intermediate 116) in EtOAc (36 mL) was added 1-propanephosphonic anhydride (26 mL, 43 mmol, 50% in EtOAc). The reaction was stirred for 2 minutes at room temperature then 2-hydroxyisoindoline-1,3-dione (5.8 g, 36 mmol) was added. The reaction was stirred at room temperature for 5 h then diluted with EtOAc. The mixture was washed with 1 M HCl, half saturated aqueous NaHCO₃, and brine, then dried over anhydrous Na₂SO₄, filtered, and concentrated to dryness to afford the title compound (62%).

### Intermediate 118: (R,E)-2,4,6-Trimethyl-N-((1-((2-(trimethylsilyl)ethoxy)methyl)-1H-benzo[d]imidazol-5-yl)methylene)benzenesulfinamide

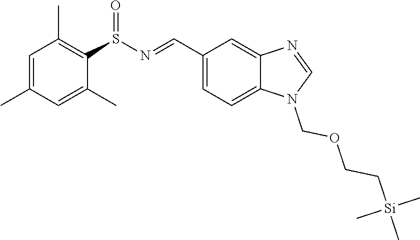

To a solution of 1-((2-(trimethylsilyl)ethoxy)methyl)-1H-benzo[d]imidazole-5-carbaldehyde (550 mg, 2.0 mmol, Intermediate 10) and (R)-2,4,6-trimethylbenzenesulfinamide (440 mg, 2.4 mmol) in anhydrous THF (10 mL) was added titanium isopropoxide (1.1 mL, 3.6 mmol). The reaction was stirred at room temperature for 20 h then quenched with saturated aqueous NaHCO₃ (0.29 mL) and diluted with EtOAc. The resulting suspension was filtered through a pad of Celite© and concentrated to dryness to afford the title compound that was used without further purification.

### Intermediate 119: (R)—N—((S)-2-Cyclopropoxy-1-(1-((2-(trimethylsilyl)ethoxy)methyl)-1H-benzo[d]imidazol-5-yl)ethyl)-2,4,6-trimethylbenzenesulfinamide

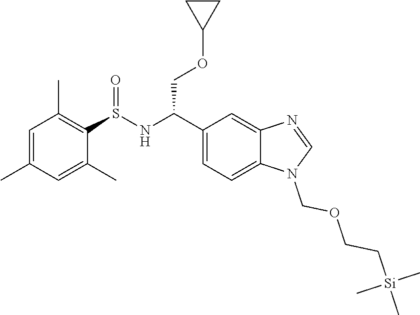

A mixture of (R,E)-2,4,6-trimethyl-N-((1-((2-(trimethylsilyl)ethoxy)methyl)-1H-benzo[d]imidazol-5-yl)methylene)benzenesulfinamide (442 mg, 1.00 mmol, Intermediate 118), 1,3-dioxoisoindolin-2-yl 2-cyclopropoxyacetate (522 mg, 2.00 mmol, Intermediate 117), and diethyl 1,4-dihydro-2,6-dimethyl-3,5-pyridinecarboxylate (507 mg, 2.00 mmol) in DMSO (10 mL) was degassed by sparging with N₂. DIPEA (0.345 mL, 2.00 mmol) was added then the reaction was irradiated in a Penn Optical Coatings M1 photoreactor at 25% LED intensity for 6 h. At this point 1,3-dioxoisoindolin-2-yl 2-cyclopropoxyacetate (261 mg, 1.00 mmol, Intermediate 117) and diethyl 1,4-dihydro-2,6-dimethyl-3,5-pyridinecarboxylate (254 mg, 1.00 mmol) were added and the reaction was irradiated under the conditions listed above for another 4 h. The mixture was diluted with aqueous 5% LiCl then extracted 3× with EtOAc. The combined organic layers were washed with aqueous 5% LiCl and brine, dried over anhydrous Na₂SO₄, filtered, and concentrated to dryness. The residue was triturated in CH₂Cl₂ then filtered and concentrated to dryness. Purification by silica gel chromatography (10-100% EtOAc:hexanes) afforded the title compound (56% yield).

### Intermediate 120: (S)-2-Cyclopropoxy-1-(1-((2-(trimethylsilyl)ethoxy)methyl)-1H-benzo[d]imidazol-5-yl)ethan-1-amine

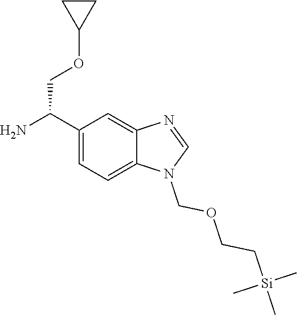 [SMILES: C[Si](C)(C)CCOCn1cnc2cc([C@H](N)COC3CC3)ccc21]

The title compound was prepared as described for the synthesis of Intermediate 39 using (R)—N—((S)-2-cyclopropoxy-1-(1-((2-(trimethylsilyl)ethoxy)methyl)-1H-benzo[d]imidazol-5-yl)ethyl)-2,4,6-trimethylbenzenesulfinamide (Intermediate 119) in place of (S)-2-methyl-N—((R)-1-(1-((2-(trimethylsilyl)ethoxy)methyl)-1H-benzo[d]imidazol-6-yl)ethyl)propane-2-sulfinamide and (S)-2-methyl-N—((R)-1-(1-((2-(trimethylsilyl)ethoxy)methyl)-1H-benzo[d]imidazol-5-yl)ethyl)propane-2-sulfinamide in 82% yield.

### Intermediate 121: (S)-2-(2-Cyclopropoxy-1-(1-((2-(trimethylsilyl)ethoxy)methyl)-1H-benzo[d]imidazol-5-yl)ethyl)isoindoline-1,3-dione

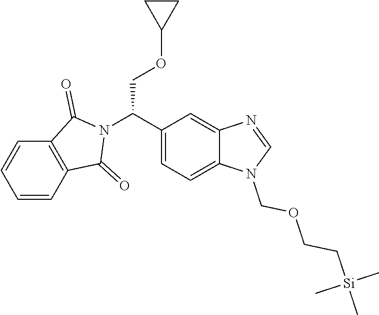 [SMILES: C[Si](C)(C)CCOCn1cnc2cc([C@@H](COC3CC3)N3C(=O)c4ccccc4C3=O)ccc21]

The title compound was prepared as described for the synthesis of Intermediate 30 Step C using (S)-2-cyclopropoxy-1-(1-((2-(trimethylsilyl)ethoxy)methyl)-1H-benzo[d]imidazol-5-yl)ethan-1-amine (Intermediate 120) in place of (R)-cyclopropyl(1-((2-(trimethylsilyl)ethoxy)methyl)-1H-benzo[d]imidazol-5-yl)methanamine in 71% yield.

### Intermediate 122: N—((R*)-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

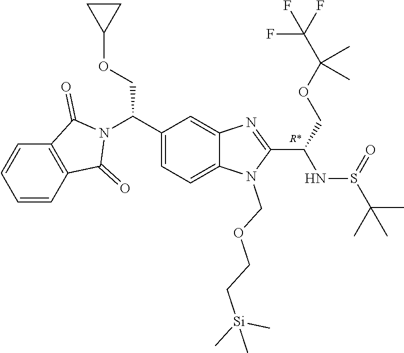 [SMILES: CC(C)(C)S(=O)N[C@@H](COC(C)(C)C(F)(F)F)c1nc2cc([C@@H](COC3CC3)N3C(=O)c4ccccc4C3=O)ccc2n1COCC[Si](C)(C)C]

The title compound was prepared as described for the synthesis of Intermediate 31 using (S)-2-(2-cyclopropoxy-1-(1-((2-(trimethylsilyl)ethoxy)methyl)-1H-benzo[d]imidazol-5-yl)ethyl)isoindoline-1,3-dione (Intermediate 121) in place of (R)-2-(cyclopropyl(1-((2-(trimethylsilyl)ethoxy)methyl)-1H-benzo[d]imidazol-5-yl)methyl)isoindoline-1,3-dione in 57% yield.

### Intermediate 123: N—((R*)-1-(5-((S)-1-Amino-2-cyclopropoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

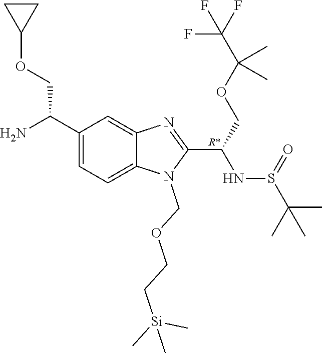 [SMILES: CC(C)(C)S(=O)N[C@@H](COC(C)(C)C(F)(F)F)c1nc2cc([C@H](N)COC3CC3)ccc2n1COCC[Si](C)(C)C]

The title compound was prepared as described for the synthesis of Intermediate 32 using N—((R*)-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 122) in place of (R)—N—((R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 124: N—((R*)-1-(5-((S)-2-cyclopropoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

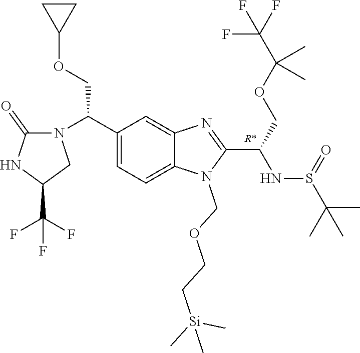 [SMILES: CC(C)(C)S(=O)N[C@@H](COC(C)(C)C(F)(F)F)c1nc2cc([C@@H](COC3CC3)N3C[C@@H](C(F)(F)F)NC3=O)ccc2n1COCC[Si](C)(C)C]

The title compound was prepared as described for the synthesis of Intermediate 33 using N—((R*)-1-(5-((S)-1-amino-2-cyclopropoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 123) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 39% yield.

### Intermediate 125: (S)-1-((S)-1-(2-((R*)-1-Amino-2-((1,1,1-trifluoro-2-methylpropan-2-yl)oxy)ethyl)-1H-benzo[d]imidazol-5-yl)-2-cyclopropoxyethyl)-4-(trifluoromethyl)imidazolidin-2-one

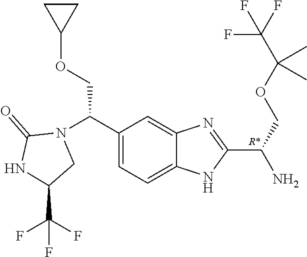 [SMILES: CC(C)(OC[C@H](N)c1nc2cc([C@@H](COC3CC3)N3C[C@@H](C(F)(F)F)NC3=O)ccc2[nH]1)C(F)(F)F]

The title was prepared in a manner analogous to the synthesis of Intermediate 34 using N—((R*)-1-(5-((S)-2-cyclopropoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 124) in place of (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 126: 1,3-Dioxoisoindolin-2-yl 4,4,4-trifluoro-2-methoxybutanoate

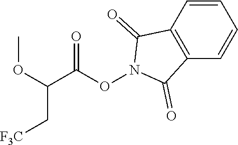 [SMILES: COC(CC(F)(F)F)C(=O)ON1C(=O)c2ccccc2C1=O]

Step A: Dimethyl 2-methoxy-2-(2,2,2-trifluoroethyl)malonate. A solution of dimethyl 2-methoxymalonate (7.5 g, 46 mmol) in THF (150 mL) was charged with potassium tert-butoxide (6.2 g, 56 mmol). The reaction was stirred for 2 h at 60° C., then 2,2,2-trifluoroethyl trifluoromethanesulfonate (12 g, 51 mmol) was added and heated to reflux for 2 d. The reaction was then cooled to rt, filtered, and concentrated under reduced pressure. The residue was purified by silica gel chromatography (0-30% EtOAc/petroleum ether) to afford the title compound in 48% yield.

Step B: 4,4,4-Trifluoro-2-methoxybutanoic acid. Dimethyl 2-methoxy-2-(2,2,2-trifluoroethyl)malonate (12 g, 49 mmol, Step A) was dissolved in conc. HCl (25 mL) and heated at 100° C. for 5 h. The reaction mixture was then diluted with water (30 mL) and extracted with EtOAc (50 mL×2). The combined extracts were washed with brine, dried over anhydrous Na₂SO₄, filtered, and concentrated under reduced pressure. The crude product was purified by silica gel chromatography (0-30% EtOAc/petroleum ether) to afford the title compound in 70% yield.

Step C: 1,3-Dioxoisoindolin-2-yl 4,4,4-trifluoro-2-methoxybutanoate. A round bottom flask was charged with 4,4,4-trifluoro-2-methoxybutanoic acid (1.0 g, 5.8 mmol, Step B) was diluted with EtOAc (5 mL) and had DIPEA (1.2 mL, 6.8 mmol) and T₃P (3.5 mL, 5.8 mmol, 50% solution in THF) and stirred for 2 min at rt. Then, 2-hydroxyisoindoline-1,3-dione (0.79 g, 4.8 mmol) was added and the reaction was allowed to stir overnight at rt. The solution was then diluted with EtOAc and washed sequentially with 1 N aqueous HCl, saturated aqueous NaHCO₃, water, and brine. The organic layer was then dried over anhydrous Na₂SO₄, filtered, and concentrated under reduced pressure to afford the title compound in 85% yield.

### Intermediate 127: (S)-2,4,6-Trimethyl-N—((E)-(5-(((S)-2-oxo-4-(trifluoromethyl)imidazolidin-1-yl)methyl)-1-((2-(trimethylsilyl)ethoxy)methyl)-1H-benzo[d]imidazol-2-yl)methylene)benzenesulfinamide

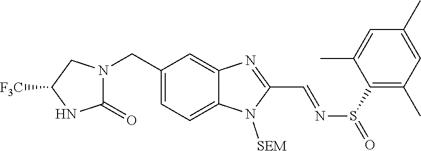 [SMILES: Cc1cc(C)c([S@](=O)/N=C/c2nc3cc(CN4C[C@@H](C(F)(F)F)NC4=O)ccc3n2COCC[Si](C)(C)C)c(C)c1]

Step A: (S)-5-((2-Oxo-4-(trifluoromethyl)imidazolidin-1-yl)methyl)-1-((2-(trimethylsilyl)ethoxy)methyl)-1H-benzo[d]imidazole-2-carbaldehyde. (S)-4-(Trifluoromethyl)-1-((1-((2-(trimethylsilyl)ethoxy)methyl)-1H-benzo[d]imidazol-5-yl)methyl)imidazolidin-2-one (1.5 g, 3.6 mmol, Intermediate 65) was dissolved in THF (50 mL) and cooled to −40° C. Then, n-BuLi (4.3 mL, 11 mmol, 2.5 M in hexanes) was added dropwise over 5 min and allowed to stir at −40° C. for 1 h. DMF (1.4 mL, 18 mmol) was then added dropwise and the resulting mixture was allowed to warm to rt for 1 h. The reaction was quenched with MeOH, diluted with water and extracted with EtOAc (3×). The combined organic layers were washed with brine, dried over anhydrous Na₂SO₄, filtered, and concentrated under reduced pressure. The residue was purified by silica gel chromatography (0-100% EtOAc/petroleum ether) to afford the title compound in 62% yield.

Step B: (S)-2,4,6-Trimethyl-N—((E)-(5-(((S)-2-oxo-4-(trifluoromethyl)imidazolidin-1-yl)methyl)-1-((2-(trimethylsilyl)ethoxy)methyl)-1H-benzo[d]imidazol-2-yl)methylene)benzenesulfinamide. A vial was charged with (S)-5-((2-oxo-4-(trifluoromethyl)imidazolidin-1-yl)methyl)-1-((2-(trimethylsilyl)ethoxy)methyl)-1H-benzo[d]imidazole-2-carbaldehyde (1.0 g, 2.3 mmol, Step A), (S)-2,4,6-trimethylbenzenesulfinamide (500 mg, 2.7 mmol), Cs₂CO₃ (884 mg, 2.7 mmol), and DCM (10 mL). The resulting mixture was allowed to stir at rt overnight. After this time the reaction was diluted with DCM and was washed sequentially with water and brine. The organic layer was then dried over anhydrous Na₂SO₄, filtered, and concentrated under reduced pressure. The crude product was purified by silica gel chromatography (0-70% EtOAc/petroleum ether) to afford the title compound in 44% yield.

### Intermediate 128: (4S)-1-((2-(1-Amino-4,4,4-trifluoro-2-methoxybutyl)-1H-benzo[d]imidazol-5-yl)methyl)-4-(trifluoromethyl)imidazolidin-2-one

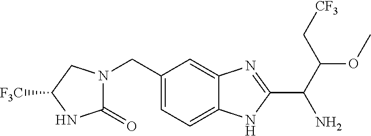 [SMILES: COC(CC(F)(F)F)C(N)c1nc2cc(CN3C[C@@H](C(F)(F)F)NC3=O)ccc2[nH]1]

Step A: (S)-2,4,6-Trimethyl-N-(4,4,4-trifluoro-2-methoxy-1-(5-(((S)-2-oxo-4-(trifluoromethyl)imidazolidin-1-yl)methyl)-1-((2-(trimethylsilyl)ethoxy)methyl)-1H-benzo[d]imidazol-2-yl)butyl)benzenesulfinamide. A vial was charged with (S)-2,4,6-trimethyl-N—((E)-(5-(((S)-2-oxo-4-(trifluoromethyl)imidazolidin-1-yl)methyl)-1-((2-(trimethylsilyl)ethoxy)methyl)-1H-benzo[d]imidazol-2-yl)methylene)benzenesulfinamide (600 mg, 0.99 mmol, Intermediate 127), 1,3-dioxoisoindolin-2-yl 4,4,4-trifluoro-2-methoxybutanoate (940 mg, 3.0 mmol, Intermediate 126), diethyl 2,6-dimethyl-1,4-dihydropyridine-3,5-dicarboxylate (750 mg, 3.0 mmol), and DIPEA (0.34 mL, 2.0 mmol) in DMSO (12 mL). The solution was sparged with Ar for 10 min, sealed, and irradiated with blue LEDs (450 nm, 40% intensity) for 2 h. After this time, the reaction was diluted with water and extracted with EtOAc (50 mL×4). The combined organic layers were washed sequentially with water and brine, dried over anhydrous Na₂SO₄, filtered, and concentrated under reduced pressure. The crude product was purified by silica gel chromatography (0-100% EtOAc/petroleum ether) to afford the title compound in 55% yield.

Step B: (4S)-1-((2-(1-Amino-4,4,4-trifluoro-2-methoxybutyl)-1H-benzo[d]imidazol-5-yl)methyl)-4-(trifluoromethyl)imidazolidin-2-one. (S)-2,4,6-Trimethyl-N-(4,4,4-trifluoro-2-methoxy-1-(5-(((S)-2-oxo-4-(trifluoromethyl)imidazolidin-1-yl)methyl)-1-((2-(trimethylsilyl)ethoxy)methyl)-1H-benzo[d]imidazol-2-yl)butyl)benzenesulfinamide (400 mg, 0.54 mmol, Step A) was dissolved in DCM (3 mL). TFA (3 mL) was added in one portion and the reaction was heated at 40° C. for 3 h. After this time the reaction was concentrated under reduced pressure and the resulting residue was diluted with water, extracted with DCM. The aqueous layer basified to pH>10 with NH₄OH. The basified aqueous layer was extracted with DCM and the combined organic layers were dried over anhydrous Na₂SO₄, filtered, and concentrated under reduced pressure. The residue was dissolved in MeOH (2 mL) and DIPEA (2 mL), heated to 80° C., and stirred for 3 h. After this time the reaction mixture was concentrated under reduced pressure. The residue was diluted with DCM and washed with 1 N aqueous HCl. The aqueous wash was basified to pH>10 with NH₄OH and then was extracted with DCM. The combined organic layers were dried over anhydrous Na₂SO₄, filtered, and concentrated under reduced pressure to afford the title compound in 29% yield.

### Intermediate 129: (R)—N—((R,E)-2-Cyclopropoxypropylidene)-2-methylpropane-2-sulfinamide

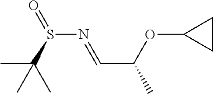

Step A: (R)-2-Cyclopropoxy-N-methoxy-N-methylpropanamide. The title compound was prepared as described for the synthesis of Intermediate 3 using (R)-2-cyclopropoxypropanoic acid in place of (R)-2-(((R)-1,1,1-trifluoropropan-2-yl)oxy)propanoic acid in 43% yield.

Step B: (R)-2-Cyclopropoxypropanal. The title compound was prepared as described for the synthesis of Intermediate 4 using (R)-2-cyclopropoxy-N-methoxy-N-methylpropanamide (Step A) in place of (R)—N-methoxy-N-methyl-2-(((R)-1,1,1-trifluoropropan-2-yl)oxy)propanamide in 100% yield.

Step C: (R)—N—((R,E)-2-Cyclopropoxypropylidene)-2-methylpropane-2-sulfinamide The title compound was prepared as described for the synthesis of Intermediate 5 using (R)-2-cyclopropoxypropanal (Step B) in place of (R)-2-(((R)-1,1,1-trifluoropropan-2-yl)oxy)propanal in 47% yield.

### Intermediate 130: (S)-1-((S)-1-(2-((1R,2R)-1-Amino-2-cyclopropoxypropyl)-1H-benzo[d]imidazol-5-yl)-2-methoxyethyl)-4-(trifluoromethyl)imidazolidin-2-one

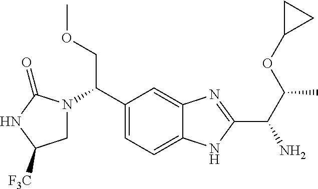 [SMILES: COC[C@H](c1ccc2[nH]c([C@@H](N)[C@@H](C)OC3CC3)nc2c1)N1C[C@@H](C(F)(F)F)NC1=O]

Step A: (R)—N-((1R,2R)-2-Cyclopropoxy-1-(5-((S)-1-(1,3-dioxoisoindolin-2-yl)-2-methoxyethyl)-1-((2-(trimethylsilyl)ethoxy)methyl)-1H-benzo[d]imidazol-2-yl)propyl)-2-methylpropane-2-sulfinamide. (S)-2-(2-Methoxy-1-(1-((2-(trimethylsilyl)ethoxy)methyl)-1H-benzo[d]imidazol-5-yl)ethyl)isoindoline-1,3-dione (1.55 g, 3.42 mmol, Intermediate 76) and THF (80 mL) were combined and cooled to −78° C. under a nitrogen atmosphere. LDA (5.2 mL, 5.2 mmol, 1 M in THF/hexanes) was then added dropwise and the contents were stirred at −78° C. for 10 minutes. (R)—N—((R,E)-2-Cyclopropoxypropylidene)-2-methylpropane-2-sulfinamide (1.32 g, 6.07 mmol, Intermediate 129) in THF (3 mL) was then added and the contents were stirred for 30 min at −78° C. Additional LDA (1.7 mL, 1.7 mmol, 1 M in THF/hexanes) was then added 3 times at 30 min intervals, and stirred at −78° C. for 15 min after the final addition of LDA. The the reaction was then quenched at −78° C. with the addition of acetic acid (2 mL) in THF (10 mL). The reaction solution was stirred for 2 min at −78° C., then the contents were allowed to warm to rt. The reaction solution was then transferred to a separatory funnel with ethyl acetate and extracted twice with deionized water. The organic phase was separated, dried over anhydrous magnesium sulfate, filtered and concentrated under reduced pressure. The product was purified in batches by preparative HPLC (XBridge Phenyl Prep 5 μm C18 50×250 mm, 40-100% MeCN with 0.05% TFA/water with 0.05% TFA) to afford, after lyophilization, the title compound in 13% yield.

Step B: (R)—N-((1R,2R)-1-(5-((S)-1-Amino-2-methoxyethyl)-1-((2-(trimethylsilyl)ethoxy)methyl)-1H-benzo[d]imidazol-2-yl)-2-cyclopropoxypropyl)-2-methylpropane-2-sulfinamide. The title compound was prepared as described for the synthesis of Intermediate 32 using (R)—N-((1R,2R)-2-cyclopropoxy-1-(5-((S)-1-(1,3-dioxoisoindolin-2-yl)-2-methoxyethyl)-1-((2-(trimethylsilyl)ethoxy)methyl)-1H-benzo[d]imidazol-2-yl)propyl)-2-methylpropane-2-sulfinamide (Step A) in place of (R)—N—((R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 95% yield.

Step C: (R)—N-((1R,2R)-2-Cyclopropoxy-1-(5-((S)-2-methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)propyl)-2-methylpropane-2-sulfinamide. The title compound was prepared as described for the synthesis of Intermediate 33 using (R)—N-((1R,2R)-1-(5-((S)-1-amino-2-methoxyethyl)-1-((2-(trimethylsilyl)ethoxy)methyl)-1H-benzo[d]imidazol-2-yl)-2-cyclopropoxypropyl)-2-methylpropane-2-sulfinamide (Step B) in place of benzyl ((S)-3-(((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)amino)-1,1,1-trifluoropropan-2-yl)carbamate in 39% yield over 3 steps.

Step D: (S)-1-((S)-1-(2-((1R,2R)-1-Amino-2-cyclopropoxypropyl)-1H-benzo[d]imidazol-5-yl)-2-methoxyethyl)-4-(trifluoromethyl)imidazolidin-2-one. The title compound was prepared as described for the synthesis of Intermediate 34 using (R)—N-((1R,2R)-2-cyclopropoxy-1-(5-((S)-2-methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)propyl)-2-methylpropane-2-sulfinamide (Step C) in place of (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide in 53% yield.

### Intermediate 131: 1-((S)-1-(2-((1R,2R)-1-Amino-2-cyclopropoxypropyl)-1H-benzo[d]imidazol-5-yl)-2-methoxyethyl)-5,5-difluorotetrahydropyrimidin-2(1H)-one

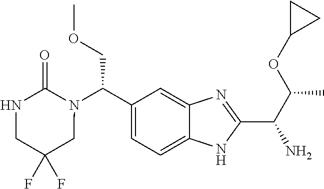 [SMILES: COC[C@H](c1ccc2[nH]c([C@@H](N)[C@@H](C)OC3CC3)nc2c1)N1CC(F)(F)CNC1=O]

Step A: (R)—N-((1R,2R)-2-Cyclopropoxy-1-(5-((S)-1-(5,5-difluoro-2-oxotetrahydropyrimidin-1(2H)-yl)-2-methoxyethyl)-1-((2-(trimethylsilyl)ethoxy)methyl)-1H-benzo[d]imidazol-2-yl)propyl)-2-methylpropane-2-sulfinamide. The title compound was prepared as described for the synthesis of Intermediate 36 using (R)—N-((1R,2R)-1-(5-((S)-1-amino-2-methoxyethyl)-1-((2-(trimethylsilyl)ethoxy)methyl)-1H-benzo[d]imidazol-2-yl)-2-cyclopropoxypropyl)-2-methylpropane-2-sulfinamide (Intermediate 130 Step B) in place of (R)—N—((R)-1-(5-((R)-cyclopropyl((3-(1,3-dioxoisoindolin-2-yl)-2,2-difluoropropyl)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 70% yield over 3 steps.

Step B. 1-((S)-1-(2-((1R,2R)-1-Amino-2-cyclopropoxypropyl)-1H-benzo[d]imidazol-5-yl)-2-methoxyethyl)-5,5-difluorotetrahydropyrimidin-2(1H)-one. The title compound was prepared as described for the synthesis of Intermediate 37 using (R)—N-((1R,2R)-2-cyclopropoxy-1-(5-((S)-1-(5,5-difluoro-2-oxotetrahydropyrimidin-1(2H)-yl)-2-methoxyethyl)-1-((2-(trimethylsilyl)ethoxy)methyl)-1H-benzo[d]imidazol-2-yl)propyl)-2-methylpropane-2-sulfinamide (Step A) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 68% yield.

### Intermediate 132: 5-Bromo-4-fluoro-1-((2-(trimethylsilyl)ethoxy)methyl)-1H-benzo[d]imidazole

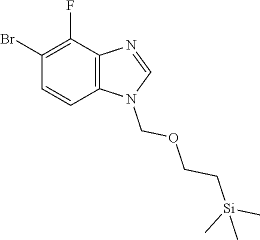 [SMILES: C[Si](C)(C)CCOCn1cnc2c(F)c(Br)ccc21]

A solution of 5-bromo-4-fluoro-1H-benzo[d]imidazole (1.00 g, 4.65 mmol) in THF (20 mL) was cooled to 0° C. and then NaH (179 mg, 7.44 mmol, 60% dispersion in mineral oil) was added portion-wise over 20 min. The mixture was stirred at 0° C. for 1 h, then (2-(chloromethoxy)ethyl)trimethylsilane (0.99 mL, 5.6 mmol) was added over 5 min. The resulting mixture was allowed to warm to rt and stirred at rt for 2 h. The reaction mixture was cooled to 0° C. and quenched with saturated aqueous NH₄Cl (40 mL) and water (60 mL). Then the mixture was extracted with EtOAc (2×30 mL), and the organic layers combined, washed with brine (100 mL), dried over anhydrous Na₂SO₄, filtered and concentrated to dryness. The residue was purified by silica gel chromatography (0-50% EtOAc/petroleum ether) to provide the title compound as a black oil (88% yield).

### Intermediate 133: 4-Fluoro-1-((2-(trimethylsilyl)ethoxy)methyl)-5-vinyl-1H-benzo[d]imidazole

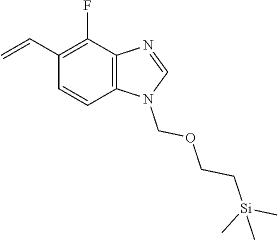 [SMILES: C=Cc1ccc2c(ncn2COCC[Si](C)(C)C)c1F]

5-Bromo-4-fluoro-1-((2-(trimethylsilyl)ethoxy)methyl)-1H-benzo[d]imidazole (7.00 g, 20.3 mmol, Intermediate 132), potassium trifluoro(vinyl)borate (5.43 g, 40.5 mmol), K₃PO₄ (12.9 g, 60.8 mmol), Pd(dppf)Cl₂·CH₂Cl₂ (828 mg, 1.01 mmol), 1,4-dioxane (70 mL) and H₂O (20 mL) were added to a three-neck round-bottomed flask. The reaction mixture was purged with N₂ three times and then heated at 95° C. for 16 h. The mixture was diluted with water (50 mL) and extracted with ethyl acetate (100 mL×2). The combined extracts were dried over anhydrous Na₂SO₄, filtered, and concentrated to dryness to give a brown oil. The oil was subjected to silica gel chromatography (0-30% EtOAc/petroleum ether) to provide the title compound as a yellow oil. This compound was used without further purification.

### Intermediate 134: 4-Fluoro-1-((2-(trimethylsilyl)ethoxy)methyl)-1H-benzo[d]imidazole-5-carbaldehyde

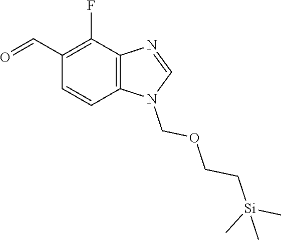 [SMILES: C[Si](C)(C)CCOCn1cnc2c(F)c(C=O)ccc21]

4-Fluoro-1-((2-(trimethylsilyl)ethoxy)methyl)-5-vinyl-1H-benzo[d]imidazole (17.8 g, 60.9 mmol, Intermediate 133), potassium osmate (VI) dihydrate (1.12 g, 3.04 mmol), THF (200 mL) and H₂O (50 mL) were added to a three-round-bottomed flask that was subsequently cooled to 0° C. The mixture was stirred at 0° C. for 30 min and then sodium periodate (39.1 g, 183 mmol) was added portion-wise over 2 min at 0° C. The mixture was stirred at 0° C. for 30 min, then the reaction vessel was removed from the cooling bath and stirred at rt for 16 h. The mixture was filtered through diatomaceous earth (e.g., Celite©) and H₂O (150 mL) was added to the filtrate. The mixture was extracted with EtOAc (150 mL×2), the organic layers combined, washed with brine (150 mL), dried over anhydrous Na₂SO₄, filtered, and concentrated to dryness to provide the title compound as a black oil. This compound was used without further purification.

### Intermediate 135: (R,E)-N-((4-Fluoro-1-((2-(trimethylsilyl)ethoxy)methyl)-1H-benzo[d]imidazol-5-yl)methylene)-2-methylpropane-2-sulfinamide

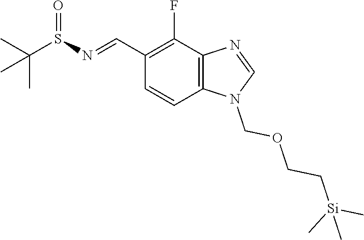 [SMILES: CC(C)(C)[S@@](=O)/N=C/c1ccc2c(ncn2COCC[Si](C)(C)C)c1F]

4-Fluoro-1-((2-(trimethylsilyl)ethoxy)methyl)-1H-benzo[d]imidazole-5-carbaldehyde (17.8 g, 60.5 mmol, Intermediate 134), (R)-2-methylpropane-2-sulfinamide (11.0 g, 90.7 mmol), Cs₂CO₃ (39.4 g, 121 mmol) and DCM (250 mL) were added to a three-necked round-bottomed flask. The resulting mixture was heated at 40° C. for 16 h. The reaction mixture was filtered, and diluted with H₂O (200 mL). The mixture was extracted with CH₂Cl₂ (150 mL×3), and the combined organic layers were washed with brine (150 mL×2), dried over anhydrous Na₂SO₄, filtered, and concentrated to dryness to give a black oil. The oil was subjected to silica gel chromatography (0-50% EtOAc/petroleum ether) to provide the title compound in 61% yield.

### Intermediate 136: (R)—N—((S)-1-(4-Fluoro-1-((2-(trimethylsilyl)ethoxy)methyl)-1H-benzo [d]imidazol-5-yl)-2-methoxyethyl)-2-methylpropane-2-sulfinamide

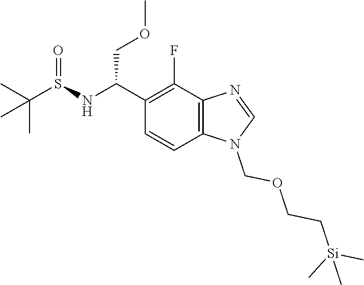 [SMILES: COC[C@@H](N[S@](=O)C(C)(C)C)c1ccc2c(ncn2COCC[Si](C)(C)C)c1F]

Tributyl(methoxymethyl)stannane (4.21 g, 12.6 mmol) and THF (40 mL) were added to an oven-dried and nitrogen-purged three-neck round-bottomed flask, which was subsequently cooled to −65° C. (dry ice/EtOH). Then, n-BuLi (12.6 mmol) was added dropwise over 30 min. The resulting mixture was stirred for 1 h at −65° C. Then a solution of (R,E)-N-((4-fluoro-1-((2-(trimethylsilyl)ethoxy)methyl)-1H-benzo[d]imidazol-5-yl)methylene)-2-methylpropane-2-sulfinamide (1.00 g, 2.52 mmol, Intermediate 135) in THF (10 mL) was added dropwise over 40 min. The resulting mixture was stirred for 1 h at −65° C. The reaction mixture was treated with saturated aqueous NH₄Cl solution (10 mL) and then diluted with EtOAc (30 mL). The mixture was washed with H₂O (30 mL×2) and brine (30 mL), dried over anhydrous Na₂SO₄, filtered, and concentrated to dryness to give a yellow oil. The oil was then subjected to silica gel chromatography (0-30% MeOH/DCM) to provide the title compound as a yellow oil (68% yield).

### Intermediate 137: (S)-1-(4-Fluoro-1-((2-(trimethylsilyl)ethoxy)methyl)-1H-benzo[d]imidazol-5-yl)-2-methoxyethanamine

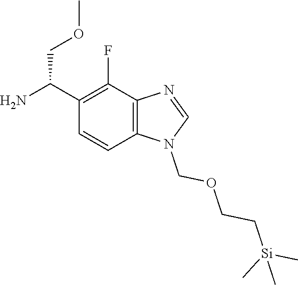 [SMILES: COC[C@@H](N)c1ccc2c(ncn2COCC[Si](C)(C)C)c1F]

To a solution of (R)—N—((S)-1-(4-fluoro-1-((2-(trimethylsilyl)ethoxy)methyl)-1H-benzo[d]imidazol-5-yl)-2-methoxyethyl)-2-methylpropane-2-sulfinamide (588 mg, 1.33 mmol, Intermediate 136) in EtOAc (5 mL) was added 4 M HCl in 1,4-dioxane (1.04 mL, 4.15 mmol) dropwise over 1 min. The resulting mixture was stirred at rt for 0.5 h. The reaction was diluted with H₂O (10 mL), and the aqueous phase was washed with EtOAc (10 mL×3). The combined organic phases were extracted with 2 M aqueous HCl (5 mL×2), and the pH of the combined aqueous phases was adjusted to pH 8 by the addition of saturated aqueous NaHCO₃ solution. The mixture was extracted with EtOAc (20 mL×2). The combined organic extracts were washed with brine (50 mL), dried over anhydrous Na₂SO₄, filtered, and concentrated to dryness to give the title compound as a yellow oil (79% yield), which was used without further purification.

### Intermediate 138: (S)-2-(1-(4-Fluoro-1-((2-(trimethylsilyl)ethoxy)methyl)-1H-benzo[d]imidazol-5-yl)-2-methoxyethyl)isoindoline-1,3-dione

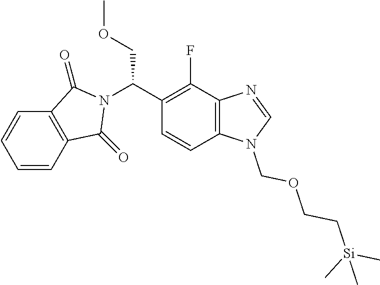 [SMILES: COC[C@H](c1ccc2c(ncn2COCC[Si](C)(C)C)c1F)N1C(=O)c2ccccc2C1=O]

A mixture of(S)-1-(4-fluoro-1-((2-(trimethylsilyl)ethoxy)methyl)-1H-benzo[d]imidazol-5-yl)-2-methoxyethanamine (408 mg, 1.20 mmol, Intermediate 137), ethyl 1,3-dioxoisoindoline-2-carboxylate (294 mg, 1.34 mmol) and DIPEA (0.70 mL, 4.0 mmol) in THF (7 mL) was heated at 75° C. for 48 h. The reaction mixture was then treated with H₂O (50 mL) and extracted with EtOAc (100 mL×3). The combined organic layers were washed with brine (100 mL), dried over anhydrous Na₂SO₄, filtered, and concentrated to dryness to give a yellow oil. The oil was then subjected to silica gel chromatography (0-50% EtOAc/petroleum ether) to give the title compound as a yellow oil (80% yield).

### Intermediate 139: (S)—N-((1R,2R)-1-(5-((S)-1-(1,3-Dioxoisoindolin-2-yl)-2-methoxyethyl)-4-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

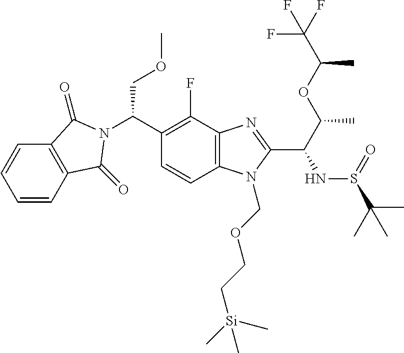 [SMILES: COC[C@H](c1ccc2c(nc([C@@H](N[S@@](=O)C(C)(C)C)[C@@H](C)O[C@H](C)C(F)(F)F)n2COCC[Si](C)(C)C)c1F)N1C(=O)c2ccccc2C1=O]

(S)-2-(1-(4-Fluoro-1-((2-(trimethylsilyl)ethoxy)methyl)-1H-benzo[d]imidazol-5-yl)-2-methoxyethyl)isoindoline-1,3-dione (500 mg, 1.07 mmol, Intermediate 138), (R,E)-2-methyl-N—((R)-2-(((R)-1,1,1-trifluoropropan-2-yl)oxy)propylidene)propane-2-sulfinamide (466 mg, 1.70 mmol, Intermediate 5), and dry THF (35 mL) were added to an oven-dried and nitrogen-purged three neck round-bottomed flask, which was subsequently cooled to −70° C. Then LDA (3.2 mL, 3.2 mmol, 1.0 M in THF) was added dropwise over 25 min. The resulting mixture was stirred at −70° C. for 1 h, and then quenched by the addition of a solution of HOAc/THF (3 mL; a solution of 0.5 mL acetic acid in 25 mL of THF). The mixture was stirred for 2 min at −78° C., then the ice bath was removed and the contents were warmed to rt. The reaction mixture was diluted with H₂O (50 mL) and extracted with EtOAc (100 mL×3). The combined organic layers were washed with brine (100 mL), dried over anhydrous MgSO₄, filtered, and concentrated to dryness to provide a yellow oil. The oil was subjected to silica gel chromatography (0-0.5% MeOH/DCM) to provide the title compound as a yellow oil (27% yield).

### Intermediate 140: (S)—N-((1R,2R)-1-(5-((S)-1-Amino-2-methoxyethyl)-4-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

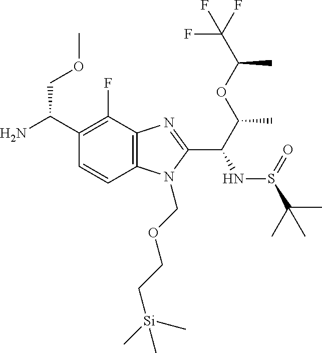 [SMILES: COC[C@@H](N)c1ccc2c(nc([C@@H](N[S@@](=O)C(C)(C)C)[C@@H](C)O[C@H](C)C(F)(F)F)n2COCC[Si](C)(C)C)c1F]

(S)—N-((1R,2R)-1-(5-((S)-1-(1,3-Dioxoisoindolin-2-yl)-2-methoxyethyl)-4-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (244 mg, 0.328 mmol, Intermediate 139), EtOH (2 mL) and N₂H₄·H₂O (194 mg, 3.28 mmol) were combined and stirred at rt for 4 h. Then EtOAc (20 mL) was added and the mixture was washed with H₂O (20 mL×2) and brine (50 mL), dried over anhydrous Na₂SO₄, filtered, and concentrated to dryness to give the title compound as a light yellow oil which was used without further purification.

### Intermediate 141: Benzyl ((S)-3-(((S)-1-(2-((1R,2R)-1-(((S)-tert-butylsulfinyl)amino)-2-(((R)-1,1,1-trifluoropropan-2-yl)oxy)propyl)-4-fluoro-1-((2-(trimethylsilyl)ethoxy)methyl)-1H-benzo[d]imidazol-5-yl)-2-methoxyethyl)amino)-1,1,1-trifluoropropan-2-yl)carbamate

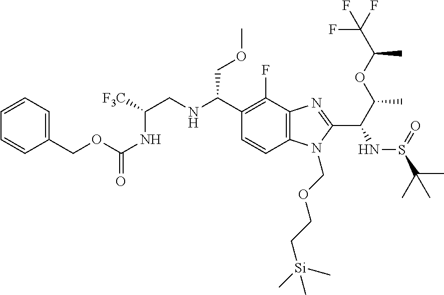 [SMILES: COC[C@@H](NC[C@H](NC(=O)OCc1ccccc1)C(F)(F)F)c1ccc2c(nc([C@@H](N[S@@](=O)C(C)(C)C)[C@@H](C)O[C@H](C)C(F)(F)F)n2COCC[Si](C)(C)C)c1F]

(S)—N-((1R,2R)-1-(5-((S)-1-Amino-2-methoxyethyl)-4-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (620 mg, 1.01 mmol, Intermediate 140), Na₂CO₃ (322 mg, 3.04 mmol), (S)-benzyl 4-(trifluoromethyl)-1,2,3-oxathiazolidine-3-carboxylate 2,2-dioxide (987 mg, 3.04 mmol, Intermediate 24) and THF/MeCN (1:1, 10 mL) were added to a vial. The reaction mixture was heated at 35° C. for 16 h. The reaction was diluted with EtOAc (50 mL), washed with H₂O (50 mL×2) and brine (70 mL), dried over anhydrous Na₂SO₄, filtered, and concentrated to dryness to give a light yellow oil. The oil was then subjected to silica gel chromatography (0-10% MeOH/DCM) to give the title compound as a colorless oil (80% yield).

### Intermediate 142: (S)—N-((1R,2R)-1-(5-((S)-1-(((S)-2-Amino-3,3,3-trifluoropropyl)amino)-2-methoxyethyl)-4-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

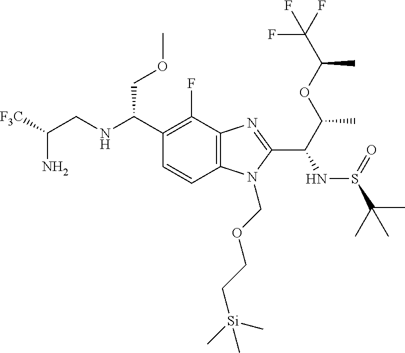 [SMILES: COC[C@@H](NC[C@H](N)C(F)(F)F)c1ccc2c(nc([C@@H](N[S@@](=O)C(C)(C)C)[C@@H](C)O[C@H](C)C(F)(F)F)n2COCC[Si](C)(C)C)c1F]

Benzyl ((S)-3-(((S)-1-(2-((1R,2R)-1-(((S)-tert-butylsulfinyl)amino)-2-(((R)-1,1,1-trifluoropropan-2-yl)oxy)propyl)-4-fluoro-1-((2-(trimethylsilyl)ethoxy)methyl)-1H-benzo[d]imidazol-5-yl)-2-methoxyethyl)amino)-1,1,1-trifluoropropan-2-yl)carbamate (820 mg, 0.956 mmol, Intermediate 141), MeOH (10 mL), and 10% wet Pd/C (820 mg, 10% Pd and 50% water) were added to a hydrogenation bottle. The resultant mixture was stirred under H₂ (45 psi) at 25° C. for 2 h. The mixture was filtered, and the filter cake was washed with MeOH (20 mL). The filtrate was concentrated to dryness to give the title compound as a white solid that was used without further purification.

### Intermediate 143: (S)-1-((S)-1-(2-((1R,2R)-1-Amino-2-(((R)-1,1,1-trifluoropropan-2-yl)oxy)propyl)-4-fluoro-1-((2-(trimethylsilyl)ethoxy)methyl)-1H-benzo[d]imidazol-5-yl)-2-methoxyethyl)-4-(trifluoromethyl)imidazolidin-2-one

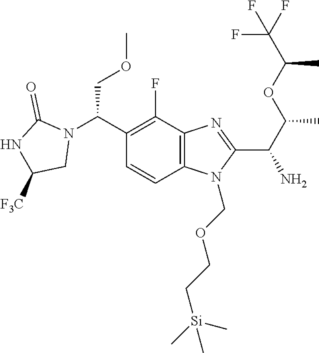 [SMILES: COC[C@H](c1ccc2c(nc([C@@H](N)[C@@H](C)O[C@H](C)C(F)(F)F)n2COCC[Si](C)(C)C)c1F)N1C[C@@H](C(F)(F)F)NC1=O]

(S)—N-((1R,2R)-1-(5-((S)-1-(((S)-2-Amino-3,3,3-trifluoropropyl)amino)-2-methoxyethyl)-4-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (740 mg, 1.02 mmol, Intermediate 142), DIPEA (0.7 mL, 4 mmol) and DCM (12 mL) were added to a vial that was cooled to 0° C. Then, bis(trichloromethyl) carbonate (160 mg, 0.539 mmol) was added in one portion and the resulting mixture was stirred at 0° C. for 0.5 h. The reaction mixture was poured into water (50 mL) and extracted with DCM (100 mL×2). The combined organic extracts were washed with saturated aqueous NaHCO₃ (50 mL) and brine (50 mL), dried over anhydrous MgSO₄, filtered and concentrated to dryness to afford a yellow oil. The oil was subjected to silica gel chromatography (0-10% MeOH/DCM) to give a mixture of the title compound and the de-silylated product, ((S)-1-((S)-1-(2-((1R,2R)-1-amino-2-(((R)-1,1,1-trifluoropropan-2-yl)oxy)propyl)-7-fluoro-1H-benzo[d]imidazol-6-yl)-2-methoxyethyl)-4-(trifluoromethyl)imidazolidin-2-one), as a light yellow oil that was used without further purification.

### Intermediate 144: (S)-1-((S)-1-(2-((1R,2R)-1-Amino-2-(((R)-1,1,1-trifluoropropan-2-yl)oxy)propyl)-4-fluoro-1H-benzo[d]imidazol-5-yl)-2-methoxyethyl)-4-(trifluoromethyl)imidazolidin-2-one

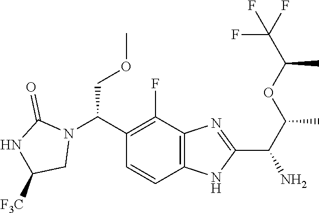 [SMILES: COC[C@H](c1ccc2[nH]c([C@@H](N)[C@@H](C)O[C@H](C)C(F)(F)F)nc2c1F)N1C[C@@H](C(F)(F)F)NC1=O]

To a solution of (S)-1-((S)-1-(2-((1R,2R)-1-amino-2-(((R)-1,1,1-trifluoropropan-2-yl)oxy)propyl)-4-fluoro-1-((2-(trimethylsilyl)ethoxy)methyl)-1H-benzo[d]imidazol-5-yl)-2-methoxyethyl)-4-(trifluoromethyl)imidazolidin-2-one (250 mg, 0.387 mmol, Intermediate 143) in 1,4-dioxane (1.5 mL) was added HCl (4 M in 1,4-dioxane, 3.0 mL, 12 mmol) dropwise over 1 min. The resulting mixture was heated at 55° C. for 1 h. The reaction mixture was then concentrated to dryness, dissolved in H₂O (20 mL), washed with DCM (20 mL×2) and the combined organic phases were extracted with 2 M aqueous HCl (20 mL×2). The pH of the combined aqueous phases was adjusted to pH 7-8 by the addition of a saturated aqueous NaHCO₃ solution. The resulting mixture was extracted with EtOAc (25 mL×3), the EtOAc extracts were combined and washed with brine (30 mL), dried over anhydrous Na₂SO₄, filtered, and concentrated to dryness to give a mixture of partially and fully deprotected products as a yellow solid. To this mixture, DIPEA (1.0 mL, 5.7 mmol) and MeOH (1.0 mL) were added. The resulting mixture was heated at 80° C. for 2 h. The reaction mixture was then concentrated to dryness, dissolved in DCM (5 mL), and washed with H₂O (10 mL). The organic phase was extracted with 2 M aqueous HCl (10 mL×2), then the pH of the combined aqueous phases was adjusted to pH 7-8 with a saturated aqueous NaHCO₃ solution. The resulting mixture was then extracted with EtOAc (15 mL×3) and the combined EtOAc layers were washed with brine (30 mL), dried over anhydrous Na₂SO₄, filtered, and concentrated to dryness to give the title compound as a light yellow solid that was used without further purification.

### Intermediate 145: tert-Butyl (R)-(1-(5-bromo-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl (R)-(1-(6-bromo-1-((2-(trimethylsilyl)ethoxy)methyl)-1H-benzo[d]imidazol-2-yl)-2-((1,1,1-trifluoro-2-methylpropan-2-yl)oxy)ethyl)carbamate

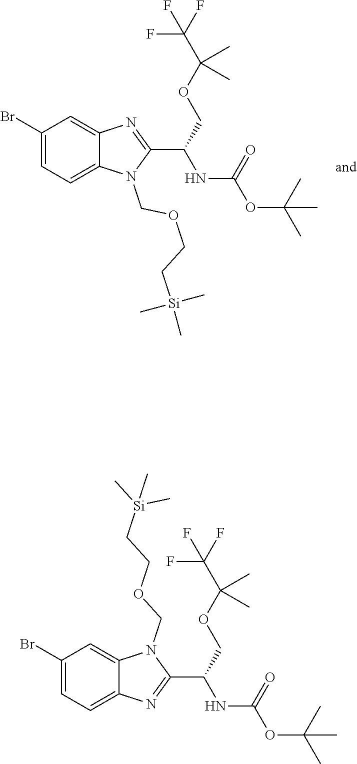 [SMILES: CC(C)(C)OC(=O)N[C@@H](COC(C)(C)C(F)(F)F)c1nc2cc(Br)ccc2n1COCC[Si](C)(C)C ; CC(C)(C)OC(=O)N[C@@H](COC(C)(C)C(F)(F)F)c1nc2ccc(Br)cc2n1COCC[Si](C)(C)C]

To a solution of (R)—N—((R)-1-(5-bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R)-1-(6-bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (6.95 g, 11.6 mmol, Intermediate 68) in THF:H₂O (ratio 4:1, 192 mL) was added iodine (587 mg, 2.32 mmol) and the resulting mixture was heated at/at 50° C. for 2 h. After this time, additional iodine (587 mg, 2.32 mmol) was added and the mixture stirred for an additional 3.5 h at 50° C. The reaction mixture was then allowed to cool to rt, quenched with sodium thiosulfate pentahydrate (25.4 mL, 5.07 mmol) and extracted with EtOAc (3×60 mL). The combined organic layers were dried over anhydrous MgSO₄, filtered, and concentrated to dryness. The material was dissolved in DCM (58 mL). Et₃N (3.54 mL, 25.5 mmol) was added followed by di-tert-butyl dicarbonate (3.06 g, 14.0 mmol). The reaction mixture was stirred overnight at rt. The reaction was then diluted with saturated aqueous NH₄Cl and extracted with EtOAc (3×60 mL). The combined organic layers were washed with brine, dried over anhydrous MgSO₄, filtered, and concentrated to dryness. The residue was purified by silica gel chromatography (0-60% EtOAc/hexanes) to afford a mixture of title compounds in 92% yield.

### Intermediate 146: tert-Butyl (R)-(1-(5-(3-methoxyprop-1-en-2-yl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl (R)-(1-(6-(3-methoxyprop-1-en-2-yl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

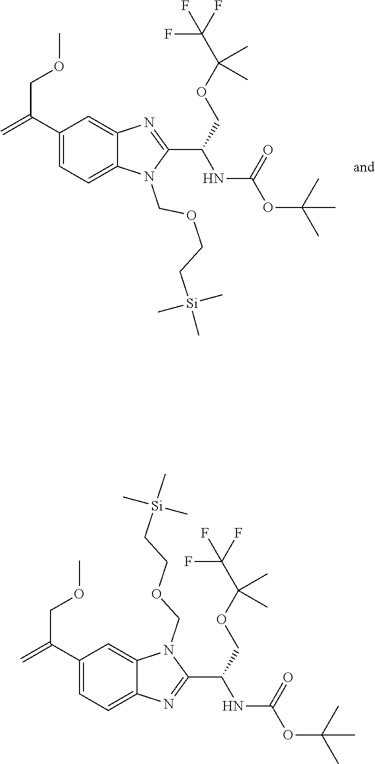

To a suspension of tert-butyl (R)-(1-(5-bromo-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl (R)-(1-(6-bromo-1-((2-(trimethylsilyl)ethoxy)methyl)-1H-benzo[d]imidazol-2-yl)-2-((1,1,1-trifluoro-2-methylpropan-2-yl)oxy)ethyl)carbamate (905 mg, 1.52 mmol, Intermediate 145), 4,4,4′,4′,5,5,5′,5′-octamethyl-2,2′-bi(1,3,2-dioxaborolane) (404 mg, 1.59 mmol) and potassium acetate (449 mg, 4.58 mmol) in 1,4-dioxane (15 mL) was added chloro(2-dicyclohexylphosphino-2′,4′,6′-triisopropyl-1,1′-biphenyl)[2-(2′-amino-1,1′-biphenyl)]palladium(II) (114 mg, 0.145 mmol). The mixture was purged with N₂ for 10 min and heated in a microwave reactor at 100° C. for 1 h. The reaction mixture was cooled to rt and then 2-bromo-3-methoxyprop-1-ene (0.33 mL, 3.04 mmol), K₃PO₄ (1.02 g, 4.55 mmol), mesylate[(di(1-adamantyl)-n-butylphosphine)-2-(2′-amino-1,1′-biphenyl)]palladium(II) (79.3 mg, 0.107 mmol), and H₂O (1.5 mL) were added. The resulting mixture was stirred and purged with N₂ for 10 min. The reaction mixture was heated at 100° C. for 18 h using a heating block. The reaction was cooled to rt, filtered through diatomaceous earth (e.g., Celite©), and the filtrate was diluted with H₂O (10 mL) and extracted with EtOAc (3×15 mL). The combined organic layers were washed with brine, dried over anhydrous Na₂SO₄, filtered, and concentrated to dryness. Purification by silica gel chromatography (0-100% EtOAc (10% MeOH)/hexanes) provided a mixture of title compounds in 49% yield.

### Intermediate 147: tert-Butyl (R)-(1-(5-(2-methoxyacetyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl (R)-(1-(6-(2-methoxyacetyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

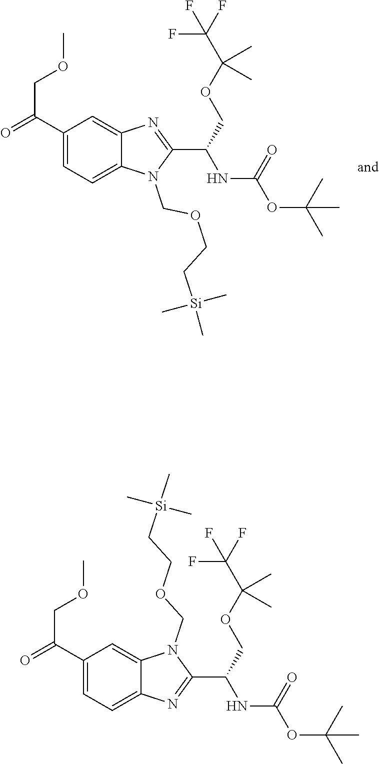 [SMILES: COCC(=O)c1ccc2c(c1)nc([C@H](COC(C)(C)C(F)(F)F)NC(=O)OC(C)(C)C)n2COCC[Si](C)(C)C ; COCC(=O)c1ccc2nc([C@H](COC(C)(C)C(F)(F)F)NC(=O)OC(C)(C)C)n(COCC[Si](C)(C)C)c2c1]

To tert-butyl (R)-(1-(5-(3-methoxyprop-1-en-2-yl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl (R)-(1-(6-(3-methoxyprop-1-en-2-yl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (439 mg, 0.747 mmol, Intermediate 146) in 1,4-dioxane (1 mL) was added a suspension of sodium metaperiodate (959 mg, 4.48 mmol) in H₂O (1 mL). The mixture was stirred rigorously and then potassium osmate(VI) dihydrate (11 mg, 0.030 mmol) was added. The resulting mixture was stirred at rt for 1 h, then the reaction was diluted with H₂O (5 mL) and EtOAc (5 mL) and extracted with EtOAc (3×10 mL). The combined organic layers were washed with brine, dried over anhydrous Na₂SO₄, filtered, and concentrated to dryness. Purification by silica gel chromatography (10-100% EtOAc/hexanes) provided a mixture of title compounds in 37% yield.

### Intermediate 148: tert-Butyl ((1R)-1-(5-(1-((2-amino-2-methylpropyl)amino)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl ((1R)-1-(6-(1-((2-amino-2-methylpropyl)amino)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

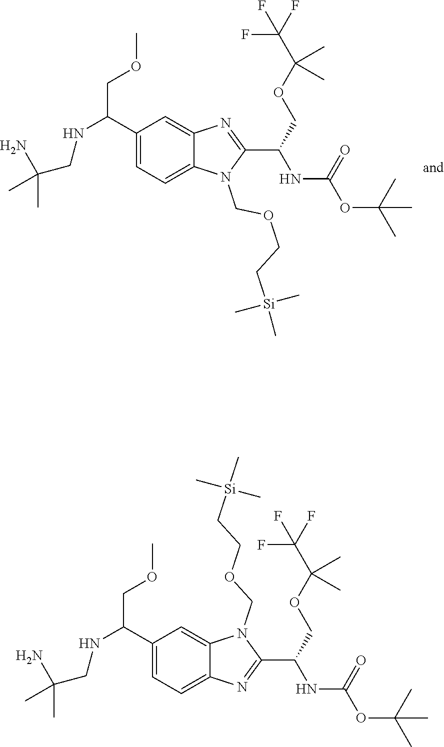

To tert-butyl (R)-(1-(5-(2-methoxyacetyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl (R)-(1-(6-(2-methoxyacetyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (200 mg, 0.339 mmol, Intermediate 147) in DCM (3.3 mL) were added 2-methylpropane-1,2-diamine (0.092 mL, 0.848 mmol) and TEA (0.19 mL, 1.36 mmol) and the resulting mixture was stirred at rt for 45 min. Then, acetic acid (0.116 mL, 2.04 mmol) and MeOH (1.2 mL) were added. After stirring the mixture for 30 min at rt, sodium cyanoborohydride (74.6 mg, 1.19 mmol) was added and the mixture was stirred at rt for 45 h. The reaction mixture was diluted with saturated aqueous NaHCO₃ and extracted with DCM (3×5 mL). The combined organic layers were dried over anhydrous Na₂SO₄, filtered, and concentrated to dryness to afford the mixture of title compounds that was used without further purification.

### Intermediate 149: tert-Butyl ((1R)-1-(5-(1-(4,4-dimethyl-2-oxoimidazolidin-1-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl ((1R)-1-(6-(1-(4,4-dimethyl-2-oxoimidazolidin-1-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

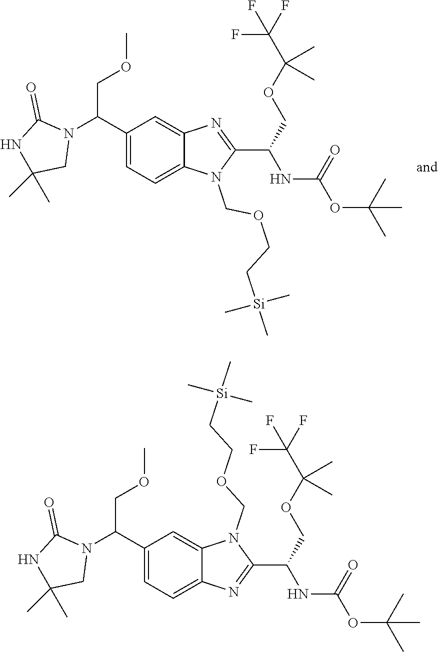

A solution of tert-butyl ((1R)-1-(5-(1-((2-amino-2-methylpropyl)amino)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl ((1R)-1-(6-(1-((2-amino-2-methylpropyl)amino)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (120 mg, 0.18 mmol, Intermediate 148) and DIPEA (96 mL, 0.55 mmol) in DCM (1.8 mL) was cooled to 0° C. under N₂. The resulting mixture was treated with triphosgene (18 mg, 0.060 mmol) and stirred at 0° C. for 20 min. The reaction mixture was then treated with water (2 mL), extracted with DCM (2×5 mL), and the combined organic extracts were concentrated to dryness. The isolated material was purified by silica gel chromatography (5-100% EtOAc (10% MeOH)/DCM) to afford the mixture of title compounds in 33% yield as a clear oil.

### Intermediate 150: 1-(1-(2-((R)-1-Amino-2-((1,1,1-trifluoro-2-methylpropan-2-yl)oxy)ethyl)-1H-benzo[d]imidazol-5-yl)-2-methoxyethyl)-4,4-dimethylimidazolidin-2-one

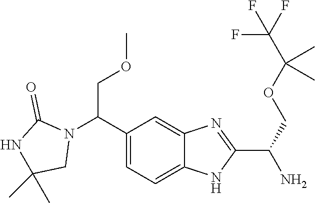 [SMILES: COCC(c1ccc2[nH]c([C@@H](N)COC(C)(C)C(F)(F)F)nc2c1)N1CC(C)(C)NC1=O]

The title compound was prepared as described for the synthesis of Intermediate 37 using tert-butyl ((1R)-1-(5-(1-(4,4-dimethyl-2-oxoimidazolidin-1-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 149) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and used directly in the next step without further purification.

### Intermediate 151: (R)-2-(((S)-1,1,1-Trifluoropropan-2-yl)oxy)propanal

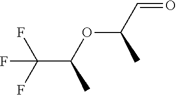

The title compound was prepared as described in Intermediate 4 using (R)—N-methoxy-N-methyl-2-(((S)-1,1,1-trifluoropropan-2-yl)oxy)propanamide (Intermediate 7) in place of (R)—N-methoxy-N-methyl-2-(((R)-1,1,1-trifluoropropan-2-yl)oxy)propanamide and used without further purification (98% yield).

### Intermediate 152: (R)—N-((1R,2R)-1-(5-((S)-1-(1,3-Dioxoisoindolin-2-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide and (R)—N-((1R,2R)-1-(6-((S)-1-(1,3-dioxoisoindolin-2-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

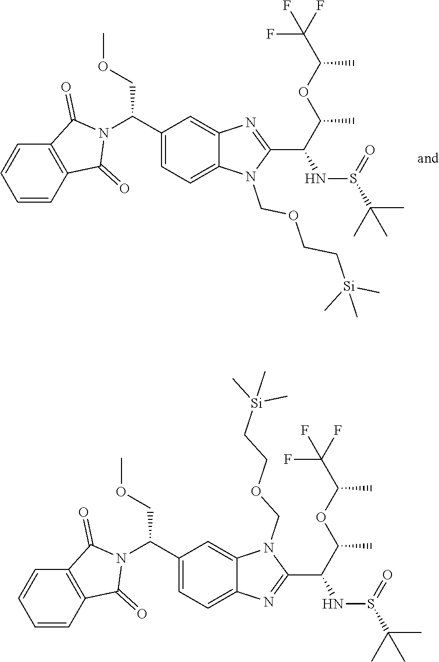 [SMILES: COC[C@H](c1ccc2c(c1)nc([C@@H](N[S@](=O)C(C)(C)C)[C@@H](C)O[C@@H](C)C(F)(F)F)n2COCC[Si](C)(C)C)N1C(=O)c2ccccc2C1=O ; COC[C@H](c1ccc2nc([C@@H](N[S@](=O)C(C)(C)C)[C@@H](C)O[C@@H](C)C(F)(F)F)n(COCC[Si](C)(C)C)c2c1)N1C(=O)c2ccccc2C1=O]

The title compounds (27% yield) were prepared as described for the synthesis of Intermediate 31 using (S)-2-(2-methoxy-1-(1-((2-(trimethylsilyl)ethoxy)methyl)-1H-benzo[d]imidazol-5-yl)ethyl)isoindoline-1,3-dione (Intermediate 76) in place of (R)-cyclopropyl(1-((2-(trimethylsilyl)ethoxy)methyl)-1H-benzo[d]imidazol-5-yl)methanamine and (R)-2-methyl-N—((R,E)-2-(((S)-1,1,1-trifluoropropan-2-yl)oxy)propylidene)propane-2-sulfinamide (Intermediate 9) in place of (R,E)-2-methyl-N-(2-((1,1,1-trifluoro-2-methylpropan-2-yl)oxy)ethylidene)propane-2-sulfinamide.

### Intermediate 153: (R)—N-((1R,2R)-1-(5-((S)-1-Amino-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide and (R)—N-((1R,2R)-1-(6-((S)-1-amino-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

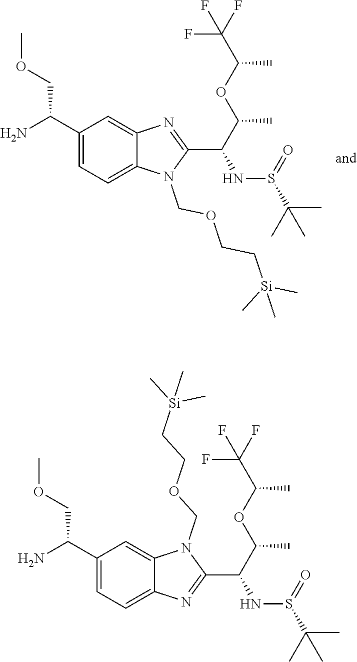 [SMILES: COC[C@@H](N)c1ccc2c(c1)nc([C@@H](N[S@](=O)C(C)(C)C)[C@@H](C)O[C@@H](C)C(F)(F)F)n2COCC[Si](C)(C)C ; COC[C@@H](N)c1ccc2nc([C@@H](N[S@](=O)C(C)(C)C)[C@@H](C)O[C@@H](C)C(F)(F)F)n(COCC[Si](C)(C)C)c2c1]

The title compounds (98% yield) were prepared as described for the synthesis of Intermediate 32 using ((R)—N-((1R,2R)-1-(5-((S)-1-(1,3-dioxoisoindolin-2-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide and (R)—N-((1R,2R)-1-(6-((S)-1-(1,3-dioxoisoindolin-2-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 152) in place of (R)—N—((R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 154: (R)—N-((1R,2R)-1-(5-((S)-1-(5,5-Difluoro-2-oxotetrahydropyrimidin-1(2H)-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide and (R)—N-((1R,2R)-1-(6-((S)-1-(5,5-difluoro-2-oxotetrahydropyrimidin-1(2H)-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

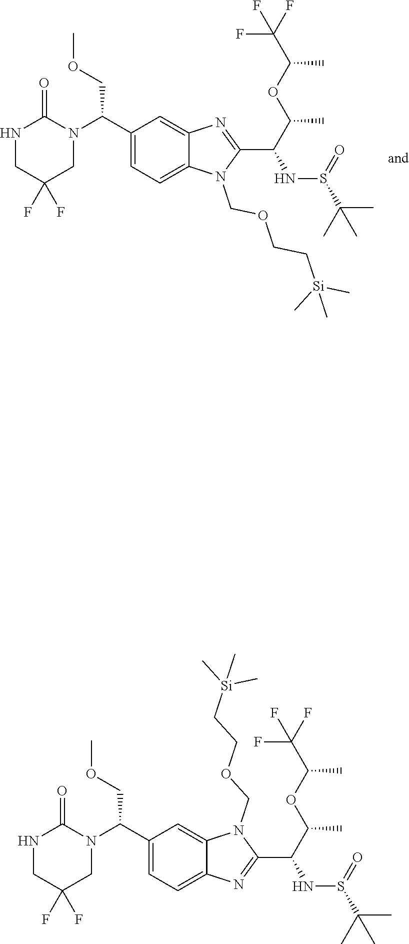 [SMILES: COC[C@H](c1ccc2c(c1)nc([C@@H](N[S@](=O)C(C)(C)C)[C@@H](C)O[C@@H](C)C(F)(F)F)n2COCC[Si](C)(C)C)N1CC(F)(F)CNC1=O ; COC[C@H](c1ccc2nc([C@@H](N[S@](=O)C(C)(C)C)[C@@H](C)O[C@@H](C)C(F)(F)F)n(COCC[Si](C)(C)C)c2c1)N1CC(F)(F)CNC1=O]

The title compounds (78% yield) were prepared as described for the synthesis of Intermediate 36 using (R)—N-((1R,2R)-1-(5-((S)-1-amino-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide and (R)—N-((1R,2R)-1-(6-((S)-1-amino-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 153) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 155: 1-((S)-1-(2-((1R,2R)-1-Amino-2-(((S)-1,1,1-trifluoropropan-2-yl)oxy)propyl)-1H-benzo[d]imidazol-5-yl)-2-methoxyethyl)-5,5-difluorotetrahydropyrimidin-2(1H)-one

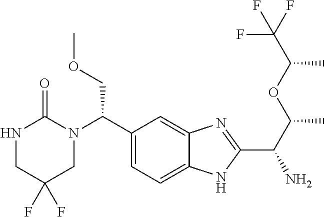 [SMILES: COC[C@H](c1ccc2[nH]c([C@@H](N)[C@@H](C)O[C@@H](C)C(F)(F)F)nc2c1)N1CC(F)(F)CNC1=O]

The title compound was prepared as described in Intermediate 37 using (R)—N-((1R,2R)-1-(5-((S)-1-(5,5-difluoro-2-oxotetrahydropyrimidin-1(2H)-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide and (R)—N-((1R,2R)-1-(6-((S)-1-(5,5-difluoro-2-oxotetrahydropyrimidin-1(2H)-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 154) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and used without further purification (assuming theoretical yield).

### Intermediate 156: tert-Butyl ((1R)-1-(5-(1-(((S)-2-amino-3,3,3-trifluoro-2-methylpropyl)amino)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl ((1R)-1-(6-(1-(((S)-2-amino-3,3,3-trifluoro-2-methylpropyl)amino)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

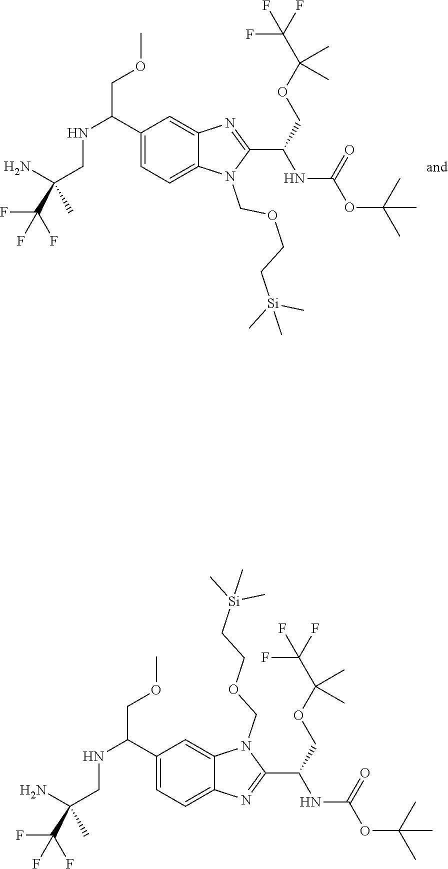 [SMILES: COCC(NC[C@](C)(N)C(F)(F)F)c1ccc2c(c1)nc([C@H](COC(C)(C)C(F)(F)F)NC(=O)OC(C)(C)C)n2COCC[Si](C)(C)C ; COCC(NC[C@](C)(N)C(F)(F)F)c1ccc2nc([C@H](COC(C)(C)C(F)(F)F)NC(=O)OC(C)(C)C)n(COCC[Si](C)(C)C)c2c1]

To tert-butyl (R)-(1-(5-(2-methoxyacetyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl (R)-(1-(6-(2-methoxyacetyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (150 mg, 0.254 mmol, Intermediate 147) and (2S)-3,3,3-trifluoro-2-methylpropane-1,2-diamine dihydrochloride (65.6 mg, 0.305 mmol) were added DCE (2.5 mL), Et₃N (0.106 mL, 0.763 mmol) and tetraisopropoxytitanium (0.149 mL, 0.509 mmol). The resulting mixture was heated at 50° C. for 4.5 h under N₂. The reaction was cooled to rt and AcOH (0.073 mL, 1.27 mmol), sodium cyanoborohydride (48.0 mg, 0.763 mmol) and MeOH (0.29 mL, 7.05 mmol) were added, and the resulting mixture was heated at 50° C. for 18 h. The reaction was diluted with saturated aqueous NaHCO₃ (5 mL) and EtOAc (5 mL) and filtered through diatomaceous earth (e.g., Celite©). The filtrate layers were separated and the organic layer was washed with brine, dried over anhydrous Na₂SO₄, filtered, and concentrated to dryness to afford the mixture of title compounds that was used without further purification.

### Intermediate 157: tert-Butyl ((1R)-1-(5-(2-methoxy-1-((S)-4-methyl-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl ((1R)-1-(6-(2-methoxy-1-((S)-4-methyl-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

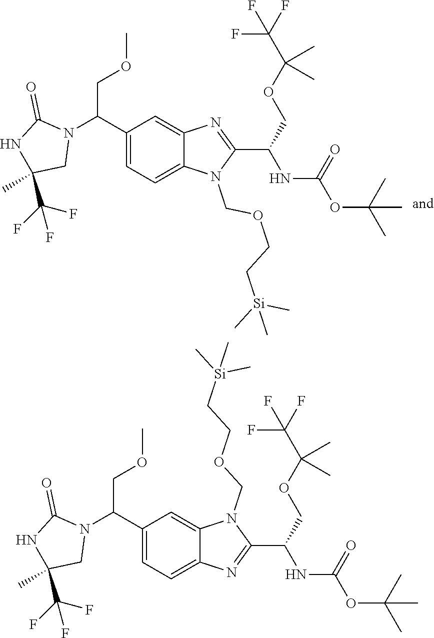

A solution of tert-butyl ((1R)-1-(5-(1-(((S)-2-amino-3,3,3-trifluoro-2-methylpropyl)amino)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl ((1R)-1-(6-(1-(((S)-2-amino-3,3,3-trifluoro-2-methylpropyl)amino)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (182 mg, 0.254 mmol, Intermediate 156) and DIPEA (0.135 mL, 0.775 mmol) in DCM (2.5 mL) under N₂ was cooled to 0° C. To the mixture was added dropwise a solution of bis(trichloromethyl) carbonate (30.2 mg, 0.102 mmol) in DCM (0.5 mL). After stirring at 0° C. for 20 min, the reaction was quenched with MeOH (2 mL) and the mixture was concentrated to dryness. Purification by silica gel chromatography (10-100% EtOAc (10% MeOH)/DCM) provided a mixture of the title compounds in 24% yield.

### Intermediate 158: (4S)-1-(1-(2-((R)-1-Amino-2-((1,1,1-trifluoro-2-methylpropan-2-yl)oxy)ethyl)-1H-benzo[d]imidazol-5-yl)-2-methoxyethyl)-4-methyl-4-(trifluoromethyl)imidazolidin-2-one

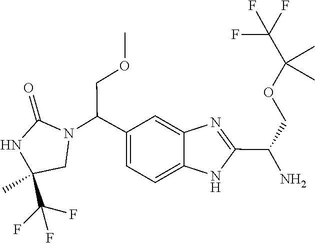 [SMILES: COCC(c1ccc2[nH]c([C@@H](N)COC(C)(C)C(F)(F)F)nc2c1)N1C[C@@](C)(C(F)(F)F)NC1=O]

The title compound was prepared as described in Intermediate 37 using tert-butyl ((1R)-1-(5-(2-methoxy-1-((S)-4-methyl-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate and tert-butyl ((1R)-1-(6-(2-methoxy-1-((S)-4-methyl-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 157) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and used without further purification (97% yield).

### Intermediate 159: (R,E)-2-Methyl-N-(4,4,4-trifluoro-3,3-dimethylbutylidene)propane-2-sulfinamide

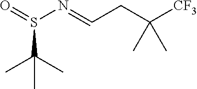 [SMILES: CC(C)(C)[S@@](=O)/N=C/CC(C)(C)C(F)(F)F]

To a solution of 3,3,3-trifluoropropanal (140.0 g, 908.3 mmol) in DCM (1500 mL) were added (R)-2-methylpropane-2-sulfinamide (132.1 g, 1.09 mol), PPTS (23.1 g, 91.7 mmol) and CuSO₄ (430 g, 2.74 mol), and the resulting mixture was heated at 30° C. for 12 h. The reaction was filtered through diatomaceous earth (e.g. Celite©), then the filtrate was concentrated to give a yellow oil. The material was purified by silica gel chromatography (0-10% EtOAc/petroleum ether) to obtain the title compound as a yellow oil.

### Intermediate 160: (R)—N—((S)-1-(5-((R)-Amino(cyclopropyl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

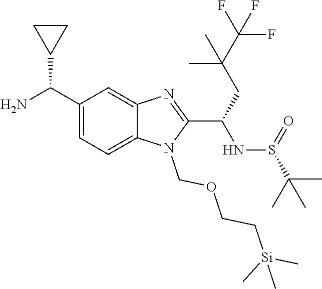 [SMILES: CC(C)(C)[S@@](=O)N[C@@H](CC(C)(C)C(F)(F)F)c1nc2cc([C@H](N)C3CC3)ccc2n1COCC[Si](C)(C)C]

Step A: N—((S)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide. A solution of (R)-2-(cyclopropyl(1-((2-(trimethylsilyl)ethoxy)methyl)-1H-benzo[d]imidazol-5-yl)methyl)isoindoline-1,3-dione (10.7 g, 23.34 mmol, Intermediate 30) and (R,E)-2-methyl-N-(4,4,4-trifluoro-3,3-dimethylbutylidene)propane-2-sulfinamide (10.09 g, 35.29 mmol, Intermediate 159) in THF (150 mL) was cooled to −78° C. under a nitrogen atmosphere. Then, LDA (48 mL, 1 M in hexane, 48 mmol) was added dropwise over 10 minutes. The resulting mixture was stirred at −78° C. for ˜1 h. The reaction was then quenched with a solution of AcOH (4 mL, 69.94 mmol) in THF (50 mL, 617.14 mmol) at −78° C., then gradually allowed to warm to rt under nitrogen. The contents were then transferred to a separatory funnel with ethyl acetate and extracted twice with deionized water. The organic phase was then separated, dried over anhydrous magnesium sulfate, filtered and concentrated under reduced pressure to yield an off-white foam. Purification was accomplished by silica gel chromatography (25-40% DCM/EtOAc) to afford the title compound (50% yield).

Step B: (R)—N—((S)-1-(5-((R)-Amino(cyclopropyl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide. To a solution of N—((S)-1-(5-((R)-cyclopropyl(1,3-dioxoisoindolin-2-yl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (1.18 g, 1.68 mmol, Step A) dissolved in EtOH (10 mL) was added hydrazine monohydrate (0.8 mL, 16.5 mmol) to produce a clear and colorless solution. The reaction was stirred for 30 h at rt then concentrated under reduced pressure. The residue was dissolved in EtOAc, the precipitate removed by filtration, and then the filtrate was condensed to provide the title compound (99% yield).

### Intermediate 161: (R)—N—((S)-1-(5-((R)-Cyclopropyl((S*)-2-oxo-4-(trifluoromethyl)imidazolidin-1-yl-5,5-d₂)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

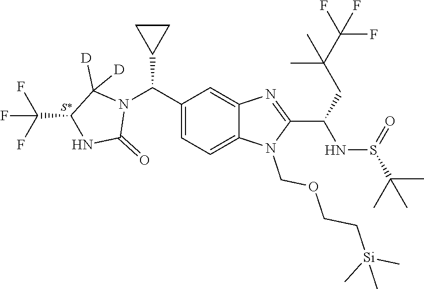 [SMILES: [2H]C1([2H])[C@@H](C(F)(F)F)NC(=O)N1[C@@H](c1ccc2c(c1)nc([C@H](CC(C)(C)C(F)(F)F)N[S@](=O)C(C)(C)C)n2COCC[Si](C)(C)C)C1CC1]

The title compound was prepared as described for the synthesis of Intermediate 33 using (R)—N—((S)-1-(5-((R)-amino(cyclopropyl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (Intermediate 160) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and benzyl (S*)-4-(trifluoromethyl)-1,2,3-oxathiazolidine-3-carboxylate-5,5-d₂ 2,2-dioxide (Intermediate 26) in place of (S)-benzyl 4-(trifluoromethyl)-1,2,3-oxathiazolidine-3-carboxylate 2,2-dioxide to afford the title compound in 3 steps (26% yield).

### Intermediate 162: (S)-1-((R)-(2-((S*)-1-Amino-4,4,4-trifluoro-3,3-dimethylbutyl)-1H-benzo[d]imidazol-5-yl) (cyclopropyl)methyl)-4-(trifluoromethyl)imidazolidin-2-one-5,5-d₂

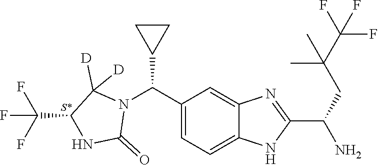 [SMILES: [2H]C1([2H])[C@@H](C(F)(F)F)NC(=O)N1[C@@H](c1ccc2[nH]c([C@@H](N)CC(C)(C)C(F)(F)F)nc2c1)C1CC1]

The title compound (26% yield) was prepared as described for the synthesis of Intermediate 34 using (R)—N—((S)-1-(5-((R)-cyclopropyl((S*)-2-oxo-4-(trifluoromethyl)imidazolidin-1-yl-5,5-d₂)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (Intermediate 161) in place of (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide.

### Intermediate 163: (R)—N-((1R,2R)-1-(5-((S)-1-Amino-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

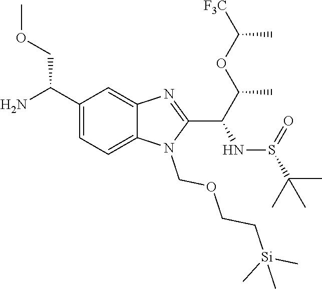 [SMILES: COC[C@@H](N)c1ccc2c(c1)nc([C@@H](N[S@](=O)C(C)(C)C)[C@@H](C)O[C@@H](C)C(F)(F)F)n2COCC[Si](C)(C)C]

Step A: (R)—N-((1R,2R)-1-(5-((S)-1-(1,3-Dioxoisoindolin-2-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. (S)-2-(2-Methoxy-1-(1-((2-(trimethylsilyl)ethoxy)methyl)-1H-benzo[d]imidazol-5-yl)ethyl)isoindoline-1,3-dione (1.45 g, 3.21 mmol, Intermediate 76) and THF (70 mL) were combined and cooled to −78° C. under nitrogen. LDA (5 mL, 1 M in THF, 5 mmol) was then added and the reaction was stirred at −78° C. for approximately 5 min followed by the addition of (R)-2-methyl-N—((R,E)-2-(((S)-1,1,1-trifluoropropan-2-yl)oxy)propylidene)propane-2-sulfinamide (1.38 g, 5.03 mmol, Intermediate 9) in THF (4 mL). The reaction was stirred for 30 min then additional LDA was added (1.6 mL, 1 M in THF, 1.6 mmol) and the contents were allowed to stir at −78° C. for another 30 min. The reaction was then quenched with a solution of acetic acid (2 mL in THF (10 mL)) at −78° C. and the reaction solution was stirred for approximately 2 min, then the cold bath was removed and the contents allowed to warm to rt. The reaction solution was then transferred to a separatory funnel with ethyl acetate and extracted twice with deionized water. The organic phase was separated, dried over anhydrous magnesium sulfate, filtered and concentrated under reduced pressure. The product was purified in batches by preparative HPLC (XBridge Phenyl Prep 5 μm C18 50×250 mm, 0-100% MeCN with 0.05% TFA/water with 0.05% TFA) to afford, after lyophilization, the title compound in 27% yield.

Step B: (R)—N-((1R,2R)-1-(5-((S)-1-Amino-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. (R)—N-((1R,2R)-1-(5-((S)-1-(1,3-Dioxoisoindolin-2-yl)-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (715 mg, 0.85 mmol, Step A), hydrazine monohydrate (650 μL, 8.69 mmol) and EtOH (8 mL) were combined and stirred at rt overnight. The reaction mixture was transferred to a separatory funnel with deionized water and ethyl acetate. The organic phase was separated and the aqueous layer was salted with sodium chloride and extracted twice with ethyl acetate. The combined organic layers were dried over anhydrous magnesium sulfate, filtered and concentrated to dryness to provide the title compound in quantitative yield.

### Intermediate 164: N-((1R*)-1-(5-((1S)-2-Methoxy-1-(2-oxo-4-(trifluoromethyl)imidazolidin-1-yl-5,5-d₂)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

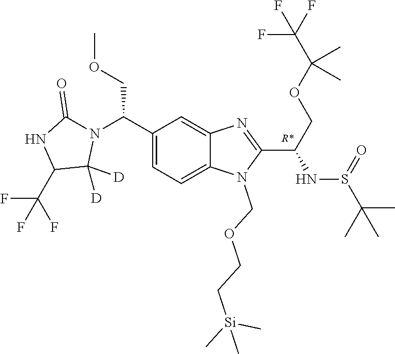 [SMILES: [2H]C1([2H])C(C(F)(F)F)NC(=O)N1[C@H](COC)c1ccc2c(c1)nc([C@H](COC(C)(C)C(F)(F)F)NS(=O)C(C)(C)C)n2COCC[Si](C)(C)C]

The title compound was prepared as described for the synthesis of Intermediate 33 using (R)—N—((R*)-1-(5-((S)-1-amino-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 78) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and benzyl (trifluoromethyl)-1,2,3-oxathiazolidine-3-carboxylate-5,5-d₂ 2,2-dioxide (Intermediate 28) in place of (S)-benzyl 4-(trifluoromethyl)-1,2,3-oxathiazolidine-3-carboxylate 2,2-dioxide in Step A to afford the title compound in 28% yield.

### Intermediate 165: 1-((S)-1-(2-((R*)-1-Amino-2-((1,1,1-trifluoro-2-methylpropan-2-yl)oxy)ethyl)-1H-benzo[d]imidazol-5-yl)-2-methoxyethyl)-4-(trifluoromethyl)imidazolidin-2-one-5,5-d₂

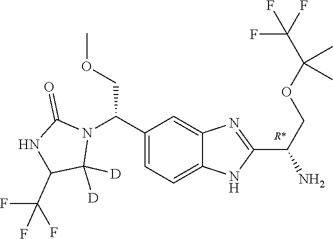 [SMILES: [2H]C1([2H])C(C(F)(F)F)NC(=O)N1[C@H](COC)c1ccc2[nH]c([C@@H](N)COC(C)(C)C(F)(F)F)nc2c1]

The title compound was prepared as described for the synthesis of Intermediate 34 using N-((1R*)-1-(5-((1S)-2-methoxy-1-(2-oxo-4-(trifluoromethyl)imidazolidin-1-yl-5,5-d₂)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 166) in place of (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide to afford the title compound that was used without further purification.

### Intermediate 166: N—((R*)-1-(5-((S)-2-Cyclopropoxy-1-(5,5-difluoro-2-oxotetrahydropyrimidin-1(2H)-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

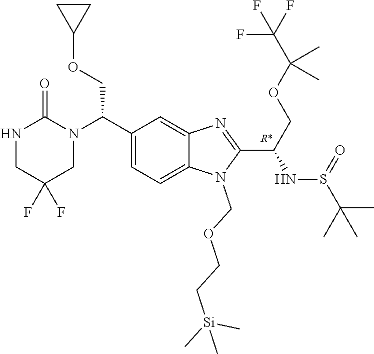 [SMILES: CC(C)(C)S(=O)N[C@@H](COC(C)(C)C(F)(F)F)c1nc2cc([C@@H](COC3CC3)N3CC(F)(F)CNC3=O)ccc2n1COCC[Si](C)(C)C]

The title compound was prepared as described for the synthesis of Intermediate 36 using N—((R*)-1-(5-((S)-1-amino-2-cyclopropoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 123) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in Step A, and was used without further purification.

### Intermediate 167: 1-((S)-1-(2-((R*)-1-Amino-2-((1,1,1-trifluoro-2-methylpropan-2-yl)oxy)ethyl)-1H-benzo[d]imidazol-5-yl)-2-cyclopropoxyethyl)-5,5-difluorotetrahydropyrimidin-2(1H)-one

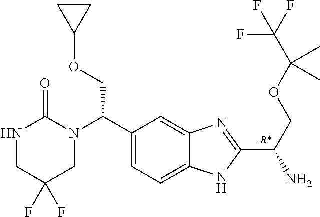 [SMILES: CC(C)(OC[C@H](N)c1nc2cc([C@@H](COC3CC3)N3CC(F)(F)CNC3=O)ccc2[nH]1)C(F)(F)F]

The title compound was prepared as described for the synthesis of Intermediate 37 using N—((R)-1-(5-((S)-2-cyclopropoxy-1-(5,5-difluoro-2-oxotetrahydropyrimidin-1(2H)-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 166) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and was used without further purification.

### Intermediate 168: 5-(1,3-Dioxolan-2-yl)-1-((2-(trimethylsilyl)ethoxy)methyl)-1H-benzo[d]imidazole

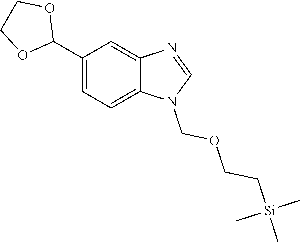 [SMILES: C[Si](C)(C)CCOCn1cnc2cc(C3OCCO3)ccc21]

A mixture of 1-((2-(trimethylsilyl)ethoxy)methyl)-1H-benzo[d]imidazole-5-carbaldehyde (1.1 g, 3.9 mmol, Intermediate 10), ethylene glycol (0.28 mL, 5.0 mmol), and p-toluenesulfonic acid monohydrate (37 mg, 0.19 mmol) in toluene (10 mL) was heated at 120° C. for 16 h utilizing a Dean-Stark apparatus. The reaction was removed from heating and ethylene glycol (0.15 mL, 2.7 mmol) was added. The mixture was heated at 60° C. under 150 torr vacuum for 3 h then concentrated under reduced pressure. Purification by silica gel column chromatography (20-100% EtOAc/hexanes) afforded the title compound in 75% yield.

### Intermediate 169: N—((R*)-1-(5-(1,3-Dioxolan-2-yl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

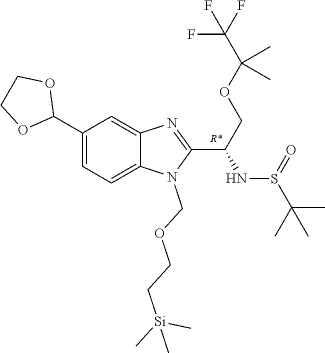 [SMILES: CC(C)(C)S(=O)N[C@@H](COC(C)(C)C(F)(F)F)c1nc2cc(C3OCCO3)ccc2n1COCC[Si](C)(C)C]

The title compound was prepared as described for the synthesis of Intermediate 31 using 5-(1,3-dioxolan-2-yl)-1-((2-(trimethylsilyl)ethoxy)methyl)-1H-benzo[d]imidazole (Intermediate 168) in place of (R)-2-(cyclopropyl(1-((2-(trimethylsilyl)ethoxy)methyl)-1H-benzo[d]imidazol-5-yl)methyl)isoindoline-1,3-dione in 56% yield.

### Intermediate 170: tert-Butyl (R*)-(1-(5-formyl-1-((2-(trimethylsilyl)ethoxy)methyl)-1H-benzo[d]imidazol-2-yl)-2-((1,1,1-trifluoro-2-methylpropan-2-yl)oxy)ethyl)carbamate

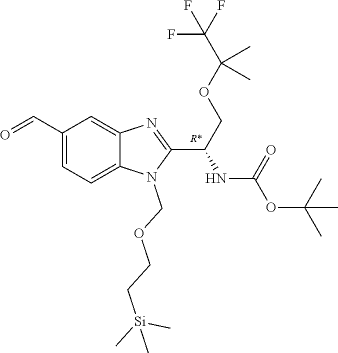 [SMILES: CC(C)(C)OC(=O)N[C@@H](COC(C)(C)C(F)(F)F)c1nc2cc(C=O)ccc2n1COCC[Si](C)(C)C]

To a solution of N—((R*)-1-(5-(1,3-dioxolan-2-yl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (3.4 g, 5.7 mmol, Intermediate 169) in THF (76 mL) and H₂O (19 mL) was added 12 (0.58 g, 2.2 mmol) and the reaction was heated at 50° C. for 6 h. The mixture was cooled to rt then quenched with saturated aqueous sodium thiosulfate (11 mL), diluted with H₂O, and extracted with EtOAc. The combined organic layers were washed with brine, dried over anhydrous Na₂SO₄, filtered and concentrated under reduced pressure. The residue was taken up in CH₂Cl₂, then triethylamine (1.7 mL, 13 mmol) and di-tert-butyl dicarbonate (1.7 g, 8.0 mmol) were added and the reaction was stirred at rt for 16 h. The mixture was diluted with saturated aqueous ammonium chloride and extracted with EtOAc. The organic layer was washed with brine, dried over anhydrous Na₂SO₄, filtered, and concentrated under reduced pressure. Purification by silica gel column chromatography (20-80% EtOAc/hexanes) afforded the title compound in 65% yield.

### Intermediate 171: tert-Butyl ((1R*)-1-(5-((4-methyl-2-oxo-4-(trifluoromethyl)imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

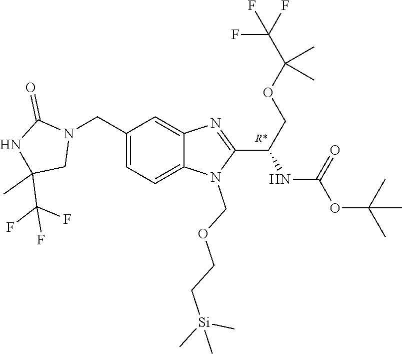 [SMILES: CC(C)(C)OC(=O)N[C@@H](COC(C)(C)C(F)(F)F)c1nc2cc(CN3CC(C)(C(F)(F)F)NC3=O)ccc2n1COCC[Si](C)(C)C]

Step A: tert-Butyl ((1R*)-1-(5-(((2-amino-3,3,3-trifluoro-2-methylpropyl)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate. The title compound was prepared as described for the synthesis of Intermediate 64 using tert-butyl (R*)-(1-(5-formyl-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 170) in place of 1-((2-(trimethylsilyl)ethoxy)methyl)-1H-benzo[d]imidazole-5-carbaldehyde that was used without further purification.

Step B: tert-Butyl ((1R*)-1-(5-((4-methyl-2-oxo-4-(trifluoromethyl)imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate. The title compound was prepared as described for the synthesis of Intermediate 33 Step C using tert-butyl ((1R*)-1-(5-(((2-amino-3,3,3-trifluoro-2-methylpropyl)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Step A) in place of (R)—N—((R)-1-(5-((R)—(((S)-2-amino-3,3,3-trifluoropropyl)amino) (cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 55% yield.

### Intermediate 172: 1-((2-((R*)-1-Amino-2-((1,1,1-trifluoro-2-methylpropan-2-yl)oxy)ethyl)-1H-benzo[d]imidazol-5-yl)methyl)-4-methyl-4-(trifluoromethyl)imidazolidin-2-one

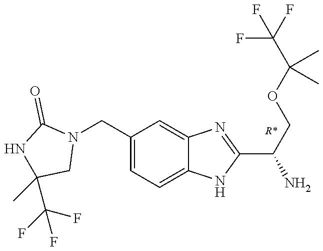 [SMILES: CC1(C(F)(F)F)CN(Cc2ccc3[nH]c([C@@H](N)COC(C)(C)C(F)(F)F)nc3c2)C(=O)N1]

The title compound was prepared as described for the synthesis of Intermediate 37 using tert-butyl ((1R*)-1-(5-((4-methyl-2-oxo-4-(trifluoromethyl)imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 171) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide that was used without further purification.

### Intermediate 173: tert-Butyl ((R*)-1-(5-((E)-(((R)-mesitylsulfinyl)imino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

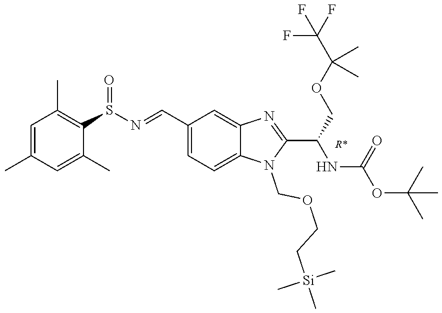 [SMILES: Cc1cc(C)c([S@@](=O)/N=C/c2ccc3c(c2)nc([C@H](COC(C)(C)C(F)(F)F)NC(=O)OC(C)(C)C)n3COCC[Si](C)(C)C)c(C)c1]

The title compound was prepared as described for the synthesis of Intermediate 118 using tert-butyl (R*)-(1-(5-formyl-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 170) in place of 1-((2-(trimethylsilyl)ethoxy)methyl)-1H-benzo[d]imidazole-5-carbaldehyde in 91% yield.

### Intermediate 174: tert-Butyl ((R*)-1-(5-((S*)-2-cyclopropoxy-1-(((R)-mesitylsulfinyl)amino)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

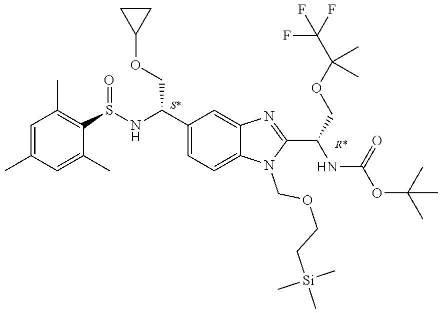 [SMILES: Cc1cc(C)c([S@@](=O)N[C@H](COC2CC2)c2ccc3c(c2)nc([C@H](COC(C)(C)C(F)(F)F)NC(=O)OC(C)(C)C)n3COCC[Si](C)(C)C)c(C)c1]

The title compound was prepared as described for the synthesis of Intermediate 119 using tert-butyl ((R*)-1-(5-((E)-(((R)-mesitylsulfinyl)imino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 173) in place of (R,E)-2,4,6-trimethyl-N-((1-((2-(trimethylsilyl)ethoxy)methyl)-1H-benzo[d]imidazol-5-yl)methylene)benzenesulfinamide in 48% yield.

### Intermediate 175: tert-Butyl ((R*)-1-(5-((S*)-1-amino-2-cyclopropoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

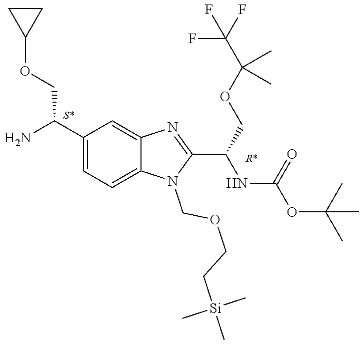 [SMILES: CC(C)(C)OC(=O)N[C@@H](COC(C)(C)C(F)(F)F)c1nc2cc([C@H](N)COC3CC3)ccc2n1COCC[Si](C)(C)C]

The title compound was prepared as described for the synthesis of Intermediate 120 using tert-butyl ((R*)-1-(5-((S*)-2-cyclopropoxy-1-(((R)-mesitylsulfinyl)amino)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 174) in place of (R)—N—((S*)-2-cyclopropoxy-1-(1-((2-(trimethylsilyl)ethoxy)methyl)-1H-benzo[d]imidazol-5-yl)ethyl)-2,4,6-trimethylbenzenesulfinamide in 80% yield.

### Intermediate 176: tert-Butyl ((R*)-1-(5-((S*)-2-cyclopropoxy-1-((S*)-2-oxo-4-(trifluoromethyl)imidazolidin-1-yl-5,5-d₂)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate

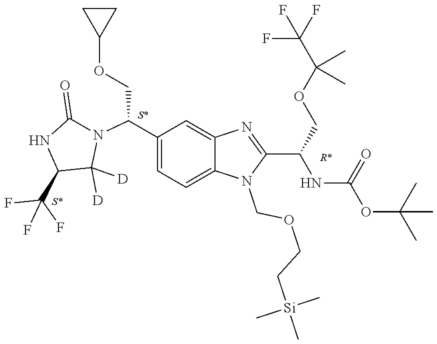 [SMILES: [2H]C1([2H])[C@@H](C(F)(F)F)NC(=O)N1[C@H](COC1CC1)c1ccc2c(c1)nc([C@H](COC(C)(C)C(F)(F)F)NC(=O)OC(C)(C)C)n2COCC[Si](C)(C)C]

The title compound was prepared as described for the synthesis of Intermediate 33 using tert-butyl ((R*)-1-(5-((S*)-1-amino-2-cyclopropoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 175) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and benzyl (S*)-4-(trifluoromethyl)-1,2,3-oxathiazolidine-3-carboxylate-5,5-d₂ 2,2-dioxide (Intermediate 26) in place of (S)-benzyl 4-(trifluoromethyl)-1,2,3-oxathiazolidine-3-carboxylate 2,2-dioxide.

### Intermediate 177: (S*)-1-((S*)-1-(2-((R*)-1-Amino-2-((1,1,1-trifluoro-2-methylpropan-2-yl)oxy)ethyl)-1H-benzo[d]imidazol-5-yl)-2-cyclopropoxyethyl)-4-(trifluoromethyl)imidazolidin-2-one-5,5-d₂

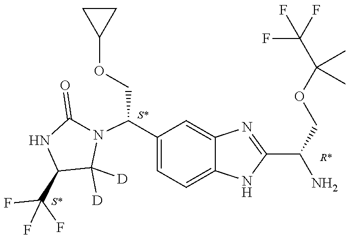 [SMILES: [2H]C1([2H])[C@@H](C(F)(F)F)NC(=O)N1[C@H](COC1CC1)c1ccc2[nH]c([C@@H](N)COC(C)(C)C(F)(F)F)nc2c1]

The title compound was prepared as described for the synthesis of Intermediate 37 using tert-butyl ((R*)-1-(5-((S*)-2-cyclopropoxy-1-((S*)-2-oxo-4-(trifluoromethyl)imidazolidin-1-yl-5,5-d₂)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)carbamate (Intermediate 176) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide that was used without further purification.

### Intermediate 178: (R)—N-((1R,2R)-1-(5-((S)-2-Methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

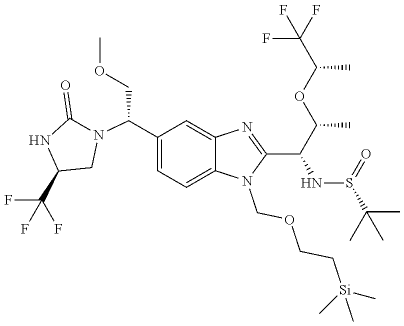 [SMILES: COC[C@H](c1ccc2c(c1)nc([C@@H](N[S@](=O)C(C)(C)C)[C@@H](C)O[C@@H](C)C(F)(F)F)n2COCC[Si](C)(C)C)N1C[C@@H](C(F)(F)F)NC1=O]

The title compound was prepared as described for the synthesis of Intermediate 33 using (R)—N-((1R,2R)-1-(5-((S)-1-amino-2-methoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 163) in place of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 55% yield.

### Intermediate 179: (S)-1-((S)-1-(2-((1R,2R)-1-Amino-2-(((S)-1,1,1-trifluoropropan-2-yl)oxy)propyl)-1H-benzo[d]imidazol-5-yl)-2-methoxyethyl)-4-(trifluoromethyl)imidazolidin-2-one

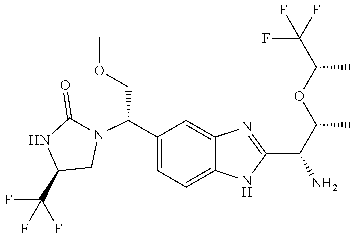 [SMILES: COC[C@H](c1ccc2[nH]c([C@@H](N)[C@@H](C)O[C@@H](C)C(F)(F)F)nc2c1)N1C[C@@H](C(F)(F)F)NC1=O]

The title compound was prepared as described for the synthesis of Intermediate 37 using (R)—N-((1R,2R)-1-(5-((S)-2-methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 178) in place of (R)—N—((R)-1-(5-((R)-cyclopropyl(5,5-difluoro-2-oxotetrahydropyrimidin-1(2H)-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in 54% yield.

### Intermediate 180: 1-((S)-1-(2-((1R,2R)-1-Amino-2-(((S)-1,1,1-trifluoropropan-2-yl)oxy)propyl)-1H-benzo[d]imidazol-5-yl)-2-cyclopropoxyethyl)-5,5-difluorotetrahydropyrimidin-2(1H)-one

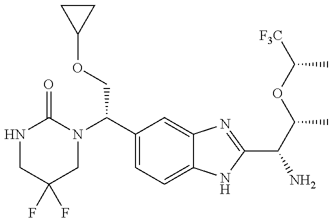 [SMILES: C[C@H](O[C@H](C)[C@H](N)c1nc2cc([C@@H](COC3CC3)N3CC(F)(F)CNC3=O)ccc2[nH]1)C(F)(F)F]

Step A: (R)—N-((1R,2R)-1-(5-((S)-2-Cyclopropoxy-1-(1,3-dioxoisoindolin-2-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. (S)-2-(2-Cyclopropoxy-1-(1-((2-(trimethylsilyl)ethoxy)methyl)-1H-benzo[d]imidazol-5-yl)ethyl)isoindoline-1,3-dione (310 mg, 0.65 mmol, Intermediate 121), (R)-2-methyl-N—((R,E)-2-(((S)-1,1,1-trifluoropropan-2-yl)oxy)propylidene)propane-2-sulfinamide (273 mg, 0.998 mmol, Intermediate 9) and THF (8 mL) were combined and cooled to −78° C. under nitrogen. LDA (1 mL, 1 M in THF, 1 mmol) was then added and the reaction was stirred at −78° C. for approximately 45 min. Additional LDA was then added (0.4 mL, 1 M in THF, 0.4 mmol) and the contents were allowed to stir at −78° C. for another 45 min. The reaction was then quenched with a solution of acetic acid (0.5 mL in THF (3 mL)) at −78° C. and the reaction solution was stirred for approximately 2 min, then the cold bath was removed and the contents allowed to warm to rt. The reaction solution was then transferred to a separatory funnel with ethyl acetate and extracted twice with deionized water. The organic phase was separated, dried over anhydrous magnesium sulfate, filtered and concentrated under reduced pressure. The product was purified by silica gel chromatography (0-100% (10% MeOH in EtOAc)/hexanes) to afford the title compound in 84% yield.

Step B: (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. (R)—N-((1R,2R)-1-(5-((S)-2-Cyclopropoxy-1-(1,3-dioxoisoindolin-2-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (407 mg, 0.54 mmol, Step A), hydrazine monohydrate (500 μL, 6.69 mmol) and EtOH (5 mL) were combined and stirred at rt overnight. The reaction mixture was transferred to a separatory funnel with deionized water and ethyl acetate. The organic phase was separated and the aqueous layer was salted with sodium chloride and extracted twice with ethyl acetate. The combined organic layers were dried over anhydrous magnesium sulfate, filtered and concentrated to dryness to provide the title compound in 98% yield.

Step C: (R)—N-((1R,2R)-1-(5-((S)-2-Cyclopropoxy-1-((3-(1,3-dioxoisoindolin-2-yl)-2,2-difluoropropyl)amino)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (329 mg, 0.53 mmol, Step B), acetonitrile (10 mL), DIPEA (200 μL, 1.16 mmol) and 3-(1,3-dioxoisoindolin-2-yl)-2,2-difluoropropyl trifluoromethanesulfonate (262 mg, 0.70 mmol) were combined and heated at 55° C. under a nitrogen atmosphere overnight. Additional 3-(1,3-dioxoisoindolin-2-yl)-2,2-difluoropropyl trifluoromethanesulfonate (60.6 mg, 0.16 mmol) was added and the mixture was heated at 55° C. overnight. The reaction mixture was cooled to rt and transferred to a separatory funnel with EtOAc and deionized water. The layers were separated, then the aqueous phase was salted with sodium chloride and extracted three times with EtOAc. The combined organic layers were dried over anhydrous magnesium sulfate, filtered and concentrated under reduced pressure. The product was purified by silica gel chromatography (0-100% (10% MeOH in EtOAc)/hexanes) to afford the title compound in 76% yield.

Step D: (R)—N-((1R,2R)-1-(5-((S)-1-((3-Amino-2,2-difluoropropyl)amino)-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. (R)—N-((1R,2R)-1-(5-((S)-2-Cyclopropoxy-1-((3-(1,3-dioxoisoindolin-2-yl)-2,2-difluoropropyl)amino)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (340 mg, 0.40 mmol, Step C) and EtOH (6 mL) were combined followed by the addition of hydrazine monohydrate (350 μL, 4.68 mmol) and the reaction was stirred at rt overnight. The mixture was then transferred to a separatory funnel with deionized water and ethyl acetate. The organic phase was separated and the aqueous layer was salted with sodium chloride and extracted twice with ethyl acetate. The combined organic layers were dried over anhydrous magnesium sulfate, filtered and concentrated to dryness to afford the title compound in 98% yield.

Step E: (R)—N-((1R,2R)-1-(5-((S)-2-Cyclopropoxy-1-(5,5-difluoro-2-oxotetrahydropyrimidin-1(2H)-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. (R)—N-((1R,2R)-1-(5-((S)-1-((3-Amino-2,2-difluoropropyl)amino)-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (283 mg, 0.40 mmol, Step D), THF (15 mL), DIPEA (250 μL, 1.45 mmol) and carbonyldiimidazole (198 mg, 1.22 mmol) were combined then the reaction mixture was heated at 60° C. under nitrogen for 1 h. The reaction solution was then transferred to a separatory funnel with EtOAc and deionized water. The layers were separated and the aqueous phase was salted with sodium chloride and extracted three times with EtOAc. The combined organic layers were dried over anhydrous magnesium sulfate, filtered and concentrated under reduced pressure. The product was purified twice by silica gel chromatography (0-100% (10% MeOH in EtOAc)/hexanes followed by 0-100% (10% MeOH in EtOAc)/hexanes) to afford the title compound in 43% yield.

Step F: 1-((S)-1-(2-((1R,2R)-1-Amino-2-(((S)-1,1,1-trifluoropropan-2-yl)oxy)propyl)-1H-benzo[d]imidazol-5-yl)-2-cyclopropoxyethyl)-5,5-difluorotetrahydropyrimidin-2(1H)-one. (R)—N-((1R,2R)-1-(5-((S)-2-Cyclopropoxy-1-(5,5-difluoro-2-oxotetrahydropyrimidin-1(2H)-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (115 mg, 0.16 mmol, Step E), a solution of 1,4-dioxane/MeOH (2 mL, 4:1) and HCl (350 μL, 4 M in dioxane, 1.4 mmol) were combined and heated at 60° C. in a sealed vial for approximately 4.5 h. The reaction solution was concentrated under reduced pressure then dissolved in deionized water and extracted with hexanes. The hexane wash was discarded. The pH of the aqueous phase was adjusted to approximately pH 8 by the addition of saturated aqueous sodium bicarbonate and then was extracted with ethyl acetate three times. The combined EtOAc layers were washed with brine, dried over anhydrous magnesium sulfate, filtered and concentrated under reduced pressure to afford the title compound in 65% yield.

### Intermediate 181: (S)-1-((S)-1-(2-((1R,2R)-1-Amino-2-(((S)-1,1,1-trifluoropropan-2-yl)oxy)propyl)-1H-benzo[d]imidazol-5-yl)-2-cyclopropoxyethyl)-4-(trifluoromethyl)imidazolidin-2-one

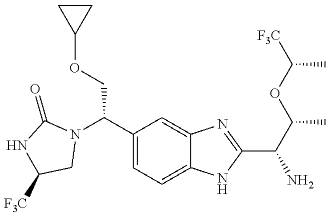 [SMILES: C[C@H](O[C@H](C)[C@H](N)c1nc2cc([C@@H](COC3CC3)N3C[C@@H](C(F)(F)F)NC3=O)ccc2[nH]1)C(F)(F)F]

Step A: Benzyl ((S)-3-(((S)-1-(2-((1R,2R)-1-(((R)-tert-butylsulfinyl)amino)-2-(((S)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)amino)-1,1,1-trifluoropropan-2-yl)carbamate. (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (272 mg, 0.44 mmol, Intermediate 180 Step B), DMF (4 mL), cesium carbonate (453 mg, 1.39 mmol) and benzyl (S)-4-(trifluoromethyl)-1,2,3-oxathiazolidine-3-carboxylate 2,2-dioxide (223.4 mg, 0.69 mmol, Intermediate 24) were combined and stirred at rt overnight. The reaction solution was then diluted with 5% LiCl (aqueous) and extracted twice with EtOAc. Then, the combined organic layers were washed with 5% LiCl (aqueous) and brine, dried over anhydrous sodium sulfate, filtered and condensed under reduced pressure. The product was purified by silica gel chromatography (0-100% (10% MeOH in EtOAc)/hexanes)) to afford the title compound in 27% yield.

Step B: (R)—N-((1R,2R)-1-(5-((S)-1-(((S)-2-Amino-3,3,3-trifluoropropyl)amino)-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. Benzyl ((S)-3-(((S)-1-(2-((1R,2R)-1-(((R)-tert-butylsulfinyl)amino)-2-(((S)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)amino)-1,1,1-trifluoropropan-2-yl)carbamate (103 mg, 0.12 mmol, Step A) and MeOH (4 mL) were combined in a large scintillation vial followed by the addition of 10% palladium on charcoal (55.6 mg, 0.052 mmol, wet). The vial was then placed in a Parr shaker bottle and placed under hydrogen (3 atm) and shaken for approximately 2.5 h. The reaction was filtered through diatomaceous earth (e.g., Celite©), rinsed with MeOH, then concentrated under reduced pressure to afford the title compound in quantitative yield.

Step C: (R)—N-((1R,2R)-1-(5-((S)-2-Cyclopropoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. (R)—N-((1R,2R)-1-(5-((S)-1-(((S)-2-Amino-3,3,3-trifluoropropyl)amino)-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (103 mg, 0.14 mmol, Step B), DCM (1.5 mL) and DIPEA (75 μL, 0.43 mmol) were combined and cooled to 0° C. under nitrogen. In a separate flask, triphosgene (19 mg, 0.064 mmol) and DCM (1.5 mL) were combined and cooled to 0° C. under nitrogen. The triphosgene solution was then added dropwise to the main reaction solution and the resulting mixture was stirred at 0° C. for approximately 45 min. Additional triphosgene (8.8 mg, 0.03 mmol) in DCM (1 mL) was then added dropwise to the main reaction solution and at the mixture was stirred at 0° C. for approximately 30 min. The reaction was then quenched by the addition of methanol, warmed to rt, then concentrated under reduced pressure. The product was purified by silica gel chromatography (0-100% (10% MeOH in EtOAc)/hexanes) to afford the title compound in 51% yield.

Step D: (S)-1-((S)-1-(2-((1R,2R)-1-Amino-2-(((S)-1,1,1-trifluoropropan-2-yl)oxy)propyl)-1H-benzo[d]imidazol-5-yl)-2-cyclopropoxyethyl)-4-(trifluoromethyl)imidazolidin-2-one. (R)—N-((1R,2R)-1-(5-((S)-2-Cyclopropoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (54 mg, 0.071 mmol, Step C), a solution of 1,4-dioxane/MeOH (1.5 mL, 4:1) and HCl (150 μL, 4 M in 1,4-dioxane, 0.60 mmol) were combined and heated at 60° C. in a sealed vial for approximately 4.5 h. The reaction was cooled to rt and concentrated under reduced pressure. The residue was dissolved in water, washed with hexanes and the hexane wash was discarded. The pH of the aqueous phase was adjusted to approximately pH 8 by the addition of saturated aqueous sodium bicarbonate solution, then salted with sodium chloride and extracted three times with ethyl acetate. The combined organic phases were dried over anhydrous magnesium sulfate, filtered and concentrated under reduced pressure to afford the title compound in 86% yield.

### Intermediate 182: (S)-1-((S)-1-(2-((1R,2R)-1-Amino-2-cyclopropoxypropyl)-1H-benzo[d]imidazol-5-yl)-2-cyclopropoxyethyl)-4-(trifluoromethyl)imidazolidin-2-one

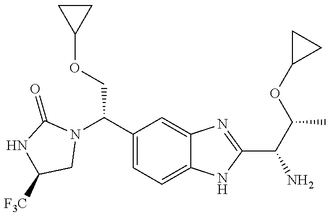 [SMILES: C[C@@H](OC1CC1)[C@H](N)c1nc2cc([C@@H](COC3CC3)N3C[C@@H](C(F)(F)F)NC3=O)ccc2[nH]1]

Step A: (R)—N-((1R,2R)-2-Cyclopropoxy-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)propyl)-2-methylpropane-2-sulfinamide. (S)-2-(2-Cyclopropoxy-1-(1-((2-(trimethylsilyl)ethoxy)methyl)-1H-benzo[d]imidazol-5-yl)ethyl)isoindoline-1,3-dione (412 mg, 0.86 mmol, Intermediate 121), (R)—N—((R,E)-2-cyclopropoxypropylidene)-2-methylpropane-2-sulfinamide (370 mg, 1.7 mmol, Intermediate 129) and THF (11 mL) were combined and cooled to −78° C. under nitrogen. LDA (1 mL, 1 M in hexane/THF, 1 mmol) was then added dropwise and the mixture stirred at −78° C. for approximately 30 min. Additional LDA (0.25 mL, 1 M in hexane/THF, 0.25 mmol) was then added and the mixture was allowed to stir at −78° C. for approximately 25 min. Additional LDA (0.25 mL, 1 M in hexane/THF, 0.25 mmol) was then added and the mixture was stirred at −78° C. for approximately 20 min. The reaction was then quenched by the addition of acetic acid (0.8 mL) in THF (3 mL). The contents were stirred at −78° C. for approximately 5 min, then the cold bath was removed and the reaction solution was allowed to warm to rt. The contents were then transferred to a separatory funnel with ethyl acetate and extracted twice with deionized water. The organic phase was separated, dried over anhydrous magnesium sulfate, filtered and concentrated under reduced pressure. The product was purified by silica gel chromatography (0-100% (10% MeOH in EtOAc)/hexanes) to afford the title compound in 98% yield.

Step B: (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide. (R)—N-((1R,2R)-2-Cyclopropoxy-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)propyl)-2-methylpropane-2-sulfinamide (584 mg, 0.84 mmol, Step A), EtOH (10 mL) and hydrazine monohydrate (700 μL, 9.36 mmol) were combined and stirred at rt overnight. The reaction mixture was transferred to a separatory funnel with deionized water and ethyl acetate. The organic phase was separated and the aqueous layer was salted with sodium chloride and extracted twice with ethyl acetate. The combined organic layers were dried over anhydrous magnesium sulfate, filtered and concentrated to dryness to afford the title compound in quantitative yield.

Step C: Benzyl ((S)-3-(((S)-1-(2-((1R,2R)-1-(((R)-tert-butylsulfinyl)amino)-2-cyclopropoxypropyl)-1-((2-(trimethylsilyl)ethoxy)methyl)-1H-benzo[d]imidazol-5-yl)-2-cyclopropoxyethyl)amino)-1,1,1-trifluoropropan-2-yl)carbamate. (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide (527 mg, 0.93 mmol, Step B), DMF (10 mL), cesium carbonate (1.01 g, 3.09 mmol) and benzyl (S)-4-(trifluoromethyl)-1,2,3-oxathiazolidine-3-carboxylate 2,2-dioxide (489 mg, 1.5 mmol, Intermediate 24) were combined and stirred at rt overnight under nitrogen. The reaction solution was then diluted with 5% LiCl (aqueous) and extracted twice with EtOAc. Then, the combined organic layers were washed with 5% LiCl (aqueous) and brine, dried over anhydrous magnesium sulfate, filtered and condensed under reduced pressure. The product was purified by silica gel chromatography (0-100% (10% MeOH in EtOAc)/hexanes) to afford the title compound in 58% yield.

Step D: (R)—N-((1R,2R)-1-(5-((S)-1-(((S)-2-Amino-3,3,3-trifluoropropyl)amino)-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide. Benzyl ((S)-3-(((S)-1-(2-((1R,2R)-1-(((R)-tert-butylsulfinyl)amino)-2-cyclopropoxypropyl)-1-((2-(trimethylsilyl)ethoxy)methyl)-1H-benzo[d]imidazol-5-yl)-2-cyclopropoxyethyl)amino)-1,1,1-trifluoropropan-2-yl)carbamate (438 mg, 0.54 mmol, Step C) and MeOH (15 mL) were combined in a Parr reaction tube followed by the addition of palladium on charcoal (252 mg, 0.24 mmol, 10% wet). The vial was then put in a Parr shaker bottle, placed under hydrogen (3 atm) and shaken for approximately 2.5 h. The reaction was filtered through diatomaceous earth (e.g., Celite©) and the pad rinsed with MeOH. The filtrate was concentrated under reduced pressure to afford the title compound in quantitative yield.

Step E: (R)—N-((1R,2R)-2-Cyclopropoxy-1-(5-((S)-2-cyclopropoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)propyl)-2-methylpropane-2-sulfinamide. (R)—N-((1R,2R)-1-(5-((S)-1-(((S)-2-Amino-3,3,3-trifluoropropyl)amino)-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide (372 mg, 0.55 mmol, Step D) and DCM (10 mL) were combined and cooled to 0° C. under nitrogen followed by the addition of DIPEA (300 μL, 1.72 mmol) under nitrogen. In a separate flask, triphosgene (77.2 mg, 0.26 mmol) and DCM (3 mL) were combined and cooled to 0° C. under nitrogen. The triphosgene solution was then added dropwise to the main reaction solution and continued to stir at 0° C. for 45 min. Additional triphosgene (18.5 mg, 0.062 mmol) in DCM (3 mL) was then added and the reaction was continued to stir at 0° C. for 45 min. The reaction was then quenched with the addition of methanol, warmed to rt and concentrated under reduced pressure. The product was purified by silica gel chromatography (0-100% (10% MeOH in EtOAc)/hexanes) to afford the title compound in 65% yield.

Step F: (S)-1-((S)-1-(2-((1R,2R)-1-Amino-2-cyclopropoxypropyl)-1H-benzo[d]imidazol-5-yl)-2-cyclopropoxyethyl)-4-(trifluoromethyl)imidazolidin-2-one. (R)—N-((1R,2R)-2-Cyclopropoxy-1-(5-((S)-2-cyclopropoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)propyl)-2-methylpropane-2-sulfinamide (253 mg, 0.36 mmol, Step E), a solution of 1,4-dioxane/MeOH (5 mL, 4:1) and HCl (750 μL, 4 M in 1,4-dioxane, 3 mmol) were combined and heated at 60° C. in a sealed vial for approximately 4.5 h. The reaction was cooled to rt, concentrated under reduced pressure, dissolved in deionized water and washed with hexanes, then the hexanes wash was discarded. The pH of the aqueous phase was adjusted to approximately pH 8 by the addition of saturated aqueous sodium bicarbonate solution. The aqueous layer was then salted with sodium chloride and extracted three times with ethyl acetate. The combined EtOAc layers were dried over anhydrous magnesium sulfate, filtered and concentrated under reduced pressure to afford the title compound in 81% yield.

### Intermediate 183: 1-((S)-1-(2-((1R,2R)-1-Amino-2-cyclopropoxypropyl)-1H-benzo[d]imidazol-5-yl)-2-cyclopropoxyethyl)-5,5-difluorotetrahydropyrimidin-2(1H)-one

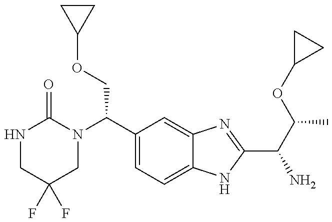 [SMILES: C[C@@H](OC1CC1)[C@H](N)c1nc2cc([C@@H](COC3CC3)N3CC(F)(F)CNC3=O)ccc2[nH]1]

Step A: (R)—N-((1R,2R)-2-Cyclopropoxy-1-(5-((S)-2-cyclopropoxy-1-((3-(1,3-dioxoisoindolin-2-yl)-2,2-difluoropropyl)amino)ethyl)-1-((2-(trimethylsilyl)ethoxy)methyl)-1H-benzo[d]imidazol-2-yl)propyl)-2-methylpropane-2-sulfinamide. (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide (1.19 g, 2.11 mmol, Intermediate 182 Step B), MeCN (25 mL), DIPEA (0.8 mL, 4.64 mmol) and 3-(1,3-dioxoisoindolin-2-yl)-2,2-difluoropropyl trifluoromethanesulfonate (1.22 g, 3.27 mmol) were combined and heated at 55° C. under nitrogen over the weekend. The contents were cooled to rt and transferred to a separatory funnel with EtOAc and deionized water. The layers were separated and the aqueous phase was salted with sodium chloride and extracted three times with EtOAc. The combined organic layers were dried over anhydrous magnesium sulfate, filtered and concentrated under reduced pressure. The product was purified by silica gel chromatography (0-100% (10% MeOH in EtOAc)/hexanes) to afford the title compound in approximately 25% yield.

Step B: (R)—N-((1R,2R)-1-(5-((S)-1-((3-Amino-2,2-difluoropropyl)amino)-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide. (R)—N-((1R,2R)-2-Cyclopropoxy-1-(5-((S)-2-cyclopropoxy-1-((3-(1,3-dioxoisoindolin-2-yl)-2,2-difluoropropyl)amino)ethyl)-1-((2-(trimethylsilyl)ethoxy)methyl)-1H-benzo[d]imidazol-2-yl)propyl)-2-methylpropane-2-sulfinamide (417 mg, 0.53 mmol, Step A), EtOH (10 mL) and hydrazine monohydrate (500 μL, 6.69 mmol) were combined and stirred at rt overnight under nitrogen. The reaction mixture was transferred to a separatory funnel with deionized water and ethyl acetate. The organic phase was separated and the aqueous layer was salted with sodium chloride and extracted twice with ethyl acetate. The combined organic layers were dried over anhydrous magnesium sulfate, filtered and concentrated to dryness to afford the title compound in 90% yield.

Step C: (R)—N-((1R,2R)-2-Cyclopropoxy-1-(5-((S)-2-cyclopropoxy-1-(5,5-difluoro-2-oxotetrahydropyrimidin-1(2H)-yl)ethyl)-1-((2-(trimethylsilyl)ethoxy)methyl)-1H-benzo[d]imidazol-2-yl)propyl)-2-methylpropane-2-sulfinamide. (R)—N-((1R,2R)-1-(5-((S)-1-((3-Amino-2,2-difluoropropyl)amino)-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide (314 mg, 0.48 mmol, Step B), THF (30 mL), DIPEA (300 μL, 1.74 mmol) and carbonyldiimidazole (240.3 mg, 1.48 mmol) were combined then heated at 60° C. under nitrogen for 2 d. The contents were transferred to a separatory funnel with EtOAc and deionized water. The layers were separated and the aqueous phase was salted with sodium chloride and extracted three times with EtOAc. The combined organic layers were dried over anhydrous magnesium sulfate, filtered and concentrated under reduced pressure. The product was purified by silica gel chromatography (0-100% (10% MeOH in EtOAc)/hexanes) to afford the title compound in quantitative yield.

Step D: 1-((S)-1-(2-((1R,2R)-1-Amino-2-cyclopropoxypropyl)-1H-benzo[d]imidazol-5-yl)-2-cyclopropoxyethyl)-5,5-difluorotetrahydropyrimidin-2(1H)-one. (R)—N-((1R,2R)-2-Cyclopropoxy-1-(5-((S)-2-cyclopropoxy-1-(5,5-difluoro-2-oxotetrahydropyrimidin-1(2H)-yl)ethyl)-1-((2-(trimethylsilyl)ethoxy)methyl)-1H-benzo[d]imidazol-2-yl)propyl)-2-methylpropane-2-sulfinamide (408 mg, 0.48 mmol, Step C), a solution of 1,4-dioxane/MeOH (10 mL, 4:1) and HCl (1 mL, 4 M in 1,4-dioxane, 4 mmol) were combined and heated at 60° C. in a sealed vial for approximately 4.5 h. The reaction was cooled to rt then concentrated under reduced pressure, dissolved in deionized water and washed with hexanes, then the hexanes wash was discarded. The pH of the aqueous phase was adjusted to approximately pH 8 by the addition of saturated aqueous sodium bicarbonate solution. The aqueous layer was then salted with sodium chloride and extracted three times with ethyl acetate. The combined organic layers were dried over anhydrous magnesium sulfate, filtered and concentrated under reduced pressure to afford the title compound in 81% yield.

### Intermediate 184: (R)-2-Methyl-N—((R,E)-2-((1,1,1-trifluoro-2-methylpropan-2-yl)oxy)propylidene)propane-2-sulfinamide

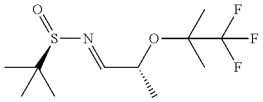 [SMILES: C[C@H](/C=N/[S@](=O)C(C)(C)C)OC(C)(C)C(F)(F)F]

Step A: (R)-2-((1,1,1-Trifluoro-2-methylpropan-2-yl)oxy)propanoic acid. The title compound was synthesized in a manner analogous to Intermediate 1 Step A using (S)-2-bromopropanoic acid in place of allyl bromide (20% yield).

Step B: (R)—N-Methoxy-N-methyl-2-((1,1,1-trifluoro-2-methylpropan-2-yl)oxy)propanamide. The title compound was synthesized in a manner analogous to Intermediate 3 using (R)-2-((1,1,1trifluoro-2-methylpropan-2-yl)oxy)propanoic acid (Step A) in place of (R)-2-(((R)-1,1,1-trifluoropropan-2-yl)oxy)propanoic acid (26% yield).

Step C: (R)-2-((1,1,1-Trifluoro-2-methylpropan-2-yl)oxy)propanal. The title compound was synthesized in a manner analogous to Intermediate 4 using (R)—N-methoxy-N-methyl-2-((1,1,1-trifluoro-2-methylpropan-2-yl)oxy)propanamide (Step B) in place of (R)—N-methoxy-N-methyl-2-(((R)-1,1,1-trifluoropropan-2-yl)oxy)propanamide in quantitative yield.

Step D: (R)-2-Methyl-N—((R,E)-2-((1,1,1-trifluoro-2-methylpropan-2-yl)oxy)propylidene)propane-2-sulfinamide. The title compound was synthesized in a manner analogous to Intermediate 5 using (R)-2-((1,1,1-trifluoro-2-methylpropan-2-yl)oxy)propanal (Step C) in place of (R)-2-(((R)-1,1,1-trifluoropropan-2-yl)oxy)propanal (60% yield).

### Intermediate 185: 3-(1,3-Dioxoisoindolin-2-yl)-2,2-difluoropropyl-1,1-d₂ trifluoromethanesulfonate

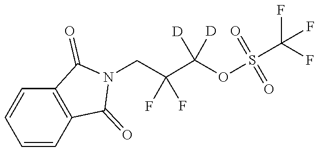 [SMILES: [2H]C([2H])(OS(=O)(=O)C(F)(F)F)C(F)(F)CN1C(=O)c2ccccc2C1=O]

Step A: Methyl 3-(benzylamino)-2,2-difluoro-3-oxopropanoate. A solution of dimethyl difluoromalonate (14 g, 83.3 mmol) in MeOH (555 mL) was cooled to 0° C. in an ice-bath and then benzylamine (7.31 mL, 66.6 mmol) was added dropwise and the resulting mixture was allowed to warm to rt overnight. The mixture was concentrated to dryness and the resulting material was purified by silica gel chromatography (2-25% EtOAc/toluene) to provide the title compound (46% yield).

Step B: N-Benzyl-2,2-difluoro-3-hydroxy-3,3-dideuteropropanamide. A solution of methyl 3-(benzylamino)-2,2-difluoro-3-oxopropanoate (7.7 g, 31.7 mmol, Step A) and MeOH (158 mL) was cooled to 0° C. and then sodium borodeuteride (3.31 g, 79.2 mmol) was added portion-wise. The resulting mixture was stirred at 0° C. for 1 h. The reaction was then quenched by the dropwise addition of water (15 mL) and concentrated to dryness. The residue was partitioned between water and EtOAc and the aqueous phase further extracted with EtOAc. The organic layers were combined, washed with brine, dried over anhydrous MgSO₄, filtered and concentrated to dryness to provide the title compound as an off-white solid (98% yield).

Step C: 3-(Benzylamino)-2,2-difluoropropan-1,1-d₂-1-ol. To a mixture of N-benzyl-2,2-difluoro-3-hydroxy-3,3-dideuteropropanamide (250 mg, 1.15 mmol, Step B), sodium borohydride (96 mg, 2.5 mmol) and THF (7.5 mL) was added boron trifluoride diethyl etherate (0.61 mL, 2.3 mmol) dropwise. The resulting mixture was heated at reflux for 1 h and then cooled to rt. Then, MeOH was added and the mixture stirred at rt for 15 min. The mixture was concentrated to dryness to provide the title compound, which was used in the next step without further purification.

Step D: 3-Amino-2,2-difluoropropan-1,1-d₂-1-ol. A mixture of 3-(benzylamino)-2,2-difluoropropan-1,1-d₂-1-ol (234 mg, 1.15 mmol, Step C), 10% Pd/C (123 mg, 0.12 mmol), ammonium formate (726 mg, 11.5 mmol) and MeOH (23 mL) was heated at 60° C. for 1 h. After that time, the mixture was filtered through diatomaceous earth (e.g., Celite©), the filter-cake was rinsed with EtOAc (30 mL), and the filtrate concentrated to dryness to provide the title compound as a white solid, which was used in the next step without further purification.

Step E: 2-(2,2-Difluoro-3-hydroxypropyl-3,3-d₂)isoindoline-1,3-dione. A mixture of 3-amino-2,2-difluoropropan-1,1-d₂-1-ol (3.28 g, 29 mmol, Step D), N-carbethoxyphthalimide (7.32 g, 33.4 mmol), DIPEA (20 mL, 116 mmol) and THF (73 mL) was heated at 65° C. overnight. The mixture was then concentrated to dryness and the residue dissolved in water (250 mL) and stirred for 30 min. The precipitate that formed was collected by filtration and dried under vacuum to provide the title compound as a white solid (66% yield), which was used in the next step without further purification.

Step F: 3-(1,3-Dioxoisoindolin-2-yl)-2,2-difluoropropyl-1,1-d₂ trifluoromethanesulfonate. To a mixture of 2-(2,2-difluoro-3-hydroxypropyl-3,3-d₂)isoindoline-1,3-dione (1 g, 4.1 mmol, Step E), pyridine (0.36 mL, 4.5 mmol) and DCM (13.7 mL) was added trifluoromethanesulfonic anhydride (0.73 mL, 4.3 mmol) dropwise. The resulting mixture was stirred at rt for 30 min and then partitioned between water and DCM. The layers were separated and the aqueous phase further extracted with DCM. The organic layers were combined, washed with brine, dried over anhydrous MgSO₄, filtered and concentrated to dryness. The orange residue was purified by silica gel chromatography (0-100% EtOAc/hexanes) to provide the title compound as an off-white solid.

### Intermediate 186: 1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-6,6-d₂

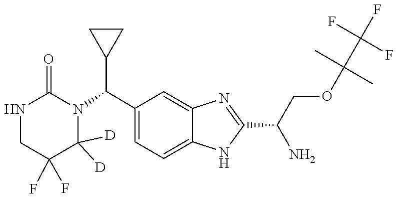 [SMILES: [2H]C1([2H])N([C@@H](c2ccc3[nH]c([C@@H](N)COC(C)(C)C(F)(F)F)nc3c2)C2CC2)C(=O)NCC1(F)F]

Step A: (R)—N—((R)-1-(5-((R)-Cyclopropyl((3-(1,3-dioxoisoindolin-2-yl)-2,2-difluoropropyl-1,1-d₂)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound was prepared as described for the synthesis of Intermediate 95 Step B using (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 32) in place of (R)—N—((R*)-1-(5-((R)-amino(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide and 3-(1,3-dioxoisoindolin-2-yl)-2,2-difluoropropyl-1,1-d₂ trifluoromethanesulfonate (Intermediate 185) in place of 3-(1,3-dioxoisoindolin-2-yl)-2,2-difluoropropyl trifluoromethanesulfonate, and stirring the mixture overnight at 50° C. instead of 1 h at rt.

Step B: (R)—N—((R)-1-(5-((R)-((3-Amino-2,2-difluoropropyl-1,1-d₂)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound was prepared as described for the synthesis of Intermediate 96 using (R)—N—((R)-1-(5-((R)-cyclopropyl((3-(1,3-dioxoisoindolin-2-yl)-2,2-difluoropropyl-1,1-d₂)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step A) in place of (R)—N—((R*)-1-(5-((R)-cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide.

Step C: (R)—N—((R)-1-(5-((R)-Cyclopropyl(5,5-difluoro-2-oxotetrahydropyrimidin-1(2H)-yl-6,6-d₂)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound was prepared as described for the synthesis of Intermediate 97 using (R)—N—((R)-1-(5-((R)-((3-amino-2,2-difluoropropyl-1,1-d₂)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step B) in place of (R)—N—((R*)-1-(5-((R)-((3-amino-2,2-difluoropropyl)amino)(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide, to provide the title compound as a glassy solid (60% yield).

Step D: 1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-6,6-d₂. The title compound was prepared as described for the synthesis of Intermediate 57 using (R)—N—((R)-1-(5-((R)-cyclopropyl(5,5-difluoro-2-oxotetrahydropyrimidin-1(2H)-yl-6,6-d₂)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step C) in place of (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, and stirring the mixture at 60° C. for 1 h instead of 3 h.

### Intermediate 187: 3-(1,3-Dioxoisoindolin-2-yl)-2,2-difluoropropyl-1,1,3,3-d₄ trifluoromethanesulfonate

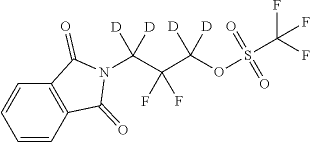 [SMILES: [2H]C([2H])(OS(=O)(=O)C(F)(F)F)C(F)(F)C([2H])([2H])N1C(=O)c2ccccc2C1=O]

The title compound was prepared as described for Intermediate 185 using sodium borodeuteride in place of sodium borohydride in Step C to provide the title compound as a white foam.

### Intermediate 188: 1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-4,4,6,6-d₄

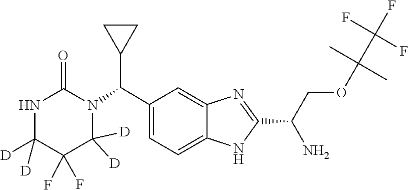 [SMILES: [2H]C1([2H])NC(=O)N([C@@H](c2ccc3[nH]c([C@@H](N)COC(C)(C)C(F)(F)F)nc3c2)C2CC2)C([2H])([2H])C1(F)F]

Step A: (R)—N—((R)-1-(5-((R)-Cyclopropyl((3-(1,3-dioxoisoindolin-2-yl)-2,2-difluoropropyl-1,1,3,3-d₄)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound was prepared as described for the synthesis of Intermediate 95 Step B using (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 32) in place of (R)—N—((R*)-1-(5-((R)-amino(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide and 3-(1,3-dioxoisoindolin-2-yl)-2,2-difluoropropyl-1,1,3,3-d₄ trifluoromethanesulfonate (Intermediate 187) in place of 3-(1,3-dioxoisoindolin-2-yl)-2,2-difluoropropyl trifluoromethanesulfonate.

Step B: (R)—N—((R)-1-(5-((R)-((3-Amino-2,2-difluoropropyl-1,1,3,3-d₄)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound was prepared as described for the synthesis of Intermediate 96 using (R)—N—((R)-1-(5-((R)-cyclopropyl((3-(1,3-dioxoisoindolin-2-yl)-2,2-difluoropropyl-1,1,3,3-d₄)amino)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step A) in place of (R)—N—((R*)-1-(5-((R)-cyclopropyl((3-(1,3-dioxoisoindolin-2-yl)-2,2-difluoropropyl)amino)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide, to provide the title compound (85% yield).

Step C: (R)—N—((R)-1-(5-((R)-Cyclopropyl(5,5-difluoro-2-oxotetrahydropyrimidin-1(2H)-yl-4,4,6,6-d₄)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound was prepared as described for the synthesis of Intermediate 97 using (R)—N—((R)-1-(5-((R)-((3-amino-2,2-difluoropropyl-1,1,3,3-d₄)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step B) in place of (R)—N—((R*)-1-(5-((R)-((3-amino-2,2-difluoropropyl)amino)(cyclopropyl)methyl)-1-((2-(trimethylsilyl)ethoxy)methyl)-1H-imidazo[4,5-b]pyridin-2-yl)-2-((1,1,1-trifluoro-2-methylpropan-2-yl)oxy)ethyl)-2-methylpropane-2-sulfinamide, to provide the title compound as a glassy solid (83% yield).

Step D: 1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-4,4,6,6-d₄. The title compound was prepared as described for the synthesis of Intermediate 57 using (R)—N—((R)-1-(5-((R)-cyclopropyl(5,5-difluoro-2-oxotetrahydropyrimidin-1(2H)-yl-4,4,6,6-d₄)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step C) in place of (R)—N-((1R*,2R)-1-(5-((1R)-cyclopropyl(2-oxo-4-(trifluoromethyl)imidazolidin-1-yl-5,5-d₂)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide, and stirring the mixture at 60° C. for 1 h instead of 3 h.

### Intermediate 189: 3-(1,3-Dioxoisoindolin-2-yl)-2,2-difluoropropyl-3,3-d₂ trifluoromethanesulfonate

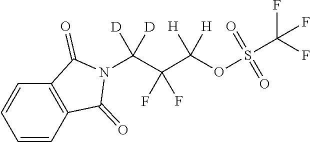 [SMILES: [2H]C([2H])(N1C(=O)c2ccccc2C1=O)C(F)(F)COS(=O)(=O)C(F)(F)F]

The title compound was prepared as described for Intermediate 185 using sodium borohydride in place of sodium borodeuteride and stirring at rt instead of 0° C. for 1 h in Step B, and using sodium borodeuteride in place of sodium borohydride in Step C to provide the title compound (84% yield).

### Intermediate 190: 1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-4,4-d₂

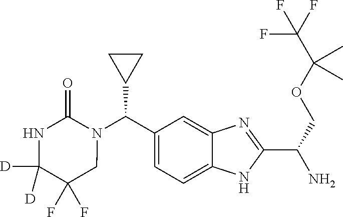 [SMILES: [2H]C1([2H])NC(=O)N([C@@H](c2ccc3[nH]c([C@@H](N)COC(C)(C)C(F)(F)F)nc3c2)C2CC2)CC1(F)F]

The title compound was prepared as described for Intermediate 188 using 3-(1,3-dioxoisoindolin-2-yl)-2,2-difluoropropyl-3,3-d₂ trifluoromethanesulfonate (Intermediate 189) in place of 3-(1,3-dioxoisoindolin-2-yl)-2,2-difluoropropyl-1,1,3,3-d₄ trifluoromethanesulfonate in Step A.

### Intermediate 191: Benzyl (R)-4-isopropyl-1,2,3-oxathiazolidine-3-carboxylate 2,2-dioxide

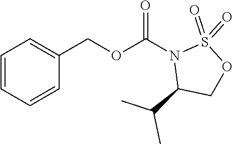 [SMILES: CC(C)[C@@H]1COS(=O)(=O)N1C(=O)OCc1ccccc1]

The title compound was prepared as described for Intermediate 24 using (R)-2-amino-3-methylbutan-1-ol in place of (S)-2-amino-3,3,3-trifluoropropan-1-ol hydrochloride in Step A.

### Intermediate 192: (R)-1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-4-isopropylimidazolidin-2-one

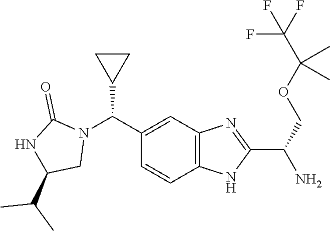 [SMILES: CC(C)[C@@H]1CN([C@@H](c2ccc3[nH]c([C@@H](N)COC(C)(C)C(F)(F)F)nc3c2)C2CC2)C(=O)N1]

Step A: Benzyl ((R)-1-(((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)amino)-3-methylbutan-2-yl)carbamate. A mixture of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (370 mg, 0.63 mmol, Intermediate 32) and benzyl (R)-4-isopropyl-1,2,3-oxathiazolidine-3-carboxylate 2,2-dioxide (375 mg, 1.25 mmol, Intermediate 191) in ACN (7 mL) was heated in a sealed tube at 60° C. overnight. The resulting suspension was concentrated to dryness and the residue purified by silica gel chromatography (10-20% MeOH/DCM) to provide the title compound as a white solid (99% yield).

Step B: (R)—N—((R)-1-(5-((R)—(((R)-2-Amino-3-methylbutyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound (93% yield) was prepared as described for Intermediate 33 Step B using benzyl ((R)-1-(((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)amino)-3-methylbutan-2-yl)carbamate (Step A) in place of benzyl ((S)-3-(((R)-cyclopropyl(2-((R)-1-((R)-1,1-dimethylethylsulfinamido)-2-((1,1,1-trifluoro-2-methylpropan-2-yl)oxy)ethyl)-1-((2-(trimethylsilyl)ethoxy)methyl)-1H-benzo [d]imidazol-5-yl)methyl)amino)-1,1,1-trifluoropropan-2-yl)carbamate.

Step C: (R)—N—((R)-1-(5-((R)-Cyclopropyl((R)-4-isopropyl-2-oxoimidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound (98% yield) was prepared as described for Intermediate 33 Step C using (R)—N—((R)-1-(5-((R)—(((R)-2-amino-3-methylbutyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step B) in place of (R)—N—((R)-1-(5-((R)—(((S)-2-amino-3,3,3-trifluoropropyl)amino) (cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

Step D: (R)-1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-4-isopropylimidazolidin-2-one. The title compound was prepared as described in Intermediate 34 using (R)—N—((R)-1-(5-((R)-cyclopropyl((R)-4-isopropyl-2-oxoimidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step C) in place of (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide. After concentrating to dryness, the title compound was used without further purification (79% yield).

### Intermediate 193: Benzyl (R)-4-cyclopropyl-1,2,3-oxathiazolidine-3-carboxylate 2,2-dioxide

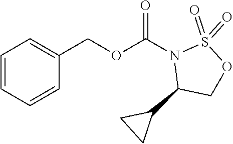 [SMILES: O=C(OCc1ccccc1)N1[C@H](C2CC2)COS1(=O)=O]

The title compound was prepared as described for Intermediate 24 using (R)-2-amino-2-cyclopropylethanol hydrochloride in place of (S)-2-amino-3,3,3-trifluoropropan-1-ol hydrochloride in Step A. In Step A, the residue was purified by silica gel chromatography (0-50% EtOAc/petroleum ether).

### Intermediate 194: (R)-1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-4-cyclopropylimidazolidin-2-one

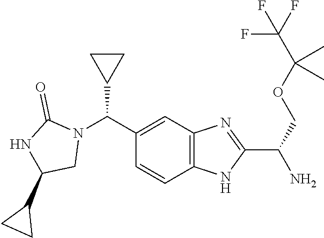 [SMILES: CC(C)(OC[C@H](N)c1nc2cc([C@@H](C3CC3)N3C[C@@H](C4CC4)NC3=O)ccc2[nH]1)C(F)(F)F]

The title compound was prepared as described in Intermediate 192 using benzyl (R)-4-cyclopropyl-1,2,3-oxathiazolidine-3-carboxylate 2,2-dioxide (Intermediate 193) in place of benzyl (R)-4-isopropyl-1,2,3-oxathiazolidine-3-carboxylate 2,2-dioxide in Step A. In Step D, the residue, a yellow oil (94% yield), was used without further purification.

### Intermediate 195: (S)-1-((R)-(2-((1R,2R)-1-Amino-2-(((R)-1,1,1-trifluoropropan-2-yl)oxy)propyl)-4-fluoro-1H-benzo[d]imidazol-6-yl)(cyclopropyl)methyl)-4-(trifluoromethyl)imidazolidin-2-one hydrochloride

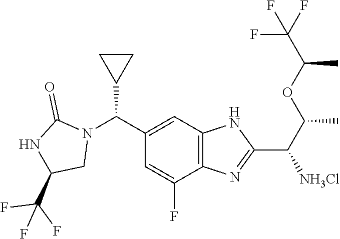

Step A: 5-Bromo-7-fluoro-1-((2-(trimethylsilyl)ethoxy)methyl)-1H-benzo[d]imidazole. The title compound was prepared as described in Intermediate 13 using 5-bromo-7-fluoro-1H-benzo[d]imidazole in place of 5-bromo-1H-benzo[d]imidazole, DMF in place of THF and stirring at rt for 0.5 h instead of 4 h. The residue was purified by silica gel chromatography (50% EtOAc/petroleum ether, isocratic) to provide the title compound as a brown oil (71% yield).

Step B: 7-Fluoro-1-((2-(trimethylsilyl)ethoxy)methyl)-5-vinyl-1H-benzo[d]imidazole. The title compound was prepared as described in Intermediate 14 using 5-bromo-7-fluoro-1-((2-(trimethylsilyl)ethoxy)methyl)-1H-benzo[d]imidazole (Step A) in place of 5-bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole and stirring at 100° C. for 16 h instead of 85° C. for 3 h. The residue was purified by silica gel chromatography (0-70% EtOAc/petroleum ether) to provide the title compound as a yellow oil (84% yield).

Step C: 7-Fluoro-1-((2-(trimethylsilyl)ethoxy)methyl)-1H-benzo[d]imidazole-5-carbaldehyde. A solution of 7-fluoro-1-((2-(trimethylsilyl)ethoxy)methyl)-5-vinyl-1H-benzo[d]imidazole (4.65 g, 15.9 mmol, Step B), NaIO₄ (11.0 g, 51.4 mol), K₂OsO₄·2H₂O (235 mg, 0.638 mmol), 1,4-dioxane (50 mL) and water (50 mL) was stirred at rt overnight. Then, the mixture was quenched by the addition of saturated aqueous Na₂SO₃ (50 mL) and the resulting suspension filtered. The filter cake was washed with EtOAc (80 mL) and then the filtrate was diluted with water (100 mL) and extracted with EtOAc (2×80 mL). The organic layers were combined, washed with brine (100 mL), dried over anhydrous Na₂SO₄, filtered and concentrated to dryness to provide the title compound as a brown oil (99% yield) which was used without further purification.

Step D: (S,E)-N-((7-Fluoro-1-((2-(trimethylsilyl)ethoxy)methyl)-1H-benzo[d]imidazol-5-yl)methylene)-2-methylpropane-2-sulfinamide. The title compound was prepared as described for Intermediate 105 using 7-fluoro-1-((2-(trimethylsilyl)ethoxy)methyl)-1H-benzo[d]imidazole-5-carbaldehyde (Step C) in place of 1-((2-(trimethylsilyl)ethoxy)methyl)-1H-imidazo[4,5-b]pyridine-5-carbaldehyde and 3-((2-(trimethylsilyl)ethoxy)methyl)-3H-imidazo[4,5-b]pyridine-5-carbaldehyde and (S)-2-methylpropane-2-sulfinamide in place of (R)-2-methylpropane-2-sulfinamide. The residue was purified by silica gel chromatography (0-2% MeOH/DCM) to provide the title compound as a yellow solid (84% yield).

Step E: (S)—N—((R)-Cyclopropyl(7-fluoro-1-((2-(trimethylsilyl)ethoxy)methyl)-1H-benzo[d]imidazol-5-yl)methyl)-2-methylpropane-2-sulfinamide. The title compound was prepared as described in Intermediate 30 Step A using (S,E)-N-((7-fluoro-1-((2-(trimethylsilyl)ethoxy)methyl)-1H-benzo[d]imidazol-5-yl)methylene)-2-methylpropane-2-sulfinamide (Step D) in place of (S,E)-2-methyl-N-((1-((2-(trimethylsilyl)ethoxy)methyl)-1H-benzo[d]imidazol-5-yl)methylene)propane-2-sulfinamide. The residue was purified by silica gel chromatography (0-5% MeOH/DCM) to provide the title compound as a yellow gum (72% yield).

Step F: (R)-Cyclopropyl(7-fluoro-1-((2-(trimethylsilyl)ethoxy)methyl)-1H-benzo[d]imidazol-5-yl)methanamine. The title compound (63% yield, yellow oil) was prepared as described in Intermediate 30 Step B using (S)—N—((R)-cyclopropyl(7-fluoro-1-((2-(trimethylsilyl)ethoxy)methyl)-1H-benzo[d]imidazol-5-yl)methyl)-2-methylpropane-2-sulfinamide (Step E) in place of (S)—N—((R)-cyclopropyl(1-((2-(trimethylsilyl)ethoxy)methyl)-1H-benzo[d]imidazol-5-yl)methyl)-2-methylpropane-2-sulfinamide, and the mixture was stirred at rt for 2 h instead of 16 h.

Step G: (R)-2-(Cyclopropyl(7-fluoro-1-((2-(trimethylsilyl)ethoxy)methyl)-1H-benzo[d]imidazol-5-yl)methyl)isoindoline-1,3-dione. The title compound (82% yield, light yellow gum) was prepared as described in Intermediate 30 Step C using (R)-cyclopropyl(7-fluoro-1-((2-(trimethylsilyl)ethoxy)methyl)-1H-benzo[d]imidazol-5-yl)methanamine (Step F) in place of (R)-cyclopropyl(1-((2-(trimethylsilyl)ethoxy)methyl)-1H-benzo[d]imidazol-5-yl)methanamine.

Step H: (R)—N-((1R,2R)-1-(5-((R)-Cyclopropyl(1,3-dioxoisoindolin-2-yl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. The title compound was prepared as described in Intermediate 31 using (R)-2-(cyclopropyl(7-fluoro-1-((2-(trimethylsilyl)ethoxy)methyl)-1H-benzo[d]imidazol-5-yl)methyl)isoindoline-1,3-dione (Step G) in place of (R)-2-(cyclopropyl(1-((2-(trimethylsilyl)ethoxy)methyl)-1H-benzo[d]imidazol-5-yl)methyl)isoindoline-1,3-dione and (R,E)-2-methyl-N—((R)-2-(((R)-1,1,1-trifluoropropan-2-yl)oxy)propylidene)propane-2-sulfinamide (Intermediate 5) in place of (R,E)-2-methyl-N-(2-((1,1,1-trifluoro-2-methylpropan-2-yl)oxy)ethylidene)propane-2-sulfinamide. The residue was purified by silica gel chromatography twice (0-80% EtOAc/petroleum ether followed by 0-2% MeOH/DCM) to provide the title compound as a colorless oil (40% yield).

Step I: (R)—N-((1R,2R)-1-(5-((R)-Amino(cyclopropyl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. A flask was charged with (R)—N-((1R,2R)-1-(5-((R)-cyclopropyl(1,3-dioxoisoindolin-2-yl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (450 mg, 0.61 mmol, Step H), EtOH (6 mL) and hydrazine monohydrate (0.32 mL, 6.2 mmol). The resulting mixture was stirred at rt overnight, during which time a white solid was formed. After that time, the mixture was filtered and the filtrate concentrated to dryness. The residue was dissolved in DCM (20 mL), washed with water (10 mL) followed by brine (10 mL), dried over anhydrous Na₂SO₄, filtered and concentrated to dryness to give a colorless oil. The oil was purified by silica gel chromatography (0-6% MeOH/DCM) to provide the title compound as a colorless gum (66% yield).

Step J: Benzyl ((S)-3-(((R)-(2-((1R,2R)-1-(((R)-tert-butylsulfinyl)amino)-2-(((R)-1,1,1-trifluoropropan-2-yl)oxy)propyl)-7-fluoro-1-((2-(trimethylsilyl)ethoxy)methyl)-1H-benzo[d]imidazol-5-yl)(cyclopropyl)methyl)amino)-1,1,1-trifluoropropan-2-yl)carbamate. A mixture of (R)—N-((1R,2R)-1-(5-((R)-amino(cyclopropyl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (244 mg, 0.401 mmol, Step I), (S)-benzyl 4-(trifluoromethyl)-1,2,3-oxathiazolidine-3-carboxylate 2,2-dioxide (263 mg, 0.809 mmol, Intermediate 24) and Cs₂CO₃ (393 mg, 1.21 mmol) in THF/MeCN (1:1, 8 mL) was heated at 60° C. overnight. The reaction was allowed to cool to rt, the mixture was filtered and the filtrate concentrated to dryness to give a light yellow oil. The oil was dissolved in EtOAc (50 mL), washed with water (50 mL) and brine (50 mL), dried over anhydrous Na₂SO₄, filtered and concentrated to dryness. The residue was purified by silica gel chromatography (0-5% MeOH/DCM) followed by preparative HPLC (Xtimate C18 column 10 mm, 40×150 mm, 35-65% CH₃CN/H₂O with NH₄OH) to provide the title compound as a yellow solid (48% yield).

Step K: (R)—N-((1R,2R)-1-(5-((R)—(((S)-2-Amino-3,3,3-trifluoropropyl)amino)(cyclopropyl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. The title compound (100% yield, white solid) was prepared as described in Intermediate 33 Step B using benzyl ((S)-3-(((R)-(2-((1R,2R)-1-(((R)-tert-butylsulfinyl)amino)-2-(((R)-1,1,1-trifluoropropan-2-yl)oxy)propyl)-7-fluoro-1-((2-(trimethylsilyl)ethoxy)methyl)-1H-benzo[d]imidazol-5-yl)(cyclopropyl)methyl)amino)-1,1,1-trifluoropropan-2-yl)carbamate (Step J) in place of benzyl ((S)-3-(((R)-cyclopropyl(2-((R)-1-((R)-1,1-dimethylethylsulfinamido)-2-((1,1,1-trifluoro-2-methylpropan-2-yl)oxy)ethyl)-1-((2-(trimethylsilyl)ethoxy)methyl)-1H-benzo [d]imidazol-5-yl)methyl)amino)-1,1,1-trifluoropropan-2-yl)carbamate, and stirring for 2 h at 45 psi H₂ instead of 1.5 h. The title compound was used directly in the next step without any purification.

Step L: (R)—N-((1R,2R)-1-(5-((R)-Cyclopropyl((S)-2-oxo-4-(trifluoromethyl)imidazolidin-1-yl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. The title compound was prepared as described in Intermediate 33 Step C using (R)—N-((1R,2R)-1-(5-((R)—(((S)-2-amino-3,3,3-trifluoropropyl)amino)(cyclopropyl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (Step K) in place of (R)—N—((R)-1-(5-((R)—(((S)-2-amino-3,3,3-trifluoropropyl)amino) (cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The isolated material was purified by silica gel chromatography (0-6% MeOH/DCM) followed by SFC (DAICEL CHIRALPAK AS column, m, 250 mm×30 mm; 15% (v/v) EtOH (containing 0.1% of 25% aqueous NH₃)/CO₂) to provide the title compound as a white solid (37% yield).

Step M: (S)-1-((R)-(2-((1R,2R)-1-Amino-2-(((R)-1,1,1-trifluoropropan-2-yl)oxy)propyl)-4-fluoro-1H-benzo[d]imidazol-6-yl)(cyclopropyl)methyl)-4-(trifluoromethyl)imidazolidin-2-one hydrochloride. A mixture of (R)—N-((1R,2R)-1-(5-((R)-Cyclopropyl((S)-2-oxo-4-(trifluoromethyl)imidazolidin-1-yl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (80 mg, 0.11 mmol, Step L), MeOH (3 mL) and HCl (2 mL, 8 mmol, 4 M in 1,4-dioxane) was heated at 55° C. for 3 h. Then the mixture was concentrated to dryness to provide the title compound as a light yellow solid (100% yield) which was used without further purification.

### Intermediate 196: Benzyl 6-oxa-5-thia-4-azaspiro[2.4]heptane-4-carboxylate 5,5-dioxide

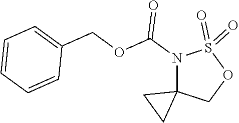 [SMILES: O=C(OCc1ccccc1)N1C2(CC2)COS1(=O)=O]

The title compound was prepared as described for Intermediate 24 using (1-aminocyclopropyl)methanol in place of (S)-2-amino-3,3,3-trifluoropropan-1-ol hydrochloride in Step A. In Step A, the reaction mixture was stirred at rt overnight instead of 30° C. for 16 h, and the residue was purified by silica gel chromatography (0-80% EtOAc/petroleum ether). In Step B, the residue was purified by silica gel chromatography (0-100% EtOAc/petroleum ether). In Step C, the residue was purified by silica gel chromatography (0-40% EtOAc/petroleum ether) followed by trituration with n-hexanes to provide the title compound as a grey-white solid.

### Intermediate 197: 6-((R)-(2-((R)-1-Amino-2-((1,1,1-trifluoro-2-methylpropan-2-yl)oxy)ethyl)-1H-benzo[d]imidazol-5-yl)(cyclopropyl)methyl)-4,6-diazaspiro[2.4]heptan-5-one

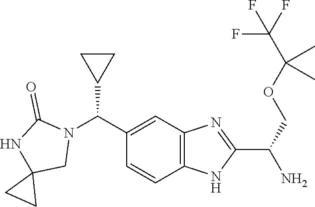 [SMILES: CC(C)(OC[C@H](N)c1nc2cc([C@@H](C3CC3)N3CC4(CC4)NC3=O)ccc2[nH]1)C(F)(F)F]

The title compound was prepared as described in Intermediate 192 using benzyl 6-oxa-5-thia-4-azaspiro[2.4]heptane-4-carboxylate 5,5-dioxide (Intermediate 196) in place of benzyl (R)-4-isopropyl-1,2,3-oxathiazolidine-3-carboxylate 2,2-dioxide in Step A, and stirring at 60° C. for 2 h instead of overnight in Step A. In Step D, the residue, a yellow oil, was purified by preparative HPLC (Phenomenex C18 3 μm, 75×30 mm, 28-58% MeCN/water with 0.05% NH₄OH+10 mM NH₄HCO₃).

### Intermediate 198: Benzyl 4-(2,2,2-trifluoroethyl)-1,2,3-oxathiazolidine-3-carboxylate 2,2-dioxide

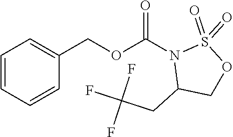 [SMILES: O=C(OCc1ccccc1)N1C(CC(F)(F)F)COS1(=O)=O]

The title compound was prepared as described for Intermediate 24 using 2-amino-4,4,4-trifluorobutan-1-ol in place of (S)-2-amino-3,3,3-trifluoropropan-1-ol hydrochloride in Step A. In Step A, the residue was purified by silica gel chromatography (0-25% EtOAc/petroleum ether). In Step B, the mixture was stirred for 3 h at −30° C. instead of 2 h, and the oxathiazolidine oxide stereoisomers were separated by silica gel chromatography (0-30% EtOAc/petroleum ether). In Step C, the benzyl (2R*)-4-(2,2,2-trifluoroethyl)-1,2,3-oxathiazolidine-3-carboxylate 2-oxide isomer was utilized, and the residue was purified by silica gel chromatography (0-13% EtOAc/petroleum ether) to provide the title compound as a white solid.

### Intermediate 199: (R)—N—((R)-1-(5-((R)—(((R*)-2-Amino-4,4,4-trifluorobutyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

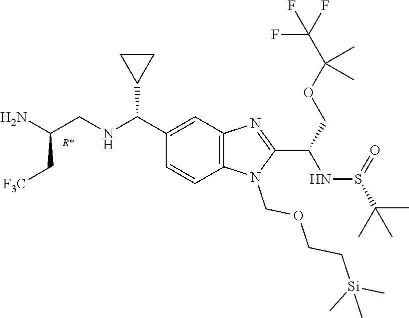 [SMILES: CC(C)(C)[S@@](=O)N[C@@H](COC(C)(C)C(F)(F)F)c1nc2cc([C@H](NC[C@H](N)CC(F)(F)F)C3CC3)ccc2n1COCC[Si](C)(C)C]

Intermediate 200: (R)—N—((R)-1-(5-((R)—(((S*)-2-Amino-4,4,4-trifluorobutyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

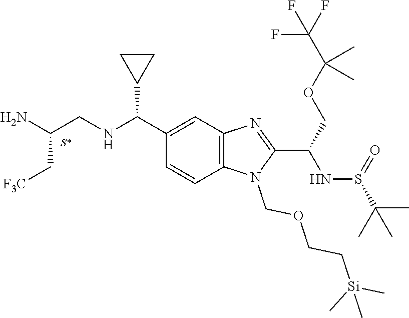 [SMILES: CC(C)(C)[S@@](=O)N[C@@H](COC(C)(C)C(F)(F)F)c1nc2cc([C@H](NC[C@@H](N)CC(F)(F)F)C3CC3)ccc2n1COCC[Si](C)(C)C]

Step A: Benzyl (1-(((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)amino)-4,4,4-trifluorobutan-2-yl)carbamate. The title compound was prepared as described in Intermediate 195 Step J using (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 32) in place of (R)—N-((1R,2R)-1-(5-((R)-amino(cyclopropyl)methyl)-7-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide and benzyl 4-(2,2,2-trifluoroethyl)-1,2,3-oxathiazolidine-3-carboxylate 2,2-dioxide (Intermediate 198) in place of (S)-benzyl 4-(trifluoromethyl)-1,2,3-oxathiazolidine-3-carboxylate 2,2-dioxide. The mixture was heated at 60° C. for 2 h instead of overnight, and the material was purified by silica gel chromatography (20-23% MeOH/DCM) to provide the title compound as a yellow oil.

Step B: (R)—N-((1R)-1-(5-((1R)-((2-Amino-4,4,4-trifluorobutyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound was prepared as described in Intermediate 54 Step B using benzyl (1-(((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)amino)-4,4,4-trifluorobutan-2-yl)carbamate (Step A) in place of benzyl ((S)-3-(((R)-(2-((1R*,2R)-1-(((R)-tert-butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)(cyclopropyl)methyl)amino)-1,1,1-trifluoropropan-2-yl)carbamate. The product was purified by silica gel chromatography (10-12% MeOH/DCM) to afford the racemic title compound as a yellow oil.

The racemic mixture was separated by SFC ((s,s) Whelk-O1 5 mm, 250×30 mm, isocratic elution: 0.1% NH₁₃H₂O/IPA) to provide two diastereomers after lyophilization. The first eluting isomer, designated R* (Intermediate 199) was isolated as a white solid. The second eluting isomer, designated S* (Intermediate 200) was isolated as a white solid.

### Intermediate 201: (S)-1-((2-((1R,2R)-1-Amino-2-(((R)-1,1,1-trifluoropropan-2-yl)oxy)propyl)-4-fluoro-1H-benzo[d]imidazol-5-yl)methyl)-4-(trifluoromethyl)imidazolidin-2-one

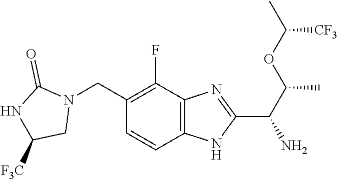 [SMILES: C[C@@H](O[C@H](C)C(F)(F)F)[C@H](N)c1nc2c(F)c(CN3C[C@@H](C(F)(F)F)NC3=O)ccc2[nH]1]

Step A: (S)—N-((1R,2R)-1-(5-Bromo-4-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide. A solution of 5-bromo-4-fluoro-1-((2-(trimethylsilyl)ethoxy)methyl)-1H-benzo[d]imidazole (2.00 g, 5.79 mmol, Intermediate 132) and (R,E)-2-methyl-N—((R)-2-(((R)-1,1,1-trifluoropropan-2-yl)oxy) propylidene)propane-2-sulfinamide (2.53 g, 9.26 mmol, Intermediate 5) in THF (40 mL) was cooled to −65° C. Then, LDA (15.1 mL, 15.1 mmol, 1 M in THF) was added dropwise over 20 min and the resulting mixture stirred at −65° for 1 h. The reaction was quenched with a 1:50 AcOH/THF mixture (20 mL), then extracted with EtOAc (3×50 mL). The organic layers were combined, washed with brine (200 mL), dried over anhydrous Na₂SO₄, filtered and concentrated to dryness to give a yellow oil. The oil was purified by silica gel chromatography (0-0.6% MeOH/DCM) to provide the title compound as a light yellow oil (75% yield).

Step B: (1R,2R)-1-(5-Bromo-4-fluoro-1-((2-(trimethylsilyl)ethoxy)methyl)-1H-benzo[d]imidazol-2-yl)-2-(((R)-1,1,1-trifluoropropan-2-yl)oxy)propan-1-amine. A mixture of (S)—N-((1R,2R)-1-(5-bromo-4-fluoro-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (500 mg, 0.808 mmol, Step A), iodine (102 mg, 0.404 mmol), water (5 mL) and THF (15 mL) was heated at 60° C. for 1.5 h. Then, water (20 mL) and saturated aqueous sodium thiosulfate (20 mL) were added and the mixture extracted with EtOAc (3×50 mL). The organic layers were combined, washed with brine (200 mL), dried over anhydrous Na₂SO₄, filtered and concentrated to dryness to provide the title compound as a yellow solid, which was used without further purification.

Step C: tert-Butyl ((1R,2R)-1-(5-bromo-4-fluoro-1-((2-(trimethylsilyl)ethoxy)methyl)-1H-benzo[d]imidazol-2-yl)-2-(((R)-1,1,1-trifluoropropan-2-yl)oxy)propyl)carbamate. A mixture of (1R,2R)-1-(5-bromo-4-fluoro-1-((2-(trimethylsilyl)ethoxy)methyl)-1H-benzo[d]imidazol-2-yl)-2-(((R)-1,1,1-trifluoropropan-2-yl)oxy)propan-1-amine (400 mg, 0.778 mmol, Step B), Boc₂O (254 mg, 1.17 mmol), DMAP (9.5 mg, 0.078 mmol) and DIPEA (0.406 mL, 2.33 mmol) in DCM (5 mL) was stirred at rt for 1 h. Then, water (10 mL) was added and the mixture extracted with DCM (3×30 mL). The organic layers were combined, washed with brine (100 mL), dried over anhydrous Na₂SO₄, filtered and concentrated to dryness to give a yellow oil. The oil was purified by silica gel chromatography (0-30% EtOAc/petroleum ether) to provide the title compound as a light yellow solid (65% yield).

Step D: tert-Butyl ((1R,2R)-1-(4-fluoro-1-((2-(trimethylsilyl)ethoxy)methyl)-5-vinyl-1H-benzo[d]imidazol-2-yl)-2-(((R)-1,1,1-trifluoropropan-2-yl)oxy)propyl)carbamate. The title compound (60% yield) was prepared as described for the synthesis of Intermediate 133 using tert-butyl ((1R,2R)-1-(5-bromo-4-fluoro-1-((2-(trimethylsilyl)ethoxy)methyl)-1H-benzo[d]imidazol-2-yl)-2-(((R)-1,1,1-trifluoropropan-2-yl)oxy)propyl)carbamate (Step C) in place of 5-bromo-4-fluoro-1-((2-(trimethylsilyl)ethoxy)methyl)-1H-benzo[d]imidazole.

Step E: tert-Butyl ((1R,2R)-1-(4-fluoro-5-formyl-1-((2-(trimethylsilyl)ethoxy)methyl)-1H-benzo[d]imidazol-2-yl)-2-(((R)-1,1,1-trifluoropropan-2-yl)oxy)propyl)carbamate. The title compound was prepared as described for the synthesis of Intermediate 134 using tert-butyl ((1R,2R)-1-(4-fluoro-1-((2-(trimethylsilyl)ethoxy)methyl)-5-vinyl-1H-benzo[d]imidazol-2-yl)-2-(((R)-1,1,1-trifluoropropan-2-yl)oxy)propyl)carbamate (Step D) in place of 4-fluoro-1-((2-(trimethylsilyl)ethoxy)methyl)-5-vinyl-1H-benzo[d]imidazole. The residue was purified by silica gel chromatography (0-30% EtOAc/petroleum ether) to provide the title compound as a white solid (61% yield).

Step F: tert-Butyl ((1R,2R)-1-(5-((((S)-2-amino-3,3,3-trifluoropropyl)amino)methyl)-4-fluoro-1-((2-(trimethylsilyl)ethoxy)methyl)-1H-benzo[d]imidazol-2-yl)-2-(((R)-1,1,1-trifluoropropan-2-yl)oxy)propyl)carbamate. The title compound (99% yield) was prepared as described for the synthesis of Intermediate 64 using tert-butyl ((1R,2R)-1-(4-fluoro-5-formyl-1-((2-(trimethylsilyl)ethoxy)methyl)-1H-benzo[d]imidazol-2-yl)-2-(((R)-1,1,1-trifluoropropan-2-yl)oxy)propyl)carbamate (Step E) in place of 1-((2-(trimethylsilyl)ethoxy)methyl)-1H-benzo[d]imidazole-5-carbaldehyde.

Step G: tert-Butyl ((1R,2R)-1-(4-fluoro-5-(((S)-2-oxo-4-(trifluoromethyl)imidazolidin-1-yl)methyl)-1-((2-(trimethylsilyl)ethoxy)methyl)-1H-benzo[d]imidazol-2-yl)-2-(((R)-1,1,1-trifluoropropan-2-yl)oxy)propyl)carbamate. A mixture of tert-butyl ((1R,2R)-1-(5-((((S)-2-amino-3,3,3-trifluoropropyl)amino)methyl)-4-fluoro-1-((2-(trimethylsilyl)ethoxy)methyl)-1H-benzo[d]imidazol-2-yl)-2-(((R)-1,1,1-trifluoropropan-2-yl)oxy)propyl)carbamate (500 mg, 0.74 mmol, Step F) and CDI (360 mg, 2.22 mmol) in THF (8.2 mL) was heated at 65° C. for 1 h. Then, 1 M aqueous NaOH (2 mL) and water (10 mL) were added and the mixture extracted with EtOAc. The organic layers were combined, washed with brine, dried over anhydrous Na₂SO₄, filtered and concentrated to dryness. The residue was purified by silica gel chromatography (0-30% EtOAc/petroleum ether) to provide the title compound as a white solid (50% yield).

Step H: (S)-1-((2-((1R,2R)-1-Amino-2-(((R)-1,1,1-trifluoropropan-2-yl)oxy)propyl)-4-fluoro-1H-benzo[d]imidazol-5-yl)methyl)-4-(trifluoromethyl)imidazolidin-2-one. A mixture of tert-butyl ((1R,2R)-1-(4-fluoro-5-(((S)-2-oxo-4-(trifluoromethyl)imidazolidin-1-yl)methyl)-1-((2-(trimethylsilyl)ethoxy)methyl)-1H-benzo[d]imidazol-2-yl)-2-(((R)-1,1,1-trifluoropropan-2-yl)oxy)propyl)carbamate (200 mg, 0.285 mmol, Step G) and HCl (3.66 mL, 14.6 mmol, 4 M in 1,4-dioxane) in a sealed tube was heated at 55° C. for 2 h. The mixture was concentrated to dryness and the residue was partitioned between water (10 mL) and MTBE (15 mL). The layers were separated and the aqueous further extracted with MTBE (15 mL). The pH of the aqueous layer was adjusted to pH>10 by the addition of NH₄OH (0.5 mL), and then was extracted with DCM (4×10 mL). The organic layers were combined, dried over anhydrous Na₂SO₄, filtered and concentrated to dryness to give a yellow oil (160 mg). To the yellow oil (160 mg) were added DIPEA (2 mL, 11.5 mmol) and MeOH (2 mL) and the resulting mixture was heated at 80° C. for 1 h. The mixture was concentrated to dryness to provide the title compound as a light yellow solid, which was used without further purification.

### Example 1: 4-Cyclopropyl-N—((R)-1-(5-((R)-cyclopropyl((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide

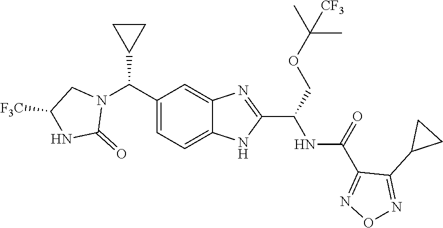 [SMILES: CC(C)(OC[C@H](NC(=O)c1nonc1C1CC1)c1nc2cc([C@@H](C3CC3)N3C[C@@H](C(F)(F)F)NC3=O)ccc2[nH]1)C(F)(F)F]

A round-bottom flask was charged with 4-cyclopropyl-1,2,5-oxadiazole-3-carboxylic acid (19 mg, 0.12 mmol), T₃P (50% in EtOAc) (0.15 mL, 0.24 mmol), DIPEA (0.06 mL, 0.37 mmol), and anhydrous THF (2 mL). (S)-1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one (60 mg, 0.12 mmol, Intermediate 34) was then added to the reaction mixture and the resulting mixture heated at 30° C. for 16 h. After this time, the reaction mixture was poured into water (20 mL) and extracted with EtOAc (10 mL×3). The combined extracts were washed with brine (20 mL), dried over anhydrous Na₂SO₄, filtered, and concentrated to dryness under reduced pressure to give a residue. This residue was purified by preparative HPLC (Welch Xtimate C18 5 μm, 150×30 mm (45-75% CH₃CN/H₂O with 0.05% NH₃₊₁₀ mM NH₄HCO₃)) to afford, after lyophilization, the title compound in 40% yield as a colorless solid. ¹H NMR (400 MHz, DMSO-d₆) δ 12.46 (br s, 1H), 9.63 (br s, 1H), 7.74-7.35 (m, 3H), 7.17 (d, J=5.6 Hz, 1H), 5.47 (br s, 1H), 4.55-4.35 (m, 1H), 4.22 (dd, J=4.4, 9.6 Hz, 1H), 4.15 (d, J=10.0 Hz, 1H), 4.04 (m, 1H), 3.84 (m, 1H), 3.10 (dd, J=3.6, 9.6 Hz, 1H), 2.45-2.37 (m, 1H), 1.55-1.45 (m, 1H), 1.35 (d, J=3.6 Hz, 6H), 1.21-1.12 (m, 2H), 1.06-0.98 (m, 2H), 0.82-0.70 (m, 1H), 0.63-0.54 (m, 1H), 0.47-0.31 (m, 2H). MS (ESI) m/z: [M+H]⁺ Found 630.1.

### Example 2: N—((R)-1-(5-((R)-Cyclopropyl((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

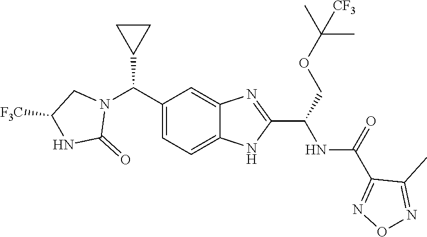 [SMILES: Cc1nonc1C(=O)N[C@@H](COC(C)(C)C(F)(F)F)c1nc2cc([C@@H](C3CC3)N3C[C@@H](C(F)(F)F)NC3=O)ccc2[nH]1]

A solution of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one (34 mg, 0.069 mmol, Intermediate 34), in acetonitrile (1.4 mL) was treated with DIPEA (15 μL, 0.090 mmol) and 2,5-dioxopyrrolidin-1-yl 4-methyl-1,2,5-oxadiazole-3-carboxylate (582 mg, 0.26 mmol, Intermediate 19) and the resulting mixture was stirred at rt for 1 h. After that time, the reaction was quenched with water and extracted three times with EtOAc. The combined organic layers were washed with brine, dried over anhydrous MgSO₄, filtered, and concentrated under reduced pressure to afford a residue. This residue was purified by silica gel chromatography (0-100% EtOAc (10% MeOH)/hexanes) to afford the title compound in 69% yield, which was lyophilized into an off-white solid. ¹H NMR (600 MHz, DMSO-d₆) δ 12.40 (d, J=8.7 Hz, 1H), 9.54 (dd, J=8.4, 4.7 Hz, 1H), 7.71-7.51 (m, 1H), 7.51-7.41 (m, 2H), 7.21-7.09 (m, 1H), 5.49-5.38 (m, 1H), 4.53-4.35 (m, 1H), 4.21 (dd, J=9.7, 4.7 Hz, 1H), 4.15 (dd, J=10.2, 4.1 Hz, 1H), 4.04 (t, J=9.2 Hz, 1H), 3.87-3.80 (m, 1H), 3.14-3.06 (m, 1H), 2.52 (s, 3H), 1.55-1.45 (m, 1H), 1.35 (s, 3H), 1.34 (s, 3H), 0.79-0.72 (m, 1H), 0.63-0.56 (m, 1H), 0.45-0.33 (m, 2H). MS (ESI) m/z: [M+H]⁺ Found 604.3.

### Example 3: N—((R)-1-(5-((R)-Cyclopropyl((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-4-fluoro-1-isopropyl-1H-pyrazole-5-carboxamide

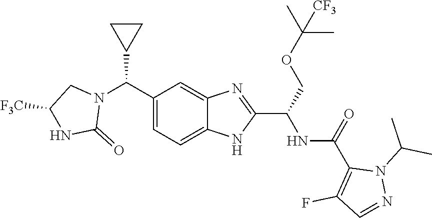 [SMILES: CC(C)n1ncc(F)c1C(=O)N[C@@H](COC(C)(C)C(F)(F)F)c1nc2cc([C@@H](C3CC3)N3C[C@@H](C(F)(F)F)NC3=O)ccc2[nH]1]

To a stirred solution of 4-fluoro-1-isopropyl-1H-pyrazole-5-carboxylic acid (33.5 mg, 0.195 mmol) and 1-propanephosphonic anhydride (0.116 mL, 0.195 mmol, 50% in EtOAc) in EtOAc (0.6 mL) at rt was added N,N-diisopropylethylamine (67.8 μL, 0.389 mmol). After 3 min, (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one (60.0 mg, 0.122 mmol, Intermediate 34) and DCM (0.6 mL) were added. After stirring for 2 h at rt, the reaction mixture was diluted with water (5 mL) and EtOAc (5 mL). The aqueous portion was extracted with EtOAc (3×5 mL). The combined organic layers were washed with saturated aqueous NaHCO₃ (10 mL) and brine (20 mL), dried over anhydrous Na₂SO₄, filtered, and concentrated to afford the product. This product was initially purified by preparative HPLC using Waters XBridge BEH C18, 5 μm, 19×100 mm, 40-70% MeCN/water (with 24 mM NH₄OH). The resulting product was further purified by preparative HPLC using Waters XSelect CSH C18, 5 μm, 19×100 mm, 25-50% MeCN (0.16% TFA)/water (0.16% TFA). The product containing fractions were concentrated to remove MeCN, the pH adjusted to pH 7 with saturated aqueous NaHCO₃, extracted three times with EtOAc, concentrated under reduced pressure, and lyophilized to afford the title compound in 34% yield as a white powder. ¹H NMR (600 MHz, DMSO-d₆) δ 12.40 (s, 1H), 8.52-8.41 (m, 1H), 7.64 (d, J=4.5 Hz, 1H), 7.59-7.38 (m, 2H), 7.16 (d, J=8.3 Hz, 1H), 5.53-5.35 (m, 1H), 5.24-5.12 (m, 1H), 4.50-4.39 (m, 1H), 4.18-4.08 (m, 2H), 4.04 (dd, J=9.5, 7.6 Hz, 1H), 3.84 (t, J=10.0 Hz, 1H), 3.11 (dd, J=10.2, 4.2 Hz, 1H), 2.48-2.46 (m, 1H), 1.55-1.44 (m, 1H), 1.36 (dd, J=6.6, 4.3 Hz, 6H), 1.32 (d, J=9.7 Hz, 6H), 0.82-0.70 (m, 1H), 0.63-0.53 (m, 1H), 0.48-0.31 (m, 2H). MS (ESI) m/z: [M+H]⁺ Found 648.2.

### Example 4: N—((R)-1-(5-((R)-Cyclopropyl((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1-(ethyl-d₅)-1H-pyrazole-5-carboxamide

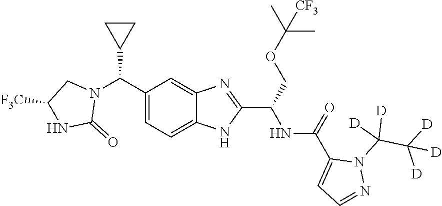 [SMILES: [2H]C([2H])([2H])C([2H])([2H])n1nccc1C(=O)N[C@@H](COC(C)(C)C(F)(F)F)c1nc2cc([C@@H](C3CC3)N3C[C@@H](C(F)(F)F)NC3=O)ccc2[nH]1]

The title compound was prepared as described for the synthesis of Example 3 using ethyl 1-(ethyl-d₅)-1H-pyrazole-5-carboxylate (Intermediate 21) in place of 4-fluoro-1-isopropyl-1H-pyrazole-5-carboxylic acid. This product was purified by preparative HPLC (Waters XBridge BEH C18, 5 μm, 19×100 mm, 35-65% MeCN/water (with 24 mM NH₄OH)) to afford the title compound in 44% yield as a white powder after lyophilization. ¹H NMR (600 MHz, DMSO-d₆) δ 12.40 (d, J=3.3 Hz, 1H), 8.90 (dd, J=8.5, 6.3 Hz, 1H), 7.65-7.55 (m, 1H), 7.51-7.42 (m, 3H), 7.20-7.12 (m, 1H), 6.96 (t, J=2.2 Hz, 1H), 5.43 (td, J=8.5, 5.0 Hz, 1H), 4.51-4.37 (m, 1H), 4.20 (dd, J=9.6, 5.1 Hz, 1H), 4.16 (dd, J=10.2, 3.7 Hz, 1H), 4.00 (t, J=9.1 Hz, 1H), 3.84 (td, J=10.0, 3.4 Hz, 1H), 3.17-3.06 (m, 1H), 1.58-1.44 (m, 1H), 1.34 (d, J=2.5 Hz, 6H), 0.76 (tt, J=8.4, 4.6 Hz, 1H), 0.66-0.54 (m, 1H), 0.47-0.31 (m, 2H). MS (ESI) m/z: [M+H]⁺ Found 621.3.

### Example 5: N—((R)-1-(5-((R)-Cyclopropyl((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1-isopropyl-1H-1,2,4-triazole-5-carboxamide

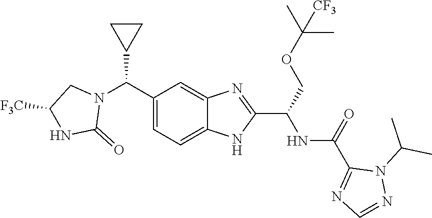 [SMILES: CC(C)n1ncnc1C(=O)N[C@@H](COC(C)(C)C(F)(F)F)c1nc2cc([C@@H](C3CC3)N3C[C@@H](C(F)(F)F)NC3=O)ccc2[nH]1]

To a stirred solution of 1-isopropyl-1H-1,2,4-triazole-5-carboxylic acid (37.7 mg, 0.243 mmol) and 1-propanephosphonic anhydride (0.145 mL, 0.243 mmol, 50% in EtOAc) in EtOAc (0.76 mL) at rt was added N,N-diisopropylethylamine (84.7 μL, 0.486 mmol). After 3 min, (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one (75.0 mg, 0.152 mmol, Intermediate 34) and DCM (0.76 mL) were added. After stirring for 20 min at rt, additional 1-isopropyl-1H-1,2,4-triazole-5-carboxylic acid (37.7 mg, 0.243 mmol), 1-propanephosphonic anhydride (0.145 mL, 0.243 mmol, 50% in EtOAc), and N,N-diisopropylethylamine (84.7 μL, 0.486 mmol) were sequentially added. After 2 h at rt, the reaction mixture was diluted with water (5 mL) and EtOAc (5 mL). The aqueous portion was extracted with EtOAc (3×5 mL). The combined organic layers were washed with saturated aqueous NaHCO₃ twice and brine (10 mL), dried over anhydrous Na₂SO₄, filtered, and concentrated to afford the product. This product was purified by preparative HPLC (Waters XBridge BEH C18, 5 μm, 19×100 mm, 35-65% MeCN/water (with 24 mM NH₄OH)) and the product containing fractions were lyophilized to afford the title compound in 28% yield as a white powder. ¹H NMR (600 MHz, DMSO-d₆) δ 12.38 (d, J=6.5 Hz, 1H), 9.02 (d, J=8.5 Hz, 1H), 8.14 (s, 1H), 7.71-7.53 (m, 1H), 7.51-7.39 (m, 2H), 7.15 (dd, J=19.5, 8.4 Hz, 1H), 5.62 (hept, J=6.7 Hz, 1H), 5.46-5.39 (m, 1H), 4.50-4.38 (m, 1H), 4.20-4.11 (m, 2H), 4.09 (t, J=8.7 Hz, 1H), 3.83 (t, J=10.0 Hz, 1H), 3.14-3.01 (m, 1H), 1.58-1.45 (m, 1H), 1.42 (dd, J=6.6, 4.0 Hz, 6H), 1.31 (d, J=15.5 Hz, 6H), 0.80-0.69 (m, 1H), 0.63-0.54 (m, 1H), 0.46-0.29 (m, 2H). MS (ESI) m/z: [M+H]⁺ Found 631.2.

### Example 6: 4-Methyl-N—((R*)-1-(5-((R)-1-((S)-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-1,2,5-oxadiazole-3-carboxamide

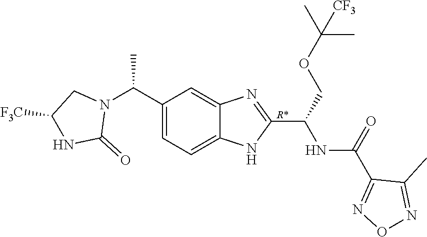 [SMILES: Cc1nonc1C(=O)N[C@@H](COC(C)(C)C(F)(F)F)c1nc2cc([C@@H](C)N3C[C@@H](C(F)(F)F)NC3=O)ccc2[nH]1]

A round-bottom flask was charged with (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one (100 mg, 0.186 mmol, Intermediate 42), 4-methyl-1,2,5-oxadiazole-3-carboxylic acid (35.7 mg, 0.278 mmol), MeCN (5 mL), HOBt (37.6 mg, 0.278 mmol), and DIPEA (92.0 μL, 0.557 mmol) and the mixture was stirred at 45° C. for 5 min. EDCI (53.4 mg, 0.278 mmol) was then added, and the reaction mixture was stirred for an additional 2 h at 50° C. After cooling to rt, the reaction was treated with saturated aqueous NaHCO₃ (1 mL), water (10 mL), and EtOAc (5 mL). The layers were separated and the aqueous layers were extracted with EtOAc (10 mL×2). The combined organic layers were washed with brine (10 mL), dried over anhydrous Na₂SO₄, filtered, and concentrated to dryness under reduced pressure. This product was purified by silica gel chromatography (0-0.8% MeOH/DCM) followed by SFC (DAICEL CHIRALPAK AD, 10 μm, 250×30 mm, 75% CO₂ in EtOH (0.1% of 25% aqueous NH₃)) to give, after lyophilization, the title compound in 49% yield as a white powder. ¹H NMR (400 MHz, DMSO-d₆) δ 12.43-12.41 (m, 1H), 9.59-9.56 (m, 1H), 7.59-7.51 (m, 1H), 7.49-7.34 (m, 2H), 7.11-7.09 (m, 1H), 5.47-5.42 (m, 1H), 5.16-5.10 (m, 1H), 4.40-4.35 (m, 1H), 4.25-4.18 (m, 1H), 4.06-4.01 (m, 1H), 3.73-3.64 (m, 1H), 2.93-2.87 (m, 1H), 2.52 (s, 3H), 1.52-1.50 (m, 3H), 1.37-1.32 (m, 6H). MS (ESI) m/z: [M+H]⁺ Found 578.3.

### Example 7: 4-Cyclopropyl-N—((R*)-1-(5-((R)-1-((S)-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-1,2,5-oxadiazole-3-carboxamide

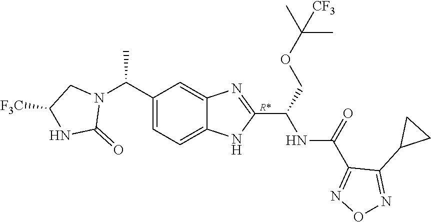 [SMILES: C[C@H](c1ccc2[nH]c([C@H](COC(C)(C)C(F)(F)F)NC(=O)c3nonc3C3CC3)nc2c1)N1C[C@@H](C(F)(F)F)NC1=O]

The title compound was prepared as described for the synthesis of Example 2 using (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one (Intermediate 42) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate provided the title compound in 88% yield. ¹H NMR (600 MHz, DMSO-d₆) δ 12.43 (d, J=12.9 Hz, 1H), 9.64-9.53 (m, 1H), 7.60-7.31 (m, 3H), 7.16-7.06 (m, 1H), 5.52-5.42 (m, 1H), 5.19-5.07 (m, 1H), 4.44-4.29 (m, 1H), 4.26-4.16 (m, 1H), 4.04 (t, J=9.2 Hz, 1H), 3.74-3.62 (m, 1H), 2.95-2.87 (m, 1H), 2.43-2.38 (m, 1H), 1.51 (d, J=7.2 Hz, 3H), 1.34 (d, J=4.9 Hz, 6H), 1.19-1.13 (m, 2H), 1.05-0.98 (m, 2H). MS (ESI) m/z: [M+H]⁺ Found 604.2.

### Example 8: N-((1R*,2R)-1-(5-((R)-Cyclopropyl((R*)-2-oxo-4-(trifluoromethyl)imidazolidin-1-yl-5,5-d₂)methyl)-1H-benzo[d]imidazol-2-yl)-2-(((R)-1,1,1-trifluoropropan-2-yl)oxy)propyl)-4-methyl-1,2,5-oxadiazole-3-carboxamide

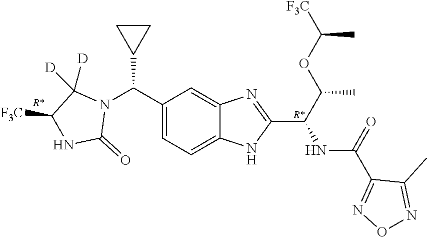 [SMILES: [2H]C1([2H])[C@H](C(F)(F)F)NC(=O)N1[C@@H](c1ccc2[nH]c([C@@H](NC(=O)c3nonc3C)[C@@H](C)O[C@H](C)C(F)(F)F)nc2c1)C1CC1]

### Example 9: N-((1R*,2R)-1-(5-((R)-Cyclopropyl((S*)-2-oxo-4-(trifluoromethyl)imidazolidin-1-yl-5,5-d₂)methyl)-1H-benzo[d]imidazol-2-yl)-2-(((R)-1,1,1-trifluoropropan-2-yl)oxy)propyl)-4-methyl-1,2,5-oxadiazole-3-carboxamide

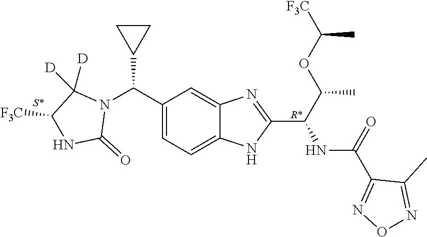 [SMILES: [2H]C1([2H])[C@@H](C(F)(F)F)NC(=O)N1[C@@H](c1ccc2[nH]c([C@@H](NC(=O)c3nonc3C)[C@@H](C)O[C@H](C)C(F)(F)F)nc2c1)C1CC1]

N-((1R*,2R)-1-(5-((R)-Cyclopropyl((R*)-2-oxo-4-(trifluoromethyl)imidazolidin-1-yl-5,5-d₂)methyl)-1H-benzo[d]imidazol-2-yl)-2-(((R)-1,1,1-trifluoropropan-2-yl)oxy)propyl)-4-methyl-1,2,5-oxadiazole-3-carboxamide (Example 8) and N-((1R*,2R)-1-(5-((R)-cyclopropyl((S*)-2-oxo-4-(trifluoromethyl)imidazolidin-1-yl-5,5-d₂)methyl)-1H-benzo[d]imidazol-2-yl)-2-(((R)-1,1,1-trifluoropropan-2-yl)oxy)propyl)-4-methyl-1,2,5-oxadiazole-3-carboxamide (Example 9) were prepared as described for the synthesis of Example 3 using 1-((R)-(2-((1R*,2R)-1-amino-2-(((R)-1,1,1-trifluoropropan-2-yl)oxy)propyl)-1H-benzo[d]imidazol-5-yl)(cyclopropyl)methyl)-4-(trifluoromethyl)imidazolidin-2-one-5,5-d₂ (Intermediate 57) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 4-methyl-1,2,5-oxadiazole-3-carboxylic acid in place of 4-fluoro-1-isopropyl-1H-pyrazole-5-carboxylic acid. These diastereomers were purified by silica gel chromatography (0-100% EtOAc (10% MeOH)/hexanes) and then separated by chiral SFC (Chiralpak IB N₃ 5 μm, 250×21 mm, 85% CO₂ in MeOH) to afford N-((1R*,2R)-1-(5-((R)-cyclopropyl((R*)-2-oxo-4-(trifluoromethyl)imidazolidin-1-yl-5,5-d₂)methyl)-1H-benzo[d]imidazol-2-yl)-2-(((R)-1,1,1-trifluoropropan-2-yl)oxy)propyl)-4-methyl-1,2,5-oxadiazole-3-carboxamide (Example 8) as the first eluting isomer in 15% yield and N-((1R*,2R)-1-(5-((R)-cyclopropyl((S*)-2-oxo-4-(trifluoromethyl)imidazolidin-1-yl-5,5-d₂)methyl)-1H-benzo[d]imidazol-2-yl)-2-(((R)-1,1,1-trifluoropropan-2-yl)oxy)propyl)-4-methyl-1,2,5-oxadiazole-3-carboxamide (Example 9) as the second eluting isomer in 20% yield. Data for Example 8: ¹H NMR (400 MHz, DMSO-d₆) δ 12.44 (d, J=8.2 Hz, 1H), 9.43 (dd, J=9.0, 6.5 Hz, 1H), 7.67-7.37 (m, 3H), 7.26-7.15 (m, 1H), 5.35-5.28 (m, 1H), 4.49-4.26 (m, 3H), 4.14 (dd, J=10.1, 3.0 Hz, 1H), 2.50 (s, 3H), 1.64-1.51 (m, 1H), 1.15 (d, J=6.2 Hz, 3H), 1.09 (d, J=6.5 Hz, 3H), 0.73-0.63 (m, 1H), 0.62-0.51 (m, 1H), 0.44-0.32 (m, 1H), 0.32-0.25 (m, 1H). MS (ESI) m/z: [M+H]⁺ Found 606.3. Data for Example 9: ¹H NMR (400 MHz, DMSO-d₆) δ 12.45 (d, J=4.4 Hz, 1H), 9.49-9.34 (m, 1H), 7.71-7.41 (m, 3H), 7.23-7.11 (m, 1H), 5.38-5.25 (m, 1H), 4.47-4.36 (m, 2H), 4.34-4.24 (m, 1H), 4.15 (dd, J=10.2, 3.5 Hz, 1H), 2.50 (s, 3H), 1.62-1.40 (m, 1H), 1.15 (dd, J=6.3, 2.3 Hz, 3H), 1.06 (dd, J=6.5, 3.0 Hz, 3H), 0.82-0.70 (m, 1H), 0.64-0.52 (m, 1H), 0.49-0.28 (m, 2H). MS (ESI) m/z: [M+H]⁺ Found 606.3.

### Example 10: N-((1R*,2R)-1-(5-((R)-Cyclopropyl((S)-2-oxo-4-(trifluoromethyl)imidazolidin-1-yl)methyl)-1H-benzo[d]imidazol-2-yl)-2-(((R)-1,1,1-trifluoropropan-2-yl)oxy)propyl)-4-methyl-1,2,5-oxadiazole-3-carboxamide

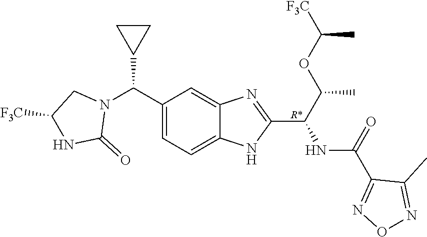 [SMILES: Cc1nonc1C(=O)N[C@H](c1nc2cc([C@@H](C3CC3)N3C[C@@H](C(F)(F)F)NC3=O)ccc2[nH]1)[C@@H](C)O[C@H](C)C(F)(F)F]

The title compound was prepared as described for the synthesis of Example 3 using (S)-1-((R)-(2-((1R*,2R)-1-amino-2-(((R)-1,1,1-trifluoropropan-2-yl)oxy)propyl)-1H-benzo[d]imidazol-5-yl)(cyclopropyl)methyl)-4-(trifluoromethyl)imidazolidin-2-one (Intermediate 55) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 4-methyl-1,2,5-oxadiazole-3-carboxylic acid in place of 4-fluoro-1-isopropyl-1H-pyrazole-5-carboxylic acid. The product was purified by silica gel chromatography (0-100% EtOAc (10% MeOH)/hexanes) to afford the title compound in 48% yield as an off-white solid. ¹H NMR (400 MHz, DMSO-d₆) δ 12.64-12.22 (m, 1H), 9.55-9.28 (m, 1H), 7.73-7.37 (m, 3H), 7.30-7.07 (m, 1H), 5.32 (dd, J=9.0, 6.7 Hz, 1H), 4.55-4.37 (m, 2H), 4.37-4.25 (m, 1H), 4.15 (dd, J=10.4, 3.2 Hz, 1H), 3.97-3.60 (m, 1H), 3.20-3.00 (m, 1H), 2.50 (s, 3H), 1.64-1.36 (m, 1H), 1.20-1.11 (m, 3H), 1.10-1.02 (m, 3H), 0.82-0.68 (m, 1H), 0.64-0.52 (m, 1H), 0.49-0.30 (m, 2H). MS (ESI) m/z: [M+H]⁺ Found 604.2.

### Example 11: N-((1R*,2R)-1-(5-((R)-Cyclopropyl((R*)-2-oxo-4-(trifluoromethyl)imidazolidin-1-yl-5,5-d₂)methyl)-1H-benzo[d]imidazol-2-yl)-2-(((S)-1,1,1-trifluoropropan-2-yl)oxy)propyl)-4-methyl-1,2,5-oxadiazole-3-carboxamide

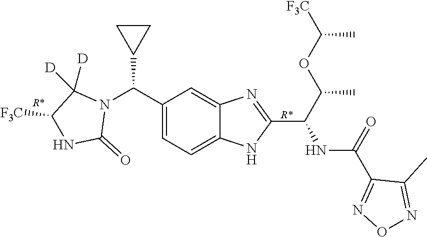 [SMILES: [2H]C1([2H])[C@@H](C(F)(F)F)NC(=O)N1[C@@H](c1ccc2[nH]c([C@@H](NC(=O)c3nonc3C)[C@@H](C)O[C@@H](C)C(F)(F)F)nc2c1)C1CC1]

### Example 12: N-((1R*,2R)-1-(5-((R)-Cyclopropyl((S*)-2-oxo-4-(trifluoromethyl)imidazolidin-1-yl-5,5-d₂)methyl)-1H-benzo[d]imidazol-2-yl)-2-(((S)-1,1,1-trifluoropropan-2-yl)oxy)propyl)-4-methyl-1,2,5-oxadiazole-3-carboxamide

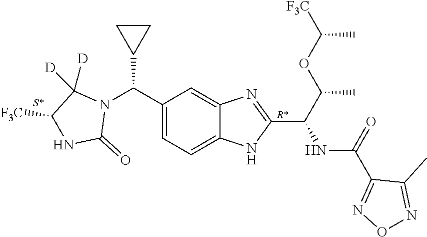 [SMILES: [2H]C1([2H])[C@@H](C(F)(F)F)NC(=O)N1[C@@H](c1ccc2[nH]c([C@@H](NC(=O)c3nonc3C)[C@@H](C)O[C@@H](C)C(F)(F)F)nc2c1)C1CC1]

N-((1R*,2R)-1-(5-((R)-Cyclopropyl((R*)-2-oxo-4-(trifluoromethyl)imidazolidin-1-yl-5,5-d₂)methyl)-1H-benzo[d]imidazol-2-yl)-2-(((S)-1,1,1-trifluoropropan-2-yl)oxy)propyl)-4-methyl-1,2,5-oxadiazole-3-carboxamide (Example 11) and N-((1R*,2R)-1-(5-((R)-cyclopropyl((S*)-2-oxo-4-(trifluoromethyl)imidazolidin-1-yl-5,5-d₂)methyl)-1H-benzo[d]imidazol-2-yl)-2-(((S)-1,1,1-trifluoropropan-2-yl)oxy)propyl)-4-methyl-1,2,5-oxadiazole-3-carboxamide (Example 12) were prepared as described for the synthesis of Example 3 using 1-((R)-(2-((1R*,2R)-1-amino-2-(((S)-1,1,1-trifluoropropan-2-yl)oxy)propyl)-1H-benzo[d]imidazol-5-yl)(cyclopropyl)methyl)-4-(trifluoromethyl)imidazolidin-2-one-5,5-d₂ (Intermediate 61) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 4-methyl-1,2,5-oxadiazole-3-carboxylic acid in place of 4-fluoro-1-isopropyl-1H-pyrazole-5-carboxylic acid. These diastereomers were purified by silica gel chromatography (0-100% EtOAc (10% MeOH)/hexanes) and then separated by chiral SFC (Chiralpak IB N3, 5 μm, 250×21 mm, 85% CO₂ in MeOH) to afford N-((1R*,2R)-1-(5-((R)-cyclopropyl((R*)-2-oxo-4-(trifluoromethyl)imidazolidin-1-yl-5,5-d₂)methyl)-1H-benzo[d]imidazol-2-yl)-2-(((S)-1,1,1-trifluoropropan-2-yl)oxy)propyl)-4-methyl-1,2,5-oxadiazole-3-carboxamide (Example 11) as the first eluting isomer in 10% yield and N-((1R*,2R)-1-(5-((R)-cyclopropyl((S*)-2-oxo-4-(trifluoromethyl)imidazolidin-1-yl-5,5-d₂)methyl)-1H-benzo[d]imidazol-2-yl)-2-(((S)-1,1,1-trifluoropropan-2-yl)oxy)propyl)-4-methyl-1,2,5-oxadiazole-3-carboxamide (Example 12) as the second eluting isomer in 12% yield. Data for Example 11: ¹H NMR (400 MHz, DMSO-d₆) δ 12.39 (d, J=7.5 Hz, 1H), 9.31-9.15 (m, 1H), 7.73-7.33 (m, 3H), 7.20 (d, J=8.3 Hz, 1H), 5.32 (dd, J=8.9, 7.2 Hz, 1H), 4.46-4.23 (m, 3H), 4.13 (d, J=10.2 Hz, 1H), 2.48 (s, 3H), 1.65-1.50 (m, 1H), 1.26 (d, J=6.4 Hz, 3H), 1.15 (d, J=6.2 Hz, 3H), 0.74-0.63 (m, 1H), 0.62-0.52 (m, 1H), 0.37 (s, 1H), 0.33-0.23 (m, 1H). MS (ESI) m/z: [M+H]⁺ Found 606.2. Data for Example 12: ¹H NMR (400 MHz, DMSO-d₆) δ 12.53-12.27 (m, 1H), 9.20 (d, J=8.9 Hz, 1H), 7.67-7.40 (m, 3H), 7.30-7.11 (m, 1H), 5.33 (dd, J=9.0, 6.9 Hz, 1H), 4.57-4.25 (m, 3H), 4.25-4.04 (m, 1H), 2.49 (s, 3H), 1.57-1.42 (m, 1H), 1.26 (d, J=6.5 Hz, 3H), 1.17 (d, J=6.2 Hz, 3H), 0.83-0.69 (m, 1H), 0.66-0.54 (m, 1H), 0.50-0.30 (m, 2H). MS (ESI) m/z: [M+H]⁺ Found 606.2.

### Example 13: N-((1R*,2R)-1-(5-((R)-Cyclopropyl((S)-2-oxo-4-(trifluoromethyl)imidazolidin-1-yl)methyl)-1H-benzo[d]imidazol-2-yl)-2-(((S)-1,1,1-trifluoropropan-2-yl)oxy)propyl)-4-methyl-1,2,5-oxadiazole-3-carboxamide

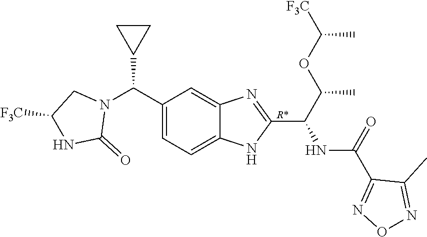 [SMILES: Cc1nonc1C(=O)N[C@H](c1nc2cc([C@@H](C3CC3)N3C[C@@H](C(F)(F)F)NC3=O)ccc2[nH]1)[C@@H](C)O[C@@H](C)C(F)(F)F]

The title compound was prepared as described for the synthesis of Example 2 using (S)-1-((R)-(2-((1R*,2R)-1-amino-2-(((S)-1,1,1-trifluoropropan-2-yl)oxy)propyl)-1H-benzo[d]imidazol-5-yl)(cyclopropyl)methyl)-4-(trifluoromethyl)imidazolidin-2-one (Intermediate 63) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one providing the title compound in 91% yield. ¹H NMR (500 MHz, DMSO-d₆) δ 12.40 (d, J=8.0 Hz, 1H), 9.19 (d, J=8.7 Hz, 1H), 7.68-7.40 (m, 3H), 7.23-7.12 (m, 1H), 5.33 (dd, J=9.0, 7.0 Hz, 1H), 4.50-4.39 (m, 1H), 4.38-4.28 (m, 2H), 4.15 (dd, J=10.3, 4.0 Hz, 1H), 3.84 (t, J=10.0 Hz, 1H), 3.21-3.05 (m, 1H), 2.49 (s, 3H), 1.58-1.43 (m, 1H), 1.25 (d, J=6.5 Hz, 3H), 1.18-1.13 (m, 3H), 0.82-0.71 (m, 1H), 0.65-0.54 (m, 1H), 0.49-0.31 (m, 2H). MS (ESI) m/z: [M+H]⁺ Found 604.4.

### Example 14: N-((1R*,2R)-1-(5-((R)-Cyclopropyl((S)-2-oxo-4-(trifluoromethyl)imidazolidin-1-yl)methyl)-1H-benzo[d]imidazol-2-yl)-2-(((S)-1,1,1-trifluoropropan-2-yl)oxy)propyl)-1-methyl-1H-pyrazole-5-carboxamide

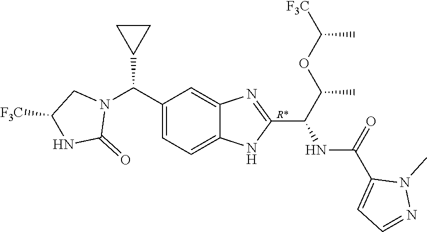 [SMILES: C[C@H](O[C@H](C)[C@H](NC(=O)c1ccnn1C)c1nc2cc([C@@H](C3CC3)N3C[C@@H](C(F)(F)F)NC3=O)ccc2[nH]1)C(F)(F)F]

To a stirred solution of 1-methyl-1H-pyrazole-5-carboxylic acid (12.3 mg, 97.3 μmol) and 1-propanephosphonic anhydride (57.9 μL, 97.3 μmol, 50% in EtOAc) in EtOAc (0.3 mL) at rt was added DIPEA (33.9 μL, 0.195 mmol). After 3 min, a mixture of (S)-1-((R)-(2-((1R*,2R)-1-amino-2-(((S)-1,1,1-trifluoropropan-2-yl)oxy)propyl)-1H-benzo[d]imidazol-5-yl)(cyclopropyl)methyl)-4-(trifluoromethyl)imidazolidin-2-one (30.0 mg, 60.8 μmol, Intermediate 63) in DCM (0.3 mL) was added. After stirring for 1 h at rt, additional 1-methyl-1H-pyrazole-5-carboxylic acid (12.3 mg, 97.3 μmol), 1-propanephosphonic anhydride (57.9 μL, 97.3 μmol, 50% in EtOAc), and DIPEA (33.9 μL, 0.195 mmol) were added. After an additional 1 h, 1-methyl-1H-pyrazole-5-carboxylic acid (12.3 mg, 97.3 μmol), 1-propanephosphonic anhydride (57.9 μL, 97.3 μmol, 50% in EtOAc), and DIPEA (33.9 μL, 0.195 mmol) were added and stirred for 2 h. The reaction mixture was partitioned between water (10 mL) and EtOAc (5 mL). The aqueous portion was further extracted with EtOAc (2×10 mL). The combined organic layers were washed with saturated aqueous NaHCO₃ solution twice followed by brine (10 mL), dried over anhydrous Na₂SO₄, filtered, and concentrated under reduced pressure to afford the product. This product was purified by silica gel chromatography (0-100% EtOAc (10% MeOH)/hexanes) followed by preparative HPLC (Waters XSelect CSH C18, 5 μm, 19×100 mm, 10-45% MeCN (0.16% TFA)/water (0.16% TFA)) and the product containing fractions were concentrated to remove MeCN, the pH adjusted to pH 7 with saturated aqueous NaHCO₃, extracted three times with EtOAc, concentrated under reduced pressure and lyophilized to afford the title compound in 19% yield. ¹H NMR (600 MHz, DMSO-d₆) δ 12.57-12.19 (m, 1H), 8.73 (d, J=9.1 Hz, 1H), 7.63-7.43 (m, 4H), 7.21-7.14 (m, 1H), 6.97 (s, 1H), 5.34-5.24 (m, 1H), 4.49-4.40 (m, 1H), 4.41-4.33 (m, 1H), 4.33-4.27 (m, 1H), 4.19-4.11 (m, 1H), 4.02 (s, 3H), 3.84 (t, J=10.0 Hz, 1H), 3.16-3.07 (m, 1H), 1.53-1.45 (m, 1H), 1.26 (d, J=6.5 Hz, 3H), 1.13 (d, J=6.1 Hz, 3H), 0.82-0.67 (m, 1H), 0.64-0.31 (m, 3H). MS (ESI) m/z: [M+H]⁺ Found 602.3.

### Example 15: N-((1R*,2R)-1-(5-((R)-Cyclopropyl((S)-2-oxo-4-(trifluoromethyl)imidazolidin-1-yl)methyl)-1H-benzo[d]imidazol-2-yl)-2-(((S)-1,1,1-trifluoropropan-2-yl)oxy)propyl)-1-isopropyl-1H-pyrazole-5-carboxamide

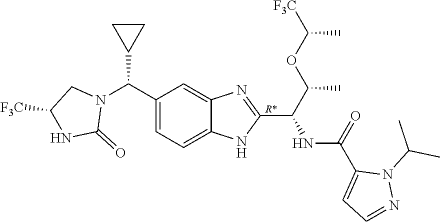 [SMILES: CC(C)n1nccc1C(=O)N[C@H](c1nc2cc([C@@H](C3CC3)N3C[C@@H](C(F)(F)F)NC3=O)ccc2[nH]1)[C@@H](C)O[C@@H](C)C(F)(F)F]

The title compound was prepared as described for the synthesis of Example 3 using (S)-1-((R)-(2-((1R*,2R)-1-amino-2-(((S)-1,1,1-trifluoropropan-2-yl)oxy)propyl)-1H-benzo[d]imidazol-5-yl)(cyclopropyl)methyl)-4-(trifluoromethyl)imidazolidin-2-one (Intermediate 63) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 1-isopropyl-1H-pyrazole-5-carboxylic acid in place of 4-fluoro-1-isopropyl-1H-pyrazole-5-carboxylic acid. The product was purified by silica gel chromatography (0-100% EtOAc (10% MeOH)/hexanes) to afford the title compound in 36% yield, after lyophilization, as a white solid. ¹H NMR (500 MHz, DMSO-d₆) δ 12.49-12.28 (m, 1H), 8.68 (d, J=9.0 Hz, 1H), 7.68-7.37 (m, 4H), 7.23-7.07 (m, 1H), 6.89 (d, J=2.0 Hz, 1H), 5.48-5.35 (m, 1H), 5.35-5.26 (m, 1H), 4.49-4.39 (m, 1H), 4.39-4.25 (m, 2H), 4.15 (d, J=10.2 Hz, 1H), 3.92-3.77 (m, 1H), 3.13 (dd, J=10.0, 4.2 Hz, 1H), 1.55-1.43 (m, 1H), 1.34 (dd, J=15.4, 6.6 Hz, 6H), 1.26 (d, J=6.5 Hz, 3H), 1.13 (d, J=6.2 Hz, 3H), 0.82-0.70 (m, 1H), 0.64-0.55 (m, 1H), 0.53-0.40 (m, 1H), 0.40-0.27 (m, 1H). MS (ESI) m/z: [M+H]⁺ Found 630.5.

### Example 16: 4-Cyclopropyl-N—((S)-1-(5-(((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide

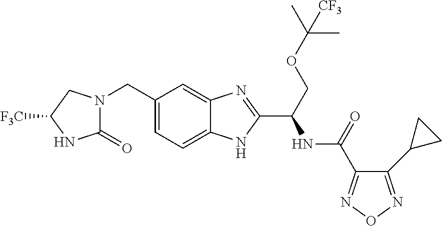 [SMILES: CC(C)(OC[C@@H](NC(=O)c1nonc1C1CC1)c1nc2cc(CN3C[C@@H](C(F)(F)F)NC3=O)ccc2[nH]1)C(F)(F)F]

### Example 17: 4-Cyclopropyl-N—((R)-1-(5-(((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide

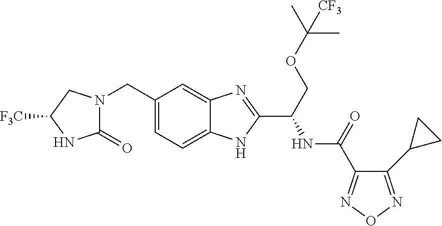 [SMILES: CC(C)(OC[C@H](NC(=O)c1nonc1C1CC1)c1nc2cc(CN3C[C@@H](C(F)(F)F)NC3=O)ccc2[nH]1)C(F)(F)F]

4-Cyclopropyl-N—((S)-1-(5-(((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide (Example 16) and 4-cyclopropyl-N—((R)-1-(5-(((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide (Example 17) were prepared as described for the synthesis of Example 2 using (S)-1-((2-((R)-1-amino-2-((1,1,1-trifluoro-2-methylpropan-2-yl)oxy)ethyl)-1H-benzo[d]imidazol-5-yl)methyl)-4-(trifluoromethyl)imidazolidin-2-one (Intermediate 67) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. These diastereomers were purified by silica gel chromatography (0-100% EtOAc (with 10% MeOH)/hexanes) and then separated by chiral SFC (Chiralpak IA3, 5 μm, 250×21 mm, 80% CO₂ in MeOH) to afford 4-cyclopropyl-N—((S)-1-(5-(((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide (Example 16) as the first eluting fraction in 3% yield and 4-cyclopropyl-N—((R)-1-(5-(((S)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide (Example 17) in 28% yield as the second eluting fraction. Data for Example 16: ¹H NMR (600 MHz, DMSO-d₆) δ 12.44 (d, J=15.6 Hz, 1H), 9.60 (d, J=8.3 Hz, 1H), 7.60-7.30 (m, 3H), 7.12-7.01 (m, 1H), 5.50-5.41 (m, 1H), 4.45-4.28 (m, 3H), 4.23-4.17 (m, 1H), 4.04 (t, J=9.2 Hz, 1H), 3.50 (t, J=10.0 Hz, 1H), 3.26-3.19 (m, 1H), 2.43-2.38 (m, 1H), 1.34 (d, J=4.9 Hz, 6H), 1.18-1.12 (m, 2H), 1.04-0.98 (m, 2H). MS (ESI) m/z: [M+H]⁺ Found 590.2. Data for Example 17: ¹H NMR (600 MHz, DMSO-d₆) δ 12.44 (d, J=14.7 Hz, 1H), 9.60 (d, J=8.4 Hz, 1H), 7.61-7.32 (m, 3H), 7.10-7.02 (m, 1H), 5.46 (td, J=8.5, 4.7 Hz, 1H), 4.46-4.27 (m, 3H), 4.21 (dd, J=9.7, 4.8 Hz, 1H), 4.04 (t, J=9.2 Hz, 1H), 3.50 (t, J=10.0 Hz, 1H), 3.26-3.19 (m, 1H), 2.43-2.37 (m, 1H), 1.34 (d, J=5.1 Hz, 6H), 1.19-1.13 (m, 2H), 1.05-0.99 (m, 2H). MS (ESI) m/z: [M+H]⁺ Found 590.2.

### Example 18: N—((R*)-1-(5-((R)-Cyclopropyl(5,5-difluoro-2-oxotetrahydropyrimidin-1(2H)-yl)methyl)-1H-imidazo[4,5-b]pyridin-2-yl)-2-((1,1,1-trifluoro-2-methylpropan-2-yl)oxy)ethyl)-4-methyl-1,2,5-oxadiazole-3-carboxamide

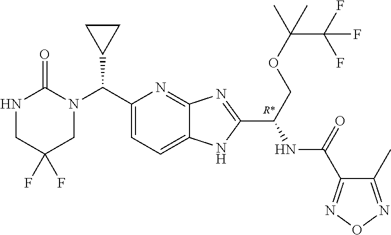 [SMILES: Cc1nonc1C(=O)N[C@@H](COC(C)(C)C(F)(F)F)c1nc2nc([C@@H](C3CC3)N3CC(F)(F)CNC3=O)ccc2[nH]1]

A vial was charged with 1-((R)-(2-((R*)-1-amino-2-((1,1,1-trifluoro-2-methylpropan-2-yl)oxy)ethyl)-1H-imidazo[4,5-b]pyridin-5-yl)(cyclopropyl)methyl)-5,5-difluorotetrahydropyrimidin-2(1H)-one (50 mg, 0.10 mmol, Intermediate 98), MeCN (3 mL), DIPEA (24 μL, 0.136 mmol), and 2,5-dioxopyrrolidin-1-yl 4-methyl-1,2,5-oxadiazole-3-carboxylate (24 mg, 0.11 mmol, Intermediate 19). The reaction was stirred for 15 min at rt. The reaction mixture was purified by silica gel chromatography (0-100% ethyl acetate (with 10% MeOH)/hexanes). The diastereomeric mixture was separated by SFC with a chiral stationary phase (Stationary phase: Chiralpak IA 5 μm, 250×21 mm, Mobile phase: 15% Methanol, 85% CO₂). The second eluting peak afforded the title compound as a white solid (0.09 mmol, 89% yield). ¹H NMR (400 MHz, DMSO-d₆) δ 13.11-12.67 (m, 1H), 9.70-9.41 (m, 1H), 8.05-7.77 (m, 1H), 7.21 (d, J=8.1 Hz, 1H), 6.76-6.60 (m, 1H), 5.54-5.41 (m, 1H), 4.70 (d, J=10.0 Hz, 1H), 4.32-4.22 (m, 1H), 4.10-4.00 (m, 1H), 3.98-3.84 (m, 1H), 3.85-3.69 (m, 1H), 3.60-3.39 (m, 2H), 2.55-2.52 (m, 3H), 1.64-1.50 (m, 1H), 1.40-1.31 (m, 6H), 0.69-0.55 (m, 2H), 0.49-0.41 (m, 1H), 0.39-0.29 (m, 1H). MS (ESI) m/z: [M+H]⁺ Found 587.3.

### Example 19: N-((1R*,2R)-1-(5-((R)-Cyclopropyl(5,5-difluoro-2-oxotetrahydropyrimidin-1(2H)-yl)methyl)-1H-imidazo[4,5-b]pyridin-2-yl)-2-(((R)-1,1,1-trifluoropropan-2-yl)oxy)propyl)-4-methyl-1,2,5-oxadiazole-3-carboxamide

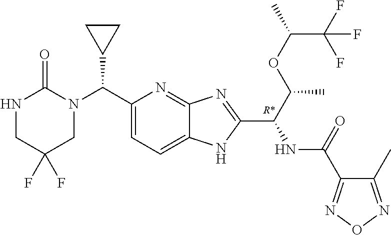 [SMILES: Cc1nonc1C(=O)N[C@H](c1nc2nc([C@@H](C3CC3)N3CC(F)(F)CNC3=O)ccc2[nH]1)[C@@H](C)O[C@H](C)C(F)(F)F]

The title compound was prepared as described for the synthesis of Example 18 using 1-((R)-(2-((1R*,2R)-1-amino-2-(((R)-1,1,1-trifluoropropan-2-yl)oxy)propyl)-1H-imidazo[4,5-b]pyridin-5-yl)(cyclopropyl)methyl)-5,5-difluorotetrahydropyrimidin-2(1H)-one (Intermediate 104) in place of 1-((R)-(2-((R*)-1-amino-2-((1,1,1-trifluoro-2-methylpropan-2-yl)oxy)ethyl)-1H-imidazo[4,5-b]pyridine-5-yl)(cyclopropyl)methyl)-5,5-difluorotetrahydropyrimidin-2(1H)-one to afford the product as a glassy solid. The material was purified by SFC using a chiral stationary phase (Stationary phase: Chiralpak IH, 5 μm, 250×21 mm, Mobile phase: 10% Methanol, 90% CO₂). The second eluting component afforded the title compound, Example 19, as a white powder (50% yield). ¹H NMR (400 MHz, DMSO-d₆) δ 13.29-12.65 (m, 1H), 9.65-9.24 (m, 1H), 8.08-7.76 (m, 1H), 7.31-7.12 (m, 1H), 6.83-6.60 (m, 1H), 5.40-5.30 (m, 1H), 4.69 (d, J=10.1 Hz, 1H), 4.49-4.39 (m, 1H), 4.39-4.26 (m, 1H), 3.98-3.68 (m, 2H), 3.60-3.40 (m, 2H), 3.32 (s, 3H), 1.62-1.49 (m, 1H), 1.27-1.14 (m, 3H), 1.12-1.00 (m, 3H), 0.72-0.56 (m, 2H), 0.51-0.42 (m, 1H), 0.39-0.26 (m, 1H). MS (ESI) m/z: [M+H]⁺ Found 587.2.

### Example 20: 4-Cyclopropyl-N-((1R*,2R)-1-(5-((R)-cyclopropyl(5,5-difluoro-2-oxotetrahydropyrimidin-1(2H)-yl)methyl)-1H-imidazo[4,5-b]pyridin-2-yl)-2-(((R)-1,1,1-trifluoropropan-2-yl)oxy)propyl)-1,2,5-oxadiazole-3-carboxamide

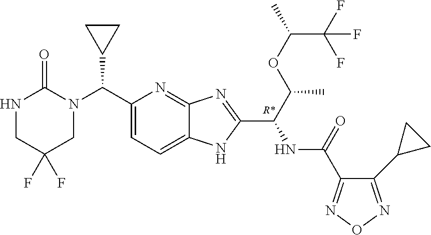 [SMILES: C[C@@H](O[C@H](C)C(F)(F)F)[C@H](NC(=O)c1nonc1C1CC1)c1nc2nc([C@@H](C3CC3)N3CC(F)(F)CNC3=O)ccc2[nH]1]

The title compound was prepared as described for Example 6 using 1-((R)-(2-((1R*,2R)-1-amino-2-(((R)-1,1,1-trifluoropropan-2-yl)oxy)propyl)-1H-imidazo[4,5-b]pyridin-5-yl)(cyclopropyl)methyl)-5,5-difluorotetrahydropyrimidin-2(1H)-one (Intermediate 104) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one, and 4-cyclopropyl-1,2,5-oxadiazole-3-carboxylic acid in place of 4-methyl-1,2,5-oxadiazole-3-carboxylic acid to afford a mixture of diastereomers. These diastereomers were separated by SFC with a chiral stationary phase (Stationary phase: Chiralpak IH, 5 μm, 250×21 mm, Mobile phase: 10% Methanol, 90% CO₂). The second eluting peak was isolated to afford the title compound, Example 20, as a white powder (41% yield). ¹H NMR (400 MHz, DMSO-d₆) δ 13.37-12.65 (m, 1H), 9.73-9.26 (m, 1H), 8.09-7.78 (m, 1H), 7.35-7.09 (m, 1H), 6.81-6.61 (m, 1H), 5.43-5.33 (m, 1H), 4.77-4.65 (m, 1H), 4.48-4.28 (m, 2H), 3.99-3.68 (m, 2H), 3.60-3.41 (m, 2H), 2.42-2.32 (m, 1H), 1.63-1.49 (m, 1H), 1.23-1.18 (m, 3H), 1.18-1.13 (m, 2H), 1.13-1.07 (m, 3H), 1.06-0.96 (m, 2H), 0.69-0.56 (m, 2H), 0.53-0.42 (m, 1H), 0.40-0.28 (m, 1H). MS (ESI) m/z: [M+H]⁺ Found 613.2.

### Example 21: N-((1R*,2R)-1-(5-((S)-1-(5,5-Difluoro-2-oxotetrahydropyrimidin-1(2H)-yl)-2-methoxyethyl)-1H-imidazo[4,5-b]pyridin-2-yl)-2-(((R)-1,1,1-trifluoropropan-2-yl)oxy)propyl)-4-methyl-1,2,5-oxadiazole-3-carboxamide

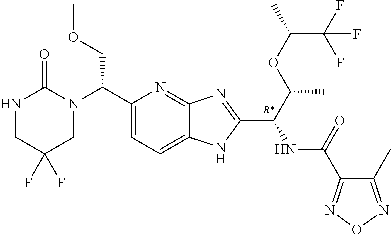 [SMILES: COC[C@H](c1ccc2[nH]c([C@@H](NC(=O)c3nonc3C)[C@@H](C)O[C@H](C)C(F)(F)F)nc2n1)N1CC(F)(F)CNC1=O]

The title compound was prepared as described for the synthesis of Example 18 using 1-((S)-1-(2-((1R*,2R)-1-amino-2-(((R)-1,1,1-trifluoropropan-2-yl)oxy)propyl)-1H-imidazo[4,5-b]pyridin-5-yl)-2-methoxyethyl)-5,5-difluorotetrahydropyrimidin-2(1H)-one (Intermediate 114) in place of 1-((R)-(2-((R*)-1-amino-2-((1,1,1-trifluoro-2-methylpropan-2-yl)oxy)ethyl)-1H-imidazo[4,5-b]pyridin-5-yl)(cyclopropyl)methyl)-5,5-difluorotetrahydropyrimidin-2(1H)-one to afford the title compound as a white powder (16% yield). ¹H NMR (400 MHz, DMSO-d₆) δ 13.25-12.64 (m, 1H), 9.58-9.19 (m, 1H), 8.10-7.82 (m, 1H), 7.20 (d, J=8.1 Hz, 1H), 6.86-6.76 (m, 1H), 5.79-5.71 (m, 1H), 5.39-5.30 (m, 1H), 4.50-4.39 (m, 1H), 4.38-4.27 (m, 1H), 3.97-3.87 (m, 2H), 3.75-3.38 (m, 5H), 2.62-2.59 (m, 1H), 2.51 (br s, 3H), 1.28-1.13 (m, 4H), 1.10-1.00 (m, 3H). MS (ESI) m/z: [M+H]⁺ Found 591.2.

### Example 22: 4-Cyclopropyl-N—((R)-1-(5-((R)-cyclopropyl(5,5-difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)-1,2,5-oxadiazole-3-carboxamide

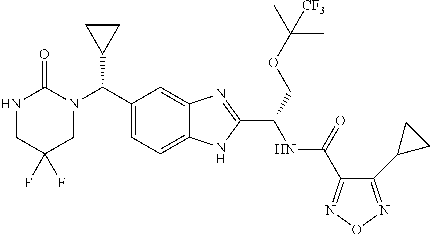 [SMILES: CC(C)(OC[C@H](NC(=O)c1nonc1C1CC1)c1nc2cc([C@@H](C3CC3)N3CC(F)(F)CNC3=O)ccc2[nH]1)C(F)(F)F]

To a stirred solution of 4-cyclopropyl-1,2,5-oxadiazole-3-carboxylic acid (87.5 mg, 0.568 mmol) and 1-propanephosphonic anhydride (0.313 mL, 0.526 mmol, 50% in EtOAc) in EtOAc (1.05 mL) was added N,N-diisopropylethylamine (0.180 mL, 1.05 mmol). After 5 min, 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one (100 mg, 0.21 mmol, Intermediate 37) was added as a solution in DCM (2.0 mL). After 16 h, the reaction mixture was diluted with aqueous HCl (15 mL, 0.05 M) and EtOAc (8 mL). The aqueous layer was extracted with EtOAc (3×8 mL). The combined organics were washed with aqueous 0.1 M NaOH (10 mL) and brine (10 mL), dried over anhydrous Na₂SO₄, filtered and concentrated to give a residue which was dissolved in DMSO (3.5 mL) and purified by preparative HPLC (C18, 5 μm, 50×250 mm, 20-85% MeCN/water (20 mM NH₄OH)) to give the title compound (32.5% yield) as a white solid. ¹H NMR (600 MHz, DMSO-d₆) δ 12.42 (d, J=5.7 Hz, 1H), 9.59 (dd, J=4.6, 8.4 Hz, 1H), 7.71-7.41 (m, 2H), 7.23-7.17 (m, 1H), 6.75 (d, J=13.7 Hz, 1H), 5.46 (dt, J=4.8, 8.4 Hz, 1H), 4.72 (d, J=10.3 Hz, 1H), 4.22 (dd, J=4.6, 9.5 Hz, 1H), 4.05 (t, J=9.2 Hz, 1H), 3.79-3.65 (m, 1H), 3.61-3.51 (m, 1H), 3.51-3.42 (m, 1H), 3.26-3.17 (m, 1H), 2.43-2.38 (m, 1H), 1.54-1.45 (m, 1H), 1.36 (s, 3H), 1.35 (s, 3H), 1.19-1.14 (m, 2H), 1.03-1.00 (m, 2H), 0.81-0.76 (m, 1H), 0.61-0.55 (m, 1H), 0.43-0.34 (m, 2H). MS (ESI) m/z: [M+H]⁺ Found 612.2.

### Example 23: N—((R)-1-(5-((R)-Cyclopropyl(5,5-difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

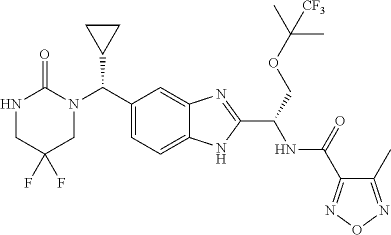 [SMILES: Cc1nonc1C(=O)N[C@@H](COC(C)(C)C(F)(F)F)c1nc2cc([C@@H](C3CC3)N3CC(F)(F)CNC3=O)ccc2[nH]1]

The title compound was prepared as described for the synthesis of Example 22 using 4-methyl-1,2,5-oxadiazole-3-carboxylic acid in place of 4-cyclopropyl-1,2,5-oxadiazole-3-carboxylic acid to provide the title compound as a white solid (26.6% yield). ¹H NMR (600 MHz, DMSO-d₆) δ 12.38 (d, J=8.2 Hz, 1H), 9.54 (dd, J=4.2, 8.4 Hz, 1H), 7.74-7.39 (m, 2H), 7.26-7.16 (m, 1H), 6.74 (d, J=13.9 Hz, 1H), 5.44 (dt, J=4.8, 8.4 Hz, 1H), 4.71 (d, J=10.3 Hz, 1H), 4.26-4.17 (m, 1H), 4.04 (t, J=9.2 Hz, 1H), 3.79-3.68 (m, 1H), 3.62-3.51 (m, 1H), 3.50-3.42 (m, 1H), 3.26-3.17 (m, 1H), 2.52 (s, 3H), 1.54-1.44 (m, 1H), 1.36 (s, 3H), 1.35 (s, 3H), 0.82-0.76 (m, 1H), 0.60-0.54 (m, 1H), 0.44-0.32 (m, 2H). MS (ESI) m/z: [M+H]⁺ Found 586.2.

### Example 24: N—((R)-1-(5-((R)-Cyclopropyl(5,5-difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)-1-isopropyl-1H-pyrazole-5-carboxamide

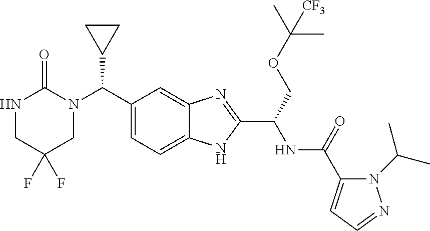 [SMILES: CC(C)n1nccc1C(=O)N[C@@H](COC(C)(C)C(F)(F)F)c1nc2cc([C@@H](C3CC3)N3CC(F)(F)CNC3=O)ccc2[nH]1]

The title compound was prepared as described for the synthesis of Example 22 using 1-isopropyl-1H-pyrazole-5-carboxylic acid in place of 4-cyclopropyl-1,2,5-oxadiazole-3-carboxylic acid to provide the title compound as a white solid (26.8% yield). ¹H NMR (600 MHz, DMSO-d₆) δ 12.37 (d, J=3.2 Hz, 1H), 8.87 (dd, J=7.0, 8.3 Hz, 1H), 7.71-7.39 (m, 3H), 7.23-7.16 (m, 1H), 6.92-6.89 (m, 1H), 6.74 (d, J=14.3 Hz, 1H), 5.53-5.39 (m, 2H), 4.72 (d, J=10.3 Hz, 1H), 4.22-4.15 (m, 1H), 3.99 (t, J=9.1 Hz, 1H), 3.78-3.66 (m, 1H), 3.61-3.50 (m, 1H), 3.50-3.43 (m, 1H), 3.26-3.15 (m, 1H), 1.54-1.44 (m, 1H), 1.38 (dd, J=4.5, 6.6 Hz, 6H), 1.34 (br. s, 3H), 1.34 (br s, 3H), 0.82-0.75 (m, 1H), 0.61-0.54 (m, 1H), 0.44-0.32 (m, 2H). MS (ESI) m/z: [M+H]⁺ Found 612.2.

### Example 25: 4-Cyclopropyl-N—((R)-1-(5-((R*)-1-(5,5-difluoro-2-oxotetrahydropyrimidin-1(2H)-yl)ethyl)-1H-benzo[d]imidazol-2-yl)-2-((1,1,1-trifluoro-2-methylpropan-2-yl)oxy)ethyl)-1,2,5-oxadiazole-3-carboxamide

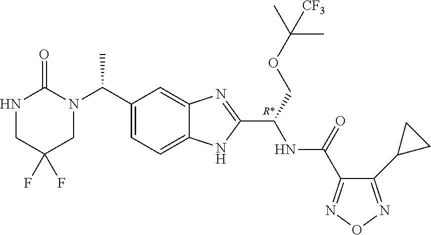 [SMILES: C[C@H](c1ccc2[nH]c([C@H](COC(C)(C)C(F)(F)F)NC(=O)c3nonc3C3CC3)nc2c1)N1CC(F)(F)CNC1=O]

The title compound was prepared as described for the synthesis of Example 22 using 1-((R)-1-(2-((R*)-1-amino-2-((1,1,1-trifluoro-2-methylpropan-2-yl)oxy)ethyl)-1H-benzo[d]imidazol-5-yl)ethyl)-5,5-difluorotetrahydropyrimidin-2(1H)-one (Intermediate 44) in place of 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one to provide the title compound as a white solid (21.1% yield). ¹H NMR (500 MHz, DMSO-d₆) δ 12.43 (d, J=9.8 Hz, 1H), 9.62-9.56 (m, 1H), 7.59-7.53 (m, 1H), 7.46-7.37 (m, 1H), 7.16-7.10 (m, 1H), 6.78-6.72 (m, 1H), 5.75-5.68 (m, 1H), 5.50-5.43 (m, 1H), 4.24-4.18 (m, 1H), 4.07-4.01 (m, 1H), 3.61-3.36 (m, 3H), 3.05-2.93 (m, 1H), 2.44-2.37 (m, 1H), 1.48 (d, J=7.1 Hz, 3H), 1.35 (d, J=4.4 Hz, 6H), 1.19-1.14 (m, 2H), 1.03-0.99 (m, 2H). MS (ESI) m/z: [M+H]⁺ Found 586.3.

### Example 26: N—((R)-1-(5-((R*)-1-(5,5-Difluoro-2-oxotetrahydropyrimidin-1(2H)-yl)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

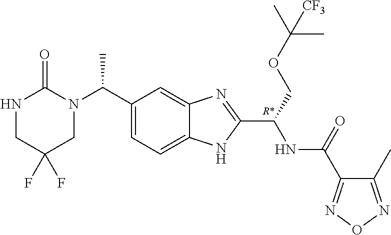 [SMILES: Cc1nonc1C(=O)N[C@@H](COC(C)(C)C(F)(F)F)c1nc2cc([C@@H](C)N3CC(F)(F)CNC3=O)ccc2[nH]1]

The title compound was prepared as described for the synthesis of Example 22 using 1-((R)-1-(2-((R*)-1-amino-2-((1,1,1-trifluoro-2-methylpropan-2-yl)oxy)ethyl)-1H-benzo[d]imidazol-5-yl)ethyl)-5,5-difluorotetrahydropyrimidin-2(1H)-one (Intermediate 44) in place of 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one and 4-methyl-1,2,5-oxadiazole-3-carboxylic acid in place of 4-cyclopropyl-1,2,5-oxadiazole-3-carboxylic acid as the starting material to provide the title compound as a white solid (23% yield). ¹H NMR (500 MHz, DMSO-d₆) δ 12.39 (d, J=11.2 Hz, 1H), 9.57-9.52 (m, 1H), 7.57-7.53 (m, 1H), 7.46-7.37 (m, 1H), 7.16-7.09 (m, 1H), 6.79-6.72 (m, 1H), 5.75-5.68 (m, 1H), 5.47-5.40 (m, 1H), 4.24-4.19 (m, 1H), 4.07-4.00 (m, 1H), 3.60-3.37 (m, 3H), 3.05-2.93 (m, 1H), 2.52 (s, 3H), 1.48 (d, J=7.2 Hz, 3H), 1.35 (d, J=4.3 Hz, 6H). MS (ESI) m/z: [M+H]⁺ Found 560.3.

### Example 27: N—((R)-1-(5-((R*)-1-(5,5-Difluoro-2-oxotetrahydropyrimidin-1(2H)-yl)ethyl)-1H-benzo[d]imidazol-2-yl)-2-((1,1,1-trifluoro-2-methylpropan-2-yl)oxy)ethyl)-1-isopropyl-1H-pyrazole-5-carboxamide

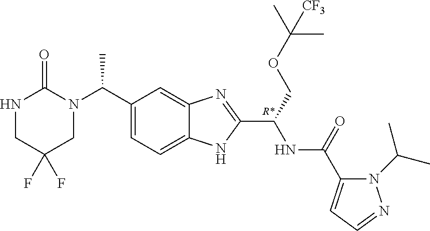 [SMILES: CC(C)n1nccc1C(=O)N[C@@H](COC(C)(C)C(F)(F)F)c1nc2cc([C@@H](C)N3CC(F)(F)CNC3=O)ccc2[nH]1]

The title compound was prepared as described for the synthesis of Example 22 using 1-((R)-1-(2-((R*)-1-amino-2-((1,1,1-trifluoro-2-methylpropan-2-yl)oxy)ethyl)-1H-benzo[d]imidazol-5-yl)ethyl)-5,5-difluorotetrahydropyrimidin-2(1H)-one (Intermediate 44) in place of 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one and 1-isopropyl-1H-pyrazole-5-carboxylic acid in place of 4-cyclopropyl-1,2,5-oxadiazole-3-carboxylic acid as the starting material to provide the title compound as a white solid (15.5% yield). ¹H NMR (500 MHz, DMSO-d₆) δ 12.38 (d, J=7.8 Hz, 1H), 8.92-8.84 (m, 1H), 7.57-7.49 (m, 2H), 7.45-7.36 (m, 1H), 7.16-7.08 (m, 1H), 6.90 (d, J=2.0 Hz, 1H), 6.78-6.71 (m, 1H), 5.75-5.69 (m, 1H), 5.51-5.40 (m, 2H), 4.22-4.15 (m, 1H), 4.03-3.96 (m, 1H), 3.60-3.36 (m, 3H), 3.03-2.91 (m, 1H), 1.48 (d, J=7.1 Hz, 3H), 1.39-1.36 (m, 6H), 1.35-1.33 (m, 6H). MS (ESI) m/z: [M+H]⁺ Found 586.3.

### Example 28: (R)—N-(1-(6-((5,5-Difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

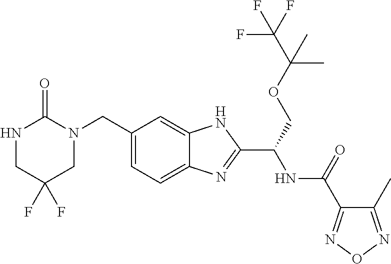 [SMILES: Cc1nonc1C(=O)N[C@@H](COC(C)(C)C(F)(F)F)c1nc2ccc(CN3CC(F)(F)CNC3=O)cc2[nH]1]

The title compound (35% yield) was prepared as described for the synthesis of Example 6 using (R)-1-((2-(1-amino-2-((1,1,1-trifluoro-2-methylpropan-2-yl)oxy)ethyl)-1H-benzo[d]imidazol-5-yl)methyl)-5,5-difluorotetrahydropyrimidin-2(1H)-one (Intermediate 73) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one. ¹H NMR (500 MHz, DMSO-d₆) δ 12.69-12.12 (m, 1H), 9.62-9.46 (m, 1H), 7.66-7.30 (m, 2H), 7.20-7.02 (m, 1H), 6.88-6.73 (m, 1H), 5.52-5.36 (m, 1H), 4.58-4.43 (m, 2H), 4.29-4.15 (m, 1H), 4.11-3.96 (m, 1H), 3.61-3.42 (m, 4H), 2.53-2.51 (m, 3H), 1.43-1.27 (m, 6H). MS (ESI) m/z: [M+H]⁺ Found 546.2.

### Example 29: (R)—N-(1-(6-((5,5-Difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)-1-isopropyl-1H-pyrazole-5-carboxamide

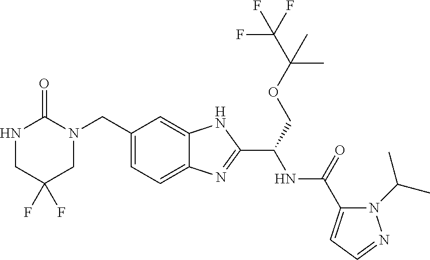 [SMILES: CC(C)n1nccc1C(=O)N[C@@H](COC(C)(C)C(F)(F)F)c1nc2ccc(CN3CC(F)(F)CNC3=O)cc2[nH]1]

The title compound (30% yield) was prepared as described for the synthesis of Example 6 using (R)-1-((2-(1-amino-2-((1,1,1-trifluoro-2-methylpropan-2-yl)oxy)ethyl)-1H-benzo[d]imidazol-5-yl)methyl)-5,5-difluorotetrahydropyrimidin-2(1H)-one (Intermediate 73) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one and 1-isopropyl-1H-pyrazole-5-carboxylic acid in place of 4-methyl-1,2,5-oxadiazole-3-carboxylic acid. ¹H NMR (500 MHz, DMSO-d₆) δ 12.52-12.25 (m, 1H), 8.92-8.84 (m, 1H), 7.60-7.34 (m, 3H), 7.16-7.01 (m, 1H), 6.93-6.86 (m, 1H), 6.83-6.75 (m, 1H), 5.54-5.36 (m, 2H), 4.55-4.47 (m, 2H), 4.23-4.14 (m, 1H), 4.04-3.85 (m, 1H), 3.57-3.44 (m, 4H), 1.41-1.35 (m, 6H), 1.35-1.31 (m, 6H). MS (ESI) m/z: [M+H]⁺ Found 572.2.

### Example 30: (R)-4-Cyclopropyl-N-(1-(6-((5,5-difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)-1,2,5-oxadiazole-3-carboxamide

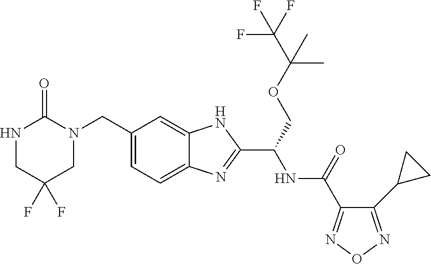 [SMILES: CC(C)(OC[C@H](NC(=O)c1nonc1C1CC1)c1nc2ccc(CN3CC(F)(F)CNC3=O)cc2[nH]1)C(F)(F)F]

The title compound (31% yield) was prepared as described for the synthesis of Example 6 using (R)-1-((2-(1-amino-2-((1,1,1-trifluoro-2-methylpropan-2-yl)oxy)ethyl)-1H-benzo[d]imidazol-5-yl)methyl)-5,5-difluorotetrahydropyrimidin-2(1H)-one (Intermediate 73) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one and 4-cyclopropyl-1,2,5-oxadiazole-3-carboxylic acid in place of 4-methyl-1,2,5-oxadiazole-3-carboxylic acid. ¹H NMR (500 MHz, DMSO-d₆) δ 12.52-12.32 (m, 1H), 9.70-9.46 (m, 1H), 7.62-7.34 (m, 2H), 7.22-7.02 (m, 1H), 6.87-6.73 (m, 1H), 5.53-5.39 (m, 1H), 4.58-4.45 (m, 2H), 4.26-4.17 (m, 1H), 4.04 (t, J=9.1 Hz, 1H), 3.57-3.44 (m, 4H), 2.45-2.37 (m, 1H), 1.40-1.30 (m, 6H), 1.19-1.13 (m, 2H), 1.06-0.98 (m, 2H). MS (ESI) m/z: [M+H]⁺ Found 572.2.

### Example 31: N—((R*)-1-(5-((S)-2-Methoxy-1-((S)-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

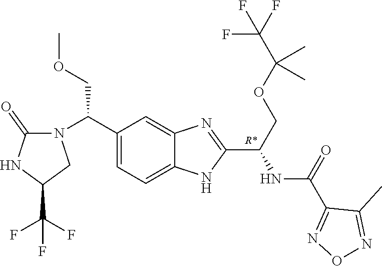 [SMILES: COC[C@H](c1ccc2[nH]c([C@H](COC(C)(C)C(F)(F)F)NC(=O)c3nonc3C)nc2c1)N1C[C@@H](C(F)(F)F)NC1=O]

The title compound (42% yield) was prepared as described for the synthesis of Example 2 using (S)-1-((S)-1-(2-((R*)-1-amino-2-((1,1,1-trifluoro-2-methylpropan-2-yl)oxy)ethyl)-1H-benzo[d]imidazol-5-yl)-2-methoxyethyl)-4-(trifluoromethyl)imidazolidin-2-one (Intermediate 80) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. ¹H NMR (500 MHz, DMSO-d₆) δ 13.11-11.9 (m, 1H), 10.14-9.01 (m, 1H), 7.61-7.32 (m, 3H), 7.13-7.00 (m, 1H), 5.52-5.38 (m, 1H), 5.22-5.08 (m, 1H), 4.48-4.36 (m, 1H), 4.24-4.17 (m, 1H), 4.08-3.99 (m, 1H), 3.94-3.86 (m, 1H), 3.81-3.71 (m, 2H), 3.33 (s, 3H), 3.23-3.18 (m, 1H), 2.52-2.51 (m, 3H), 1.37-1.31 (m, 6H). MS (ESI) m/z: [M+H]⁺ Found 608.2.

### Example 32: 4-Fluoro-1-isopropyl-N—((R)-1-(5-(((S*)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1H-pyrazole-5-carboxamide

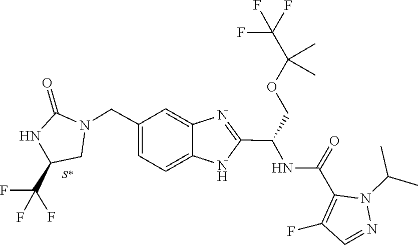 [SMILES: CC(C)n1ncc(F)c1C(=O)N[C@@H](COC(C)(C)C(F)(F)F)c1nc2cc(CN3C[C@@H](C(F)(F)F)NC3=O)ccc2[nH]1]

The title compound was prepared as described for the synthesis of Example 6 using (S*)-1-((2-((R)-1-amino-2-((1,1,1-trifluoro-2-methylpropan-2-yl)oxy)ethyl)-1H-benzo[d]imidazol-5-yl)methyl)-4-(trifluoromethyl)imidazolidin-2-one (Intermediate 85) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one and 4-fluoro-1-isopropyl-1H-pyrazole-5-carboxylic acid in place of 4-methyl-1,2,5-oxadiazole-3-carboxylic acid. Purification by preparative HPLC (XBridge C18 OBD 50×100 mm (5-95% CH₃CN/H₂O with 20 mM NH₄OH)) provided the title compound in 60% yield. ¹H NMR (400 MHz, DMSO-d₆) δ 12.41 (br s, 1H), 8.60-8.38 (m, 1H), 7.64 (d, J=4.5 Hz, 1H), 7.58-7.27 (m, 3H), 7.10-6.99 (m, 1H), 5.51-5.37 (m, 1H), 5.20-5.13 (m, 1H), 4.44-4.28 (m, 3H), 4.11 (dd, J=9.5, 4.8 Hz, 1H), 4.03 (t, J=8.6 Hz, 1H), 3.50 (t, J=10.0 Hz, 1H), 3.26-3.18 (m, 1H), 1.38-1.34 (m, 6H), 1.34-1.30 (m, 6H). MS (ESI) m/z: [M+H]⁺ Found 608.2.

### Example 33: 4-Cyclopropyl-N—((R)-1-(5-(((R*)-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide

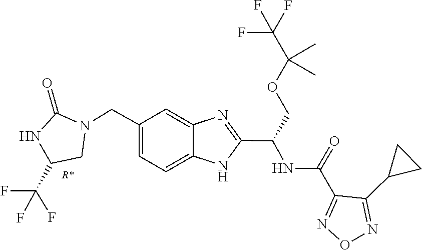

A mixture of (R*)-1-((2-((R)-1-amino-2-((1,1,1-trifluoro-2-methylpropan-2-yl)oxy)ethyl)-1H-benzo[d]imidazol-5-yl)methyl)-4-(trifluoromethyl)imidazolidin-2-one (50 mg, 0.09 mmol, Intermediate 86), 4-cyclopropyl-1,2,5-oxadiazole-3-carboxylic acid (21 mg, 0.14 mmol), HOBt (14 mg, 0.09 mmol), and DIPEA (0.047 mL, 0.27 mmol) in MeCN (0.5 mL) was heated to 45° C. EDCI (26 mg, 0.14 mmol) was added, the reaction mixture was stirred for 12 h 45° C., then quenched with H₂O and filtered. Purification by preparative HPLC (XBridge C18 OBD 50×100 mm (5-95% CH₃CN/H₂O with 20 mM NH₄OH)) provided the title compound in 45% yield. ¹H NMR (400 MHz, DMSO-d₆) δ 12.52-12.36 (m, 1H), 9.64-9.54 (m, 1H), 7.59-7.32 (m, 3H), 7.11-7.00 (m, 1H), 5.46 (td, J=8.3, 4.8 Hz, 1H), 4.46-4.27 (m, 3H), 4.23-4.17 (m, 1H), 4.04 (t, J=9.1 Hz, 1H), 3.50 (t, J=9.9 Hz, 1H), 3.28-3.18 (m, 1H), 2.49-2.36 (m, 1H), 1.34 (d, J=3.9 Hz, 6H), 1.30-1.11 (m, 2H), 1.07-0.93 (m, 2H). MS (ESI) m/z: [M+H]⁺ Found 590.1.

### Example 34: N—((R*)-1-(5-((S)-1-(5,5-Difluoro-2-oxotetrahydropyrimidin-1(2H)-yl)-2-methoxyethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

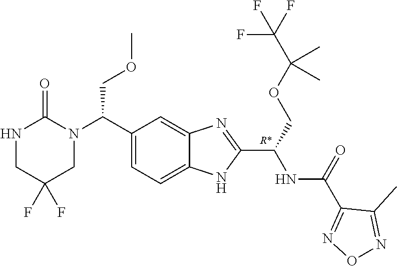 [SMILES: COC[C@H](c1ccc2[nH]c([C@H](COC(C)(C)C(F)(F)F)NC(=O)c3nonc3C)nc2c1)N1CC(F)(F)CNC1=O]

To a solution of 1-((S)-1-(2-((R*)-1-amino-2-((1,1,1-trifluoro-2-methylpropan-2-yl)oxy)ethyl)-1H-benzo[d]imidazol-5-yl)-2-methoxyethyl)-5,5-difluorotetrahydropyrimidin-2(1H)-one (67 mg, 0.14 mmol, Intermediate 82) and 4-methyl-1,2,5-oxadiazole-3-carboxylic acid (27 mg, 0.21 mmol) in CH₂Cl₂ (1.4 mL) was added DIPEA (0.072 mL, 0.42 mmol) followed by T₃P (50% in EtOAc) (0.13 mL, 0.21 mmol). The resulting mixture was stirred at rt for 1 h then diluted with CH₂Cl₂, washed with saturated aqueous NaHCO₃ solution. The combined organic extracts were dried over anhydrous Na₂SO₄, filtered, and concentrated under reduced pressure. Purification by silica gel chromatography (10-100% (10% MeOH in EtOAc)/hexanes) followed by preparative HPLC (XBridge C18 OBD 50×100 mm (5-95% CH₃CN/H₂O with 20 mM NH₄OH)) provided the title compound in 21% yield. ¹H NMR (400 MHz, DMSO-d₆) δ 12.41 (br s, 1H), 9.67-9.42 (m, 1H), 7.70-7.32 (m, 2H), 7.24-7.03 (m, 1H), 6.90-6.63 (m, 1H), 5.69 (t, J=6.9 Hz, 1H), 5.48-5.40 (m, 1H), 4.21 (dd, J=9.7, 4.7 Hz, 1H), 4.09-3.99 (m, 1H), 3.92-3.80 (m, 2H), 3.69-3.37 (m, 3H), 3.33 (s, 3H), 3.27-3.18 (m, 1H), 2.52 (s, 3H), 1.40-1.30 (m, 6H). MS (ESI) m/z: [M+H]⁺ Found 590.3.

### Example 35: 4-Cyclopropyl-N—((R*)-1-(5-((S)-1-(5,5-difluoro-2-oxotetrahydropyrimidin-1(2H)-yl)-2-methoxyethyl)-1H-benzo[d]imidazol-2-yl)-2-((1,1,1-trifluoro-2-methylpropan-2-yl)oxy)ethyl)-1,2,5-oxadiazole-3-carboxamide

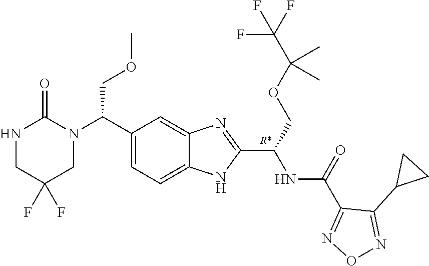 [SMILES: COC[C@H](c1ccc2[nH]c([C@H](COC(C)(C)C(F)(F)F)NC(=O)c3nonc3C3CC3)nc2c1)N1CC(F)(F)CNC1=O]

The title compound was prepared as described for the synthesis of Example 34 using 4-cyclopropyl-1,2,5-oxadiazole-3-carboxylic acid in place of 4-methyl-1,2,5-oxadiazole-3-carboxylic acid (9% yield). ¹H NMR (400 MHz, DMSO-d₆) δ 12.44 (br s, 1H), 9.71-9.44 (m, 1H), 7.63-7.34 (m, 2H), 7.18-7.06 (m, 1H), 6.84-6.73 (m, 1H), 5.75-5.63 (m, 1H), 5.53-5.39 (m, 1H), 4.21 (dd, J=9.7, 4.7 Hz, 1H), 4.09-4.00 (m, 1H), 3.92-3.78 (m, 2H), 3.67-3.41 (m, 3H), 3.33-3.33 (m, 3H), 3.26-3.18 (m, 1H), 2.46-2.34 (m, 1H), 1.39-1.30 (m, 6H), 1.20-1.12 (m, 2H), 1.04-0.96 (m, 2H). MS (ESI) m/z: [M+H]⁺ Found 616.3.

### Example 36: 1-Isopropyl-N—((R*)-1-(5-(((R*)-2-oxo-4-(trifluoromethyl)imidazolidin-1-yl)methyl)-1H-imidazo[4,5-b]pyridin-2-yl)-2-((1,1,1-trifluoro-2-methylpropan-2-yl)oxy)ethyl)-1H-pyrazole-5-carboxamide

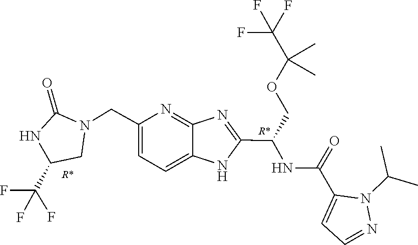 [SMILES: CC(C)n1nccc1C(=O)N[C@@H](COC(C)(C)C(F)(F)F)c1nc2nc(CN3C[C@H](C(F)(F)F)NC3=O)ccc2[nH]1]

### Example 37: 1-Isopropyl-N—((R*)-1-(5-(((S*)-2-oxo-4-(trifluoromethyl)imidazolidin-1-yl)methyl)-1H-imidazo[4,5-b]pyridin-2-yl)-2-((1,1,1-trifluoro-2-methylpropan-2-yl)oxy)ethyl)-1H-pyrazole-5-carboxamide

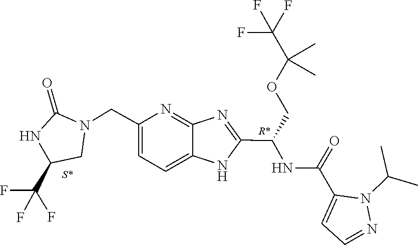 [SMILES: CC(C)n1nccc1C(=O)N[C@@H](COC(C)(C)C(F)(F)F)c1nc2nc(CN3C[C@@H](C(F)(F)F)NC3=O)ccc2[nH]1]

To a solution of 1-((2-((R*)-1-amino-2-((1,1,1-trifluoro-2-methylpropan-2-yl)oxy)ethyl)-1H-imidazo[4,5-b]pyridin-5-yl)methyl)-4-(trifluoromethyl)imidazolidin-2-one (124 mg, 0.274 mmol, Intermediate 93), 1-isopropyl-1H-pyrazole-5-carboxylic acid (63.4 mg, 0.411 mmol) and 1-hydroxybenzotriazole hydrate (65.0 mg, 0.411 mmol) in MeCN (2 mL) was added DIPEA (0.142 mL, 0.822 mmol). The mixture was heated to 45° C. then EDCI (78.8 mg, 0.411 mmol) was added, and the reaction stirred at this temperature for 1 h. At this time the reaction mixture was diluted with H₂O (1 mL), filtered, and purified by preparative HPLC (XBridge Cis OBD 50×100 mm (5-95% CH₃CN/H₂O with 20 mM NH₄OH)). This material was further purified initially by SFC using a chiral stationary phase (Stationary phase: OD-H, 2×25 cm, Mobile phase: 20% Ethanol, 80% CO₂) then again by SFC using a chiral stationary phase (Stationary phase: AD-H, 2×25 cm, Mobile phase: 20% Isopropanol, 80% CO₂) to provide 1-isopropyl-N—((R*)-1-(5-(((R*)-2-oxo-4-(trifluoromethyl)imidazolidin-1-yl)methyl)-1H-imidazo[4,5-b]pyridin-2-yl)-2-((1,1,1-trifluoro-2-methylpropan-2-yl)oxy)ethyl)-1H-pyrazole-5-carboxamide (Example 36, (13% yield, eluted at 3.9 min retention time)) and 1-isopropyl-N—((R*)-1-(5-(((S*)-2-oxo-4-(trifluoromethyl)imidazolidin-1-yl)methyl)-1H-imidazo[4,5-b]pyridin-2-yl)-2-((1,1,1-trifluoro-2-methylpropan-2-yl)oxy)ethyl)-1H-pyrazole-5-carboxamide (Example 37, (10% yield, eluted at 5.2 min retention time)). Data for Example 36: ¹H NMR (400 MHz, DMSO-d₆) δ 13.32-12.32 (m, 1H), 8.99-8.80 (m, 1H), 8.02-7.80 (m, 1H), 7.56-7.53 (m, 1H), 7.53-7.50 (m, 1H), 7.15-7.06 (m, 1H), 6.96-6.84 (m, 1H), 5.50-5.37 (m, 2H), 4.53-4.44 (m, 1H), 4.43-4.34 (m, 2H), 4.24-4.13 (m, 1H), 4.04-3.95 (m, 1H), 3.72-3.62 (m, 1H), 3.38 (dd, J=9.9, 4.5 Hz, 1H), 1.37 (d, J=6.6 Hz, 6H), 1.34-1.31 (m, 6H). MS (ESI) m/z: [M+H]⁺ Found 591.2. Data for Example 37: ¹H NMR (400 MHz, DMSO-d₆) δ 13.29-12.44 (m, 1H), 9.05-8.72 (m, 1H), 8.10-7.77 (m, 1H), 7.56-7.53 (m, 1H), 7.52-7.49 (m, 1H), 7.10 (d, J=8.3 Hz, 1H), 6.92-6.88 (m, 1H), 5.52-5.36 (m, 2H), 4.52-4.45 (m, 1H), 4.44-4.35 (m, 2H), 4.23-4.14 (m, 1H), 4.03-3.95 (m, 1H), 3.72-3.62 (m, 1H), 3.41-3.35 (m, 1H), 1.37 (d, J=6.6 Hz, 6H), 1.34-1.32 (m, 6H). MS (ESI) m/z: [M+H]⁺ Found 591.2.

### Example 38: 4-Cyclopropyl-N—((R*)-1-(5-(((R*)-2-oxo-4-(trifluoromethyl)imidazolidin-1-yl)methyl)-1H-imidazo[4,5-b]pyridin-2-yl)-2-((1,1,1-trifluoro-2-methylpropan-2-yl)oxy)ethyl)-1,2,5-oxadiazole-3-carboxamide

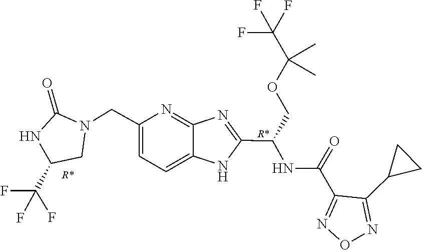 [SMILES: CC(C)(OC[C@H](NC(=O)c1nonc1C1CC1)c1nc2nc(CN3C[C@H](C(F)(F)F)NC3=O)ccc2[nH]1)C(F)(F)F]

### Example 39: 4-Cyclopropyl-N—((R*)-1-(5-(((S*)-2-oxo-4-(trifluoromethyl)imidazolidin-1-yl)methyl)-1H-imidazo[4,5-b]pyridin-2-yl)-2-((1,1,1-trifluoro-2-methylpropan-2-yl)oxy)ethyl)-1,2,5-oxadiazole-3-carboxamide

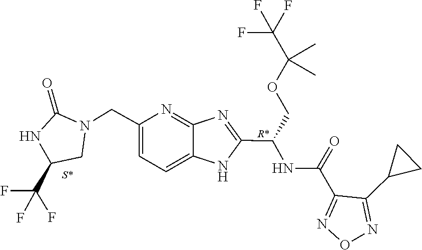 [SMILES: CC(C)(OC[C@H](NC(=O)c1nonc1C1CC1)c1nc2nc(CN3C[C@@H](C(F)(F)F)NC3=O)ccc2[nH]1)C(F)(F)F]

The title compounds were prepared as described for the synthesis of Example 36 and Example 37 using 4-cyclopropyl-1,2,5-oxadiazole-3-carboxylic acid in place of 1-isopropyl-1H-pyrazole-5-carboxylic acid. The material was purified by SFC using a chiral stationary phase (Stationary phase: AD-H, 3×25 cm, Mobile phase: 20% ethanol, 90% CO₂) to afford the 2 diastereomers. Example 38 was the first major eluting peak and Example 39 was the second major eluting peak, the title compounds were obtained in 13% and 14% yield respectively. Using the following analytical HPLC method: AD-H (25×0.46 cm), 15% isopropanol/CO₂, 100 bar, 3 mL/min, Example 38 showed a retention time of 5.53 min and Example 39 showed a retention time of 6.97 min. Data for Example 38: ¹H NMR (400 MHz, DMSO-d₆) δ 13.39-12.42 (m, 1H), 9.83-9.31 (m, 1H), 8.05-7.84 (m, 1H), 7.58-7.49 (m, 1H), 7.12 (d, J=8.1 Hz, 1H), 5.56-5.39 (m, 1H), 4.53-4.45 (m, 1H), 4.44-4.35 (m, 2H), 4.23 (dd, J=9.7, 4.8 Hz, 1H), 4.09-4.00 (m, 1H), 3.72-3.60 (m, 1H), 3.40-3.34 (m, 1H), 2.44-2.37 (m, 1H), 1.36-1.32 (m, 6H), 1.19-1.13 (m, 2H), 1.05-0.98 (m, 2H). MS (ESI) m/z: [M+H]⁺ Found 591.2. Data for Example 39: ¹H NMR (400 MHz, DMSO-d₆) δ 13.35-12.43 (m, 1H), 9.72-9.44 (m, 1H), 8.09-7.80 (m, 1H), 7.60-7.47 (m, 1H), 7.12 (d, J=8.3 Hz, 1H), 5.52-5.43 (m, 1H), 4.52-4.45 (m, 1H), 4.43-4.34 (m, 2H), 4.23 (dd, J=9.7, 4.8 Hz, 1H), 4.10-3.99 (m, 1H), 3.73-3.60 (m, 1H), 3.41-3.35 (m, 1H), 2.44-2.37 (m, 1H), 1.38-1.31 (m, 6H), 1.19-1.13 (m, 2H), 1.06-0.97 (m, 2H). MS (ESI) m/z: [M+H]⁺ Found 591.2.

### Example 40: N—((R*)-1-(5-((S*)-(1-Cyanocyclopropyl)(5,5-difluoro-2-oxotetrahydropyrimidin-1(2H)-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

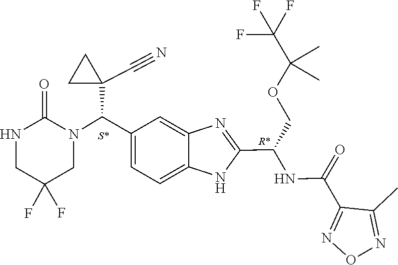 [SMILES: Cc1nonc1C(=O)N[C@@H](COC(C)(C)C(F)(F)F)c1nc2cc([C@H](N3CC(F)(F)CNC3=O)C3(C#N)CC3)ccc2[nH]1]

The title compound was prepared as described for the synthesis of Example 34 using 1-((S*)-(2-((R*)-1-amino-2-((1,1,1-trifluoro-2-methylpropan-2-yl)oxy)ethyl)-1H-benzo[d]imidazol-5-yl)(5,5-difluoro-2-oxotetrahydropyrimidin-1(2H)-yl)methyl)cyclopropane-1-carbonitrile (Intermediate 51) in place of 1-((S)-1-(2-((R*)-1-amino-2-((1,1,1-trifluoro-2-methylpropan-2-yl)oxy)ethyl)-1H-benzo[d]imidazol-5-yl)-2-methoxyethyl)-5,5-difluorotetrahydropyrimidin-2(1H)-one. Purification by silica gel chromatography (10-100% (10% MeOH in EtOAc)/hexanes) followed by further purification by SFC using a chiral stationary phase (Stationary phase: Whelk O1 SS 5 μM, 250×21 mm, Mobile phase: 20% methanol, 80% CO₂) provided the title compound in 11% yield. ¹H NMR (400 MHz, DMSO-d₆) δ 12.63-12.44 (m, 1H), 9.70-9.45 (m, 1H), 7.78-7.61 (m, 1H), 7.61-7.50 (m, 1H), 7.23-7.15 (m, 1H), 7.12-7.03 (m, 1H), 5.51-5.40 (m, 1H), 5.19 (s, 1H), 4.24 (dd, J=9.6, 4.6 Hz, 1H), 4.12-4.00 (m, 1H), 3.93-3.76 (m, 1H), 3.76-3.62 (m, 1H), 3.60-3.44 (m, 2H), 2.53 (s, 3H), 1.68-1.55 (m, 1H), 1.50-1.40 (m, 2H), 1.39-1.33 (m, 6H), 1.31-1.21 (m, 1H). MS (ESI) m/z: [M+H]⁺ Found 611.4.

### Example 41: N—((R*)-1-(5-((S)-2-cyclopropoxy-1-((S)-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

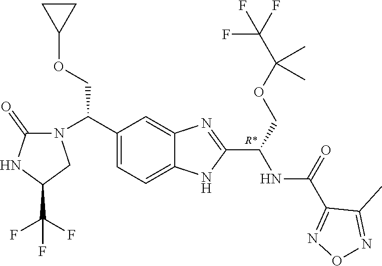 [SMILES: Cc1nonc1C(=O)N[C@@H](COC(C)(C)C(F)(F)F)c1nc2cc([C@@H](COC3CC3)N3C[C@@H](C(F)(F)F)NC3=O)ccc2[nH]1]

The title compound was prepared as described for the synthesis of Example 2 using (S)-1-((S)-1-(2-((R*)-1-amino-2-((1,1,1-trifluoro-2-methylpropan-2-yl)oxy)ethyl)-1H-benzo[d]imidazol-5-yl)-2-cyclopropoxyethyl)-4-(trifluoromethyl)imidazolidin-2-one (Intermediate 125) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one in 24% yield. ¹H NMR (400 MHz, DMSO-d₆) δ 12.49-12.31 (m, 1H), 9.62-9.47 (m, 1H), 7.61-7.33 (m, 3H), 7.14-7.05 (m, 1H), 5.51-5.37 (m, 1H), 5.18-5.05 (m, 1H), 4.49-4.32 (m, 1H), 4.27-4.15 (m, 1H), 4.10-3.93 (m, 2H), 3.91-3.84 (m, 1H), 3.78-3.68 (m, 1H), 3.40-3.37 (m, 1H), 3.21-3.16 (m, 1H), 2.52 (s, 3H), 1.38-1.30 (m, 6H), 0.60-0.38 (m, 4H). MS (ESI) m/z: [M+H]⁺ Found 634.3.

### Example 42: N-((1R,2R)-2-Cyclopropoxy-1-(5-((S)-2-methoxy-1-((S)-2-oxo-4-(trifluoromethyl)imidazolidin-1-yl)ethyl)-1H-benzo[d]imidazol-2-yl)propyl)-4-cyclopropyl-1,2,5-oxadiazole-3-carboxamide

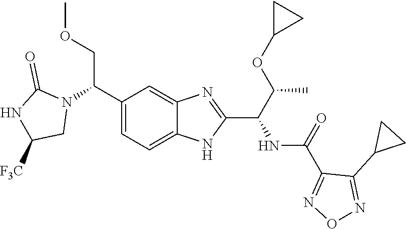 [SMILES: COC[C@H](c1ccc2[nH]c([C@@H](NC(=O)c3nonc3C3CC3)[C@@H](C)OC3CC3)nc2c1)N1C[C@@H](C(F)(F)F)NC1=O]

The title compound was prepared as described for the synthesis of Example 2 using (S)-1-((S)-1-(2-((1R,2R)-1-amino-2-cyclopropoxypropyl)-1H-benzo[d]imidazol-5-yl)-2-methoxyethyl)-4-(trifluoromethyl)imidazolidin-2-one (Intermediate 130) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. The product was purified by preparative HPLC (Sunfire™ Prep 5 μm C18 30×250 mm, 0-100% MeCN with 0.05% TFA/water with with 0.05% TFA) to afford, after lyophilization, the title compound in 43% yield. ¹H NMR (500 MHz, CD₃OD) δ 7.67-7.62 (m, 2H), 7.31 (dd, J=1.6, 8.4 Hz, 1H), 5.52 (d, J=4.0 Hz, 1H), 5.37 (dd, J=5.1, 8.4 Hz, 1H), 4.44-4.35 (m, 2H), 4.13-4.07 (m, 1H), 4.01-3.92 (m, 2H), 3.53 (s, 3H), 3.46-3.42 (m, 2H), 2.59-2.51 (m, 1H), 1.43 (d, J=6.3 Hz, 3H), 1.27-1.10 (m, 4H), 0.62-0.52 (m, 2H), 0.44-0.37 (m, 1H), 0.34-0.28 (m, 1H). MS (ESI) m/z: [M+H]⁺ Found 578.3.

### Example 43: N-((1R,2R)-2-Cyclopropoxy-1-(5-((S)-1-(5,5-difluoro-2-oxotetrahydropyrimidin-1(2H)-yl)-2-methoxyethyl)-1H-benzo[d]imidazol-2-yl)propyl)-4-cyclopropyl-1,2,5-oxadiazole-3-carboxamide

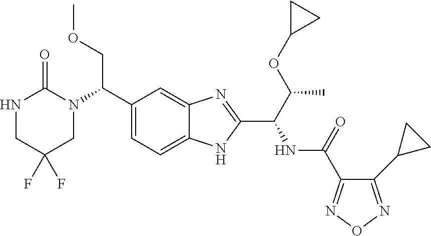 [SMILES: COC[C@H](c1ccc2[nH]c([C@@H](NC(=O)c3nonc3C3CC3)[C@@H](C)OC3CC3)nc2c1)N1CC(F)(F)CNC1=O]

The title compound was prepared as described for the synthesis of Example 2 using 1-((S)-1-(2-((1R,2R)-1-amino-2-cyclopropoxypropyl)-1H-benzo[d]imidazol-5-yl)-2-methoxyethyl)-5,5-difluorotetrahydropyrimidin-2(1H)-one (Intermediate 131) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. The product was purified by preparative HPLC (Sunfire™ Prep 5 μm C18 30×250 mm, 0-100% MeCN with 0.05% TFA/water with with 0.05% TFA) to afford, after lyophilization, the title compound in 51% yield. ¹H NMR (400 MHz, CD₃OD) δ 7.58 (s, 1H), 7.55 (d, J=8.3 Hz, 1H), 7.27-7.22 (m, 1H), 5.86-5.78 (m, 1H), 5.42 (d, J=4.3 Hz, 1H), 4.38-4.23 (m, 1H), 4.03-3.89 (m, 2H), 3.70-3.46 (m, 4H), 3.45 (s, 3H), 3.37-3.33 (m, 1H), 2.56-2.35 (m, 1H), 1.33 (d, J=6.3 Hz, 3H), 1.19-1.01 (m, 4H), 0.55-0.42 (m, 2H), 0.36-0.21 (m, 2H). MS (ESI) m/z: [M+H]⁺ Found 560.3.

### Example 44: 4-Cyclopropyl-N-((1R*,2R*)-4,4,4-trifluoro-2-methoxy-1-(5-(((S)-2-oxo-4-(trifluoromethyl)imidazolidin-1-yl)methyl)-1-((2-(trimethylsilyl)ethoxy)methyl)-1H-benzo[d]imidazol-2-yl)butyl)-1,2,5-oxadiazole-3-carboxamide

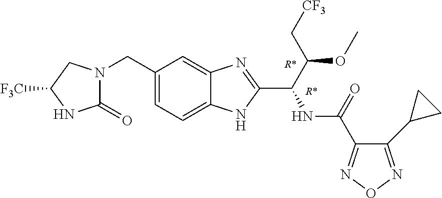 [SMILES: CO[C@H](CC(F)(F)F)[C@H](NC(=O)c1nonc1C1CC1)c1nc2cc(CN3C[C@@H](C(F)(F)F)NC3=O)ccc2[nH]1]

### Example 45: 4-Cyclopropyl-N-((1S*,2R*)-4,4,4-trifluoro-2-methoxy--(5-(((S)-2-oxo-4-(trifluoromethyl)imidazolidin-1-yl)methyl)-1H-benzo[d]imidazol-2-yl)butyl)-1,2,5-oxadiazole-3-carboxamide

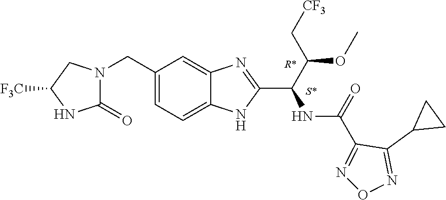 [SMILES: CO[C@H](CC(F)(F)F)[C@@H](NC(=O)c1nonc1C1CC1)c1nc2cc(CN3C[C@@H](C(F)(F)F)NC3=O)ccc2[nH]1]

### Example 46: 4-Cyclopropyl-N-((1R*,2S*)-4,4,4-trifluoro-2-methoxy-1-(5-(((S)-2-oxo-4-(trifluoromethyl)imidazolidin-1-yl)methyl)-1H-benzo[d]imidazol-2-yl)butyl)-1,2,5-oxadiazole-3-carboxamide

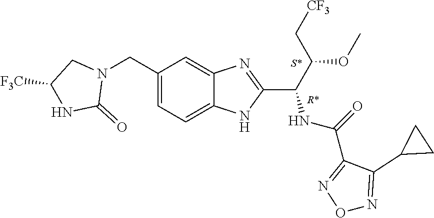 [SMILES: CO[C@@H](CC(F)(F)F)[C@H](NC(=O)c1nonc1C1CC1)c1nc2cc(CN3C[C@@H](C(F)(F)F)NC3=O)ccc2[nH]1]

The mixture of title compounds was prepared as described for the synthesis of Example 2 using (4S)-1-((2-(1-amino-4,4,4-trifluoro-2-methoxybutyl)-1H-benzo[d]imidazol-5-yl)methyl)-4-(trifluoromethyl)imidazolidin-2-one (Intermediate 128) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. The material was purified by silica gel chromatography (0-2% MeOH/DCM) and chiral SFC three times (DIACEL CHIRALPAK AD, 10 μm, 250×30 mm; 20% MeOH (containing 0.1% NH₄OH)/CO₂) to provide three stereoisomers. The first eluting isomer, designated (1R*,2R*), was isolated as Example 44 in 47% yield. The second eluting isomer, designated (1S*,2R*) was isolated as Example 45 in 6% yield. The third eluting isomer, designated (1R*,2S*), was isolated as Example 46 in 65% yield. Example 44: ¹H NMR (400 MHz, DMSO-d₆) δ 12.63-12.42 (m, 1H), 9.51 (br s, 1H), 7.70-7.31 (m, 3H), 7.08 (d, J=8.0 Hz, 1H), 5.60 (d, J=4.4 Hz, 1H), 4.46-4.21 (m, 4H), 3.52 (t, J=10.0 Hz, 1H), 3.34 (s, 3H), 3.28-3.22 (m, 1H), 2.88-2.74 (m, 1H), 2.61-2.52 (m, 1H), 2.43-2.31 (m, 1H), 1.20-1.13 (m, 2H), 1.04-0.98 (m, 2H). MS (ESI) m/z: [M+H]⁺ Found 576.2. Example 45: ¹H NMR (400 MHz, DMSO-d₆) δ 12.59 (br s, 1H), 9.73 (br s, 1H), 7.64-7.34 (m, 3H), 7.14-7.00 (m, 1H), 5.82-5.73 (m, 1H), 4.49-4.26 (m, 4H), 3.54-3.49 (m, 1H), 3.34 (s, 3H), 3.28-3.22 (m, 1H), 2.76-2.65 (m, 1H), 2.62-2.53 (m, 1H), 2.43-2.34 (m, 1H), 1.19-1.14 (m, 2H), 1.05-0.98 (m, 2H). MS (ESI) m/z: [M+H]⁺ Found 576.2. Example 46: ¹H NMR (400 MHz, CDCl₃) δ 12.05-11.21 (m, 1H), 8.37-8.11 (m, 1H), 7.78-7.41 (m, 2H), 7.15 (d, J=7.2 Hz, 1H), 6.12-5.48 (m, 2H), 4.61-4.42 (m, 2H), 4.20-4.07 (m, 2H), 3.61 (t, J=9.6 Hz, 1H), 3.53-3.33 (m, 4H), 2.72-2.54 (m, 1H), 2.53-2.38 (m, 2H), 1.19-1.04 (m, 4H). MS (ESI) m/z: [M+H]⁺ Found 576.2.

### Example 47: 4-Cyclopropyl-N—((R*)-1-(5-((S)-2-methoxy-1-((S)-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-1,2,5-oxadiazole-3-carboxamide

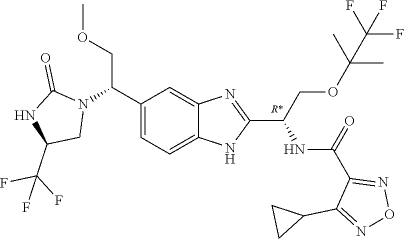 [SMILES: COC[C@H](c1ccc2[nH]c([C@H](COC(C)(C)C(F)(F)F)NC(=O)c3nonc3C3CC3)nc2c1)N1C[C@@H](C(F)(F)F)NC1=O]

The title compound (24% yield) was prepared as described for the synthesis of Example 1 using (S)-1-((S)-1-(2-((R*)-1-amino-2-((1,1,1-trifluoro-2-methylpropan-2-yl)oxy)ethyl)-1H-benzo[d]imidazol-5-yl)-2-methoxyethyl)-4-(trifluoromethyl)imidazolidin-2-one (Intermediate 80) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. ¹H NMR (500 MHz, DMSO-d₆) δ 12.56-12.37 (m, 1H), 9.68-9.51 (m, 1H), 7.61-7.33 (m, 3H), 7.17-7.03 (m, 1H), 5.54-5.40 (m, 1H), 5.22-5.06 (m, 1H), 4.53-4.35 (m, 1H), 4.27-4.15 (m, 1H), 4.11-3.99 (m, 1H), 3.95-3.85 (m, 1H), 3.84-3.69 (m, 2H), 3.33 (s, 5H), 1.37-1.32 (m, 6H), 1.19-1.13 (m, 2H), 1.04-0.98 (m, 2H). MS (ESI) m/z: [M+H]⁺ Found 634.3.

### Example 48: N—((R)-1-(5-((R*)-1-(4,4-Dimethyl-2-oxoimidazolidin-1-yl)-2-methoxyethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

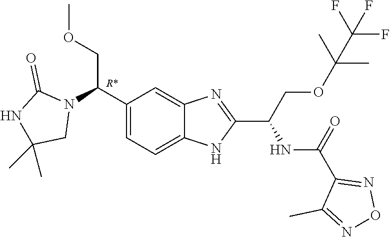 [SMILES: COC[C@@H](c1ccc2[nH]c([C@H](COC(C)(C)C(F)(F)F)NC(=O)c3nonc3C)nc2c1)N1CC(C)(C)NC1=O]

### Example 49: N—((R)-1-(5-((S*)-1-(4,4-Dimethyl-2-oxoimidazolidin-1-yl)-2-methoxyethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

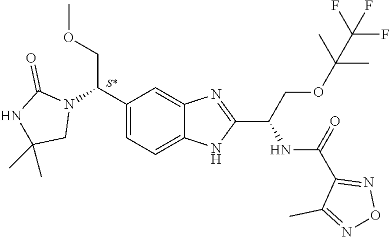 [SMILES: COC[C@H](c1ccc2[nH]c([C@H](COC(C)(C)C(F)(F)F)NC(=O)c3nonc3C)nc2c1)N1CC(C)(C)NC1=O]

The title compounds were prepared as described for the synthesis of Example 2 using 1-(1-(2-((R)-1-amino-2-((1,1,1-trifluoro-2-methylpropan-2-yl)oxy)ethyl)-1H-benzo[d]imidazol-5-yl)-2-methoxyethyl)-4,4-dimethylimidazolidin-2-one (Intermediate 150) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. These diastereomers were purified by silica gel chromatography (10-100% EtOAc (10% MeOH)/hexanes) and then separated by chiral SFC (Chiralpak IH3, 3 μm, 250×21 mm, 85% CO₂ in MeOH) to afford N—((R)-1-(5-((R*)-1-(4,4-dimethyl-2-oxoimidazolidin-1-yl)-2-methoxyethyl)-1H-benzo[d]imidazol-2-yl)-2-((1,1,1-trifluoro-2-methylpropan-2-yl)oxy)ethyl)-4-methyl-1,2,5-oxadiazole-3-carboxamide (Example 48) as the first eluting isomer in 18% yield and N—((R)-1-(5-((S*)-1-(4,4-dimethyl-2-oxoimidazolidin-1-yl)-2-methoxyethyl)-1H-benzo[d]imidazol-2-yl)-2-((1,1,1-trifluoro-2-methylpropan-2-yl)oxy)ethyl)-4-methyl-1,2,5-oxadiazole-3-carboxamide (Example 49) as the second eluting isomer in 15% yield. Example 48: ¹H NMR (500 MHz, DMSO-d₆) δ 12.37-12.04 (m, 1H), 9.23-8.91 (m, 1H), 7.93-7.88 (m, 3H), 7.63-7.52 (m, 1H), 7.46-7.35 (m, 1H), 7.19-7.09 (m, 1H), 6.18-5.89 (m, 1H), 5.56 (br s, 1H), 5.25 (br t, J=6.6 Hz, 1H), 4.21-4.06 (m, 2H), 3.92-3.77 (m, 2H), 3.39 (d, J=2.0 Hz, 3H), 3.18-3.14 (m, 1H), 2.92-2.86 (m, 1H), 1.35 (d, J=7.0 Hz, 6H), 1.27-1.24 (m, 3H), 1.14 (s, 3H). MS (ESI) m/z: [M+H]⁺ Found 568.2. Example 49: ¹H NMR (500 MHz, DMSO-d₆) δ 12.47-12.28 (m, 1H), 9.55 (t, J=7.9 Hz, 1H), 7.59-7.33 (m, 2H), 7.14-7.06 (m, 1H), 6.47-6.38 (m, 1H), 5.48-5.38 (m, 1H), 5.15-5.08 (m, 1H), 4.24-4.17 (m, 1H), 4.08-4.00 (m, 1H), 3.85-3.72 (m, 2H), 3.31 (s, 3H), 3.15-3.11 (m, 1H), 2.86-2.79 (m, 1H), 2.52 (s, 3H), 1.37-1.32 (m, 6H), 1.18-1.15 (m, 3H), 1.09-1.04 (m, 3H). MS (ESI) m/z: [M+H]⁺ Found 568.2.

### Example 50: N-((1R,2R)-1-(5-((S)-1-(5,5-Difluoro-2-oxotetrahydropyrimidin-1(2H)-yl)-2-methoxyethyl)-1H-benzo[d]imidazol-2-yl)-2-(((S)-1,1,1-trifluoropropan-2-yl)oxy)propyl)-4-methyl-1,2,5-oxadiazole-3-carboxamide

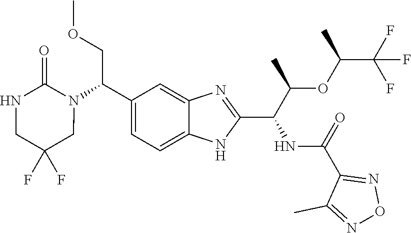 [SMILES: COC[C@H](c1ccc2[nH]c([C@@H](NC(=O)c3nonc3C)[C@@H](C)O[C@@H](C)C(F)(F)F)nc2c1)N1CC(F)(F)CNC1=O]

The title compound (67% yield) was prepared as described for the synthesis of Example 2 using 1-((S)-1-(2-((1R,2R)-1-amino-2-(((S)-1,1,1-trifluoropropan-2-yl)oxy)propyl)-1H-benzo[d]imidazol-5-yl)-2-methoxyethyl)-5,5-difluorotetrahydropyrimidin-2(1H)-one (Intermediate 155) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. ¹H NMR (500 MHz, DMSO-d₆) δ 12.48-12.34 (m, 1H), 9.27-9.13 (m, 1H), 7.60-7.53 (m, 1H), 7.50-7.41 (m, 1H), 7.19-7.07 (m, 1H), 6.83-6.73 (m, 1H), 5.74-5.63 (m, 1H), 5.38-5.26 (m, 1H), 4.41-4.27 (m, 2H), 3.92-3.80 (m, 2H), 3.69-3.38 (m, 3H), 3.34-3.32 (m, 3H), 3.29-3.21 (m, 1H), 2.49-2.47 (m, 3H), 1.28-1.23 (m, 3H), 1.17-1.13 (m, 3H). MS (ESI) m/z: [M+H]⁺ Found 590.2.

### Example 51: 2-(Cyclopropylmethyl)-N—((R*)-1-(5-((S)-1-(5,5-difluoro-2-oxotetrahydropyrimidin-1(2H)-yl)-2-methoxyethyl)-1H-benzo[d]imidazol-2-yl)-2-((1,1,1-trifluoro-2-methylpropan-2-yl)oxy)ethyl)-2H-1,2,3-triazole-4-carboxamide

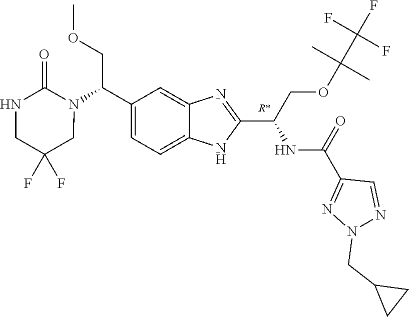 [SMILES: COC[C@H](c1ccc2[nH]c([C@H](COC(C)(C)C(F)(F)F)NC(=O)c3cnn(CC4CC4)n3)nc2c1)N1CC(F)(F)CNC1=O]

The title compound (25% yield) was prepared as described for the synthesis of Example 34 using 2-(cyclopropylmethyl)triazole-4-carboxylic acid in place of 4-methyl-1,2,5-oxadiazole-3-carboxylic acid. ¹H NMR (400 MHz, DMSO-d₆) δ 12.37 (s, 1H), 8.68 (d, J=8.4 Hz, 1H), 7.74-7.35 (m, 2H), 7.12 (t, J=7.8 Hz, 1H), 7.03 (s, 1H), 6.78 (d, J=8.5 Hz, 1H), 5.70 (t, J=7.2 Hz, 1H), 5.44 (td, J=7.7, 5.1 Hz, 1H), 4.37 (h, J=7.1 Hz, 2H), 4.17 (dd, J=9.7, 5.1 Hz, 1H), 4.10-4.04 (m, 1H), 3.94-3.78 (m, 2H), 3.51 (ddd, J=41.7, 22.7, 12.3 Hz, 3H), 3.34 (s, 3H), 3.25 (d, J=16.8 Hz, 1H), 1.43-1.28 (m, 7H), 0.62-0.53 (m, 2H), 0.49-0.40 (m, 2H). MS (ESI) m/z: [M+H]⁺ Found 629.3.

### Example 52: N—((R*)-1-(5-((S)-1-(5,5-Difluoro-2-oxotetrahydropyrimidin-1(2H)-yl)-2-methoxyethyl)-1H-benzo[d]imidazol-2-yl)-2-((1,1,1-trifluoro-2-methylpropan-2-yl)oxy)ethyl)-1-isopropyl-1H-1,2,4-triazole-5-carboxamide

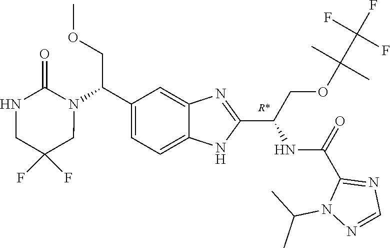 [SMILES: COC[C@H](c1ccc2[nH]c([C@H](COC(C)(C)C(F)(F)F)NC(=O)c3ncnn3C(C)C)nc2c1)N1CC(F)(F)CNC1=O]

The title compound was prepared as described for the synthesis of Example 34 using lithium 1-isopropyl-1H-1,2,4-triazole-5-carboxylate in place of 4-methyl-1,2,5-oxadiazole-3-carboxylic acid. The product was initially purified by silica gel chromatography (0-100% EtOAc (10% MeOH)/hexanes) and then further purified by preparative HPLC (XBridge C18 BEH 5 μm, 50×100 mm (30-60% CH₃CN/H₂O with 24 mM NH₄OH)) to afford the title compound in 8% yield. ¹H NMR (600 MHz, DMSO-d₆) δ 12.41 (br s, 1H), 9.04 (d, J=8.5 Hz, 1H), 8.15 (s, 1H), 7.73-7.33 (m, 2H), 7.13 (d, J=8.3 Hz, 1H), 6.89-6.72 (m, 1H), 5.74-5.67 (m, 1H), 5.67-5.58 (m, 1H), 5.49-5.40 (m, 1H), 4.17 (dd, J=9.8, 4.7 Hz, 1H), 4.10 (dd, J=9.7, 7.6 Hz, 1H), 3.91-3.79 (m, 2H), 3.64-3.39 (m, 3H), 3.34 (s, 3H), 3.28-3.20 (m, 1H), 1.44 (d, J=4.3 Hz, 3H), 1.43 (d, J=4.3 Hz, 3H), 1.33 (s, 3H), 1.31 (s, 3H). MS (ESI) m/z: [M+H]⁺ Found 617.5.

### Example 53: N—((S)-1-(5-((R)-Cyclopropyl((S*)-2-oxo-4-(trifluoromethyl)imidazolidin-1-yl-5,5-d₂)methyl)-1H-benzo[d]imidazol-2-yl)-4,4,4-trifluoro-3,3-dimethylbutyl)-4-methyl-1,2,5-oxadiazole-3-carboxamide

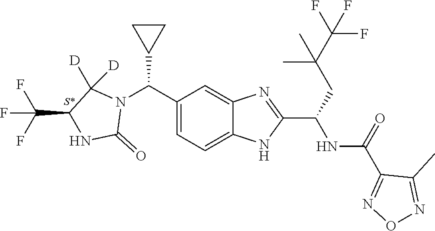 [SMILES: [2H]C1([2H])[C@H](C(F)(F)F)NC(=O)N1[C@@H](c1ccc2[nH]c([C@H](CC(C)(C)C(F)(F)F)NC(=O)c3nonc3C)nc2c1)C1CC1]

The title compound (81% yield) was prepared as described for the synthesis of Example 2 using (S)-1-((R)-(2-((S*)-1-amino-4,4,4-trifluoro-3,3-dimethylbutyl)-1H-benzo[d]imidazol-5-yl) (cyclopropyl)methyl)-4-(trifluoromethyl)imidazolidin-2-one-5,5-d₂ (Intermediate 162) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. ¹H NMR (400 MHz, Methanol-d₄) δ 7.86-7.42 (m, 2H), 7.32 (s, 1H), 5.66 (dd, J=9.0, 4.3 Hz, 1H), 4.43-4.21 (m, 2H), 2.66 (dd, J=14.8, 4.3 Hz, 1H), 2.58 (s, 3H), 2.40 (dd, J=14.8, 9.0 Hz, 1H), 1.51 (s, 1H), 1.28 (s, 5H), 1.25 (s, 3H), 0.96-0.86 (m, 1H), 0.79-0.62 (m, 1H), 0.61-0.33 (m, 2H). MS (ESI) m/z: [M+H]⁺ Found 590.3.

### Example 54: N—((R*)-1-(5-((S)-2-Methoxy-1-((R*)-2-oxo-4-(trifluoromethyl)imidazolidin-1-yl-5,5-d₂)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

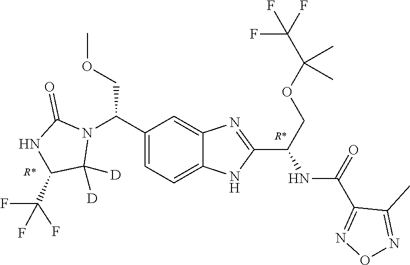 [SMILES: [2H]C1([2H])[C@H](C(F)(F)F)NC(=O)N1[C@H](COC)c1ccc2[nH]c([C@H](COC(C)(C)C(F)(F)F)NC(=O)c3nonc3C)nc2c1]

### Example 55: N—((R*)-1-(5-((S)-2-Methoxy-1-((S*)-2-oxo-4-(trifluoromethyl)imidazolidin-1-yl-5,5-d₂)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

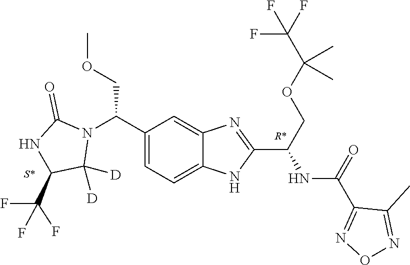 [SMILES: [2H]C1([2H])[C@@H](C(F)(F)F)NC(=O)N1[C@H](COC)c1ccc2[nH]c([C@H](COC(C)(C)C(F)(F)F)NC(=O)c3nonc3C)nc2c1]

The title compounds were prepared as described for the synthesis of Example 2 using 1-((S)-1-(2-((R)-1-amino-2-((1,1,1-trifluoro-2-methylpropan-2-yl)oxy)ethyl)-1H-benzo[d]imidazol-5-yl)-2-methoxyethyl)-4-(trifluoromethyl)imidazolidin-2-one-5,5-d₂ (Intermediate 165) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. Purification and separation by chiral SFC (Chiralpak IB N₃ 5 μm, 250×21 mm, 85% CO₂ in MeOH) afforded N—((R*)-1-(5-((S)-2-methoxy-1-((R*)-2-oxo-4-(trifluoromethyl)imidazolidin-1-yl-5,5-d₂)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide (Example 54) as the first eluting isomer in 14% yield and N—((R*)-1-(5-((S)-2-methoxy-1-((S*)-2-oxo-4-(trifluoromethyl)imidazolidin-1-yl-5,5-d₂)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide (Example 55) as the second eluting isomer in 7% yield. Data for Example 54: ¹H NMR (400 MHz, DMSO-d₆) δ 12.59-12.28 (m, 1H), 9.70-9.45 (m, 1H), 7.64-7.30 (m, 3H), 7.09 (br d, J=8.5 Hz, 1H), 5.50-5.39 (m, 1H), 5.13 (dd, J=8.6, 5.4 Hz, 1H), 4.47-4.34 (m, 1H), 4.21 (dd, J=9.7, 4.8 Hz, 1H), 4.09-3.98 (m, 1H), 3.94-3.84 (m, 1H), 3.76 (dd, J=10.4, 5.4 Hz, 1H), 3.32 (s, 3H), 2.52 (s, 3H), 1.38-1.30 (m, 6H). MS (ESI) m/z: [M+H]⁺ Found 610.3. Data for Example 55: ¹H NMR (400 MHz, DMSO-d₆) δ 12.70-12.36 (m, 1H), 9.84-9.47 (m, 1H), 7.75-7.38 (m, 3H), 7.20 (br d, J=8.0 Hz, 1H), 5.59-5.45 (m, 1H), 5.28-5.11 (m, 1H), 4.44-4.33 (m, 1H), 4.29 (dd, J=9.7, 4.7 Hz, 1H), 4.18-4.06 (m, 1H), 4.03-3.91 (m, 1H), 3.90-3.79 (m, 1H), 3.39 (br s, 3H), 2.60 (s, 3H), 1.51-1.36 (m, 6H). MS (ESI) m/z: [M+H]⁺ Found 610.3.

### Example 56: N—((R*)-1-(5-((S)-2-Cyclopropoxy-1-(5,5-difluoro-2-oxotetrahydropyrimidin-1(2H)-yl)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

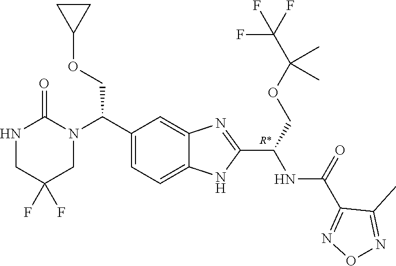 [SMILES: Cc1nonc1C(=O)N[C@@H](COC(C)(C)C(F)(F)F)c1nc2cc([C@@H](COC3CC3)N3CC(F)(F)CNC3=O)ccc2[nH]1]

The title compound (51% yield) was prepared as described for the synthesis of Example 2 using 1-((S)-1-(2-((R*)-1-amino-2-((1,1,1-trifluoro-2-methylpropan-2-yl)oxy)ethyl)-1H-benzo[d]imidazol-5-yl)-2-cyclopropoxyethyl)-5,5-difluorotetrahydropyrimidin-2(1H)-one (Intermediate 167) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. ¹H NMR (500 MHz, DMSO-d₆) δ 12.47-12.32 (m, 1H), 9.55 (d, J=8.5 Hz, 1H), 7.64-7.49 (m, 1H), 7.49-7.37 (m, 1H), 7.19-7.06 (m, 1H), 6.83-6.71 (m, 1H), 5.73-5.64 (m, 1H), 5.48-5.39 (m, 1H), 4.21 (dd, J=9.7, 4.8 Hz, 1H), 4.07-4.01 (m, 1H), 3.99-3.90 (m, 2H), 3.64-3.36 (m, 4H), 3.27-3.16 (m, 1H), 2.52 (s, 3H), 1.39-1.30 (m, 6H), 0.56-0.40 (m, 4H). MS (ESI) m/z: [M+H]⁺ Found 616.3.

### Example 57: 4-Methyl-N—((R*)-1-(5-(((R*)-4-methyl-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide

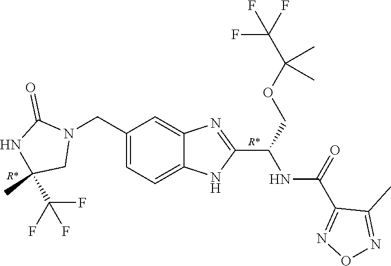

### Example 58: 4-Methyl-N—((R*)-1-(5-(((S*)-4-methyl-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide

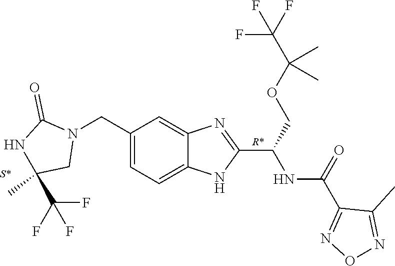

The title compounds were prepared as described for the synthesis of Example 2 using 1-((2-((R*)-1-amino-2-((1,1,1-trifluoro-2-methylpropan-2-yl)oxy)ethyl)-1H-benzo[d]imidazol-5-yl)methyl)-4-methyl-4-(trifluoromethyl)imidazolidin-2-one (Intermediate 172) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. Purification and separation by chiral SFC (Chiralpak IB N₃ 5 μm, 250×21 mm, 85% CO₂ in MeOH) afforded 4-methyl-N—((R*)-1-(5-(((R*)-4-methyl-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide (Example 57) as the first eluting isomer in 19% yield and 4-methyl-N—((R*)-1-(5-(((S*)-4-methyl-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide (Example 58) as the second eluting isomer in 14% yield. Data for Example 57: ¹H NMR (400 MHz, DMSO-d₆) δ 12.41 (d, J=13.6 Hz, 1H), 9.62-9.47 (m, 1H), 7.60-7.27 (m, 3H), 7.10-6.98 (m, 1H), 5.50-5.39 (m, 1H), 4.45 (dd, J=15.1, 6.6 Hz, 1H), 4.28 (dd, J=14.9, 5.3 Hz, 1H), 4.24-4.17 (m, 1H), 4.04 (t, J=9.1 Hz, 1H), 3.40-3.36 (m, 1H), 3.19-3.11 (m, 1H), 2.52 (s, 3H), 1.37-1.31 (m, 9H). MS (ESI) m/z: [M+H]⁺ Found 578.2. Data for Example 58: ¹H NMR (400 MHz, DMSO-d₆) δ 12.41 (d, J=13.9 Hz, 1H), 9.64-9.48 (m, 1H), 7.62-7.25 (m, 3H), 7.12-6.98 (m, 1H), 5.50-5.37 (m, 1H), 4.45 (dd, J=15.0, 4.1 Hz, 1H), 4.28 (dd, J=15.0, 7.8 Hz, 1H), 4.24-4.17 (m, 1H), 4.03 (t, J=9.0 Hz, 1H), 3.40-3.34 (m, 1H), 3.19-3.10 (m, 1H), 2.52 (s, 3H), 1.37-1.31 (m, 9H). MS (ESI) m/z: [M+H]⁺ Found 578.2.

### Example 59: N—((R*)-1-(5-((S*)-2-Cyclopropoxy-1-((S*)-2-oxo-4-(trifluoromethyl)imidazolidin-1-yl-5,5-d₂)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

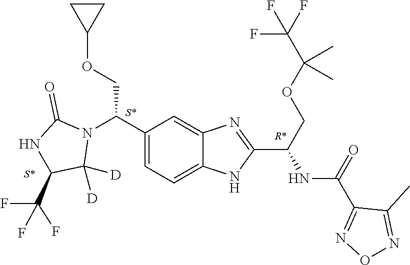 [SMILES: [2H]C1([2H])[C@@H](C(F)(F)F)NC(=O)N1[C@H](COC1CC1)c1ccc2[nH]c([C@H](COC(C)(C)C(F)(F)F)NC(=O)c3nonc3C)nc2c1]

The title compound was prepared as described for the synthesis of Example 2 using (S*)-1-((S*)-1-(2-((R*)-1-amino-2-((1,1,1-trifluoro-2-methylpropan-2-yl)oxy)ethyl)-1H-benzo[d]imidazol-5-yl)-2-cyclopropoxyethyl)-4-(trifluoromethyl)imidazolidin-2-one-5,5-d₂ (Intermediate 177) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. Purification by preparative HPLC (Waters XBridge C18, 5 μm, 50×100 mm, 20-95% MeCN/water (with 20 mM NH₄OH)) afforded the title compound in 10% yield. ¹H NMR (500 MHz, DMSO-d₆) δ 12.47-12.36 (m, 1H), 9.64-9.45 (m, 1H), 7.59-7.32 (m, 3H), 7.13-7.05 (m, 1H), 5.48-5.41 (m, 1H), 5.12 (dd, J=8.5, 5.5 Hz, 1H), 4.46-4.35 (m, 1H), 4.24-4.17 (m, 1H), 4.08-3.93 (m, 2H), 3.92-3.83 (m, 1H), 3.42-3.37 (m, 1H), 2.52 (s, 3H), 1.36-1.32 (m, 6H), 0.56-0.40 (m, 4H). MS (ESI) m/z: [M+H]⁺ Found 636.3.

### Example 60: 4-Cyclopropyl-N—((R*)-1-(5-(((R*)-4-methyl-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide

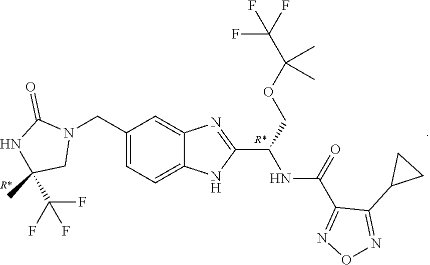 [SMILES: CC(C)(OC[C@H](NC(=O)c1nonc1C1CC1)c1nc2cc(CN3C[C@](C)(C(F)(F)F)NC3=O)ccc2[nH]1)C(F)(F)F]

### Example 61: 4-Cyclopropyl-N—((R*)-1-(5-(((S*)-4-methyl-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide

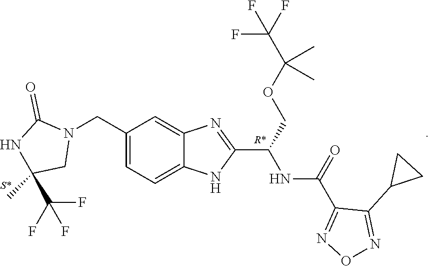 [SMILES: CC(C)(OC[C@H](NC(=O)c1nonc1C1CC1)c1nc2cc(CN3C[C@@](C)(C(F)(F)F)NC3=O)ccc2[nH]1)C(F)(F)F]

The title compounds were prepared as described for the synthesis of Example 2 using 1-((2-((R*)-1-amino-2-((1,1,1-trifluoro-2-methylpropan-2-yl)oxy)ethyl)-1H-benzo[d]imidazol-5-yl)methyl)-4-methyl-4-(trifluoromethyl)imidazolidin-2-one (Intermediate 172) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. Purification and separation by chiral SFC (Chiralpak IB N₃ 5 μm, 250×21 mm, 90% CO₂ in MeOH) afforded 4-cyclopropyl-N—((R*)-1-(5-(((R*)-4-methyl-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide (Example 60) as the first eluting isomer in 8% yield and 4-cyclopropyl-N—((R*)-1-(5-(((S*)-4-methyl-2-oxo-4-(trifluoromethyl)imidazolidin-1-yl)methyl)-1H-benzo[d]imidazol-2-yl)-2-((1,1,1-trifluoro-2-methylpropan-2-yl)oxy)ethyl)-1,2,5-oxadiazole-3-carboxamide (Example 61) as the second eluting isomer in 15% yield. Data for Example 60: ¹H NMR (500 MHz, DMSO-d₆) δ 12.57-12.33 (m, 1H), 9.70-9.51 (m, 1H), 7.61-7.26 (m, 3H), 7.12-6.98 (m, 1H), 5.52-5.41 (m, 1H), 4.50-4.39 (m, 1H), 4.33-4.24 (m, 1H), 4.20 (dd, J=9.6, 4.6 Hz, 1H), 4.08-3.98 (m, 1H), 3.40-3.36 (m, 1H), 3.18-3.12 (m, 1H), 2.45-2.35 (m, 1H), 1.37-1.32 (m, 9H), 1.18-1.14 (m, 2H), 1.04-0.98 (m, 2H). MS (ESI) m/z: [M+H]⁺ Found 604.4. Data for Example 61: ¹H NMR (500 MHz, DMSO-d₆) δ 12.56-12.27 (m, 1H), 9.73-9.52 (m, 1H), 7.60-7.29 (m, 3H), 7.11-7.00 (m, 1H), 5.52-5.41 (m, 1H), 4.51-4.41 (m, 1H), 4.34-4.24 (m, 1H), 4.23-4.16 (m, 1H), 4.09-4.00 (m, 1H), 3.41-3.35 (m, 1H), 3.18-3.12 (m, 1H), 2.44-2.37 (m, 1H), 1.37-1.31 (m, 9H), 1.19-1.14 (m, 2H), 1.04-0.98 (m, 2H). MS (ESI) m/z: [M+H]⁺ Found 604.4.

### Example 62: N-((1R,2R)-1-(5-((S)-2-Methoxy-1-((S)-2-oxo-4-(trifluoromethyl)imidazolidin-1-yl)ethyl)-1H-benzo[d]imidazol-2-yl)-2-(((S)-1,1,1-trifluoropropan-2-yl)oxy)propyl)-4-methyl-1,2,5-oxadiazole-3-carboxamide

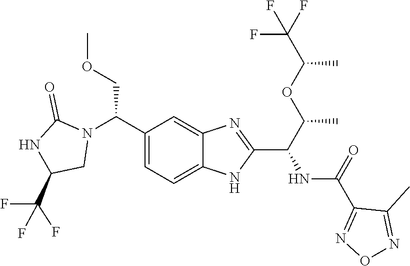 [SMILES: COC[C@H](c1ccc2[nH]c([C@@H](NC(=O)c3nonc3C)[C@@H](C)O[C@@H](C)C(F)(F)F)nc2c1)N1C[C@@H](C(F)(F)F)NC1=O]

The title compound (18% yield) was prepared as described for the synthesis of Example 2 using (S)-1-((S)-1-(2-((1R,2R)-1-amino-2-(((S)-1,1,1-trifluoropropan-2-yl)oxy)propyl)-1H-benzo[d]imidazol-5-yl)-2-methoxyethyl)-4-(trifluoromethyl)imidazolidin-2-one (Intermediate 179) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. ¹H NMR (400 MHz, DMSO-d₆) δ 12.48-12.33 (m, 1H), 9.25-9.11 (m, 1H), 7.61-7.32 (m, 3H), 7.16-7.03 (m, 1H), 5.37-5.27 (m, 1H), 5.19-5.07 (m, 1H), 4.49-4.38 (m, 1H), 4.38-4.26 (m, 2H), 3.95-3.85 (m, 1H), 3.82-3.69 (m, 2H), 3.33 (s, 3H), 3.26-3.19 (m, 1H), 2.48 (s, 3H), 1.25 (d, J=6.5 Hz, 3H), 1.15 (d, J=6.1 Hz, 3H). MS (ESI) m/z: [M+H]⁺ Found 608.3.

### Example 63: N—((R)-1-(5-((R*)-2-Methoxy-1-((S)-4-methyl-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

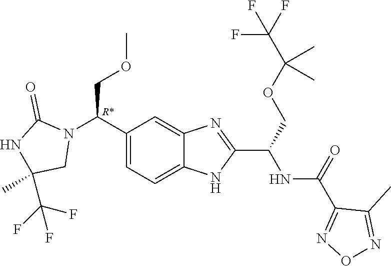

### Example 64: N—((R)-1-(5-((S*)-2-Methoxy-1-((S)-4-methyl-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide

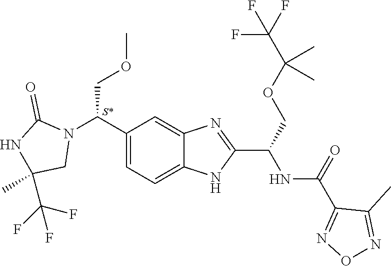

The title compounds were prepared as described for the synthesis of Example 2 using (4S)-1-(1-(2-((R)-1-amino-2-((1,1,1-trifluoro-2-methylpropan-2-yl)oxy)ethyl)-1H-benzo[d]imidazol-5-yl)-2-methoxyethyl)-4-methyl-4-(trifluoromethyl)imidazolidin-2-one (Intermediate 158) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. Purification and separation by chiral SFC (Chiralpak IB N₅ 3 μm, 250×21 mm, 85% CO₂ in MeOH) afforded N—((R)-1-(5-((R*)-2-methoxy-1-((S)-4-methyl-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide (Example 63) as the first eluting isomer in 19% yield and N—((R)-1-(5-((S*)-2-methoxy-1-((S)-4-methyl-2-oxo-4-(trifluoromethyl)imidazolidin-1-yl)ethyl)-1H-benzo[d]imidazol-2-yl)-2-((1,1,1-trifluoro-2-methylpropan-2-yl)oxy)ethyl)-4-methyl-1,2,5-oxadiazole-3-carboxamide (Example 64) as the second eluting isomer in 9% yield. Data for Example 63: ¹H NMR (400 MHz, DMSO-d₆) δ 12.51-12.31 (m, 1H), 9.65-9.45 (m, 1H), 7.59-7.33 (m, 3H), 7.12-7.04 (m, 1H), 5.49-5.39 (m, 1H), 5.19-5.09 (m, 1H), 4.26-4.15 (m, 1H), 4.03 (t, J=9.2 Hz, 1H), 3.94-3.83 (m, 1H), 3.81-3.72 (m, 1H), 3.34-3.33 (m, 5H), 2.52 (s, 3H), 1.38 (s, 3H), 1.36-1.32 (m, 6H). MS (ESI) m/z: [M+H]⁺ Found 622.3. Data for Example 64: ¹H NMR (400 MHz, DMSO-d₆) δ 12.51-12.34 (m, 1H), 9.65-9.47 (m, 1H), 7.62-7.35 (m, 3H), 7.19-7.05 (m, 1H), 5.52-5.38 (m, 1H), 5.19-5.05 (m, 1H), 4.25-4.16 (m, 1H), 4.09-4.00 (m, 1H), 3.92-3.83 (m, 1H), 3.82-3.74 (m, 1H), 3.68-3.61 (m, 1H), 3.32-3.30 (m, 5H), 2.53 (s, 3H), 1.38-1.33 (m, 5H), 1.27 (d, J=5.9 Hz, 3H). MS (ESI) m/z: [M+H]⁺ Found 622.3.

### Example 65: N-((1R,2R)-2-Cyclopropoxy-1-(5-((S)-1-(5,5-difluoro-2-oxotetrahydropyrimidin-1(2H)-yl)-2-methoxyethyl)-1H-benzo[d]imidazol-2-yl)propyl)-4-methyl-1,2,5-oxadiazole-3-carboxamide

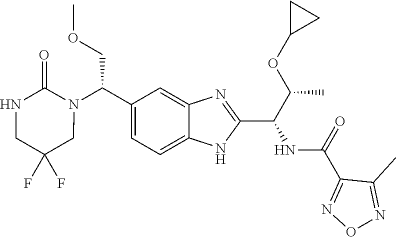 [SMILES: COC[C@H](c1ccc2[nH]c([C@@H](NC(=O)c3nonc3C)[C@@H](C)OC3CC3)nc2c1)N1CC(F)(F)CNC1=O]

The title compound was prepared as described for the synthesis of Example 2 using 1-((S)-1-(2-((1R,2R)-1-amino-2-cyclopropoxypropyl)-1H-benzo[d]imidazol-5-yl)-2-methoxyethyl)-5,5-difluorotetrahydropyrimidin-2(1H)-one (Intermediate 131) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. The product was initially purified by silica gel chromatography (0-100% (10% MeOH in DCM)/hexanes) then further purified by silica gel chromatography again (0-100% (10% MeOH in EtOAc)/hexanes) to afford, after lyophilization, the title compound in 47% yield. ¹H NMR (400 MHz, CD₃OD) δ 7.63-7.49 (m, 2H), 7.25 (dd, J=1.5, 8.5 Hz, 1H), 5.82 (t, J=6.6 Hz, 1H), 5.41 (d, J=4.0 Hz, 1H), 4.35-4.26 (m, 1H), 4.02-3.89 (m, 2H), 3.71-3.47 (m, 3H), 3.45 (s, 3H), 3.38-3.32 (m, 2H), 2.54 (s, 3H), 1.33 (d, J=6.5 Hz, 3H), 0.54-0.39 (m, 2H), 0.36-0.20 (m, 2H). MS (ESI) m/z: [M+H]⁺ Found 534.2.

### Example 66: N-((1R,2R)-1-(5-((S)-2-Cyclopropoxy-1-(5,5-difluoro-2-oxotetrahydropyrimidin-1(2H)-yl)ethyl)-1H-benzo[d]imidazol-2-yl)-2-(((S)-1,1,1-trifluoropropan-2-yl)oxy)propyl)-4-methyl-1,2,5-oxadiazole-3-carboxamide

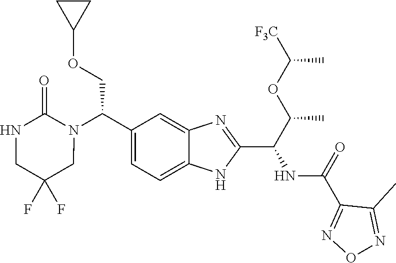 [SMILES: Cc1nonc1C(=O)N[C@H](c1nc2cc([C@@H](COC3CC3)N3CC(F)(F)CNC3=O)ccc2[nH]1)[C@@H](C)O[C@@H](C)C(F)(F)F]

The title compound was prepared as described for the synthesis of Example 2 using 1-((S)-1-(2-((1R,2R)-1-amino-2-(((S)-1,1,1-trifluoropropan-2-yl)oxy)propyl)-1H-benzo[d]imidazol-5-yl)-2-cyclopropoxyethyl)-5,5-difluorotetrahydropyrimidin-2(1H)-one (Intermediate 180) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl) imidazolidin-2-one. The product was purified by SFC (Stationary phase: Chiralpak IC, 5 μm, 250×21 mm, Mobile phase: 25% methanol, 75% CO₂) to afford, after lyophilization, the title compound in 55% yield. ¹H NMR (500 MHz, CD₃OD) δ 7.83-7.65 (m, 2H), 7.49-7.38 (m, 1H), 5.95-5.82 (m, 1H), 5.72-5.53 (m, 1H), 4.60-4.44 (m, 1H), 4.22-4.11 (m, 3H), 3.78-3.49 (m, 5H), 2.61 (s, 3H), 1.46-1.37 (m, 6H), 0.72-0.55 (m, 4H). MS (ESI) m/z: [M+H]⁺ Found 616.3.

### Example 67: N-((1R,2R)-1-(5-((S)-2-Cyclopropoxy-1-(5,5-difluoro-2-oxotetrahydropyrimidin-1(2H)-yl)ethyl)-1H-benzo[d]imidazol-2-yl)-2-(((S)-1,1,1-trifluoropropan-2-yl)oxy)propyl)-4-cyclopropyl-1,2,5-oxadiazole-3-carboxamide

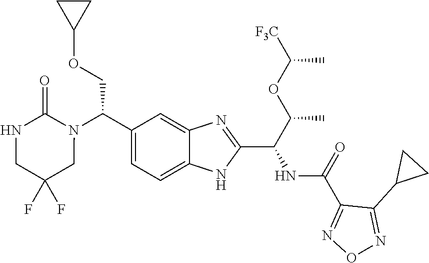 [SMILES: C[C@H](O[C@H](C)[C@H](NC(=O)c1nonc1C1CC1)c1nc2cc([C@@H](COC3CC3)N3CC(F)(F)CNC3=O)ccc2[nH]1)C(F)(F)F]

The title compound was prepared as described for the synthesis of Example 2 using 1-((S)-1-(2-((1R,2R)-1-amino-2-(((S)-1,1,1-trifluoropropan-2-yl)oxy)propyl)-1H-benzo[d]imidazol-5-yl)-2-cyclopropoxyethyl)-5,5-difluorotetrahydropyrimidin-2(1H)-one (Intermediate 180) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl) imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. The product was purified by SFC (Stationary phase: Chiralpak IC, 5 μm, 250×21 mm, Mobile phase: 25% methanol, 75% CO₂) to afford, after lyophilization, the title compound in 55% yield. ¹H NMR (500 MHz, CD₃OD) δ 7.81-7.67 (m, 2H), 7.53-7.44 (m, 1H), 5.92-5.80 (m, 1H), 5.73-5.63 (m, 1H), 4.58-4.47 (m, 1H), 4.24-4.09 (m, 3H), 3.80-3.46 (m, 5H), 2.56-2.46 (m, 1H), 1.48-1.43 (m, 3H), 1.41-1.37 (m, 3H), 1.24-1.08 (m, 4H), 0.70-0.53 (m, 4H). MS (ESI) m/z: [M+H]⁺ Found 642.2.

### Example 68: N-((1R,2R)-1-(5-((S)-2-Cyclopropoxy-1-((S)-2-oxo-4-(trifluoromethyl)imidazolidin-1-yl)ethyl)-1H-benzo[d]imidazol-2-yl)-2-(((S)-1,1,1-trifluoropropan-2-yl)oxy)propyl)-4-cyclopropyl-1,2,5-oxadiazole-3-carboxamide

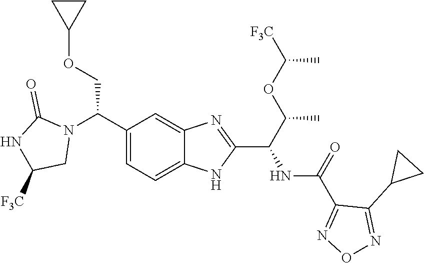 [SMILES: C[C@H](O[C@H](C)[C@H](NC(=O)c1nonc1C1CC1)c1nc2cc([C@@H](COC3CC3)N3C[C@@H](C(F)(F)F)NC3=O)ccc2[nH]1)C(F)(F)F]

The title compound was prepared as described for the synthesis of Example 2 using (S)-1-((S)-1-(2-((1R,2R)-1-amino-2-(((S)-1,1,1-trifluoropropan-2-yl)oxy)propyl)-1H-benzo[d]imidazol-5-yl)-2-cyclopropoxyethyl)-4-(trifluoromethyl)imidazolidin-2-one (Intermediate 181) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl) imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. The product was initially purified by SFC (Stationary phase: IG (2×25 cm), Mobile phase: 20% isopropanol (0.1% DEA)/80% CO₂) then subsequently purified by SFC (Stationary phase: Whelk-O1 (2×25 cm), Mobile phase: 20% isopropanol (0.1% DEA)/80% CO₂) to afford, after lyophilization, the title compound in 17% yield. ¹H NMR (500 MHz, CD₃OD) δ 7.68-7.54 (m, 1H), 7.54-7.40 (m, 1H), 7.22 (dd, J=1.4, 8.4 Hz, 1H), 5.46 (d, J=4.8 Hz, 1H), 5.27 (dd, J=5.4, 8.4 Hz, 1H), 4.46-4.35 (m, 1H), 4.34-4.25 (m, 1H), 4.13-3.97 (m, 3H), 3.84 (t, J=10.3 Hz, 1H), 3.49-3.41 (m, 1H), 2.53-2.42 (m, 1H), 1.31 (d, J=6.3 Hz, 7H), 1.19-1.01 (m, 4H), 0.63-0.46 (m, 4H). MS (ESI) m/z: [M+H]⁺ Found 660.8.

### Example 69: N-((1R,2R)-2-Cyclopropoxy-1-(5-((S)-2-cyclopropoxy-1-((S)-2-oxo-4-(trifluoromethyl)imidazolidin-1-yl)ethyl)-1H-benzo[d]imidazol-2-yl)propyl)-4-cyclopropyl-1,2,5-oxadiazole-3-carboxamide

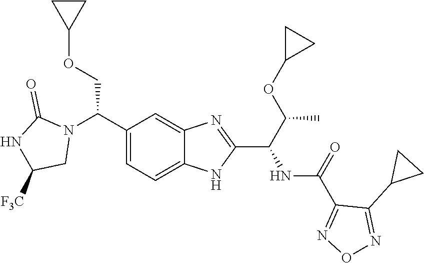 [SMILES: C[C@@H](OC1CC1)[C@H](NC(=O)c1nonc1C1CC1)c1nc2cc([C@@H](COC3CC3)N3C[C@@H](C(F)(F)F)NC3=O)ccc2[nH]1]

The title compound was prepared as described for the synthesis of Example 2 using (S)-1-((S)-1-(2-((1R,2R)-1-amino-2-cyclopropoxypropyl)-1H-benzo[d]imidazol-5-yl)-2-cyclopropoxyethyl)-4-(trifluoromethyl)imidazolidin-2-one (Intermediate 182) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl) imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. The product was initially purified by silica gel chromatography (0-100% (10% MeOH in DCM)/hexanes) then subsequently purified by SFC (Stationary phase: Whelk O1 SS 5 μm, 250×21 mm, Mobile phase: 20% isopropanol with 0.2% isopropylamine, 80% CO₂) to afford the title compound in 16% yield. ¹H NMR (500 MHz, CD₃OD) δ 7.70-7.42 (m, 2H), 7.22 (dd, J=1.5, 8.5 Hz, 1H), 5.42 (d, J=4.0 Hz, 1H), 5.29-5.23 (m, 1H), 4.36-4.25 (m, 2H), 4.14-4.05 (m, 1H), 4.04-3.98 (m, 1H), 3.85 (t, J=10.1 Hz, 1H), 3.50-3.40 (m, 1H), 3.36-3.32 (m, 2H), 2.51-2.42 (m, 1H), 1.34 (d, J=6.3 Hz, 3H), 1.19-1.02 (m, 4H), 0.63-0.42 (m, 6H), 0.34-0.28 (m, 1H), 0.24-0.18 (m, 1H). MS (ESI) m/z: [M+H]⁺ Found 604.3.

### Example 70: N-((1R,2R)-2-Cyclopropoxy-1-(5-((S)-2-cyclopropoxy-1-((S)-2-oxo-4-(trifluoromethyl)imidazolidin-1-yl)ethyl)-1H-benzo[d]imidazol-2-yl)propyl)-4-methyl-1,2,5-oxadiazole-3-carboxamide

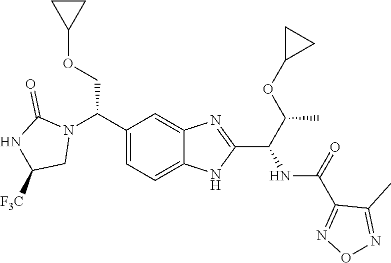 [SMILES: Cc1nonc1C(=O)N[C@H](c1nc2cc([C@@H](COC3CC3)N3C[C@@H](C(F)(F)F)NC3=O)ccc2[nH]1)[C@@H](C)OC1CC1]

The title compound was prepared as described for the synthesis of Example 2 using (S)-1-((S)-1-(2-((1R,2R)-1-amino-2-cyclopropoxypropyl)-1H-benzo[d]imidazol-5-yl)-2-cyclopropoxyethyl)-4-(trifluoromethyl)imidazolidin-2-one (Intermediate 182) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl) imidazolidin-2-one. The product was purified initially by silica gel chromatography (0-100% (10% 2 M NH₃/MeOH in DCM)/DCM) then subsequently purified by SFC (Stationary phase: Whelk O1 SS 5 μm, 250×21 mm, Mobile phase: 20% isopropanol with 0.2% isopropylamine, 80% CO₂) to afford the title compound, after lyophilization, in 19% yield. ¹H NMR (500 MHz, CD₃OD) δ 7.65-7.44 (m, 2H), 7.22 (dd, J=1.5, 8.5 Hz, 1H), 5.41 (d, J=4.0 Hz, 1H), 5.29-5.23 (m, 1H), 4.34-4.26 (m, 2H), 4.13-4.06 (m, 1H), 4.04-3.98 (m, 1H), 3.85 (t, J=10.1 Hz, 1H), 3.48-3.42 (m, 1H), 3.36-3.32 (m, 2H), 2.54 (s, 3H), 1.33 (d, J=6.3 Hz, 3H), 0.61-0.42 (m, 6H), 0.35-0.28 (m, 1H), 0.25-0.18 (m, 1H). MS (ESI) m/z: [M+H]⁺ Found 578.2.

### Example 71: N-((1R,2R)-2-Cyclopropoxy-1-(5-((S)-2-cyclopropoxy-1-(5,5-difluoro-2-oxotetrahydropyrimidin-1(2H)-yl)ethyl)-1H-benzo[d]imidazol-2-yl)propyl)-4-cyclopropyl-1,2,5-oxadiazole-3-carboxamide

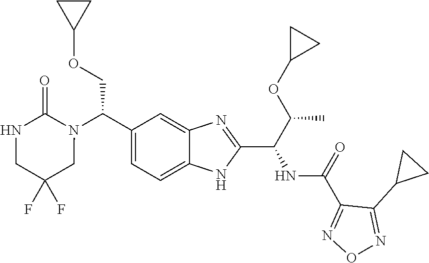 [SMILES: C[C@@H](OC1CC1)[C@H](NC(=O)c1nonc1C1CC1)c1nc2cc([C@@H](COC3CC3)N3CC(F)(F)CNC3=O)ccc2[nH]1]

The title compound was prepared as described for the synthesis of Example 2 using 1-((S)-1-(2-((1R,2R)-1-amino-2-cyclopropoxypropyl)-1H-benzo[d]imidazol-5-yl)-2-cyclopropoxyethyl)-5,5-difluorotetrahydropyrimidin-2(1H)-one (Intermediate 183) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl) imidazolidin-2-one and 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. The product was purified by silica gel chromatography (0-100% (10% MeOH in EtOAc)/hexanes) to afford the title compound in 46% yield. ¹H NMR (500 MHz, CD₃OD) δ 7.64-7.49 (m, 2H), 7.24 (dd, J=1.3, 8.5 Hz, 1H), 5.84-5.78 (m, 1H), 5.42 (d, J=4.0 Hz, 1H), 4.34-4.27 (m, 1H), 4.09-4.04 (m, 2H), 3.66-3.43 (m, 4H), 3.37-3.32 (m, 1H), 3.30-3.23 (m, 1H), 2.49-2.42 (m, 1H), 1.33 (d, J=6.3 Hz, 3H), 1.21-1.01 (m, 4H), 0.64-0.43 (m, 6H), 0.35-0.21 (m, 2H). MS (ESI) m/z: [M+H]⁺ Found 586.3.

### Example 72: N-((1R,2R)-2-Cyclopropoxy-1-(5-((S)-2-cyclopropoxy-1-(5,5-difluoro-2-oxotetrahydropyrimidin-1(2H)-yl)ethyl)-1H-benzo[d]imidazol-2-yl)propyl)-4-fluoro-1-isopropyl-1H-pyrazole-5-carboxamide

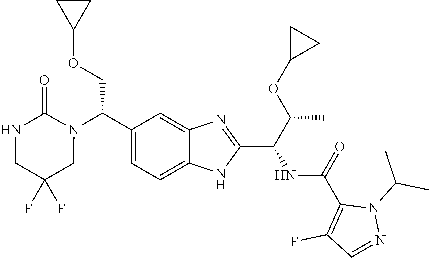 [SMILES: CC(C)n1ncc(F)c1C(=O)N[C@H](c1nc2cc([C@@H](COC3CC3)N3CC(F)(F)CNC3=O)ccc2[nH]1)[C@@H](C)OC1CC1]

1-((S)-1-(2-((1R,2R)-1-Amino-2-cyclopropoxypropyl)-1H-benzo[d]imidazol-5-yl)-2-cyclopropoxyethyl)-5,5-difluorotetrahydropyrimidin-2(1H)-one (57 mg, 0.13 mmol, Intermediate 183) and acetonitrile (3 mL) were combined under nitrogen, followed by the addition of 4-fluoro-1-isopropyl-1H-pyrazole-5-carboxylic acid (21.2 mg, 0.12 mmol), 1-methylimidazole (60 μL, 0.75 mmol) and chloro-N,N,N′N′-tetramethylformamidinium hexafluorophosphate (43.3 mg, 0.15 mmol) and the resulting mixture was allowed to stir at rt overnight. The reaction solution was then transferred to a separatory funnel with ethyl acetate and deionized water. The layers were separated then the aqueous layer was extracted two times with ethyl acetate. The combined organic phases were dried over anhydrous magnesium sulfate, filtered and concentrated to dryness under reduced pressure. The product was purified by silica gel chromatography (0-100% (10% MeOH in EtOAc)/hexanes)) to afford the title compound in 52% yield. ¹H NMR (400 MHz, CD₃OD) δ 7.47-7.37 (m, 3H), 7.13 (dd, J=1.4, 8.4 Hz, 1H), 5.67 (t, J=6.6 Hz, 1H), 5.28-5.19 (m, 2H), 4.18-4.10 (m, 1H), 3.96-3.89 (m, 2H), 3.55-3.33 (m, 3H), 3.21-3.12 (m, 3H), 1.31-1.18 (m, 9H), 0.50-0.27 (m, 6H), 0.23-0.11 (m, 1H), 0.11-0.01 (m, 1H). MS (ESI) m/z: [M+H]⁺ Found 604.3.

### Example 73: N-((1R,2R)-2-Cyclopropoxy-1-(5-((S)-2-cyclopropoxy-1-(5,5-difluoro-2-oxotetrahydropyrimidin-1(2H)-yl)ethyl)-1H-benzo[d]imidazol-2-yl)propyl)-4-methyl-1,2,5-oxadiazole-3-carboxamide

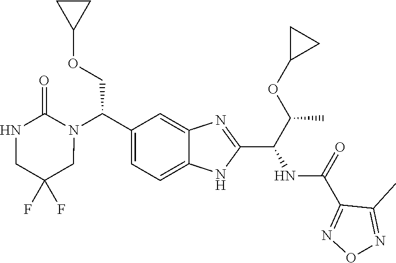 [SMILES: Cc1nonc1C(=O)N[C@H](c1nc2cc([C@@H](COC3CC3)N3CC(F)(F)CNC3=O)ccc2[nH]1)[C@@H](C)OC1CC1]

The title compound was prepared as described for the synthesis of Example 2 using 1-((S)-1-(2-((1R,2R)-1-amino-2-cyclopropoxypropyl)-1H-benzo[d]imidazol-5-yl)-2-cyclopropoxyethyl)-5,5-difluorotetrahydropyrimidin-2(1H)-one (Intermediate 183) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl) imidazolidin-2-one. The product was purified by silica gel chromatography (0-100% (10% MeOH in EtOAc)/hexanes)) to afford the title compound in 48% yield. ¹H NMR (400 MHz, CD₃OD) δ 7.65-7.48 (m, 2H), 7.25 (dd, J=1.5, 8.5 Hz, 1H), 5.81 (t, J=6.6 Hz, 1H), 5.42 (d, J=3.8 Hz, 1H), 4.39-4.22 (m, 1H), 4.16-3.98 (m, 2H), 3.71-3.55 (m, 2H), 3.53-3.41 (m, 2H), 3.38-3.32 (m, 1H), 3.30-3.22 (m, 1H), 2.53 (s, 3H), 1.33 (d, J=6.3 Hz, 3H), 0.64-0.42 (m, 6H), 0.36-0.20 (m, 2H). MS (ESI) m/z: [M+H]⁺ Found 560.3.

### Example 74: N-[(1R*,2R)-1-[5-[(1S)-2-Methoxy-1-[(4S)-2-oxo-4-(trifluoromethyl)imidazolidin-1-yl]ethyl]-1H-benzimidazol-2-yl]-2-(2,2,2-trifluoro-1,1-dimethyl-ethoxy)propyl]-4-methyl-1,2,5-oxadiazole-3-carboxamide

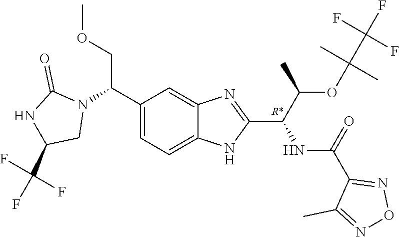 [SMILES: COC[C@H](c1ccc2[nH]c([C@@H](NC(=O)c3nonc3C)[C@@H](C)OC(C)(C)C(F)(F)F)nc2c1)N1C[C@@H](C(F)(F)F)NC1=O]

Step A: (R)—N-((1R*,2R)-1-(5-((S)-1-(1,3-Dioxoisoindolin-2-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide. The title compound (32% yield) was synthesized in a manner analogous to Intermediate 77 using (R)-2-methyl-N—((R,E)-2-((1,1,1-trifluoro-2-methylpropan-2-yl)oxy)propylidene)propane-2-sulfinamide (Intermediate 184) in place of (R,E)-2-methyl-N-(2-((1,1,1-trifluoro-2-methylpropan-2-yl)oxy)ethylidene)propane-2-sulfinamide.

Step B: (R)—N-((1R*,2R)-1-(5-((S)-1-Amino-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide. The title compound was synthesized in a manner analogous to Intermediate 78 using (R)—N-((1R*,2R)-1-(5-((S′)-1-(1,3-dioxoisoindolin-2-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide (Step A) in place of (R)—N—((R*)-1-(5-((S)-1-(1,3-dioxoisoindolin-2-yl)-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and was used without further purification (100% yield).

Step C: (R)—N-((1R*,2R)-1-(5-((S)-2-Methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide. The title compound (14% yield) was synthesized in a manner analogous to Intermediate 79 using (R)—N-((1R*,2R)-1-(5-((S)-1-amino-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide (Step B) in place of (R)—N—((R*)-1-(5-((S)-1-amino-2-methoxyethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

Step D: (S)-1-((S)-1-(2-((1R*,2R)-1-Amino-2-((1,1,1-trifluoro-2-methylpropan-2-yl)oxy)propyl)-1H-benzo[d]imidazol-5-yl)-2-methoxyethyl)-4-(trifluoromethyl)imidazolidin-2-one. The title compound (70% yield) was synthesized in a manner analogous to Intermediate 80 using (R)—N-((1R*,2R)-1-(5-((S)-2-methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide (Step C) in place of (R)—N—((R*)-1-(5-((S)-2-methoxy-1-((S)-2-oxo-4-(trifluoromethyl)imidazolidin-1-yl)ethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

Step E: N-[(1R*,2R)-1-[5-[(1S)-2-Methoxy-1-[(4S)-2-oxo-4-(trifluoromethyl)imidazolidin-1-yl]ethyl]-1H-benzimidazol-2-yl]-2-(2,2,2-trifluoro-1,1-dimethyl-ethoxy)propyl]-4-methyl-1,2,5-oxadiazole-3-carboxamide. The title compound was synthesized in a manner analogous to Example 2 using (S)-1-((S)-1-(2-((1R*,2R)-1-amino-2-((1,1,1-trifluoro-2-methylpropan-2-yl)oxy)propyl)-1H-benzo[d]imidazol-5-yl)-2-methoxyethyl)-4-(trifluoromethyl)imidazolidin-2-one (Step D) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one. The material was purified by SFC (Stationary phase: Chiralpak IB N₃ 5 μm, 250×21 mm, Mobile phase: 20% methanol with 0.2% triethylamine, 80% CO₂) to afford the title compound in 21% yield. ¹H NMR (500 MHz, DMSO-d₆) δ 12.39 (d, J=13.4 Hz, 1H), 9.06 (dd, J=9.2, 2.7 Hz, 1H), 7.58 (d, J=8.4 Hz, 1H), 7.53 (d, J=1.8 Hz, 1H), 7.51-7.45 (m, 2H), 7.42-7.36 (m, 1H), 7.11 (ddd, J=10.5, 8.5, 1.7 Hz, 1H), 5.33 (dd, J=9.1, 6.0 Hz, 1H), 5.14 (ddd, J=8.7, 5.3, 3.1 Hz, 1H), 4.48 (p, J=6.3 Hz, 1H), 4.45-4.36 (m, 1H), 3.90 (ddd, J=10.1, 8.7, 4.7 Hz, 1H), 3.83-3.69 (m, 3H), 3.33 (s, 3H), 3.25-3.17 (m, 1H), 2.59 (s, 1H), 1.29 (s, 3H), 1.15 (d, J=2.0 Hz, 5H). MS (ESI) m/z: [M+H]⁺ Found 622.0.

### Example 75: N-((1R*,2R)-1-(5-((R)-Cyclopropyl(5,5-difluoro-2-oxotetrahydropyrimidin-1(2H)-yl)methyl)-1H-imidazo[4,5-b]pyridin-2-yl)-2-(((R)-1,1,1-trifluoropropan-2-yl)oxy)propyl)-1-isopropyl-1H-pyrazole-5-carboxamide

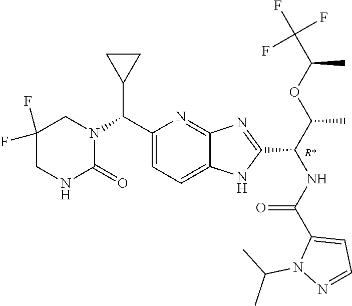 [SMILES: CC(C)n1nccc1C(=O)N[C@H](c1nc2nc([C@@H](C3CC3)N3CC(F)(F)CNC3=O)ccc2[nH]1)[C@@H](C)O[C@H](C)C(F)(F)F]

The title compound was prepared as described for Example 6 using 1-((R)-(2-((1R*,2R)-1-amino-2-(((R)-1,1,1-trifluoropropan-2-yl)oxy)propyl)-1H-imidazo[4,5-b]pyridin-5-yl)(cyclopropyl)methyl)-5,5-difluorotetrahydropyrimidin-2(1H)-one (Intermediate 104) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one and 1-isopropyl-1H-pyrazole-5-carboxylic acid in place of 4-methyl-1,2,5-oxadiazole-3-carboxylic acid. The material was initially purified by silica gel chromatography (0-100% (10% MeOH in EtOAc)/hexanes) and then subsequently purified by SFC with a chiral stationary phase (Stationary phase: Chiralpak IH, 5 μm, 250×21 mm, Mobile phase: 10% MeOH (with 0.08% IPA), 90% CO₂). The second eluting peak was further purified by preparative HPLC (basic) to provide the title compound, Example 75 (22% yield). ¹H NMR (400 MHz, DMSO-d₆, benzimidazole NH is absent) δ 8.92-8.83 (m, 1H), 7.96 (d, J=8.1 Hz, 1H), 7.53 (d, J=2.0 Hz, 1H), 7.25 (d, J=8.3 Hz, 1H), 6.99 (d, J=2.0 Hz, 1H), 6.76-6.66 (m, 1H), 5.50-5.38 (m, 1H), 5.36-5.30 (m, 1H), 4.72-4.65 (m, 1H), 4.42-4.32 (m, 2H), 3.99-3.75 (m, 2H), 3.59-3.39 (m, 2H), 1.62-1.49 (m, 1H), 1.40-1.33 (m, 6H), 1.18-1.06 (m, 6H), 0.69-0.57 (m, 2H), 0.52-0.44 (m, 1H), 0.38-0.30 (m, 1H). MS (ESI) m/z: [M+H]⁺ Found 613.2.

### Example 76: 4-Cyclopropyl-N—((R)-1-(5-((R)-cyclopropyl(5,5-difluoro-2-oxotetrahydropyrimidin-1(2H)-yl-6,6-d₂)methyl)-1H-benzo[d]imidazol-2-yl)-2-((1,1,1-trifluoro-2-methylpropan-2-yl)oxy)ethyl)-1,2,5-oxadiazole-3-carboxamide

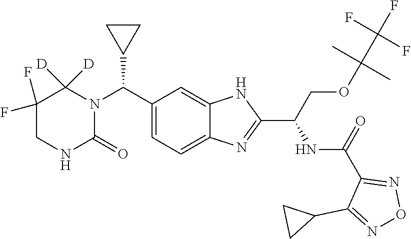 [SMILES: [2H]C1([2H])N([C@@H](c2ccc3nc([C@H](COC(C)(C)C(F)(F)F)NC(=O)c4nonc4C4CC4)[nH]c3c2)C2CC2)C(=O)NCC1(F)F]

The title compound was prepared as described for Example 6 using 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-6,6-d₂ (Intermediate 186) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one and 4-cyclopropyl-1,2,5-oxadiazole-3-carboxylic acid in place of 4-methyl-1,2,5-oxadiazole-3-carboxylic acid, and stirring at 40° C. for 30 min instead of 50° C. for 2 h. The residue was initially purified by silica gel chromatography (0-100% EtOAc (with 10% MeOH)/hexanes) and then subsequently purified by preparative HPLC (basic) to provide the title compound as a white solid (24% yield). ¹H NMR (400 MHz, DMSO-d₆) δ 12.55-12.29 (m, 1H), 9.73-9.46 (m, 1H), 7.77-7.39 (m, 2H), 7.21 (dd, J=1.2, 8.4 Hz, 1H), 6.82-6.66 (m, 1H), 5.55-5.38 (m, 1H), 4.72 (d, J=10.3 Hz, 1H), 4.27-4.18 (m, 1H), 4.12-3.99 (m, 1H), 3.64-3.40 (m, 2H), 2.47-2.38 (m, 1H), 1.56-1.44 (m, 1H), 1.39-1.34 (m, 6H), 1.21-1.14 (m, 2H), 1.05-0.99 (m, 2H), 0.84-0.76 (m, 1H), 0.63-0.54 (m, 1H), 0.44-0.34 (m, 2H). MS (ESI) m/z: [M+H]⁺ Found 614.2.

### Example 77: N—((R)-1-(5-((R)-Cyclopropyl(5,5-difluoro-2-oxotetrahydropyrimidin-1(2H)-yl-6,6-d₂)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

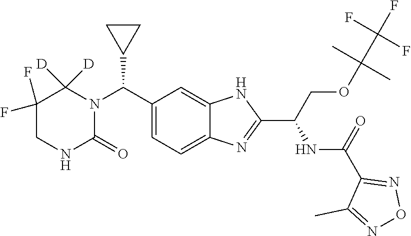 [SMILES: [2H]C1([2H])N([C@@H](c2ccc3nc([C@H](COC(C)(C)C(F)(F)F)NC(=O)c4nonc4C)[nH]c3c2)C2CC2)C(=O)NCC1(F)F]

The title compound was prepared as described for Example 2 using 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-6,6-d₂ (Intermediate 186) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and the mixture was stirred at rt for 15 min instead of 1 h. The glassy yellow solid obtained from silica gel chromatography was further purified by preparative HPLC (basic) to provide the title compound (28% yield). ¹H NMR (400 MHz, DMSO-d₆) δ 12.65-12.35 (m, 1H), 9.75-9.56 (m, 1H), 7.80-7.49 (m, 2H), 7.32-7.26 (m, 1H), 6.88-6.79 (m, 1H), 5.60-5.49 (m, 1H), 4.80 (d, J=10.5 Hz, 1H), 4.36-4.27 (m, 1H), 4.17-4.09 (m, 1H), 3.73-3.49 (m, 2H), 2.61 (s, 3H), 1.64-1.53 (m, 1H), 1.48-1.42 (m, 6H), 0.93-0.84 (m, 1H), 0.70-0.62 (m, 1H), 0.52-0.41 (m, 2H). MS (ESI) m/z: [M+H]⁺ Found 588.3.

### Example 78: 4-Cyclopropyl-N—((R)-1-(5-((R)-cyclopropyl(5,5-difluoro-2-oxotetrahydropyrimidin-1(2H)-yl-4,4,6,6-d₄)methyl)-1H-benzo[d]imidazol-2-yl)-2-((1,1,1-trifluoro-2-methylpropan-2-yl)oxy)ethyl)-1,2,5-oxadiazole-3-carboxamide

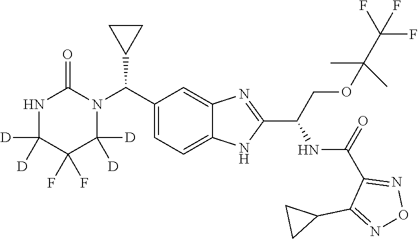 [SMILES: [2H]C1([2H])NC(=O)N([C@@H](c2ccc3[nH]c([C@H](COC(C)(C)C(F)(F)F)NC(=O)c4nonc4C4CC4)nc3c2)C2CC2)C([2H])([2H])C1(F)F]

The title compound was prepared as described for Example 6 using 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-4,4,6,6-d₄ (Intermediate 188) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one and 4-cyclopropyl-1,2,5-oxadiazole-3-carboxylic acid in place of 4-methyl-1,2,5-oxadiazole-3-carboxylic acid, and stirring at 40° C. for 30 min instead of 50° C. for 2 h. The residue was initially purified by silica gel chromatography (0-100% EtOAc (with 10% MeOH)/hexanes) and then subsequently purified by preparative HPLC (basic) to provide the title compound (19% yield). ¹H NMR (400 MHz, DMSO-d₆) δ 12.49-12.37 (m, 1H), 9.70-9.49 (m, 1H), 7.78-7.41 (m, 2H), 7.25-7.17 (m, 1H), 6.81-6.67 (m, 1H), 5.53-5.41 (m, 1H), 4.75-4.69 (m, 1H), 4.26-4.19 (m, 1H), 4.09-4.01 (m, 1H), 2.46-2.38 (m, 1H), 1.56-1.44 (m, 1H), 1.39-1.34 (m, 6H), 1.20-1.14 (m, 2H), 1.05-0.99 (m, 2H), 0.84-0.75 (m, 1H), 0.62-0.54 (m, 1H), 0.44-0.33 (m, 2H). MS (ESI) m/z: [M+H]⁺ Found 616.3.

### Example 79: N—((R)-1-(5-((R)-Cyclopropyl(5,5-difluoro-2-oxotetrahydropyrimidin-1(2H)-yl-4,4,6,6-d₄)methyl)-1H-benzo[d]imidazol-2-yl)-2-((1,1,1-trifluoro-2-methylpropan-2-yl)oxy)ethyl)-1-isopropyl-1H-pyrazole-5-carboxamide

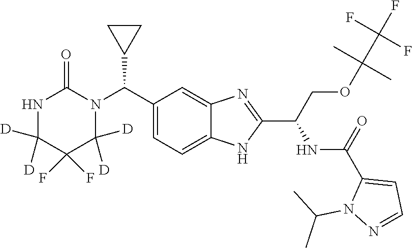 [SMILES: [2H]C1([2H])NC(=O)N([C@@H](c2ccc3[nH]c([C@H](COC(C)(C)C(F)(F)F)NC(=O)c4ccnn4C(C)C)nc3c2)C2CC2)C([2H])([2H])C1(F)F]

The title compound was prepared as described for Example 6 using 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-4,4,6,6-d₄ (Intermediate 188) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one and 1-isopropyl-1H-pyrazole-5-carboxylic acid in place of 4-methyl-1,2,5-oxadiazole-3-carboxylic acid, and stirring at 40° C. for 30 min instead of 50° C. for 2 h. The residue was initially purified by silica gel chromatography (0-100% EtOAc (with 10% MeOH)/hexanes) and then subsequently purified by preparative HPLC (basic) to provide the title compound (29% yield). ¹H NMR (400 MHz, DMSO-d₆) δ 12.55-12.39 (m, 1H), 9.03-8.92 (m, 1H), 7.79-7.48 (m, 3H), 7.31-7.26 (m, 1H), 7.00 (d, J=2.0 Hz, 1H), 6.85-6.78 (m, 1H), 5.62-5.46 (m, 2H), 4.80 (d, J=10.3 Hz, 1H), 4.32-4.24 (m, 1H), 4.14-4.03 (m, 1H), 1.64-1.53 (m, 1H), 1.50-1.45 (m, 6H), 1.43 (d, J=1.0 Hz, 6H), 0.92-0.84 (m, 1H), 0.71-0.62 (m, 1H), 0.51-0.43 (m, 2H). MS (ESI) m/z: [M+H]⁺ Found 616.3.

### Example 80: N—((R)-1-(5-((R)-Cyclopropyl(5,5-difluoro-2-oxotetrahydropyrimidin-1(2H)-yl-4,4,6,6-d₄)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

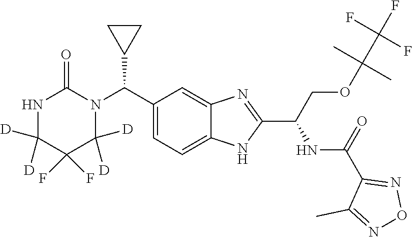 [SMILES: [2H]C1([2H])NC(=O)N([C@@H](c2ccc3[nH]c([C@H](COC(C)(C)C(F)(F)F)NC(=O)c4nonc4C)nc3c2)C2CC2)C([2H])([2H])C1(F)F]

The title compound was prepared as described for Example 2 using 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-4,4,6,6-d₄ (Intermediate 188) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and the mixture was stirred at rt for 15 min instead of 1 h. The glassy yellow solid obtained from silica gel chromatography was further purified by preparative HPLC (basic) to provide the title compound (27% yield). ¹H NMR (400 MHz, DMSO-d₆) δ 12.65-12.33 (m, 1H), 9.76-9.48 (m, 1H), 7.81-7.51 (m, 2H), 7.33-7.20 (m, 1H), 6.90-6.71 (m, 1H), 5.60-5.44 (m, 1H), 4.85-4.73 (m, 1H), 4.36-4.27 (m, 1H), 4.18-4.06 (m, 1H), 2.61 (s, 3H), 1.64-1.53 (m, 1H), 1.44 (d, J=3.6 Hz, 6H), 0.91-0.84 (m, 1H), 0.70-0.62 (m, 1H), 0.52-0.41 (m, 2H). MS (ESI) m/z: [M+H]⁺ Found 590.2.

### Example 81: N-[(1R)-1-[5-[(R)-Cyclopropyl-(4,4-dideuterio-5,5-difluoro-2-oxo-hexahydropyrimidin-1-yl)methyl]-1H-benzimidazol-2-yl]-2-(2,2,2-trifluoro-1,1-dimethyl-ethoxy)ethyl]-4-methyl-1,2,5-oxadiazole-3-carboxamide

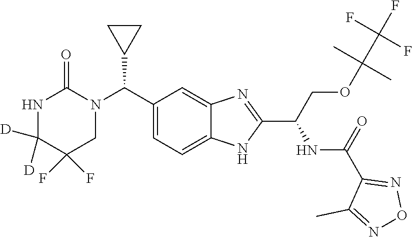 [SMILES: [2H]C1([2H])NC(=O)N([C@@H](c2ccc3[nH]c([C@H](COC(C)(C)C(F)(F)F)NC(=O)c4nonc4C)nc3c2)C2CC2)CC1(F)F]

The title compound was prepared as described for Example 2 using 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-4,4-d₂ (Intermediate 190) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one and the mixture was stirred at rt for 15 min instead of 1 h. The glassy yellow solid obtained from silica gel chromatography was further purified by preparative HPLC (basic) twice to provide the title compound (30% yield) as a white solid. ¹H NMR (600 MHz, DMSO-d₆) δ 12.41-12.36 (m, 1H), 9.58-9.53 (m, 1H), 7.73-7.40 (m, 2H), 7.23-7.18 (m, 1H), 6.76-6.70 (m, 1H), 5.49-5.41 (m, 1H), 4.75-4.69 (m, 1H), 4.26-4.19 (m, 1H), 4.08-4.03 (m, 1H), 3.78-3.67 (m, 1H), 3.27-3.18 (m, 1H), 2.53 (s, 3H), 1.53-1.45 (m, 1H), 1.38-1.34 (m, 6H), 0.82-0.77 (m, 1H), 0.62-0.55 (m, 1H), 0.43-0.36 (m, 2H). MS (ESI) m/z: [M+H]⁺ Found 588.2.

### Example 82: 4-Cyclopropyl-N-[(1R)-1-[5-[(R)-cyclopropyl-(4,4-dideuterio-5,5-difluoro-2-oxo-hexahydropyrimidin-1-yl)methyl]-1H-benzimidazol-2-yl]-2-(2,2,2-trifluoro-1,1-dimethyl-ethoxy)ethyl]-1,2,5-oxadiazole-3-carboxamide

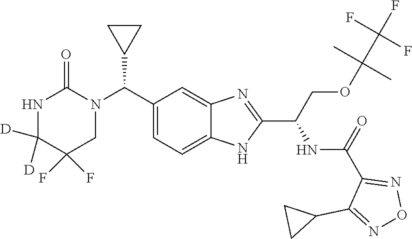 [SMILES: [2H]C1([2H])NC(=O)N([C@@H](c2ccc3[nH]c([C@H](COC(C)(C)C(F)(F)F)NC(=O)c4nonc4C4CC4)nc3c2)C2CC2)CC1(F)F]

The title compound was prepared as described for Example 6 using 1-((R)-(2-((R)-1-amino-2-((1,1,1-trifluoro-2-methylpropan-2-yl)oxy)ethyl)-1H-benzo[d]imidazol-5-yl)(cyclopropyl)methyl)-5,5-difluorotetrahydropyrimidin-2(1H)-one-4,4-d₂ (Intermediate 190) in place of (S)-1-((R)-1-(2-((R*)-1-amino-2-((1,1,1-trifluoro-2-methylpropan-2-yl)oxy)ethyl)-1H-benzo[d]imidazol-6-yl)ethyl)-4-(trifluoromethyl)imidazolidin-2-one and 4-cyclopropyl-1,2,5-oxadiazole-3-carboxylic acid in place of 4-methyl-1,2,5-oxadiazole-3-carboxylic acid, and stirring at 40° C. for 30 min instead of 50° C. for 2 h. The residue was initially purified by silica gel chromatography (0-100% EtOAc (with 10% MeOH)/hexanes) and then subsequently purified by preparative HPLC (basic) to provide the title compound (29% yield). ¹H NMR (600 MHz, DMSO-d₆) δ 12.53-12.47 (m, 1H), 9.70-9.65 (m, 1H), 7.80-7.49 (m, 2H), 7.31-7.27 (m, 1H), 6.85-6.79 (m, 1H), 5.59-5.51 (m, 1H), 4.85-4.76 (m, 1H), 4.34-4.27 (m, 1H), 4.17-4.10 (m, 1H), 3.88-3.76 (m, 1H), 3.36-3.26 (m, 1H), 2.52-2.45 (m, 1H), 1.63-1.55 (m, 1H), 1.46-1.41 (m, 6H), 1.28-1.22 (m, 2H), 1.14-1.07 (m, 2H), 0.91-0.85 (m, 1H), 0.70-0.60 (m, 1H), 0.53-0.42 (m, 2H). MS (ESI) m/z: [M+H]⁺ Found 614.3.

### Example 83: N—((R)-1-(5-((R)-Cyclopropyl((R)-4-isopropyl-2-oxoimidazolidin-1-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

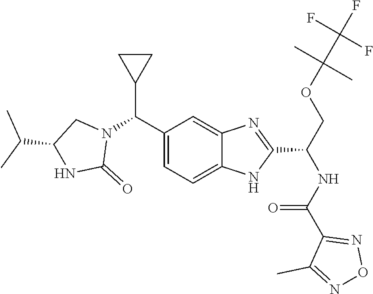 [SMILES: Cc1nonc1C(=O)N[C@@H](COC(C)(C)C(F)(F)F)c1nc2cc([C@@H](C3CC3)N3C[C@@H](C(C)C)NC3=O)ccc2[nH]1]

The title compound was prepared as described in Example 34 using (R)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-isopropylimidazolidin-2-one (Intermediate 192) in place of 1-((S)-1-(2-((R*)-1-amino-2-((1,1,1-trifluoro-2-methylpropan-2-yl)oxy)ethyl)-1H-benzo[d]imidazol-5-yl)-2-methoxyethyl)-5,5-difluorotetrahydropyrimidin-2(1H)-one and the mixture was stirred at rt for 12 h instead of 1 h. The reaction mixture was concentrated to dryness and purified by preparative HPLC (Phenomenex C18 3 μm, 75×30 mm (40-70% CH₃CN/H₂O with NH₄OH and NH₄HCO₃)). The collected material was lyophilized to provide the title compound as a white solid (25% yield). ¹H NMR (400 MHz, DMSO-d₆) δ 12.39 (d, J=9.6 Hz, 1H), 9.58-9.56 (m, 1H), 7.65-7.53 (m, 1H), 7.47-7.41 (m, 1H), 7.19-7.11 (m, 1H), 6.66-6.56 (m, 1H), 5.48-5.41 (m, 1H), 4.25-4.20 (m, 1H), 4.15-4.10 (m, 1H), 4.07-4.01 (m, 1H), 3.56-3.48 (m, 1H), 3.32-3.27 (m, 1H), 2.70-2.62 (m, 1H), 2.51-2.50 (m, 4H), 1.55-1.44 (m, 1H), 1.36-1.34 (m, 6H), 0.76-0.67 (m, 7H), 0.62-0.56 (m, 1H), 0.36-0.31 (m, 2H). MS (ESI) m/z: [M+H]⁺ Found 578.4.

### Example 84: N—((R)-1-(5-((R)-Cyclopropyl((R)-4-cyclopropyl-2-oxoimidazolidin-1-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

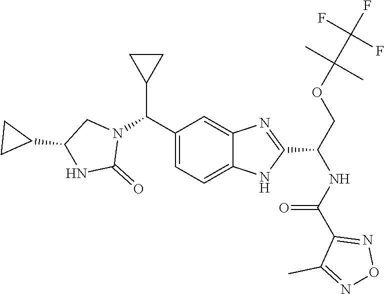 [SMILES: Cc1nonc1C(=O)N[C@@H](COC(C)(C)C(F)(F)F)c1nc2cc([C@@H](C3CC3)N3C[C@@H](C4CC4)NC3=O)ccc2[nH]1]

The title compound was prepared as described in Example 34 using (R)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-cyclopropylimidazolidin-2-one (Intermediate 194) in place of 1-((S)-1-(2-((R*)-1-amino-2-((1,1,1-trifluoro-2-methylpropan-2-yl)oxy)ethyl)-1H-benzo[d]imidazol-5-yl)-2-methoxyethyl)-5,5-difluorotetrahydropyrimidin-2(1H)-one and the mixture was stirred at rt for 3 h instead of 1 h. The reaction mixture was concentrated to dryness and purified by preparative HPLC (Phenomenex C18 3 μm, 75×30 mm (40-70% CH₃CN/H₂O with NH₄OH and NH₄HCO₃)). The collected material was lyophilized to provide the title compound as a white solid (49% yield). ¹H NMR (400 MHz, DMSO-d₆) δ 12.40 (d, J=11.2 Hz, 1H), 9.59-9.56 (m, 1H), 7.66-7.54 (m, 1H), 7.49-7.42 (m, 1H), 7.20-7.17 (m, 1H), 6.56 (d, J=8.8 Hz, 1H), 5.47-5.42 (m, 1H), 4.24-4.21 (m, 1H), 4.15-4.13 (m, 1H), 4.07-4.02 (m, 1H), 3.64-3.59 (m, 1H), 3.11-3.04 (m, 1H), 2.81-2.73 (m, 1H), 2.53 (s, 3H), 1.52-1.41 (m, 1H), 1.36 (s, 3H), 1.35 (s, 3H), 0.76-0.68 (m, 2H), 0.59-0.55 (m, 1H), 0.37-0.26 (m, 4H), 0.17-0.13 (m, 2H). MS (ESI) m/z: [M+H]⁺ Found 576.4.

### Example 85: N-[(1R,2R)-1-[6-[(R)-Cyclopropyl-[(4S)-2-oxo-4-(trifluoromethyl)imidazolidin-1-yl]methyl]-4-fluoro-1H-benzimidazol-2-yl]-2-[(1R)-2,2,2-trifluoro-1-methyl-ethoxy]propyl]-2-isopropyl-pyrazole-3-carboxamide

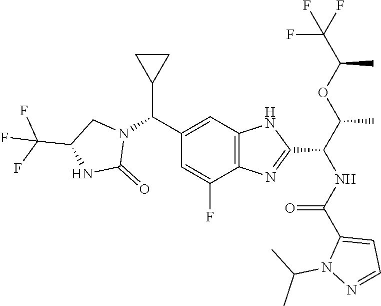 [SMILES: CC(C)n1nccc1C(=O)N[C@H](c1nc2c(F)cc([C@@H](C3CC3)N3C[C@@H](C(F)(F)F)NC3=O)cc2[nH]1)[C@@H](C)O[C@H](C)C(F)(F)F]

A mixture of(S)-1-((R)-(2-((1R,2R)-1-amino-2-(((R)-1,1,1-trifluoropropan-2-yl)oxy)propyl)-4-fluoro-1H-benzo[d]imidazol-6-yl)(cyclopropyl)methyl)-4-(trifluoromethyl)imidazolidin-2-one hydrochloride (59 mg, 0.108 mmol, Intermediate 195), 1-isopropyl-1H-pyrazole-5-carboxylic acid (16 mg, 0.104 mmol), HOBt (17 mg, 0.126 mmol), EDCI (40 mg, 0.209 mmol) and DIPEA (0.1 mL, 0.57 mmol) in DCM (5 mL) was stirred at rt overnight. The mixture was then concentrated to dryness to give a yellow oil. The oil was purified by silica gel chromatography (0-2% MeOH/DCM) and lyophilized to provide the title compound as a white solid (25% yield). ¹H NMR (400 MHz, DMSO-d₆) δ 8.99 (d, J=9.2 Hz, 1H), 7.59 (d, J=1.6 Hz, 1H), 7.53 (d, J=1.6 Hz, 1H), 7.34 (s, 1H), 7.02-6.94 (m, 2H), 5.51-5.39 (m, 1H), 5.29 (t, J=8.4 Hz, 1H), 4.53-4.44 (m, 1H), 4.42-4.33 (m, 2H), 4.12 (d, J=10.0 Hz, 1H), 3.86 (t, J=10.0 Hz, 1H), 3.24-3.15 (m, 1H), 1.52-1.41 (m, 1H), 1.35 (dd, J=6.4, 14.8 Hz, 6H), 1.13-1.08 (m, 6H), 0.81-0.74 (m, 1H), 0.64-0.55 (m, 1H), 0.52-0.44 (m, 1H), 0.39-0.30 (m, 1H). MS (ESI) m/z: [M+H]⁺ Found 648.3.

### Example 86: N-[(1R,2R)-1-[6-[(R)-Cyclopropyl-[(4S)-2-oxo-4-(trifluoromethyl)imidazolidin-1-yl]methyl]-4-fluoro-1H-benzimidazol-2-yl]-2-[(1R)-2,2,2-trifluoro-1-methyl-ethoxy]propyl]-4-methyl-1,2,5-oxadiazole-3-carboxamide

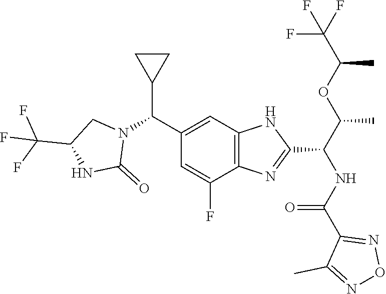 [SMILES: Cc1nonc1C(=O)N[C@H](c1nc2c(F)cc([C@@H](C3CC3)N3C[C@@H](C(F)(F)F)NC3=O)cc2[nH]1)[C@@H](C)O[C@H](C)C(F)(F)F]

A round-bottom flask was charged with 4-methyl-1,2,5-oxadiazole-3-carboxylic acid (14 mg, 0.11 mmol), T₃P (209 mg, 0.328 mmol, 50% in EtOAc), DIPEA (0.1 mL, 0.57 mmol), and DCM (5 mL) and the resulting mixture stirred at rt for 1 h. (S)-1-((R)-(2-((1R,2R)-1-Amino-2-(((R)-1,1,1-trifluoropropan-2-yl)oxy)propyl)-4-fluoro-1H-benzo[d]imidazol-6-yl)(cyclopropyl)methyl)-4-(trifluoromethyl)imidazolidin-2-one hydrochloride (59 mg, 0.11 mmol, Intermediate 195) was then added to the reaction mixture and the resulting mixture stirred at rt overnight. After this time, the reaction mixture was concentrated to dryness to give a yellow oil. The oil was purified by silica gel chromatography (0-2% MeOH/DCM) and lyophilized to provide the title compound as a white solid (28% yield). ¹H NMR (400 MHz, CD₃OD) δ 7.44 (s, 1H), 7.11 (d, J=12.0 Hz, 1H), 5.48 (d, J=4.4 Hz, 1H), 4.49-4.41 (m, 1H), 4.40-4.30 (m, 1H), 4.25 (d, J=10.0 Hz, 1H), 4.08-3.91 (m, 2H), 3.29 (d, J=4.0 Hz, 1H), 2.56 (s, 3H), 1.53-1.40 (m, 1H), 1.33 (d, J=6.4 Hz, 3H), 1.00 (d, J=6.4 Hz, 3H), 0.94-0.87 (m, 1H), 0.73-0.65 (m, 1H), 0.55-0.45 (m, 2H). MS (ESI) m/z: [M+H]⁺ Found 622.3.

### Example 87: N—((R)-1-(5-((R)-Cyclopropyl(5-oxo-4,6-diazaspiro[2.4]heptan-6-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

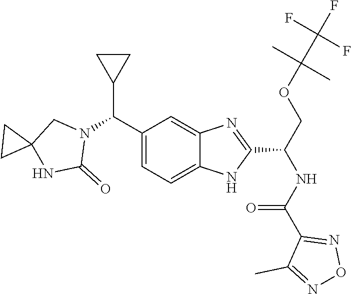 [SMILES: Cc1nonc1C(=O)N[C@@H](COC(C)(C)C(F)(F)F)c1nc2cc([C@@H](C3CC3)N3CC4(CC4)NC3=O)ccc2[nH]1]

A round-bottom flask was charged with 4-methyl-1,2,5-oxadiazole-3-carboxylic acid (17 mg, 0.13 mmol), T₃P (73 mg, 0.12 mmol, 50% in EtOAc), DIPEA (0.046 mL, 0.27 mmol), 6-((R)-(2-((R)-1-amino-2-((1,1,1-trifluoro-2-methylpropan-2-yl)oxy)ethyl)-1H-benzo[d]imidazol-5-yl)(cyclopropyl)methyl)-4,6-diazaspiro[2.4]heptan-5-one (40 mg, 0.089 mmol, Intermediate 197) and DCM (1.5 mL) and the resulting mixture stirred at rt for 12 h. After this time, the reaction mixture was concentrated to dryness to give a yellow oil. The oil was initially purified by preparative HPLC ((Phenomenex C18 3 μm, 75×30 mm, 40-70% MeCN/water with 0.05% NH₄OH+10 mM NH₄HCO₃) and then subsequently purified by silica gel chromatography (0-5% MeOH/DCM) to provide the title compound as a white solid (26% yield). ¹H NMR (400 MHz, DMSO-d₆) δ 12.40 (d, J=12.8 Hz, 1H), 9.58 (d, J=7.2 Hz, 1H), 7.65-7.54 (m, 1H), 7.49-7.43 (m, 1H), 7.22-7.16 (m, 1H), 6.50 (d, J=8.8 Hz, 1H), 5.47-5.41 (m, 1H), 4.22-4.20 (m, 2H), 4.07-4.02 (m, 1H), 3.52-3.50 (m, 1H), 3.09-3.04 (m, 1H), 2.53 (s, 3H), 1.47-1.42 (m, 1H), 1.36 (s, 3H), 1.35 (s, 3H), 0.75-0.70 (m, 2H), 0.68-0.65 (m, 1H), 0.64-0.57 (m, 2H), 0.52-0.50 (m, 1H), 0.40-0.35 (m, 2H). MS (ESI) m/z: [M+H]⁺ Found 562.3.

### Example 88: N—((R)-1-(5-((R)-Cyclopropyl((R*)-2-oxo-4-(2,2,2-trifluoroethyl)imidazolidin-1-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

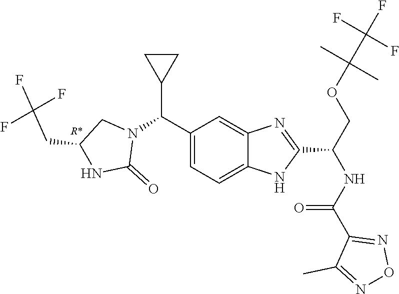 [SMILES: Cc1nonc1C(=O)N[C@@H](COC(C)(C)C(F)(F)F)c1nc2cc([C@@H](C3CC3)N3C[C@@H](CC(F)(F)F)NC3=O)ccc2[nH]1]

Step A: (R)—N—((R)-1-(5-((R)-Cyclopropyl((R*)-2-oxo-4-(2,2,2-trifluoroethyl)imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide. The title compound (97% yield) was prepared as described for Intermediate 33 Step C using (R)—N—((R)-1-(5-((R)—(((R*)-2-amino-4,4,4-trifluorobutyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 199) in place of (R)—N—((R)-1-(5-((R)—(((S)-2-amino-3,3,3-trifluoropropyl)amino) (cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide.

Step B: (R*)-1-((R)-(2-((R)-1-Amino-2-((1,1,1-trifluoro-2-methylpropan-2-yl)oxy)ethyl)-1H-benzo[d]imidazol-5-yl)(cyclopropyl)methyl)-4-(2,2,2-trifluoroethyl)imidazolidin-2-one. The title compound was prepared as described in Intermediate 34 using (R)—N—((R)-1-(5-((R)-cyclopropyl((R*)-2-oxo-4-(2,2,2-trifluoroethyl)imidazolidin-1-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Step A) in place of (R)—N—((R)-1-(5-((R)-cyclopropyl((S)-2-oxo-4-(trifluoromethyl)imidazolidin-1-yl)methyl)-1-((2-(trimethylsilyl) ethoxy)methyl)-1H-benzo[d]imidazol-2-yl)-2-((1,1,1-trifluoro-2-methylpropan-2-yl)oxy)ethyl)-2-methylpropane-2-sulfinamide and heating at 55° C. for 4 h instead of 3 h. The title compound (70% yield) was used without further purification.

Step C: N—((R)-1-(5-((R)-Cyclopropyl((R*)-2-oxo-4-(2,2,2-trifluoroethyl)imidazolidin-1-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide. The title compound was prepared as described for Example 34 using (R*)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(2,2,2-trifluoroethyl)imidazolidin-2-one (Step B) in place of 1-((S)-1-(2-((R*)-1-amino-2-((1,1,1-trifluoro-2-methylpropan-2-yl)oxy)ethyl)-1H-benzo[d]imidazol-5-yl)-2-methoxyethyl)-5,5-difluorotetrahydropyrimidin-2(1H)-one and the mixture was stirred at rt for 2 h instead of 1 h. The material was purified by silica gel chromatography (10-12% MeOH/DCM) to provide the title compound (20% yield) as a yellow oil. ¹H NMR (400 MHz, CD₃OD) δ 7.77-7.61 (m, 1H), 7.59-7.45 (m, 1H), 7.35-7.31 (m, 1H), 5.61-5.57 (m, 1H), 4.30-4.19 (m, 2H), 4.18-4.11 (m, 1H), 4.08-3.92 (m, 1H), 3.85-3.80 (m, 1H), 2.94-2.87 (m, 1H), 2.56 (s, 3H), 2.42-2.26 (m, 2H), 1.55-1.45 (m, 1H), 1.37-1.33 (m, 6H), 0.87-0.79 (m, 1H), 0.70-0.64 (m, 1H), 0.54-0.37 (m, 2H). MS (ESI) m/z: [M+H]⁺ Found 618.3.

### Example 89: N—((R)-1-(5-((R)-Cyclopropyl((S*)-2-oxo-4-(2,2,2-trifluoroethyl)imidazolidin-1-yl)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

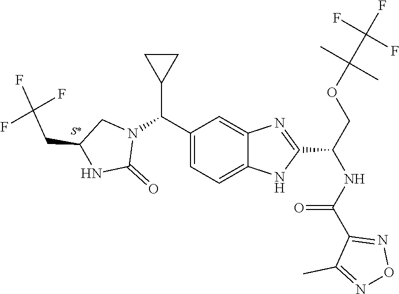 [SMILES: Cc1nonc1C(=O)N[C@@H](COC(C)(C)C(F)(F)F)c1nc2cc([C@@H](C3CC3)N3C[C@H](CC(F)(F)F)NC3=O)ccc2[nH]1]

The title compound (9% yield) was prepared as described in Example 88 using (R)—N—((R)-1-(5-((R)—(((S*)-2-amino-4,4,4-trifluorobutyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (Intermediate 200) in place of (R)—N—((R)-1-(5-((R)—(((R*)-2-amino-4,4,4-trifluorobutyl)amino)(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide in Step A. ¹H NMR (400 MHz, CD₃OD) δ 7.79-7.54 (m, 2H), 7.36-7.31 (m, 1H), 5.62-5.51 (m, 1H), 4.29-4.20 (m, 2H), 4.18-4.12 (m, 1H), 3.99-3.91 (m, 1H), 3.44-3.36 (m, 2H), 2.57-2.55 (m, 3H), 2.53-2.44 (m, 2H), 1.53-1.43 (m, 1H), 1.38-1.31 (m, 6H), 0.88-0.81 (m, 1H), 0.72-0.63 (m, 1H), 0.53-0.36 (m, 2H). MS (ESI) m/z: [M+H]⁺ Found 618.3.

### Example 90: 4-Methyl-N-((1R*,2R*)-4,4,4-trifluoro-2-methoxy-1-(5-(((S)-2-oxo-4-(trifluoromethyl)imidazolidin-1-yl)methyl)-1H-benzo[d]imidazol-2-yl)butyl)-1,2,5-oxadiazole-3-carboxamide

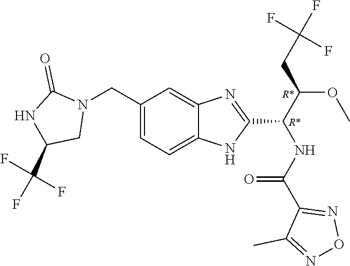 [SMILES: CO[C@H](CC(F)(F)F)[C@H](NC(=O)c1nonc1C)c1nc2cc(CN3C[C@@H](C(F)(F)F)NC3=O)ccc2[nH]1]

### Example 91: 4-Methyl-N-((1R*,2S*)-4,4,4-trifluoro-2-methoxy-1-(5-(((S)-2-oxo-4-(trifluoromethyl)imidazolidin-1-yl)methyl)-1H-benzo[d]imidazol-2-yl)butyl)-1,2,5-oxadiazole-3-carboxamide

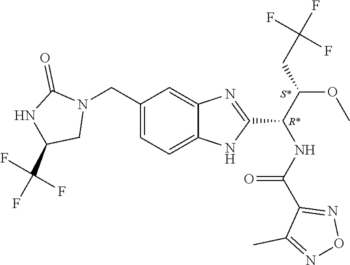 [SMILES: CO[C@@H](CC(F)(F)F)[C@H](NC(=O)c1nonc1C)c1nc2cc(CN3C[C@@H](C(F)(F)F)NC3=O)ccc2[nH]1]

The title compounds were prepared as described for the synthesis of Example 2 using (4S)-1-((2-(1-amino-4,4,4-trifluoro-2-methoxybutyl)-1H-benzo[d]imidazol-5-yl)methyl)-4-(trifluoromethyl)imidazolidin-2-one (Intermediate 128) in place of (S)-1-((R)-(2-((R)-1-amino-2-((1,1,1-trifluoro-2-methylpropan-2-yl)oxy)ethyl)-1H-benzo[d]imidazol-5-yl)(cyclopropyl)methyl)-4-(trifluoromethyl)imidazolidin-2-one, TEA in place of DIPEA and DCM in place of ACN. The mixture was stirred at rt overnight instead of 1 h and was purified by silica gel chromatography (0-1% MeOH/DCM) to afford a yellow gum. The gum was separated by SFC (DAICEL CHIRALPAK AD, 10 μm, 250×30 mm, 25% (v/v) MeOH (with 0.1% NH₄OH)/75% CO₂) twice to provide two diastereomers. The first eluting isomer was designated (1R*, 2S*) and was isolated as a white solid (41% yield, Example 91). The second eluting isomer was designated (1R*, 2R*) and was isolated as a white solid (12% yield, Example 90). Example 90: ¹H NMR (400 MHz, CDCl₃) δ 8.35-8.12 (m, 1H), 7.72-7.50 (m, 2H), 7.19 (d, J=8.0 Hz, 1H), 5.75-5.35 (m, 2H), 4.62-4.44 (m, 2H), 4.21-4.02 (m, 2H), 3.67-3.55 (m, 1H), 3.50-3.38 (m, 4H), 2.70-2.31 (m, 6H). MS (ESI) m/z: [M+H]⁺ Found 550.2. Example 91: ¹H NMR (400 MHz, CDCl₃) δ 8.33-8.14 (m, 1H), 7.74-7.44 (m, 2H), 7.16 (d, J=8.0 Hz, 1H), 5.83-5.44 (m, 2H), 4.63-4.40 (m, 2H), 4.21-4.06 (m, 2H), 3.68-3.54 (m, 1H), 3.49-3.34 (m, 4H), 2.72-2.30 (m, 6H). MS (ESI) m/z: [M+H]⁺ Found 550.2.

### Example 92: N-[(1R,2R)-1-[7-Fluoro-6-[[(4S)-2-oxo-4-(trifluoromethyl)imidazolidin-1-yl]methyl]-1H-benzimidazol-2-yl]-2-[(1R)-2,2,2-trifluoro-1-methyl-ethoxy]propyl]-4-methyl-1,2,5-oxadiazole-3-carboxamide

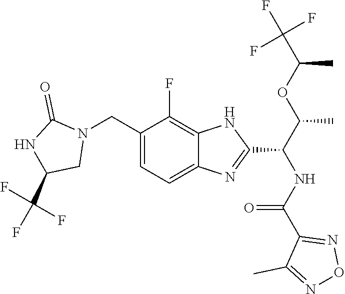 [SMILES: Cc1nonc1C(=O)N[C@H](c1nc2ccc(CN3C[C@@H](C(F)(F)F)NC3=O)c(F)c2[nH]1)[C@@H](C)O[C@H](C)C(F)(F)F]

A mixture of (S)-1-((2-((1R,2R)-1-amino-2-(((R)-1,1,1-trifluoropropan-2-yl)oxy)propyl)-4-fluoro-1H-benzo[d]imidazol-5-yl)methyl)-4-(trifluoromethyl)imidazolidin-2-one (50.0 mg, 0.106 mmol, Intermediate 201) and TEA (0.074 mL, 0.53 mmol) in DCM (1 mL) was stirred at rt for 10 min. Then, 2,5-dioxopyrrolidin-1-yl 4-methyl-1,2,5-oxadiazole-3-carboxylate (75.0 mg, 0.333 mmol, Intermediate 19) was added and the resulting mixture stirred at rt for 1 h. The mixture was then directly purified by silica gel chromatography (0-0.5% MeOH/DCM) and then subsequently purified by SFC (DAICEL CHIRALCEL OD column, 10 μm, 250×30 mm; 20% (v/v) EtOH (containing 0.1% of aqueous NH₃)/80% CO₂)) to provide the title compound as a white solid (24% yield). ¹H NMR (400 MHz, DMSO-d₆) δ 13.22-12.73 (m, 1H), 9.64-9.35 (m, 1H), 7.55 (s, 1H), 7.46-7.31 (m, 1H), 7.15-7.04 (m, 1H), 5.47-5.20 (m, 1H), 4.53-4.26 (m, 5H), 3.59-3.55 (m, 1H), 3.26-3.21 (m, 1H), 2.50 (s, 3H), 1.18-1.11 (m, 3H), 1.10-1.02 (m, 3H). MS (ESI) m/z: [M+H]⁺ Found 582.2.

### Example 93: 4-Cyclopropyl-N-[(1R,2R)-1-[7-fluoro-6-[[(4S)-2-oxo-4-(trifluoromethyl)imidazolidin-1-yl]methyl]-1H-benzimidazol-2-yl]-2-[(1R)-2,2,2-trifluoro-1-methyl-ethoxy]propyl]-1,2,5-oxadiazole-3-carboxamide

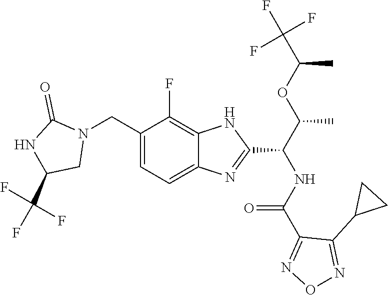 [SMILES: C[C@@H](O[C@H](C)C(F)(F)F)[C@H](NC(=O)c1nonc1C1CC1)c1nc2ccc(CN3C[C@@H](C(F)(F)F)NC3=O)c(F)c2[nH]1]

The title compound (15% yield) was prepared as described for the synthesis of Example 92 using 2,5-dioxopyrrolidin-1-yl 4-cyclopropyl-1,2,5-oxadiazole-3-carboxylate (Intermediate 23) in place of 2,5-dioxopyrrolidin-1-yl 4-methyl-1,2,5-oxadiazole-3-carboxylate. ¹H NMR (400 MHz, DMSO-d₆) δ 12.87 (s, 1H), 9.62 (s, 1H), 7.62-7.48 (m, 1H), 7.45-7.27 (m, 1H), 7.16-7.04 (m, 1H), 5.35 (s, 1H), 4.53-4.44 (m, 1H), 4.42-4.27 (m, 4H), 3.62-3.51 (m, 1H), 3.26-3.21 (m, 1H), 2.37-2.27 (m, 1H), 1.17-1.10 (m, 5H), 1.10-1.04 (m, 3H), 1.02-0.94 (m, 2H). MS (ESI) m/z: [M+H]⁺ Found 608.2.

### Example 94: 4-Cyclopropyl-N-((1S*,2R)-1-(7-fluoro-6-((S)-2-methoxy-1-((S)-2-oxo-4-(trifluoromethyl)imidazolidin-1-yl)ethyl)-1H-benzo[d]imidazol-2-yl)-2-(((R)-1,1,1-trifluoropropan-2-yl)oxy)propyl)-1,2,5-oxadiazole-3-carboxamide

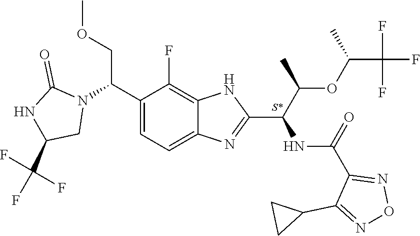 [SMILES: COC[C@H](c1ccc2nc([C@H](NC(=O)c3nonc3C3CC3)[C@@H](C)O[C@H](C)C(F)(F)F)[nH]c2c1F)N1C[C@@H](C(F)(F)F)NC1=O]

### Example 95: 4-Cyclopropyl-N-((1R*,2R)-1-(7-fluoro-6-((S)-2-methoxy-1-((S)-2-oxo-4-(trifluoromethyl)imidazolidin-1-yl)ethyl)-1H-benzo[d]imidazol-2-yl)-2-(((R)-1,1,1-trifluoropropan-2-yl)oxy)propyl)-1,2,5-oxadiazole-3-carboxamide

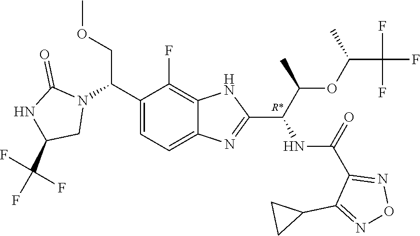 [SMILES: COC[C@H](c1ccc2nc([C@@H](NC(=O)c3nonc3C3CC3)[C@@H](C)O[C@H](C)C(F)(F)F)[nH]c2c1F)N1C[C@@H](C(F)(F)F)NC1=O]

A mixture of (S)-1-((S)-1-(2-((1R,2R)-1-amino-2-(((R)-1,1,1-trifluoropropan-2-yl)oxy)propyl)-4-fluoro-1H-benzo[d]imidazol-5-yl)-2-methoxyethyl)-4-(trifluoromethyl)imidazolidin-2-one (58 mg, 0.092 mmol, Intermediate 144), 2,5-dioxopyrrolidin-1-yl 4-cyclopropyl-1,2,5-oxadiazole-3-carboxylate (69 mg, 0.28 mmol, Intermediate 23) and triethylamine (0.064 mL, 0.46 mmol) in DCM (1 mL) was stirred at rt for 1 h. The reaction mixture was then diluted with DCM (10 mL), washed with H₂O (10 mL×2) and brine (10 mL), dried over anhydrous Na₂SO₄, filtered, and concentrated to dryness to give a yellow solid. The solid was then subjected to silica gel chromatography (0-5% MeOH/DCM) to give, after lyophilization, a mixture of stereoisomers. This mixture was then subjected to SFC (DAICEL CHIRALPAK AS column, 10 μm, 250×30 mm, 20% (v/v) EtOH (with 0.1% NH₃H₂O)/80% CO₂) to give two eluting peaks. The first eluting isomer, Example 94, was isolated in 4% yield as a white solid. The second eluting isomer, Example 95, was isolated in 22% yield as a white solid. Example 94: ¹H NMR (400 MHz, DMSO-d₆) δ 12.88 (br s, 1H), 9.74-9.43 (m, 1H), 7.51-7.42 (m, 1H), 7.38-7.25 (m, 2H), 5.44-5.38 (m, 1H), 5.37-5.31 (m, 1H), 4.41-4.30 (m, 3H), 3.93-3.79 (m, 3H), 3.75-3.69 (m, 1H), 3.29 (s, 3H), 2.37-2.31 (m, 1H), 1.16-1.11 (m, 7H), 1.04-0.96 (m, 3H). MS (ESI) m/z: [M+H]⁺ Found 652.3. Example 95: ¹H NMR (400 MHz, DMSO-d₆) δ 13.29-12.73 (m, 1H), 9.70-9.34 (m, 1H), 7.52-7.47 (m, 1H), 7.38-7.32 (m, 1H), 7.25-7.14 (m, 1H), 5.46-5.30 (m, 2H), 4.48-4.21 (m, 3H), 3.96-3.81 (m, 2H), 3.69-3.66 (m, 2H), 3.31 (s, 3H), 2.39-2.28 (m, 1H), 1.19-1.13 (m, 4H), 1.13-1.11 (m, 1H), 1.11-1.04 (m, 3H), 1.02-0.95 (m, 1H), 1.02-0.94 (m, 1H). MS (ESI) m/z: [M+H]⁺ Found 652.2.

### Example 96: N-((1S*,2R)-1-(7-Fluoro-6-((S)-2-methoxy-1-((S)-2-oxo-4-(trifluoromethyl)imidazolidin-1-yl)ethyl)-1H-benzo[d]imidazol-2-yl)-2-(((R)-1,1,1-trifluoropropan-2-yl)oxy)propyl)-4-methyl-1,2,5-oxadiazole-3-carboxamide

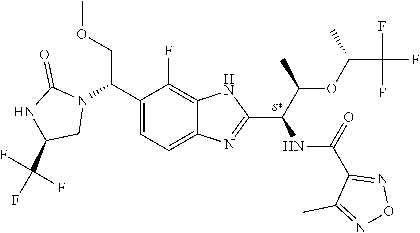 [SMILES: COC[C@H](c1ccc2nc([C@H](NC(=O)c3nonc3C)[C@@H](C)O[C@H](C)C(F)(F)F)[nH]c2c1F)N1C[C@@H](C(F)(F)F)NC1=O]

### Example 97: N-((1R*,2R)-1-(7-Fluoro-6-((S)-2-methoxy-1-((S)-2-oxo-4-(trifluoromethyl)imidazolidin-1-yl)ethyl)-1H-benzo[d]imidazol-2-yl)-2-(((R)-1,1,1-trifluoropropan-2-yl)oxy)propyl)-4-methyl-1,2,5-oxadiazole-3-carboxamide

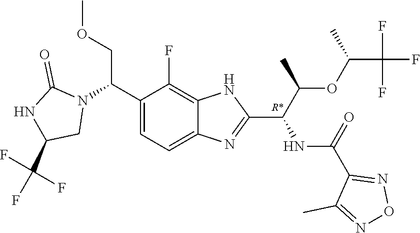 [SMILES: COC[C@H](c1ccc2nc([C@@H](NC(=O)c3nonc3C)[C@@H](C)O[C@H](C)C(F)(F)F)[nH]c2c1F)N1C[C@@H](C(F)(F)F)NC1=O]

(S)-1-((S)-1-(2-((1R,2R)-1-Amino-2-(((R)-1,1,1-trifluoropropan-2-yl)oxy)propyl)-4-fluoro-1H-benzo[d]imidazol-5-yl)-2-methoxyethyl)-4-(trifluoromethyl)imidazolidin-2-one (58 mg, 0.092 mmol, Intermediate 144), 2,5-dioxopyrrolidin-1-yl 4-methyl-1,2,5-oxadiazole-3-carboxylate (62 mg, 0.28 mmol, Intermediate 19), triethylamine (0.064 mL, 0.46 mmol) and DCM (1 mL) were added to a vial. The reaction mixture was stirred at rt for 1 h. The reaction mixture was then diluted with DCM (10 mL), washed with H₂O (10 mL×2) and brine (10 mL), dried over anhydrous Na₂SO₄, filtered, and concentrated to dryness to give a yellow solid. The solid was then subjected to silica gel chromatography (0-5% MeOH/DCM) to give, after lyophilization, a mixture of stereoisomers. This mixture was then subjected to SFC (DAICEL CHIRALPAK AS column, 10 μm, 250×30 mm (15% (v/v) EtOH (with 0.1% NH₃H₂O)/85% CO₂) to give two eluting peaks. The first eluting isomer, Example 96, was isolated in 3% yield as a white solid. The second eluting isomer, Example 97, was isolated in 25% yield as a white solid. Example 96: ¹H NMR (400 MHz, CDCl₃) δ 10.89-9.82 (m, 1H), 8.35-8.15 (m, 1H), 7.36-7.30 (m, 1H), 5.68-5.32 (m, 3H), 5.21-4.99 (m, 1H), 4.63-4.55 (m, 1H), 4.19-4.04 (m, 3H), 3.86-3.81 (m, 1H), 3.76-3.71 (m, 1H), 3.43 (s, 3H), 2.64-2.62 (m, 3H), 1.26 (br s, 3H), 1.22-1.19 (m, 3H). MS (ESI) m/z: [M+H]⁺ Found 626.3. Example 97: ¹H NMR (400 MHz, CDCl₃) δ 12.17-10.33 (m, 1H), 8.42-8.17 (m, 1H), 7.52-7.27 (m, 1H), 7.25-7.11 (m, 1H), 6.29-6.22 (m, 0.5H), 5.85-5.40 (m, 2H), 5.36-5.27 (m, 0.5H), 4.61-4.33 (m, 1H), 4.22-3.81 (m, 5H), 3.67-3.55 (m, 0.5H), 3.48-3.43 (m, 3H), 3.29-3.25 (m, 0.5H), 2.64-2.51 (m, 3H), 1.30-1.19 (m, 6H). MS (ESI) m/z: [M+H]⁺ Found 626.3.

### In Vitro Biological Data

### IL-17A(FLAG-Tagged): IL-17RA(His-Tagged) Binding Disruption Eu-HTRF Assay

An antibody directed against the FLAG tag of IL-17A (SEQ ID NO: 1) is labeled with the HTRF donor chromophore (Europium-cryptate). IL-17A is present as a dimer that is “locked into” this quaternary structure due to the formation of loop-spanning intramolecular disulfide bridges. The construct of IL-17RA used in the assay excludes the outer-membrane portion of the receptor and is fused to a C-terminal 10×His tag (SEQ ID NO:2). An antibody directed against the His tag of the IL-17RA chimera is labeled with the HTRF acceptor chromophore (“D2”). The fluorescence-resonance energy transfer (FRET) depends on the vicinity of the donor chromophore to the acceptor, and interruption of the binding between the IL-17A and IL-17RA causes the reduction/loss of FRET. Therefore, this assay allows to evaluate the compound effect on the binding IL-17A and IL-17RA by monitoring the fluorescence intensity of donor vs acceptor. The assay was run using either Protocol 1 or Protocol 2 as described below.

Protocol 1. 40 nl of 2-fold serial diluted compound solution for total 22 dilution points is added into each well of a 1536-well, white, low-volume, non-binding plate (Greiner #782904), then 2 μl of FLAG tagged IL-17A at 2× final concentration (2.5 nM) in solution of PBS+0.01% Triton-X100 is added to each well. The assay plate is briefly centrifuged then incubated for 1 h at rt. A mixed solution is prepared containing 2×5 nM 10HIS×IL-17RA, 2×2.5 nM Eu-anti-FLAG (CISBIO), 2×5 nM D2-anti-HIS (CISBIO) in PBS+0.01% Triton-X100+200 mM Potassium Fluoride (Sigma 60238) and 2 μl of mix is added to each well of the assay plate. The plate is briefly centrifuged then incubated for 2 h at rt. The HTRF intensities at the wavelength of donor (620 nm) and acceptor (665 nm) are measured using BMG Pherastar. The ratio between intensities at two wavelengths is calculated and plotted against the compound concentration and the data is fitted to a one-site competition model to yield IC₅₀ of the compound.

Protocol 2. 40 nl of 2-fold serial diluted compound solution for total 22 dilution points is added into each well of a 1536-well, white, low-volume, non-binding plate (Greiner #782904), then 2 μl of FLAG tagged IL-17A at 2× final concentration (1 nM) in solution of PBS+0.01% Triton-X100 is added to each well. The assay plate is briefly centrifuged then incubated for 1 h at rt. A mixed solution is prepared containing 2×5 nM 10HIS×IL-17RA, 2×2.5 nM Eu-anti-FLAG (CISBIO), 2×5 nM D2-anti-HIS (CISBIO) in PBS+0.01% Triton-X100+200 mM Potassium Fluoride (Sigma 60238) and 2 μl of mix is added to each well of the assay plate. The plate is briefly centrifuged then incubated for 2 h at rt. The HTRF intensities at the wavelength of donor (615 nm) and acceptor (665 nm) are measured using BMG Pherastar. The ratio between intensities at two wavelengths is calculated and plotted against the compound concentration and the data is fitted to a one-site competition model to yield IC₅₀ of the compound.

IL-17A acts directly on keratinocytes through binding to dimeric receptor IL-17RA/RC and drives the production of a number of inflammatory mediators known to be elevated in psoriasis lesional tissue. IL-17A small molecule inhibitors that block the IL-17A to interact with IL-17R would inhibit the IL-17A signaling in its targeted cells such as keratinocytes. The compound functional activity is evaluated for its impact on IL-17A-induced G-CSF production in human normal keratinocyte (NHK).

### NHK Assay

Adult normal human keratinocytes are cultured in keratinocyte growth medium (Lonza) in a flask till reaching ˜90% confluence, then cells are transferred to a 384-well plate at density of 3000-4000 cell/well. Recombinant human IL-17A (Gibco PHC9174) is pre-incubated with titrated compound or DMSO for 1 h at rt then added to the cell culture plate. The final concentration of IL-17A is 5 ng/mL and DMSO is 0.2%, in the culture containing 5% FBS. Cells are cultured/treated for 24 h at 37° C. Supernatants are collected and G-CSF production is measured through HTRF technology using Human G-CSF Kit (CisBio). G-CSF concentration was extrapolated from the standard curve and IC₅₀ is determined using GraphPad Prism. Cell viability is also evaluated using CellTiter-Glo kit (Promega) and effect of compound on cell viability is compared to DMSO control.

In cases where the compound was tested more than once, the IC₅₀ value shown is a simple average of the measured values.
A: IC₅₀<0.05 μm; B: 0.05 μm≤IC₅₀≤0.1 μm; C: IC₅₀>0.1 μm
   Not available

TABLE 2

| | HTRF IC₅₀ | HTRF IC₅₀ | |
|---|---|---|---|
| Example # | Protocol 1 | Protocol 2 | NHK IC₅₀ |
| 1 | A | — | A |
| 2 | A | — | A |
| 3 | A | — | A |
| 4 | A | — | A |
| 5 | A | — | A |
| 6 | A | — | A |
| 7 | A | A | A |
| 8 | B | — | C |
| 9 | A | — | A |
| 10 | A | — | A |
| 11 | C | — | C |
| 12 | A | — | A |
| 13 | A | — | A |
| 14 | A | — | A |
| 15 | A | — | A |
| 16 | C | — | C |
| 17 | A | A | A |
| 18 | A | — | B |
| 19 | A | — | B |
| 20 | A | — | A |
| 21 | A | — | A |
| 22 | A | — | A |
| 23 | A | — | A |
| 24 | A | — | A |
| 25 | A | — | A |
| 26 | A | — | A |
| 27 | A | — | A |
| 28 | A | — | A |
| 29 | A | — | A |
| 30 | A | — | A |
| 31 | A | — | A |
| 32 | A | A | B |
| 33 | B | — | C |
| 34 | A | — | A |
| 35 | A | — | A |
| 36 | A | — | A |
| 37 | C | — | C |
| 38 | A | — | A |
| 39 | C | — | C |
| 40 | B | — | C |
| 41 | A | — | A |
| 42 | — | — | A |
| 43 | — | — | A |
| 44 | — | — | A |
| 45 | — | — | B |
| 46 | — | — | A |
| 47 | A | — | A |
| 48 | A | — | A |
| 49 | C | — | C |
| 50 | A | — | A |
| 51 | A | — | B |
| 52 | A | A | A |
| 53 | A | — | A |
| 54 | A | A | A |
| 55 | A | — | A |
| 56 | A | — | A |
| 57 | A | — | C |
| 58 | A | — | A |
| 59 | A | — | A |
| 60 | A | — | A |
| 61 | A | — | A |
| 62 | A | — | A |
| 63 | — | — | A |
| 64 | — | — | C |
| 65 | — | — | A |
| 66 | — | — | A |
| 67 | — | — | A |
| 68 | — | — | A |
| 69 | — | A | A |
| 70 | — | A | A |
| 71 | — | A | A |
| 72 | — | A | B |
| 73 | — | A | A |
| 74 | — | A | A |
| 75 | C | C | A |
| 76 | — | — | A |
| 77 | — | — | A |
| 78 | — | — | A |
| 79 | — | — | A |
| 80 | — | — | A |
| 81 | — | — | A |
| 82 | — | — | A |
| 83 | A | A | A |
| 84 | A | — | A |
| 85 | B | — | A |
| 86 | | — | A |
| 87 | A | — | A |
| 88 | — | — | A |
| 89 | — | — | C |
| 90 | — | — | C |
| 91 | — | — | A |
| 92 | — | — | A |
| 93 | — | — | A |
| 94 | — | A | A |
| 95 | — | A | A |
| 96 | — | B | A |
| 97 | — | A | A |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All documents cited herein are incorporated by reference.

SEQ ID NO: 1
Name: IL-17A-Flag
MATGSRTSLLLAFGLLCLPWLQEGSAGSDYKDDDDKGSGSGSLEVLFQGP
GITIPRNPGCPNSEDKNFPRTVMVNLNIHNRNTNTNPKRSSDYYNRSTSP
WNLHRNEDPERYPSVIWEAQCRHLGCINADGNVDYHMNSVPIQQEILVLR
REPPHCPNSFRLEKILVSVGCTCVTPIVHHVQ
SEQ ID NO: 2
Name: IL-17RA
MKFLVNVALVFMVVYISYIYALRLLDHRALVCSQPGLNCTVKNSTCLDDS
WIHPRNLTPSSPKDLQIQLHFAHTQQGDLFPVAHIEWTLQTDASILYLEG
AELSVLQLNTNERLCVRFEFLSKLRHHHRRWRFTFSHFVVDPDQEYEVTV
HHLPKPIPDGDPNHQSKNFLVPDCEHARMKVTTPCMSSGSLWDPNITVET
LEAHQLRVSFTLWNESTHYQILLTSFPHMENHSCFEHMHHIPAPRPEEFH
QRSNVTLTLRNLKGCCRHQVQIQPFFSSCLNDCLRHSATVSCPEMPDTPE
PIPDYMPLWGSGGHHHHHHHHHH*

1. A compound of Formula I: 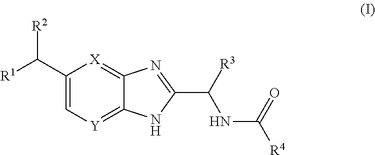
or a pharmaceutically acceptable salt thereof, wherein:
R¹ is: 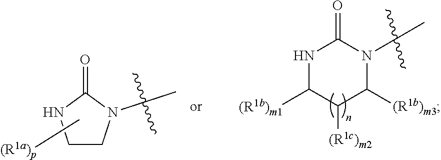
R^{1a} independently for each occurrence is —C₍₁₋₃₎alkyl or —C₍₃₋₅₎cycloalkyl, wherein the —C₍₁₋₃₎alkyl is unsubstituted or substituted with one to five fluorine atoms;
R^{1b} independently for each occurrence is —C₍₁₋₃₎alkyl or C₍₃₋₅₎cycloalkyl, wherein the —C₍₁₋₃₎alkyl is unsubstituted or substituted with one to five fluorine atoms;
R^{1c} independently for each occurrence is halo, —C₍₁₋₃₎alkyl, or C₍₃₋₅₎cycloalkyl, wherein the —C₍₁₋₃₎alkyl is unsubstituted or substituted with one to five fluorine atoms;
p is 0, 1, 2, 3, or 4;
n is 1 or 2;
m1, m2, and m3 are each independently 0, 1, or 2;
R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to six R^{2a} groups;
R^{2a} independently for each occurrence is fluorine, —CN, or —O—C₍₁₋₃₎alkyl;
R³ is C₍₁₋₈₎alkyl, —C₍₁₋₆₎alkyl-O—C₍₁₋₆₎alkyl, or —C₍₁₋₆₎alkyl-O—C₍₃₋₅₎cycloalkyl, _{wherein the} C₍₁₋₈₎alkyl and —C₍₁₋₆₎alkyl-O—C₍₁₋₆₎alkyl are unsubstituted or substituted with one to six fluorine atoms, and wherein the —C₍₁₋₆₎alkyl-O—C₍₃₋₅₎cycloalkyl is unsubstituted or substituted with one to six fluorine atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, and —C₍₀₋₂₎alkyl-C₍₃₋₆₎cycloalkyl are unsubstituted or substituted with one to six substituents independently selected from fluorine, CH₃, CH₂F, CHF₂, CF₃, and —CN;
X is CH, CF or N; and
Y is CH or CF.

2-5. (canceled)

6. The compound of claim 1, or a pharmaceutically acceptable salt thereof, wherein R¹ is: 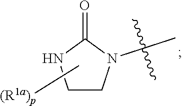
R^{1a} independently for each occurrence is CH₃, CH₂F, CHF₂, CF₃, or cyclopropyl; and
p is 0, 1, or 2.

7. The compound of claim 1, or a pharmaceutically acceptable salt thereof, wherein R¹ is: 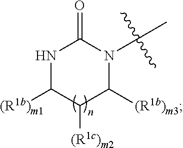
R^{1b} independently for each occurrence is CH₃, CH₂F, CHF₂, CF₃, or cyclopropyl;
R^{1c} independently for each occurrence is fluorine, CH₃, CH₂F, CHF₂, CF₃, or cyclopropyl;
n is 1 or 2; and
m1, m2, and m3, are each independently 0, 1, or 2.

8. (canceled)

9. The compound of claim 1, or a pharmaceutically acceptable salt thereof, wherein R¹ is:

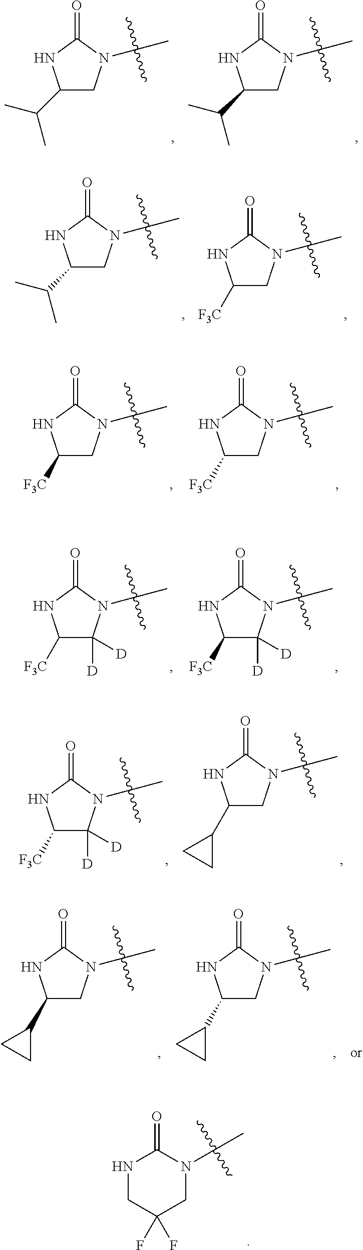

10. (canceled)

11. The compound of claim 1, or a pharmaceutically acceptable salt thereof, wherein:
R² is H, —C₍₁₋₆₎alkyl, —C₍₃₋₅₎cycloalkyl, —C₍₁₋₃₎alkyl-O—C₍₁₋₃₎alkyl, or —C₍₁₋₃₎alkyl-O—C₍₃₋₅₎cycloalkyl, wherein the —C₍₁₋₆₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to four R^{2a} groups; and
R^{2a} independently for each occurrence is fluorine or —CN.

12. (canceled)

13. (canceled)

14. The compound of claim 1, or a pharmaceutically acceptable salt thereof, which is a compound of Formula (Ia):

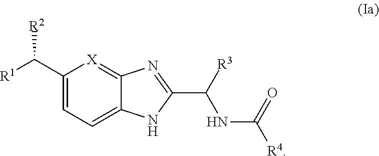

15. The compound of claim 1, or a pharmaceutically acceptable salt thereof, which is a compound of Formula (Iaa):

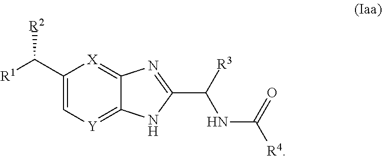

16. The compound of claim 1, or a pharmaceutically acceptable salt thereof, wherein R³ is C₍₁₋₆₎alkyl, —C₍₁₋₄₎alkyl-O—C₍₁₋₄₎alkyl, or —C₍₁₋₄₎alkyl-O—C₍₃₋₄₎cycloalkyl, wherein the C₍₁₋₆₎alkyl and —C₍₁₋₄₎alkyl-O—C₍₁₋₄₎alkyl are unsubstituted or substituted with one to six fluorine atoms, and wherein the —C₍₁₋₄₎alkyl-O—C₍₃₋₄₎cycloalkyl is unsubstituted or substituted with one to four fluorine atoms.

17-22. (canceled)

23. The compound of claim 1, or a pharmaceutically acceptable salt thereof, which is a compound of Formula (Idd-1): 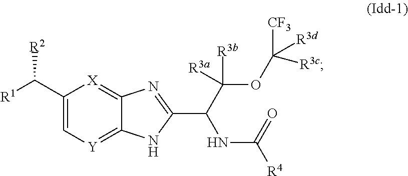
wherein R^{3a}, R^{3b}, R^{3c}, and R^{3d} are each independently H or CH₃.

24-25. (canceled)

26. The compound of claim 1, or a pharmaceutically acceptable salt thereof, wherein R³ is:

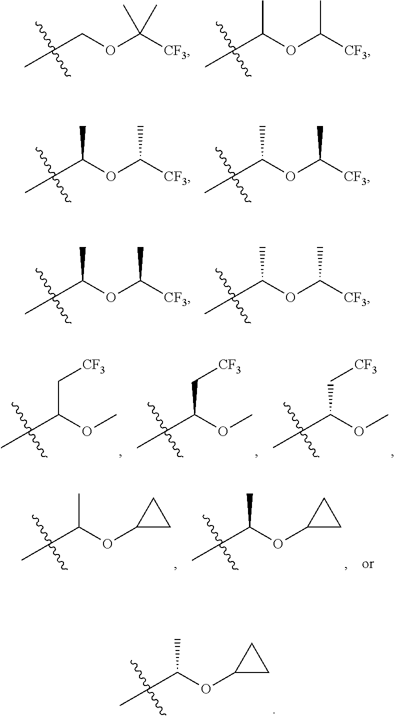

27-29. (canceled)

30. The compound of claim 1, or a pharmaceutically acceptable salt thereof, wherein R⁴ is a 5-membered heteroaryl comprising one to three heteroatoms selected from O and N, wherein the 5-membered heteroaryl is unsubstituted or substituted with one to two R^{4a} groups.

31-37. (canceled)

38. The compound of claim 1, or a pharmaceutically acceptable salt thereof, wherein R⁴ is:

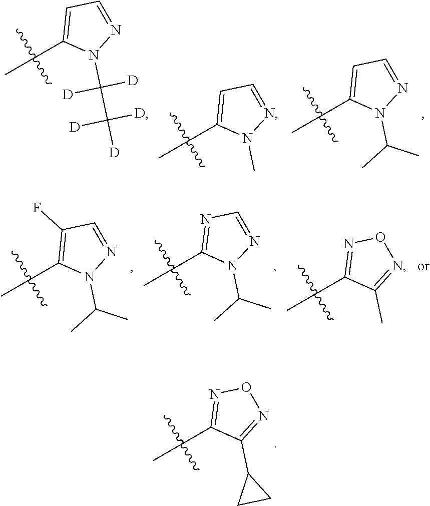

39-44. (canceled)

45. The compound of claim 1, or a pharmaceutically acceptable salt thereof, having a structure selected from the group consisting of:

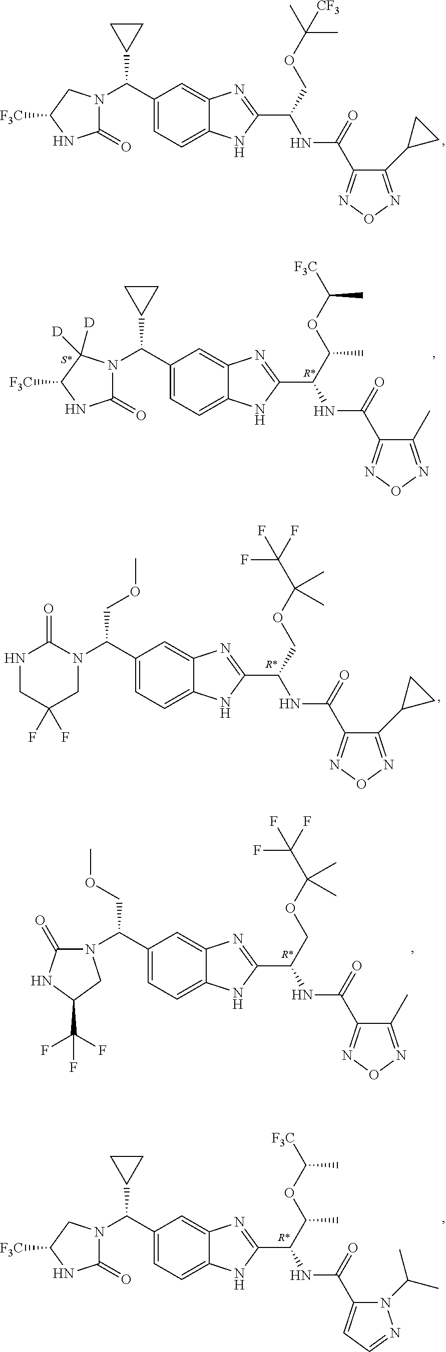

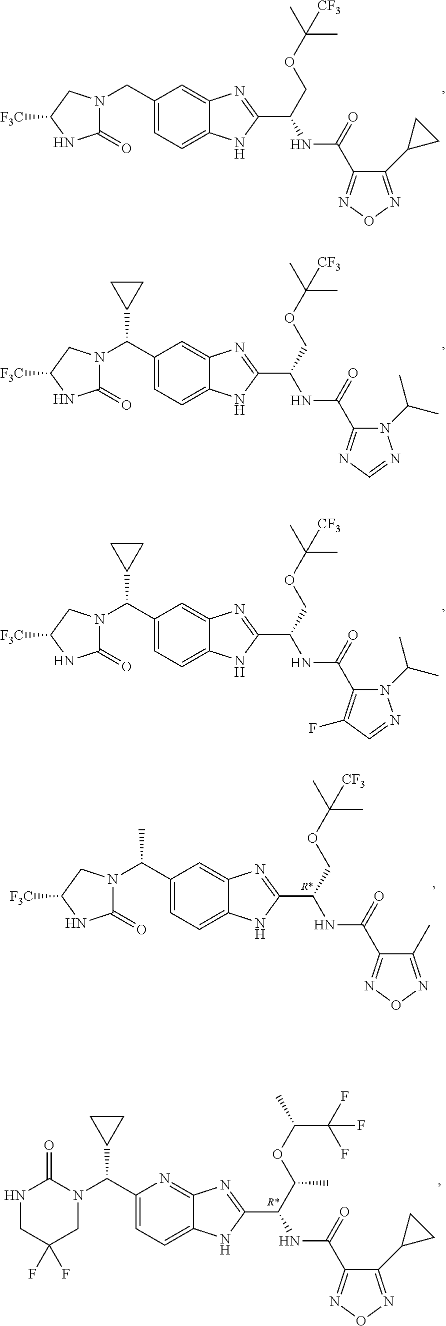 [SMILES: CC(C)(OC[C@H](NC(=O)c1nonc1C1CC1)c1nc2cc(CN3C[C@@H](C(F)(F)F)NC3=O)ccc2[nH]1)C(F)(F)F ; CC(C)n1ncnc1C(=O)N[C@@H](COC(C)(C)C(F)(F)F)c1nc2cc([C@@H](C3CC3)N3C[C@@H](C(F)(F)F)NC3=O)ccc2[nH]1 ; CC(C)n1ncc(F)c1C(=O)N[C@@H](COC(C)(C)C(F)(F)F)c1nc2cc([C@@H](C3CC3)N3C[C@@H](C(F)(F)F)NC3=O)ccc2[nH]1 ; Cc1nonc1C(=O)N[C@@H](COC(C)(C)C(F)(F)F)c1nc2cc([C@@H](C)N3C[C@@H](C(F)(F)F)NC3=O)ccc2[nH]1 ; C[C@@H](O[C@H](C)C(F)(F)F)[C@H](NC(=O)c1nonc1C1CC1)c1nc2nc([C@@H](C3CC3)N3CC(F)(F)CNC3=O)ccc2[nH]1]

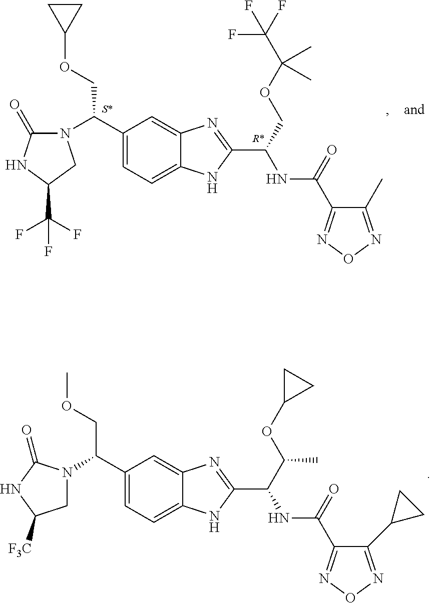 [SMILES: Cc1nonc1C(=O)N[C@@H](COC(C)(C)C(F)(F)F)c1nc2cc([C@@H](COC3CC3)N3C[C@@H](C(F)(F)F)NC3=O)ccc2[nH]1 ; COC[C@H](c1ccc2[nH]c([C@@H](NC(=O)c3nonc3C3CC3)[C@@H](C)OC3CC3)nc2c1)N1C[C@@H](C(F)(F)F)NC1=O]

46. The compound of claim 1, or a pharmaceutically acceptable salt thereof, having a structure selected from the group consisting of:

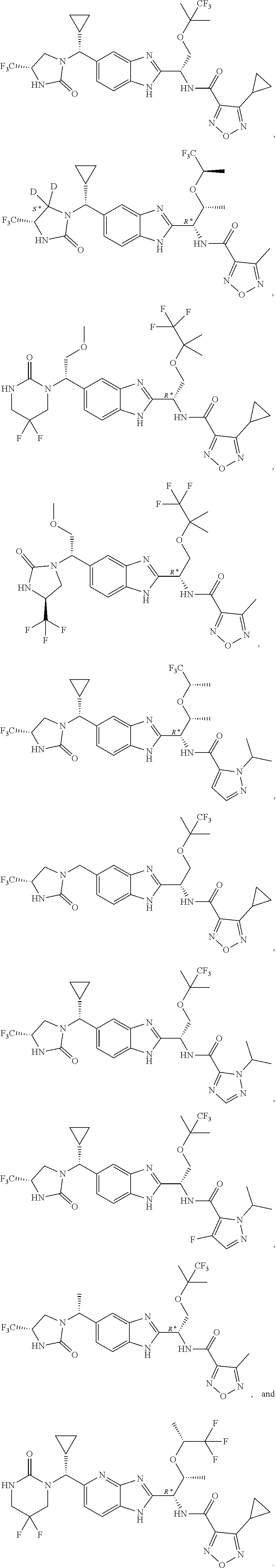

47. The compound of claim 1, or a pharmaceutically acceptable salt thereof, having a structure selected from the group consisting of:

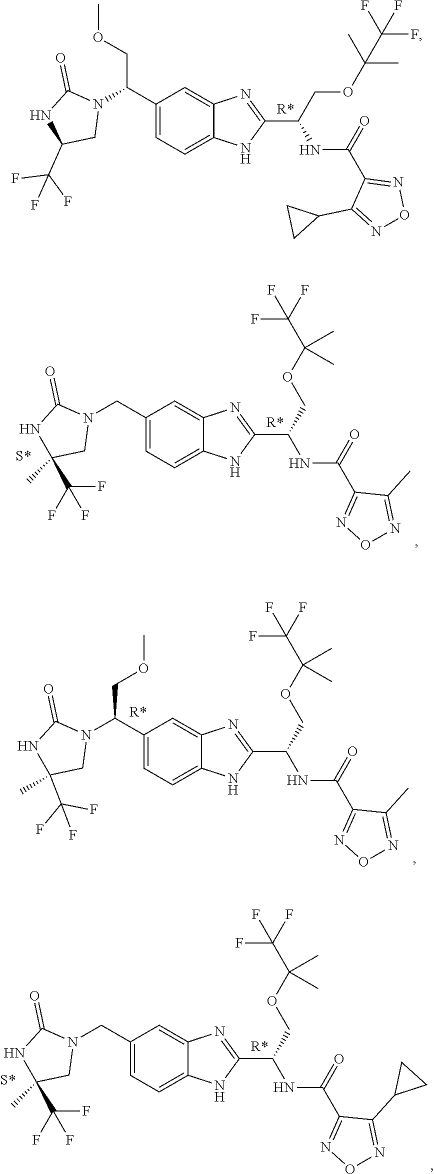

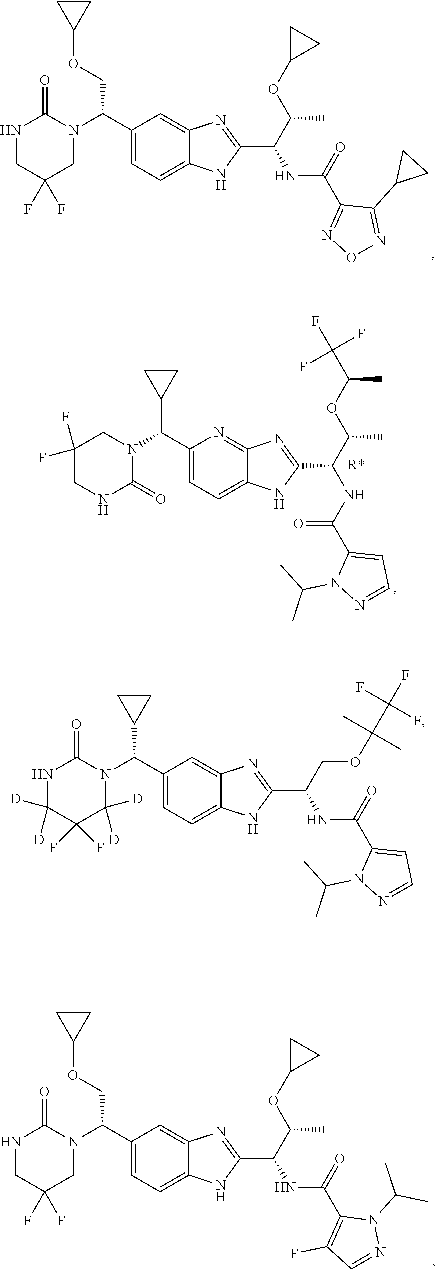 [SMILES: C[C@@H](OC1CC1)[C@H](NC(=O)c1nonc1C1CC1)c1nc2cc([C@@H](COC3CC3)N3CC(F)(F)CNC3=O)ccc2[nH]1 ; CC(C)n1nccc1C(=O)N[C@H](c1nc2nc([C@@H](C3CC3)N3CC(F)(F)CNC3=O)ccc2[nH]1)[C@@H](C)O[C@H](C)C(F)(F)F ; [2H]C1([2H])NC(=O)N([C@@H](c2ccc3[nH]c([C@H](COC(C)(C)C(F)(F)F)NC(=O)c4ccnn4C(C)C)nc3c2)C2CC2)C([2H])([2H])C1(F)F ; CC(C)n1ncc(F)c1C(=O)N[C@H](c1nc2cc([C@@H](COC3CC3)N3CC(F)(F)CNC3=O)ccc2[nH]1)[C@@H](C)OC1CC1]

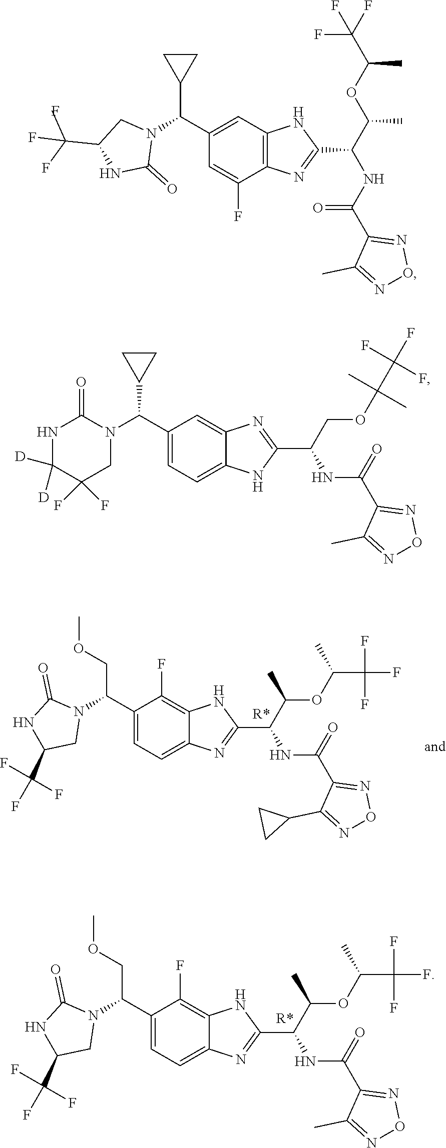

48-64. (canceled)

65. A pharmaceutical composition, comprising a compound of any one of the preceding claims, or a pharmaceutically acceptable salt thereof, and a pharmaceutically acceptable carrier.

66. (canceled)

67. The pharmaceutical composition of claim 65, or a pharmaceutically acceptable salt thereof, which is administered orally.

68. The pharmaceutical composition of claim 67, or a pharmaceutically acceptable salt thereof, which is administered as a tablet or a capsule.

69. (canceled)

70. A method for treating and/or ameliorating an IL-17A mediated inflammatory syndrome, disorder, or disease comprising administering to a subject in need thereof a therapeutically effective amount of a compound of claim 1, or a pharmaceutically acceptable salt thereof.

71. The method of claim 70, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

72-89. (canceled)
